SEQUENCE LISTING (58 sequences)

SEQ ID NO: 1 - DNA, 2343 bp, Homo sapiens
atggcagcgccaggcggcaggtcggagccgccgcagctccccgagtacagctgcagctacatggtgtcgcggccggtctactcggagctagctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcctaa

SEQ ID NO: 2 - DNA, 2343 bp, Homo sapiens
atggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcctga

SEQ ID NO: 3 - DNA, 64175 bp, Homo sapiens
cgaatgaatggaatacttaaacatccaactcgtggtaacacagcacatcagtgtttacttgaaacaatcctcaagcttcccttttctccctccattaacaaaccacagatttcatgagataagatatgcaaggataactttaaaagtgcaatacacttaaataataagaaatttggaaggatcctgaaattattctttgcttagaaaactatgccattattggctcgacatagtggcacaggcctgtaatgccagctacttcagggactgaggtgagacccccatctctaaaaaaaagaaagaaaacatccaggccaagtgcagttggctcctgcctgtaatgtcattttataatcagcattttggaaggctgaggtggtaggattatttgagcccaggagtttgagaccagcctgggtaacaaagcgagaccccatctcttgaaaaaaaaaaatcatcattaaaacaaacattaaaagaaacccacctgggtgtggtggctcacacatgtaatcccagcaccctgggaggatgaagcaggtggatctcttgagcacaggagtttgagaccagcctgagcaagatggcaaaaccctgtctctacaaaaaatacaaaaattagccgggtgtggtggcacatgcctatagtctcaactacttgggaggctgaggtggagaatggcttgagctttggaggtgaaggttgcagtgagctgagatagtgtcactgcacttcaacctgggtgacagagccagaccctgtttaaaacaaacaaacaaacacacacacacacacacacacacagaaaacccctcaaaacccaaaactatgccatcatttcgttctagcaatttactaaaccacatatcaagtttgcattcatattaaagaagaggcagatgggctctgcctgagaaatttattgagttatagttttattgacactgtaagttgtttaaaattgaatggattgccacataatatttacaaataatttcattttttgatgttttaatggttggattctcttcttttaaaactagttttttttctacccaaatatttctcaaaattccattgttaaacaatcaattgatcaataatttaacaagtctggtacctattatattgtcataatttgtcaaggtattgtgtagggaaataaagagactagcattataattttactttaataaaaatgtatacctatgtctgcatagaaagaataaataaaaaaagaagcaaaacagtgattatctggataatgagataaaatgtgattactatattatacattaatgtagtttccaaattatttactacaggcacatcttatttttgtaacaccataaaagacatatactatttatgtccctagggagcatatcttataactggagaagaaaataaagtagcatacagtaaacaaatagaaaacaattgagtgcaagataaaaataacaacaaaaaaccaagaaaacaattgagtgcaaaactgtatgattactgagaataagcccaataagttgctcagagaagaagagatcagaaatatagaaagaaagttcagttcagttctctttttgtataagtatgacaaaaatttagcatttgctatcttgagaaaattaacacgcataatgaaataaaagctcaggattaggcctggctcaagtgatcctcccgcctcagcttctcaaagtgttgggattacactgtgagccacagcgcccagccaacatgtttgcattcttttgcaattcttctgatttgagaacattttaaattgtaaaaaaaaaaaaaaaaagaaaaaatttataccacaatattcgctctcagacaaaaataatgtagcaagattgactgctcttaagttaaataaacgaaggattcaattaaatcattcttgtagcacacgtagtcactcaaaagcatagatactctgtcaggtgtggtggtgtgcacctatagtcccagctattctggagactgggagttcaagtccagcctgggcaaaatagcaagatcccatctctaaaaaagaaaaaaaaggcatagatacactataaatttatgtaaatatctatattttattaattatagaattctattataaaactcattttatgaatcacaaaatacgctatcaattaaactataatatatagtagatcatcaattataacatgaaccctgataaaatgtgaaaaatctgtattttataatcagtgaaataagatacacattttgtgaccatttgattagtgtctactgcacccataaatatgaggccagggtataagcttttgttcatcattgtacccacagccagcacaaataatttcctaataaatccttgaatgaatgaatgaatgaacaggtgaatgcgtaatcaaacaagggagtttgtcttacactgcaaatgcctaaatgataaataagaaaatgccttttaaaaaagtattaactgctaaataagtactgtaaagtatcaataatattgtctgatatttatttttaagtgatgcatctatttctttggaaagaagtcctgaatattttaaaagtctgataaataagaaccacccacctctgtgtgaaacaggagaaggttgccttataaagcccaagtgagtaatctaaggtgtttaatatcacaattaaatagggtggagagagcaagctgttatttcctcctactcttctgtttttaaaaagccaaattcaaacggtctgccttttacctatgagaaaagcagttctatacccttatagaatcctgtaacagagataccattttgttctgccccagctatttctgctatggaaaagcattgaacagaaaaaagcaattatcttcatccagatacatgcaggacttattatattgtgattattgaaccaaactcttatatagagagatgcatttcaaataagaattctattagccaagctaagttactcttttgcctcctgttgttactcaagtcttttctcttctgtccttctgccagccttaccccactccttaatcctctgaaccagcaaaccattgccaagttctgatgcaaagtggtttataggcctgactggaccagactaaaagtgttcaaaatagcaagcaacaaggagcagaaatccatattagaatgggatatggactatatttatattggtacagaatgccttcaataaagagttgtgagttgtgtaggtgagttgccatggagctacaaatatgagttgatattctgaaatcctagacagccatctccaaggttaagaaaaatccttatgcactcacttgcaaagatatccacagcatgctcttaatggagaaaaacaaagccttagatcaaatatgtaaagtaatttttagttttttgaaaaggtatgtttgggctatagataaatctgttcaaaaaacatgagagaagataataatggttgaaaggagacacagtgcttgccctcaagaagtttttgtctagtgagggagagagaacttgtatgtaaataaaattgtgttactaaggtagatagtgagaagtaacttaagagaggatcagataaggtattaagagaatacagaaaagggtctggattaattctgaacagcatcaaagaatgttcttgcaagagatagtgttttcaccagatcttgaaggtatggatgagggtatacagagtgagtatattcagattctactttaaaacaaatactttcctctgttgtagtggagttgagctatacatccaacaataatgaaaaaatacacgcatatatacatatatggagagagatacatattttagtacatgtagcaattgattaataaatgtacagtttaagtcgcatgcaaaaccttggagtgatagcaaacttcattgtaggatgtttagcagcatctctggtctctactcactagatcccaatagcatctccctaggtgtgacaaccaaaaatgtctccaggcattgacctctggaggcaaaaaaagccctttattaagaaccagtggtatacataagtaaaacatacacaagagattcctcccctcttctctgtatgtgaataaaaattgcaaagttcatgacctggattttccttttaggtttcttctttagtggttcttaacttcattgggtgaagtaagcctttgaagatctgttgaaagctgttgactcattcacttctcaggaaaacgcacatgctgactaccatttcagagaatttgcatcagggttctctggggaggagttctgagttctgtttccaggagctcgtagaattgtcatggtctgcatatgcaaggcaggtggattacggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtgagttcgccctgaagatgcccacaccgcccggcccgggctccactcccggggaggcctcgagggttgcggatgggactcttaagtggtcacggatcaggtgggcagggggcagtacagctttctttctgagacgccgagagcgaacaggctgctcggaaaacaggacgaggggagagacttgctcaataagctgaaagttctgcccccgagagggctgcgacagctgctggaatgtgcctgcagcgtccgcctcttggggacccgcggagcgcgccctgacggttccacgcctggcccgggggtctgcacctctcctccagtgcgcacctggagctgcgtcccgggtcaggtgcggggagggagggaatctcagtgtccccttccagccttgcaagcgcctttggcccctgccccagcccctcggtttgggggagatttcagaacgcggacagcgccctggctgcgggccataggggactgggtggaactcgggaagcccccagagcaggggcttactcgcttcaagtttggggaaccccgggcagcgggtgcaggccacgagacccgaaggttctcaggtgcccccctgcaggctggccgtgcgcgccgtggggcgcttgtcgcgagcgccgagggctgcaggacgcggaccagactcgcggtgcaggggggcctggctgcagctaacaggtgatcccgttctttctgttcctcgctcttcccctccgatcgtcctcgcttaccgcgtgtcctccctcctcgctgtcctctggctcgcaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcaggtagcggccgcgcgggcctgcgtagagagaagcggagcggggcgtccacgccttggggagggaagggcgtccccagcgggcgagagtggggtgcgggcggcggagcccctgggcgccagctgcttctcccagaggcccgactttcggtctccggtcctccacgccgcccttctggtgggagggtggctccatcagtctcgggcccgaaatgaacttacctgggaaactcgcctttggggagagtgggttctaggagccccgtctctctttttcctctctgaaggaaacttggagtgcctcttggggtacagtgggtccctgttgccttcttgggagcttgtttaaatgaaatgaatagggaaacccagctcttgaccaggaggagtccttgaaacactcaagctaagtaggcgggctaccattcagttagagaccaggatgcaagctagaacccaggggagcgcggggtgtgccaagtacttcatcagcaggctgtgggacccctggggaaagccaccctcagtctctaaacccaaacatgccgtaactagatgtcacaaacataaagaaattagagtttctaaaacctttcattatagaacatttcaaatatatgcataagtagacgtagtagtataatgaactccccacccccaccgtttttaacccatcactcggcctcaactgtgatcaaatccaagttactcttgtttcttgtatctacacctacttgctccctccagtattatttttaagcaaatctcatatagcatactgtctttatgtattttactgcatatctttaaagactcttaaaaaatataaccacatttcatgatcacaccttaatatctaaaaataacctcctaatatcaaaaattgagtcattattaaaatgtccaatcgtcccataaatgccttaattaaaaaacaatttattcaaatcaggagttataccacctacttttgatatttttctttcatctgtaggtcactgaatttaaaaactaccaagtagcaaggtataaggtatacattcctcatgctaagatttttgtaaaaactagctccaggcttgtattgccaaaatatactcagtgtgtttatcttctttaaagaaaataataataataaggcgcctggattaggagtctgaaaagtaatctccatttagagactccacatccatcagtctctggctggaccagaaaataggtttttgtgtaggaatattttttcagaggattaaaaatgtgagcgaggggtgggacacttaatcctgtgttctctagaagagtgcatttaaaaggatagatacagttcttggcaaaagcatggtaagcacttcagggttattattttccaggaaatacttatcctttttccaaatagttataaacatcagcagaatccagttcataactttgtgatttgcaaattggttgtgactgagattggattgaaaacccagttttcttgctttttgacagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaaggtaagatgttggcagattgagagttctggtctccagcaggagtttaacacttctccccagctaccataggtctgtgacagatggttgcttacccttcaaggcctgtatctttcctgtagagccccttagtggagagagtcacctctcttctccccttccttagagttctcttcctgggaaactgctgccccactaggtgcagaggtccaatttagaggcatatactaggcagtggcttctcaatttttttaaattttattttatttgagacgggggctcgctctgtcacctaggctggagtgcagtggcgcaatcctggctcactgcaagctccgtctcccgggttcacgccattctcccgcctcagcctccccagtagctgggactacaggcgcccgccactacgcccggctaattttttgtatttttagtagagacggggtttcaccgggttagccaggatggtatcgacctcctgacctcgtgatccgcctgccttggcctcccaaaatgctgggattacaggcgtgagccactgcacccagccagcttctctattttcattgaccacaattcaataagaaatgtgtaaagagtttcaagtcaagatttaaaaaaaaaaaagaaatgtgttacatcctgatatatacatatatatctaaagttcctgtgaaatatttattataacaatgtgctaatctttcactttgttctattatgcttaggttttttaaatgatgattgcagcacattaaattattttatatccacacgtggatcttttttgttggtttgttttgctttgctttttagagatacagtcttgctctgtcacccaggctggtgtgcagtggcacgatcttggctcaatgcaacctctctgcctctcgggttcaagtgattcttgtgcctcagccttctgagtagctggggctacaggcatgtgccaccatgtccagctaattactgtatttttagtagagatggggtttcgccatgttggccagggtggtctggaactcctaacctcaagtgatccgccctccttggtatcccaaagtactgtgattacaggcatgagccaccatgcccagcctcccacacgtggatcttgaggtgtagtttgaaactggcttaaggaaaagtgaagggagcttcctagcctggactccacatgaatgttttgcctgcccctcatccctttttaaaaaaattcatcctcttattttaatgtgtgggaaaatgtgccaacataggtggttatttgttcttttataaactgagttcataccataatattcccataagcatcttagaaatctgttgtatagttggtgcctagcagcatggcattctcctctgtcccctatccccaccatctcgtttctgccccacccccaacaccctgaccaatttcccaaagaccctgaatcatgaaaacagaaatatcgttatttattataatttgtgaaacacctaatatgttccaggcactaggtatgctgacacgaataagatgcatcctctgcttccaaggaactcatagtccaatggagaaagagaaaacaattcaatgtgataagggctgtggcaggggagtgcaaattacttgctaagggagtgcaaatgaggttcactcaaagcggtcttaggggatcttgaatagcttcctggaagttgaatgtcaaagctgagtttccctgatggctaagtctgagtttgccaagtgatgggaagtagggtggggtggggatggcttgaaaatagactatttcagaagctagtaatgaatgttgagcaatattgtgagctaaaatttggctatggagctttcaagattgctcagatcaggccaggtagatgcccgaagagaaggaggatggcctatgaacccagggtggagcctcagcctgatcaagcatgttcttaatcacaaaggtggacgggagagagactaggatgggctgtgtggttcctaagttaaacaatgccaagtattctacactgtgctctaagggctgtgacatggtgacaccctgtgactcctgggggacaggaaccatgcctcattcatgtttgttcagcaggtgcctagcacagagacaggcacataatagatggtatatttagtgaaaaagattgaattgcattcctgataatgaatcttctttatatataaaaaatggtggttcatgctccatgtctccccagttttcatagacaagtatcttataagaattcattttgtttataagcatgagacactatgctaagtaagaatgttgccagctatctcatttaatcctcataacaatcctgctaggtaggtctattattatttcattttcagtaggtgaaaaaatctattaaatcttgcccagggtcacgtggctggtaagtggtacagctgggattttaatccggtgtctattgtgtagttccttactttatgggtaagatgcttttctagaaacattatctctataaagttttctgtgtaaggtgaatttctgggaggttaatctggctcagttacacatccatttatcattcattcacttaataaatatttataaactaattctgtagaccaggtatccagccatgtgcactcagtggttcattaacaggcacaggtgctgacttcatggagctttgtaaaattattttgaggggtagggatgggaattaaataagtagataaatagaaaaataattatagattgcaatatctattgtggtggattaaatatactgcaatagggattaatttgaaaggtggatctttatttttatatttttttgagacagggtcttgctctgttgcgcaggctggagtgcagtggtgagatcacagctcaccacagccttggcctcctgggcccaggtgatcctcccatctcagcctcgccagtagctggaactacaggcatgtgccaccatgcttggctaatttttgtattttttgtagagacaaggtttcaccatgttgcccaggctggtctggaactcctaggctcaagttatctgcctgcctcggcctcccaaagtgctgggattagaggtgtgagccattgtgcccagccaggaagggaggtctattttagatataatggtcaaggaagaacttactgagaaggtgaaatttagtaaaagacaagagaatgagaaggagacagccatgtgataagcagaaaaaaagagtgtaccaggtaaaggtgcaaaggccctgaggccagaaaacttagcattttccagggaggaagaggaagaattgtagtgtggatgagtgcagtgggggagggtgagtaaaccatgaaataaactgaggcaggactgggattaggatgaggtaagggaggcactcactctcaggatgcctctttaaatattgtaccctaggaaactcatttgcctcaccctattcctggccctgtggggatattgctgtgaattaaagggagaatgggtgtgtcagcttgaaccagatgatgcaggctatgaagagagctgcaagataggaatatattgtgcagcaagaatcactgacacataatgatgagtgtggatgaaaatcactgatgcctccaaggttgttggcttgagcaaccggatagatcattacatcatttatctaaatggcaaagacaggctgaggagcaggtttgggataggaaatcaagtttccttttggatatgctaagtttgagacacagggtgagacatctaagtggagatgccacctagagaagtgggagctactgcaagtgtgaaggttagaggagaggtctaggctagacataaaaatcaggaaagcattgctgaagaggtgatatttacaagagtgggaatgcctgtgattacctaggaagaaagggcaggtgtagaagaaaagagagccagagtacttagaagttaggaaggcaaggagaggggcagccaagaacctgagaaagcctggcgcaggaagttgaagacaaagaacaaccaaaggaatgtgggatcagaagccaagagagaaggtttaaaaaaaaaaaaaaaaaaaaaaaagcatagaggggctgagagtgttgaatttgcctgaggggtggggtatgatgaaggattacagactgcttttaagaagttttgccatgaatcagagggagataagagatgaagacagagtgaagggagaatttgtttttcttaatatgggagatactagaacgtatttgtgaaccgataggaatgacctagtagaaagaaagagatctgtggtgcaagaagtacaagagaaataactagaagcaaagtagtaaaaaggggcaaacgtggaggggtttgagaatggaactaggagaactactccttaaaggcaggagggatatttcttccattttcaaaggaagatagcccaaatggtgcagatgcagacgggtttgaaattttgatgaaaagtaacttgggagcatctcatgcattctgtgttttcgattaaatgcagaatatgtcccttaactttgagtctggtggagaagggtttgggcagattgaggtaagtaggggaggtttgaatagttgtctcaaggagaggaaaggcacaccccctgaggaaatgtaactgggtccccaggcagtgttgagtgcccatctagcacttgtggtcacacattttacatgaaattggttagcccagatgtagatgccttcagtccagtttatctagctgctcaaaggctctcatggataaggtagatgtttgaggtcagccaaattggggttttgccagacaagaaaaggaaggaaagagagagtgaaaaaagggagttaagaatatttgcaaaattccatggaatctgtgtgggcaagggtggaagtgaagacaggaaggtgtggaggatagagagaaagtggatcggttaattaattagaagcctcgtgagcacaaataaatgttgcaataaggattctgcagtagatgagctggaaggtttggaggctgtagctggagagaggctcaaaattcagattttggaggtggtggtgtggtttccagtgatgacaatgtccatgccacaaccactggctgagcagctgagtagtagaacaactccgagaagaccagctcaggttactgggaagccaggaagttgctaaggatgatgggatctttgtgatttttgttattaaaacaaattcccatcagacagcaaaagatcgatgcaacccttgtagtgctatttttattcacaactttaaggagtctgctgtctgctttctccttttgacttttatgtatttgtttgtttaaacaacagaaatttatttctgcaacatgaaggtgcctgtagattcagtttctggtgacagcccgtcaccttgctgcgtcctcacatggtggaaggctttacctttagaagaaatttaaatttaatataaatttcaactgtttattagcacctgctttgccagtatttgtcaccatgctataaatcatgactaccaacagagaaattctatcatttaaatcaatggctcagatttcttctaattttttttcttttttgagactctttctcacacaggctggagtgtagtggtatgaccttggctcactgcaaccactgcctcctgggttcaaactatccttgtgcctcagcctcctcagtagctgaaattacaggcatgtaccaacacccccagctaatttgtgtatttttagtagagacgagatttcaccatgttggccgggctggtcttgaactcctggcctcagtgatcttcccacctcagcctcccgaaatgctaggattacaatcatgagccatcacacctagccccagatttctttacaaattgtgtaattttaaaactgttgtaccaagtttctttataataaagatgattccagtatatttgagtagtagtggtccctaaatgggacactctgtatgaaaactgaaaagttccccttttccaggcatattaagaactgcagagcccagtttggggaaatggaagtttgagaaggacttgaaaaatgtccatatgaaatagacaagttgtgctttcatcccctgctccttcttttcttatcttccttctgtggagtcaccttcctattttattccatgctgttgggactactgactattgcaattcctgtttgtaatttacatattaaaacaattgtactaatagatatatttttctagaatacaaactccactagggcagagactttgttttaacagtgcctgacacatggtaggtgcataatcaactgttgttgaatgaatgaatgcttattgtagaaaactagaaaatacatataaggaacatgaaaaaatacctccactacaaactagaagtctcctctgatcctaccatacagataacccattgatatttcagtaaatatcttactaggcacataaatacttatctatataaaagtataatgaaatgtctggaaataataagcaccaaattcaggataattgttgccttctgagggtagagggaggaaagaggatgtgattgaggaagagtatactgagagtttccgctgcattggcagtaatttatttcttaagctagatgatggatacatgaaagtttattatattattcattgtgtattgtgtttcttgtatatgtaaattacttcatattttattcaaaatacctccagatttttttttttttttgagatggagtcttgctctgttgcccaggctggaatgcagtggcgcaatctcggctcactgaaacctccgcctcctgggttcaagcagttctctgcctcagcctcctgagtagctgggattacaggtgcctacccccatgcctggctaatttttgtatttttagtagagacggggtttcaccatcttggccagggtggtcttgagctcctgacctcctgatccacccacttcggcctcccaaagtgctgggattacaggtgtgactcaccgcgcccggtccctccggctttttttaatgcgtacactcccacaggctggagtgggagtgtattttttacaaaagaggaagcatacatacctatcagttttgaatcttgattttactcacttttttccctcttacttcacatctctccacgttactgaatgtacttctgagcatcacatcaatttcatttcatagaattacgaagtatgaagcataacttagttaaccacttccctgcttggggacattcatctgataagtaatattgagtgtgtgggggccagcatattcaggctgttgccatttttaataaactactataaatgtaattccttattaatgactcaagagtgaaaaggcttgattttctctcattgtgagtgagtgaagggtgaggaggtaaaacggagtaagatatgctggctacctaagtatttaaccaaggaaaagaatgtcattatcctcttctaaccctcatgtaaactagaatgttgatttctctatagccaggcattaatgggtctgggggctgctgccattgcctagtggaggagttggtcaacttcatcagggtagagttgtcttgtctgctgctgagtataattatcatattttttctttgggtctaacttttcttccccaccaaaaaaggagaaagggcagtgtaacaatgactgtgtccctcagaacgcagagaaatattgagctcagacacaactgcctctgtgcaaaactggtctgagcctttggagtgaattgaccaaaacttttttttttcttttttagacataaggtcttgctctgttgcccaggctggagtgcagtggcacgatcatagcttgctgtaacctcaaactcctgggctcaaacaatcctcctgcctcagcctccttaataactaggactacaggcgtgaaccaatatgctgggctcattcattttattgtagagatggagtcttgctatgttttccaggctggtctcaaactcctgggctcaactgatactcctgcctatggcttcccaaagcactgggattataggtgtaggacaccaggcctgaccaagtttttaatcaacatttgacttgattgttttttcccactgatctgattggtggaagactgaattactgatcaactgagtccattcatttgagcaggtgatgtttgtagatgcaatcattgttccaggtgatttggatcctgttctgcatcctagcatagccagtctgtatgcaagataaggagaccaactcaggacaggctgatctgttgaaactgtaaattgagagtatctgtgtaaaggcccttctttctgcagtagaaagacagatgctcctctacttctcatgagttacatcctaataacccattgcaagttgaacatattttaagtcaaaaatgcacttaatacacctaacctatggaacatcatcgcttaggctatcctacctgaaatgtgctcagaacacttacaataacctacagttgggcaaaataatctaacgcaagcctattttataataaagtattgaatatctcatgtagtttactgaataatgtactgaaagtgaaaaacagaatggttgtatggttacttaaagtatggtttctactgaatgcatattgcttttgcatcatcataaaggcaaagtcataagtggaaccattgtaagttgaggactttctgcatactgtaactttggtttgtgaatgtaatcactttgcatgtgctttcagggatggcatatgccctactagctgcagttcctgtcggatatggtctctactctgcttttttccctatcctgacatactttatctttggaacatcaagacatatctcagttggtaattataagtatattttacaattatatttgctcatgtttaaagtgttttggctatattaagtgcattatacctctattaggttggtgcaaaagtaattgcggttttcacaattatacttttaattgtgaaaaccgcaattacttttgcaccaacctaatatatctgtgttaatgttgtcagggaaatgggatttcagtgttttgcctgctttttctattcactgatgttaggtaacttttttaatgaagtggaaaaataaaaaaactgaaaatgacagcctactttaacattttagcatgttttgctttttaaaaacgtgatcaatttgactccatttttggagtcataatgacagggaaaaaatgacacaaggtctttgaacctaccattttactggattaacttagaaattactaagtatgttcatcaaatacatgttcatgatttaattaaagagcatacttattttaaatcactaatattagaagactggcaaaatcttaccaaaacaatttagaagctaagcttggctttctttcttggtatggcttataataagttttcctgctattcctaaaatttgaattctcgtttgttagaatgtcctagtaaatgagtgttttaatgctattttgtgactttgacatttatactagagaaaattttgctcctacatccatctgctggctagacatgtctacctcgatgatcctcttgaaataaatgcaaccatctactttatccccaacccatgtttttgtccttagttgtatttccttagaaggagatcagaaagaggttttgattgaaagaacttccacaccttggagaatcaatcagtgttgagtgcttgactagatgaggaacatggaagctggtttttctctttggtattctatacattgagatattccaggtgactggggttgggttttgagacctcaaaaccataaagccttccatgatgagagggtagaatcatcatcatcgtcatggataaaaataacagtagtgaacggagcaattgctgaaaaaaaaatattttttaaaatataaaatcttacagattgacatttgatatgaaaaaatgttttgtcttacaaaaagagaaagaaacttcatactccctttgctgtttttttaatcctaacttcgaccctgtgatattgatcaaggtactcagatccacagagatcaggacattggacactctaggaaattaatgagattatatgaaataaaagttgagcggaacaagtaaacactcaatgtatgtgctacctttgccagaaaacctttcctgacaactctcatccctgtccccaccacccatacacatgaggatcacacacagacaggttagacgaccttttgatatactcacgaagcatcctgaatgttgtattctaataactacctacgtgtctctctcgcctatttctaggtcatgaatgcctcgagatatctgcgtttttagcttctttttgttgttgttgagacagaatctggctctgtcacctaggctggagtgcggtggcgtgatctcagctcactgcagcctccacctccccagctcaagcaatccccccacctcagcctcccgagtagctgggaccataggcacgcaccaccacacccagctaatttttgtcttattttggtagagatggggttttactatgttgcttaggctggtctggaactcctgggctcaagcgatcttcccacctcagtctcccaaagtgctgcggttacagatgtgagccactgggtccggctcagcttctttcgtgaacaaacaatattttcctagtcacagctaaatcttttatacattttttaaaccctatgcagacacattgaacatttgtgattaataactgattaattgttagagactttttttccccaggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacaggtaatgaacttacaagtaaaatatagatggatgtaatttttatttgaaattaactttaaagcatatagacttaaagattctactaaaaacaaaacaaagtaatttcctggaacccaaaattattttctaaattacgttgttttaggtcaggtgctaaaatagtaagcaagaccccacttattaaggctcacttatcatcaaagtcagagacagaaaaaagcacagaagaacatgtgtgattcaattgaggatgagagaagggaaatatacgggaatccataaaggagaaaaagtgttctggtgagcggagacacaggaccgaaagccacataaataagctctgggtttttgcttttctaagtacatgtatagaaatacttcaaggtttttattacacttagttttcaaattttagagtggtggaggaaggggagtgatagggtattaagaaattcatatttttttctaccagtatttttgtgctataggcaggctactagtgttttcattggtattaagcttgatgtaatatttccagagagtaggtttctatctcaggcaaacatttaatttttctttccttttccttatcgtagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatgtaagtgttgcttcttgctccagggatgggtcactgttcattccagaaacaattgtattcattctctgagtctgggccaggcgtggtggctcacacctgtaatcccagcactttggaaggccgaggtgggcagattgcttgagcccaggagtttgagacgtgagacctcatctcttaaaaaaaaaaaaaaaaaaaaagaaagaaagaaaagaaaagaaaaagaaagaaaaaatccaaaaatccgaaaatttgctgggtgtggtggtgcacacttgtagtctcagctacttgggaggctgaggtgggaagatctcctgagccctggaggttgaggctgcagtgagctgtgatcgccccactgcactccaccctgggtgacagagcaagaacctgtttcaagcaaaaacaaaatcaaaacaaaacaaaacttgagtctgggagcacccacatttctttctttctttctttttttttttttttttgagacagagtcttactctgtcacccaggctggagtgcagtggcatgatattggctcaccacaacctccacctcccgggttcaagcgattctcctgcctcagcctcctgagtagctgggattacaggcgcccgccaccacacccagctaatttttgtatatttagtagagacagggtttcaccatttgggccaggctggtcttgaactcctgaccttgtgatccacccacatcggcctcccaaagtgctgggattacaggcgtgagccaccgcgcccggcctgggagcacccacatttctctatgcatactttgggagccattgaaaacatttggttgcttctgtcagaaatactattgtacttaaaaacattaatgaaaacaaagtggtctaatgaagtaaaggctgctctggaatgtaacagacttgatttctagcctcagctttgtctctaacaggtaagtgaccctggatacatcatgtagggtgctaattgaaagctcagttttagaactggatggaccttgattcatattctggctctactgtctactagccatgtgagccaatcaccatgcctcagtttccttctctgcaaaattgggttagtaatagtatatgcctcattggattgttaaataagaatagataagcacttggcccaggggctggtacataacaagtactctaaataaaggagttattttgaaatgattatttcaagccatcttttcttttttattggtgaaatgaaatgattggagatgtatctctaaaagctttttctaaccataagagtccgtaaaatgcataatgtaaatgtctcaacaattataaatgaaaaggaacattagattcagagatgattcaccatgcaaaagaaatgcaaaagcaggatgtagatcacactaattagatttgaaaaaggttttggatctaaaaatgtgttctacataaagcatgttattgatgcttgaaaaatggtgataatagtaacatgatgtcatcctagcttggggggaatgataccatacaaaaataacgtgtcaacattaagaatggagtttgagtctttaaagacccacctattagcttctaaaacaatgtaaggccatgtacacatatctaagaattggtgagtatgtcaataattgctcaattttataacggtgccataatcccatactattacactccatccattcaacaaatattaccaagtgcttactacaggccaggtactatgctaagcagtgagaatagacagaggaggcagaataacatagtgaataagatcatgaggccaggtgtggtggctcatgcctgtaatcccagcagtttgggagactgacacaggtggatcacttgatcccatgagttcaagatcagcttgggcaacatgacaaaaccttgtccctactaataatacaaaaataattagacatgcattgtggcacgcacctgtagtcccagctacttgggaggctgaggtgagaggatcacctgagctcaggaagttgaggctgcagtgagccgagatcatgccactgccttccagcctgggtgatgggagtgagaccctgtctcaaacaacaataacagcaacaacaaacaaaaaaccaaaaacaaacaaaaaagatcatggactttgaacttcaacagacttgagttggcatcttggttctccacccactacccatgatgtttatactcaacttccctaagcctcagtgtcctcaattgtaaaatgggagtagtaatagtagctacctcttagagcagtgaggattgaaaaagaaaatacgtgtaaggcacttagcacaatatctgtcacatagtaagaattgagtttattattattattatcacagatgaatagacctcatgaagctcgccgttcatttcctcgggacccagcctattaggaaacattagcaaaagtttttgattcagcattatatgggtcattcttgatatgttttttcctgtcaaatcctcagttttctattaaaaaagaatttttttttttttttgagacagggtcttgccccattgcctgggctggagtacagtagcacagtcatagctcactgcgacctccatctccctgtgtcaagcgattcttccccctcagcctcatgagtagctggaactacaggtgtgcaccaccacacctggctaattttctttttctttgttgaattttagtagaggcaatgtcttgctatgttgcccaggttagtctcgaactcctgagttcaagtgatcctctcactttggcctcccaaagttctgggattaaaggagtgagccaccatgtccagcccatattttaaaattaaaattaaaattttaagattttattttggtagagacaggggttttgcaatgtcgcccaggctggtcttgaacccctggcctcaagcagtcatcttactgcatgcctcctaatgtgttgggattataggtgtgggccactgcacctggccaatccttagttctttttgagacctctgactgccttattttgacaggctctgtaacttttgcagctcctggcacatcttgcttttacttacttttacattttctccatcaaatgttcactcatttattccttcattctcattcattcagtttatctactcaataaacacatattgaatacttaccatgtgccaggcactgttcactccctatacaaacccagtgactcattaacctaccaagacatctcttactgagtattctcacctcactgggcaaacattgctaggccttcatctacacagatcgcaggaggtccaaactaattgttgacctgggtccatggccatttcaggacccctgcagaacaggagaaatagttcttctggtctcataggattatgtttaagaagaaactttctgcaaatgatttcactttgacagggacaaaagtgagcaggaaacactagaccaaggtcaaaaatagtttttaaaccaagtgcagtcattatgtaacttacttcctgttgaatatttttcttgaaatctcagtttagcatacagcatagaattttgcaagaaactcctcacaggccacccaagttggaaaaggtatttcagtcatccctatatggtggtgaatataagtttgctcttctgaaaaaaaaaaaaaatcagcaggtctgaaaacgattgcatagggttaaatgagccgcttagtctctgaacagagaattttgcttatgacattaccattgcaattatacatgaaagaatccttcaataaatgcagagtagtagaggattctcccagaaaagactgtaagattgttgtaaaatactttacctggagtgttaacttatctttgttctaaggttgcactttggtttgtcctgccctctcccccaaaaaggtgctgacaactagaagtaacaaatgcaaagaaataggggagaaaatattagaagaaaaagaaatcagttaatttacaaatgtcagagatactggatgtaggaggcagaagggagaatgacagggtattggcagcaatttagaattagtcaatacccttgatgctaccatgtagctactcagttatctaagtaaatcccagtccacttaggggttgatttcccaccactcaatttgaatggtttgtctcagtagacaagggtgagagctttgaaagccaccactaagcttgattagatttgttactttgtacctatttcagtatgtaaaatttatacattgtattaaagtaacactttaaggattgatctatacatgtatacacaaaagatatggaaggaaatgcctgacagcaaaaggtggtcgtgaagcatttgtgatattcgtagagataaaataaacaaaaataaattcagttccaatagtaaactgatcaattttctcatacacattattgggcataattttttcttacggtttcataaacacaggtaatcctgggcaagaacaaagaaattatctaaatgaaattgtcctaaaattttccaaacacagcaggaaatataataaaatattatctattaccaaaaagttcattatcataatttgaattatttggaatactaagctaaaatacagaattatatgtgtatccatactattaacatactttatggtattttaacagtaaactaaattagaagtactgttcagtgtttttgtagagtctaatttctatgtcgttaaatataatcttatattattatataatataatcttatattattatataatcttatattattatattatataatcttatattatataatcttatcttatataatcttatattatatgctcttatataatataatcttattatataatcttatcttatataatataatcttattatataatataatcttattatataatataatcttatcttattatataatataatcttattatataatataatcttatcttattatataatataatcttattatataatatattcttatcttattatataatcttatcttattatataatataatcttatcttattatataatataatcttatcttattatataatataatcttatcttattatataatataatcttatcttattatataatataatcttataatataatcttaacttattatataatataatcttattatataatcttatcttattatataatataatcttatcttattatataatataatcttatattattatataatcttatcttattatataatgttatattattatataatcttatcttattatataatgttatattattatataatcttatcttattatataatcttatattattatataagtataatcttttattatattattacataatatataagtataatcttatattattatataatattattattatacaataataatgtaaactgactattgcagtttacatggtgagttttatttctgcacagattaaactttctatcatagagctcttcttaaaagattctgaatgactactcaatatgtaggcaacttatttacatactcagataccttgcatgatgcaattctgtaatataaactgattttggtttttgaacctcgtgttacatttctaatccaccagacatactttttattcaaaggtctaattgattctcatggattaaactggaatcctgtagtgattatttttgtaaggtaaaaatgtttgtgtgattgggataatgattccaaatgttttgtttttcaagttttaatttttgcatcttgagttgaaaagtacacctttaatgaatagacaattcatcaaaatgttatacctagctcctgaattaattagggtcattcatcagaaaactgattatgtaaaggaccaaacagcaatgataaccttcaagcctctgggataggtagggctgtttgctcctgtgctaaatgccctaagttcttgttatcgtgtctgctggtggtagttatcccattttctttttcactctgctgtccctggcatcatcttgtttctgtcagtgtaaatgcctttagcaattttcagtgactctgaaaaccagacaaagtgaatttgttagtactggatttcaagaaagagcaaaagtaccctctgagtgaaagttcaagagtcttcctacaagttaaatgcctgaactctagtccaagtgtttggtgggaggtattttgcatgcataatagcttctcccccatttccttactcttcttgaatgtgttccagagagctgaggttgctggcaaagtgactcctagattacttaaggttcattaagtacttaaagttcattaagctcatacataattgtcaccagattatcattataattattttaaaagtagtttatctgtgaaaataagtaaaatacattttataaatgttataaactataagatgctccattattttatcttcctataaaatatgtctataaaaaagctcatttaaagaaaagttaccttcactgtgagattgtctcaaagaaatgtctcaaagataccttcttaaatcctgccttctacaggactgaagttaatataacaaagatattagtgttcttccttgggattcttccttgctaatttccttgaaaacacccttgattcccagaagtctcctatttacaaaacaaaactagtttttcttgaattgattcatctgtctacttaaagattttttttggccagttgtggtagctcatgcctgtaaccccaacactttgagaggttgggcaggaggatttcttgaggccaggagttcaagaccagtctgggcaacaaagtgagacaccatcccctgcccccacctcctcccgccccaatctctacaaaaaataaaatagcctatttattgtactctctacaataaataaaataaatatagatgcctgcctgtagctccagctactctggagggtgatgtggaaggattgcttgagctcaggagttcgaggccactgtactccagcctgggcaagagagctaaaaaaaaaaaaaaaaaaaaaaaaaattttttttatgttatcttagttcaagtttgagaaataaaattaagtaccaaatttattcccaaaaatgtatgcttgtcaggagatatacctaatgttaaatgacgagttaatgggtgcagtacaccaacatggcacacgtatacatatgtaacaaacctgcacgttgtgcacatgtaccctaaaacttaaagtataatttaaaaacaaaaaacagaaaaaaactacagattcaattcagctttaggaattaaaaaaaagatttgatgtaattatatcattataaaatcaaattttttaaataaaacatgaaattgttttaatttaaaaaaaagtatgcttggcctatatgtcaattcaatagcttttctttgccagaaagaatgaagtcagtctacctgtatccacatatgaagtattttaggattttttgtaattttttgttattatctatatcattcaggtttaggaaagaactttacctaaaatactggctaattgtgccccaaaaaaaagaaagctagaaagaaagaacacacacacacacacatacacacatatacacacacaaaaaaaacaaccaactgatcaactgggagtttcaggtttatcagcctttagtctcaatgctgtatcactgtagtaaaaatcaggctacttgatagcacctagaaacccattttaaaatataaacttgtaaagcatttctgtaagtaaaagttagtatgtttataatgacattttttcttttaagacatctttattattatcataaaaaatacagagaagcaaaaataagaatgtagaaatcacccagtttttcctttccaagtaaacaatcataaaaaacttggtgtatatatatattgcagcaattgtatgtatgtaatggtctctgtatcaaccaacacatttttatcattttactgaaacttttgagtgttgtttgatgctgatatcatggtttttcatgtgggaagattcatatgagaattgattgtgtgtgtgtgcgtgtgtgtgtgctcgtgtgcgtgtagcagcaggaagtatataaaattattttctttttatagacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtggtaagtagaatatgtagttagaaagttcagcattatttggttgacaaacaaggaattattaaaaccaatggagtttttaacatcttttgttttatttcagacgataattgctactgccatttcatatggagccaacctggaaaaaaattacaatgctggcattgttaaatccatcccaagggggtgagtgtggtgttcctcttagtactaatacattaagtcagtaagtcagtcttttttatttaaataaaaccttttattacaagcttcatttcactgatactccttcaatagtcctatttgtgtgtgatctggaagaaacaaccataagacaaacagtatgtgtgtagataaacgtcaagccatttgtttaaaagcagcccgtatatgctcttttttcttcattttcttaaaacatggcagaacatgtacggttaacaccagagtcctgatttaaacttatacataacattataatataatataaatagaatcatttctcagtaacatctattgcactgtgaacaatctgcttaaggaatcagggacgtctggcctaaattctctatcatttagtttaatcaattttggataataattatatcctcgtacccataagtaaaatgcattatcttcaagggaagttttagaaacatttggtagtttggtgaattagaagagtaaaagatcaaggtccttcattcatctctgtcaccttccagttttgtgacctgaggagattctcctatataagacaaggataatgaaaacagccgaagcctcaactcaagcttattatgaaacaccagtgagatctctcatgaaatgaagagtagtcaagacaccctacaccataattttctcatatggggttacaaaggaggcctagacgtttattatatttgtaacatacactcataacatttaagactagacttgctgttaatcaagaagtactagttttccctaagactggaagagtttggtaccggtgtttctaccaggaaagggagtggaggggagggaagggcacggagccactgtttggtaccagtgtttgttttccatgctcttaatagaaacttcctcaggcaagcttattcccagagcttattctacagggaactgctgtattcccatttgatgaatcttataatttcctctaatatctgttccaaactatttgaaaaagcacagctggctacatgggacttaatttgcatgctatccattacagtgtttctcaaacttacccttttgtaagaatcacatgggatcctttttcacacagattcctggttcccaccccaaacctattgaatcaggctttccagaggtggggctcaggaattaatatttttcaaaagtgcctcaagtgattcttgccctcaaggaaactggagaaactctttaagagaggatcaacaaatctaatctaacaactagataatttcattagttagaaacaattacagaaaaagatactttacagatctcttttgtcaggcttcctaggatgtatcaagttataggtggtccacagagcaggccctgtcccatccatccgtgttccattagttggtttagggaactcatttcttctgccctaaattatccctccaggtcctctgcagagctttcctgtgctctcctcctgagaacagatttctagcctctcgcccttgagtttaatgaagctgaatcctttgacagaccacctggtcagcttggtcagctaatttaacctgcctggccccagggactttaagctactccatgagggcagtgaccccattggtgttgttcagctctgtcccagagcacagagctcatctcacagtaagctctcagaaaacacttgttgaaggaataaaatacccaagcctatgtcattataaacaagccacaacaaaggccttggttatctgatctcacttggtagcacagaagcaattctgatgatattaacaaacctgaaaaagaaagggagtggagagattggaaaaaagcaagcagagttttaaagtggccctgaggtgagcacagtgaaaggaagagggacagagagattgtgatatgcccccttttgatctcctccctgcttcaatcctccattaaggctgctgctcgtagttccctttatcctattgagggtccataagttaggatcactcctaacctaggaccatacgttaggaccataagttagtccccaataacaatgccccattggacagccaaaaacagtggctccatgcccttccctctactccctttcctggtagaaacatacaacttttccttgtccagtcaacacctaagcgccccactaggattcactcattctcccatcctgggtgctttcttaaccagcagacactatgagggccagtcatgtgcctctcttctcatgcaactggtccttatctgcaactcgtaatctgcccagttcctcactcatcctgcccaggcttctcttttacctcttctgttatagtggtgtgtgtgtgtctgtgtgtatgtgtgtgtgtgtgctcacgcacgtgtatgtgtaaacccataacaagattgacagctctgtcctctaaattttaatccctattcactctcttgcctcctgtccttgccagttccagaggtccccttgttgtcatgtttgttactctcttttttcttttcttttctttaacagagtctcactctgtcacccagcctggaatgcagtggcatgatctctgtggtgtgtgtgtgtgggtgtgtgtgtgtgtgtataaacccataacaagattgacggctctgtcctctaaattttaatccctattcactgtcttgcctcctgtcttcgtcagttccagaggtcctcttgttgtcatgtttgttactctttctttcattctctttctttcttcccttccttccctcccttccctccctttcctaccttccctcccttccctcccttccctccctttcctacctgcactccctttcctccccttcctaccttccctccctttcctccccttccttccttccttcctctctctcttttttctttctttctttctttctttctttctttctttctttctttctttcttttctttctttccttctttctttctttttcttttcctttctttccttttctgaaacagaacctcactctgtcacccagcatggaatgcagtggcatgatctcggctcactgcaacctctgcctcccaggttcaagcaattctcctgcctcagcctcctgagtagctgggacaacaggcacacactgccatgcctggctaatttttagtagagacggggtttcaccatgttggccaggctggcctcaaactcctgacctcaggcgatctgcctgcctcagcctcccaaagtgctgggattacaggcactgttatagaccccttttgtcaggtttcctaggatgtatcaagttataggtggcccgcagaacatgccctgtcccatcaatctgggttccattcattggtttggggaactcaggcctactgttgtttttaaagtatgtgctttgcagttgtacttgaaagtgtggcacaggggcaaagccaacaaaaccacagggtttttttcctggctgctttctgagggctactcttctcctctagctggagtggtggtgcatttgccaagtgttcagacccagtctgaagtactctggtattctgtttggtattctgtttatgacgctggcctgtcgtttgtggaattttgggcccttcagggttcacttctcccggtcttcatcttttcagataagtgtgtgttcacatgctcactcccttgggctcttcatagtgttgtcttatgatcaggcctgacatgcttggctggggccaaacaggcccactgagcaaaaaatttggagagttatatccacagaaggagggcaagggcaagggcaaggacaaaaggcttacttacaggtcattagaagatatccagacgggacttcaattggtttggggcaaataattggcacacttagggttcagcaagacagcagacatgagtggcaggagtatgacagacttagattctcccaatatataccaagaacctactatatgtgtagacactaattacttacttacacaagtgattcctcattgaatcatatgagaagaatatatagtccccatttgacagctgaggaaactgggatgtggtgggtttaaataacatgcctaaagtcatttgagagtgattctggtcctttgacttaagtctttgcactttacactttgcacacctggataatcaaggatcaggtcatatttggagaactaggcagccaggcaggcaaagtgcattaggctgctatcaaaactaagctgtatgtgaaactggaaagttatcctgacaagtccgagtgacctgaagtagggttgactgggtctcaggagcaagtcaggagcctgttttgagaagtgtggtcccaagtcacatcccaactagcaggtgatgtcatgctgagtcgcccaactgttggatcagggtttccaacatcctgtctgtctacagatggggttcattccacagtctcattgtggggtaggatgggatataaggagaaaaggagtctgaaaattaacctcactggtagggagagtaaggctacaagggcagagaaccagacagatcttgttaggtaggaaatcgtctaatatctaacatttagacctggacccactaactgacatcaagcaaatcacttcacgtttccaggtctaagttcctttatcaacaagacaagacaagagtggtggattcaacaatacctaggatgctttctctatggttttctctaagaaccttatttctattttatttttaccactgttctctaattataggggaaaatgtagtttccttattaaagaaactttaggcataagtgatgtaggtcggggagacctttcatggctgggggcctctggaattataccatatggatagtcatcaagatataggtccatctcccaaattttcatatttgagagaacctcgtggtataatgtgtaggttaatttttttatatggtttgtagggtagctctggttttgtcaaacagtagtcattggaccccacaatcactgagatattttagtcactatggagcgttgtgataattacattaatttaatcttttttgccttaagaagattttaaatgtcctggctctcaaaggattttccactgagctcttttagcttcatacattctttgtctctttaacagagctgatacctcttcaaacacagtaaatctcatattatggctagaatctcttttaaattcaaagaactttaagatggaattgtggaaggagaaacactaacatgtgaaatggcatggatggggctgtatgatattaaagaatcagtaacagcagatttattcatgtgagagatagagaagagtcagcatggctaggctcaaaaatagaggacaaagaaatcagccagtaagataacaccaacttctgattaaacattataaaagctgctactatcatgtatgtatttcgtgtgctttttgttcttttggatcaagtactttcatgtctaatatgtgactgagcagatatagcatttgatgagatggggaaaaaggatggtggtcaaatcttcacagcatttttcacttaaaaactcactaggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggtatgggtgcccttttgctgaactggttttatagggctggaaacaggaaaaaaacataaatggaaaagattttggtgtcagctaaagaaggggttggattctttcaccagaccttattgggttggttttcctcttgtgtttgctaattagaaattattttggaatagacacactgcatcactttgcttttttaagaaataagttgaaataacactaattttaaaagagctaattcagtcaggtcataaataacattcaaagtgaacatattaggtcagaaagagaattcttcatgtaacccagtacctcttggcaattatttttaacggcagttataatgtgacaaaacagtgcaacaaacattaataatagttgtgaccactttaaattgaggtggttcactgtgcagttaatatatacaggcaatttttgtaggcatgtgatacattgccaggtaagctgatttcacactgcttgcaaatttgcaagttctgtatctatatgtttctgaaaatacagtatagcaataacagtagttaaatggttagtaatcaagcagaataacagcactgctagtcataatatgcttcatagtgaatattcaaccacagaaaaacactttatgtgagagctgcagacattccattcactaggcagtgaaaaagcttagcaaatgatcaggtgctatttcttgtcaactttaaaaattatcgagagcaatgagacctctctcagatggtatggcgtccaaactcctgatgtcgtacaaggaccccaagtacctatcacggtaaaaattaaattggaccaccacgcagagtaggcatgggagttttcattcttaatgtacttcctgaaatactcagcgaaggtcttgcaaagattcaatttgtaggatcgttgtcatccagtctcttccttaggaattcattgcctttgggatcagcaacatcttctcaggattcttctcttgttttgtggccaccactgctctttcccgcacggccgtccaggagagcactggaggaaagacacaggtaggaacaacagccttatgatatccatctcagagaacaagtcgaggaatggcaacagaggaaggctcgcaccgagcttagcaggacaatttgcctttcagacttgtacttcctaatctgattcacctcaggcctattcctcttgttccactccctcacctgaaatctcttaaaaaacaacatgtatggttttctgatacagtgattctcaaatctatttgtcagtgcttacctgtcatgtagctacacttacctgctgtggtcaataacaactgacaaggaatcagtaatgaaggatgctgatttttgttaatttgttacctggggagacagatggcctggtaaaagcgcttcctggctttaggagctattctattttcaggaaagtgaaaagctctattcacatttcctttagaaggatcaggaacttcctccaggggcgtttggtcccacagaacaacagtttgctagccaagaatatttttgttgtaacatttttggcatttcttcctgagtaagatttatgttgttgaagttctgcatgtgggtttaatcttatcttttaaatgtggtttgcagttgttgcctttacaaggtggccaaaaagccagcatcctagttaatctctggataatccctccttcagtgttcagaaagctcaggagccatactcaaaggccactctttccagcaggacacagtcgaagagaccacaccaagcagatgggcacaagagacagactcaggaattttgcttctgtctcttatgccttgaggtccttatcttcccaacacagaaagaactattatttaggagtaagaagtgcattgagactccaaagaacaacaaacccaaactacatagtgcaatacacacacacacacacacacacacacacacacacatgcacacacacatgcacagtcttcacagtcttcaaatgggttttactaaggcggggacacttaaaaaataaatataagtcgggcacagtggctcatgcttgtaatcccagcactttgggaggctgaggtgggcggatcaattgaggtcaggagttcgagactaacatggccaacatggtgaaaccccttctctactaaaaatgcaaaaattagccaggtgtggtggtagatgcctgtaatcccagctacttgggaggctgaggcaggagaatcacttgaacctgggaggtggaggttgcagtgagccaatatcgtgccactacattccagcctgggcgacaagagctagactctgtgtcaaatatatatatacacacacacacacacacacacacacacacaaacatatatatatatatatattctcatatatatatacgagaataactgaaaaaactaatgactccaacagtgacttgcccagggagggctttaattgtccccaatatgcatgtgatgctcctttcctatttataatggatcttgtgacagacttggaagaattggcctcttccagttttttggctgcttttcaacaaaaaattttattttcagaatttttcttaattttttattttttttgcaaatatttccagctctgagggagcaggtaaaataatacagctcatatccggaggcatagccagagatctgtggtcaaggggaagaggtcagagccaatttccaaagctgtgcacatttcaggctgcagctgtcagcagcaggaacttctgctgctttcatttgagacggagcactgacaagctctccagagcacatggtcttcagcgggtgcaggcaaatctcaaactgctgatgtccattctcctgatgttacctccaggctcaaatgaggcatgggctggctgtcccagtaagtatgcccatgtaaagtgacctccttggcacagggtttgggggataatggtgctctggtaagctaataccccctgccacatactggcatgtattatatttaggaatgaaggatgccaggatttcagcattaaagaatgaaacgagtaaagaaatggcttcatgtttaattattgaaatgtatactaaattctggggttaaaagtctaagtgcctgaactgaaatccagggactgatggacaaataaactggcagggcttctcttgtgtggcttctcgtgttttctcttatgttggaacttcagtttcagcatctacaaaatggaaatgataattgcatccatttcacagggatattgtgaggattaggcaagaaaatgtaaagccttagcacatacttgtaaatgctcattgtttcactcctttaggcaagaatagcatcttagcatctatgatacatatattgtgcctggcacataagaagcacctggcatatatttgctgaatgaatgaatggaagaatgagtattggtggacacgaactgtttttagcacatctacagattgtagaagaattaaacattggaaccttttctttccctcacccccatctgacttccactgactcacagaaaagtaattctgattacaaaccactctttagctctgattaccccctcccttaacagaataattttttagtaactgaatctggttgtagatataaagtctacagagtttctcacaaacaagccttatcaagtaagtagaaataattgatcactcaccaattttaatatagtggccaaaatgagccttggcctcccataattggagactcattatgacatataattggcaagtaggtggccactctcctggcatgagtttggcagctgactgtggtccttgtccccaaatctccatatctttctccagttcacagagctccccatccacagtgagatgcaagtggctcggtaaatactggttgagtaattgcatgcaaggaaggaagggagggataagggaaggaaagaaagaagaaaatggaaggaaggacaagggagaggaagggaagggatatgaaggaaagcgaagagaagggagggaagtaggatatcagcttctctgcttagtcaggagaagggaaagagcaccagggtactgggaatctaggatggggataggaagagcatagaaaagccagccagacatacaaagcacgctttttagtacctcatacacatcatattgcctcaggctgacccaactataatctaaatataaatgatcaatttgccaggtagagcaagtgctttctaattttctgtttatatttgctgtgtctgtctcccgtaactcagaccttttgcactagacagtacttgtgctcaacaaaatcttcccagttgtttcctctctgattgctttgcttgtagttccctgtctccttttcttctttgtttcagagcttctaatcttcatcttggctctgccccagctgtctcaacagagaattcattggtcatctaatcaaacgagagagtagtccccaccccctcagcctctctcctaactagtatggatcttaggagagctatttttgccagcgaaggtaaagtttctgaagcttccttttacccctgctattgccccgttactccatagtcactgtttcaggctgccttggggcaaagctctgagcctgcctccgtggaactgtcagaggaggcaggagacacaatcccacctctcaatgtgcaaatgtggcatttcgaatttgcaataggatgagtaaatttattttctctgagactttaatgagaccatgtgctacaagtacgaagtgttatcagagttgctattatagctgcagcttcaataatgcagaaaaatcagctatgtgccatgagaatttatttcccaagaatacgacaatgatctctgttgttgggaaaggggggattggtccatgtttcctgccatggtaaataactagaagcttggcctgaatggacgccgaaaccgcaggtgtgtactatcaccaaataaaatccctgtcaaactggctttaatttcataggatcagtccccatattttctttaacttctctgccatagcatatatagatgccatttttgttcagttttgtggcttgagcaaataactatcacttttctcgacagtattgagcagaagggggagacagggaagtatgaagtgtgtctgtgaacaggctgtctcatacacacatccagtgagctggaagacacaagggagaaggacgaatccttttcataggaggtgtgtgtcttccaggttgctggcatcatctctgctgcgattgtgatgatcgccattcttgccctggggaagcttctggaacccttgcagaaggtataaccctgcttctctgcataccgattgcataatttcccttcactactctgctaccagataaataacaggagatttaacaatcatcacatggaaaaccattccctgaataacacagccttctctgtctctcttggcagtcggtcttggcagctgttgtaattgccaacctgaaagggatgtttatgcagctgtgtgacattcctcgtctgtggagacagaataagattgatgctgtaagtcacctaccacctatatttatctgaaataagatttggttcttatatgcttcctgccatatcactatattccccccatcccctaagtctcacttgtgctttgggaactccagaggagaaattagaattgtggggataaatcaaagccatgaagtcttccatgtgaactgcattttgtgaagattctgtatctctgggaaggtcaatttccagcaagaatcccatatgaggctcattcatacacctttggccttctaaacacaaaaagcaaaaaaatgtttaaacaccctagtttagaaggacaaacaacatacaaaacaatgcaaaaacaaaatacaaaatgtaaaaacccacgctgactttaactctgactaacataacctttgcaactattctgagaaactaaaaatcagaagctaaatccttcagagttcctctctaatttaaaccataaaaagaatgagaagacttcctacatattgatatggaaaattttccaagacaaatcgttaagtgaaaaagagaccagaatagaaatgctggggaaaagcacagacacaggcagaaataaaagcggatacaaggagggagggaagttgggaatgggtggagaagggggagggttgtgagcaagagttttcccctttttaatgataattcccttatgaaccatacaaatatattagctattcaaaaatttagaaataaataaaatgggacagttggtcttaattgagaagcccagtttctacagctacttaaatataaaatgaatgaagtcacaatatttttaaaaagctgtgatttggccaggcacagtggctcaggcctgtaatcccagcaatttgggaggcaggaggatcacttgagctcaggaatttgagaccagcctgcgtaacacagtgagaccttgtctctactacaaataaaagattaaaaattagccgggtttggtggtatgtacttgtgggtagtcccagctacttgggaggctgaggcgggaggatcacttgaatccagaagatggaggctgcagtgaactatcatggtgccactgcactccagcctgggcaatagagtgtgaccctatctcaaaagaaaaaaaaaaatgtaatttgtttgtggatcattgatcttatttttataggtagttatcacatgatggtacctgatacattaatataattcttttcatttctatttttttccctaggttatctgggtgtttacgtgtatagtgtccatcattctggggctggatctcggtttactagctggccttatatttggactgttgactgtggtcctgagagttcagttgtgagtaacgtaaaacccagatttcctataaacagaacaacacactctgagcttccttataccattttgataaatatagtgaagccactttctttcgttatagttactgtatattgagtgcttctatgcattaagcagacagtgttttacagacatacttaatcttcaaaaatagcctatgaatagttttgattagtctcattctacaggtgagaaaagagaaattcagaaaagctaagaagcatccctaagatcacacagctagtacgtggcagagctaagatttgaacctatgtacctactcatgtcctctactgctgtgctctccttttagttgtggtaagactaaaaagtacatagtttagccaaaggctgaagttgctttgtagtttcttctttaaatgagatgaggaatgtaaggcgctcggccccaggcttgggacaagacagctgttatatattatagttcattttcttttttaaagctagtaaccaaaagttacataacccagaacaggaggactttaaaatagctgatgacagccaaaattcattcaaatctctcatataaccaataaggcatataaccaataaaccacacaattatgtgaaggaaacaattagctcatatgcagagtgaaggcagcaaagacatgatttaaataatggaaaacagcctgtgagggaaggtgaggagcctgaaggtggttttttggttggttttgttttgattctagagaggccttagttaagtgccctcctgggcctagaggggaacagagtctttctctccctcctcaatgtgtccatcgtctttccagacttctggcccctgctctgcctgtgcttgggccgtgaaagaagaagacacctggaaaaacatcaagatagatgtcttcagcttagcatccgctcacctctcctgtcccaccagccagttctccttcctgccctccttccctaccttggcatattggtgcctacccaaaaggcttgtgttttctacctcagcatcctaaatgattgcccaagggcctgagaaggtttgcaggctcccaaagttctcaatttgcttctaggacacgtctcagaaacgctccttagaagatgctgtctctcatgctgtatatgcctcagtgtctattttttcttgtgtggatgtggttggaaaagaagggttttacagagagagattagcttataaacttttaagttaaaaatcagcacaaacctgtaagccaaggaataactggtggcacattttagctgctagtttggtatctaggcttttagccaacaaaaagcagtatcctccacagtatttaatttccagtaattatcatgctgatccagagagcacaatgctgaaagtacactaaacagatactctttcttcaggaattgtatttagcatgtctctagtcacacatgaagagtcacggaatttatctgattgcatcaggaacaagaaatcaatagtctggttgacaggaaggacttatttctggtcctccagtctcctatttctataaaaaatgactaagtcacaaagaagcgccaggcactggaagacccaggggttaaagacagaggctctgctgccctttacctccaggccttttctcacttctcttaagaagtttagttaacaataaaaatcaggataatactgatgacctttcagggttatggcagggcttaaataagatttcacatgcgtgaaagctctatgcttatcctataacgtgttgtgcacgtgcaagtatgatgctcattatttctctcagccatcagaagagaggcacagttctcccctcccctgccgattccacacaaacaccagctgttcatttcagagttagctacaggaaaatgtcatctgcaataaagacagagtccaaaacaccagaatgatgggctctttagtagctgttgtttttaactttttattccaaaatacggctgttccaaaaaatcttgaccttgatattttttcttctagtccttcttggaatggccttggaagcatccctagcacagatatctacaaaagtaccaagaattacaaaaacgtaagtacctttgtgagacatttgctggacttgggtttactagcctgaagtttcagcagctccattttacgtacaaggtagccaaagggagaaaatgcctattgggaaagtctgttagtccacagggagtgtcatgaaaacttttgatccagtgcaccttctgacacccatggcttatgtgaattttgtctatgctagctgaatgtctttttttttttttctttttagatggagtctcactcttcacccaggctggagtgcagaggcaggatctcagctcactgcaacctctgcctcctgggttcaagtgattctcttgtctcagcctccgagtggctgggattacaggcaggtgccaccacgcctggctaatttttgtatttttagtagagatggggttgcaccatgttgcccaggctggtctcaaactcctgagctcaagtgatttgcccgtcttggcctcccaaagagttgggattacaggcgtgagccactgcacctggctagctgaatgtcattttaatataaaactgttgtaagaggaatttaaaaataataggcaatttctagatgttcatccaaaattttgtttatctggtatataattcttatcttctgtaagattttctttattcagttctaacaagagattatcaagttctctatgaccaagaccttattatttgacactttgctttatccttggtattagaagaaataaaatatgacacaggaaatgaaaaacaaagaaattaaacatcccattgatggacttgtgtcacctgcaaataaaattgcttagtggtgactaggggaaacaaagataaaaaagcagagaaagtaaatcaagaaaatacaaatttagcaaataataataataataatatcaaccctaggaagttgtagtctaattttaccttaacattaaaaaaagttggaccttgaaactgggatttcagtagctgctacttaatttatttccgtgatctttctgttatgttgagccaagtgccattaagagctaaaatgatttagagatttttttggctttggggagggagctctaagctgagttagggaattctgacttctaggctccatcttggtgagactttgcccaagtcattcagcctctcaggacctcagttttctcatctgaagcagctagactaaaattgctgattcctgaagactagaaaagtctatgattctgtgattcctgggttctattttgggtgtggccattgtatgtcagggtgagaacagatgatatcataggcctcagttctgcaaatcccattgattttgctttttttctttgctacttttaaagaatgttagcaaaatcaggagggtcagtggtatgaacttcaggcaaatcatggcactgttctaagcctcagtttcttttctggaaatcagagggttgaataaattgctttctaaggccctttctaactttgaccttctatgagactgtagaggtccaagagtttttgtaacctgttgtctccctattcaagaaaagttgtagaggccgggcacggtggttcacacctgtaatcccagcactttgggaggctgaggcgggtgaatcacttgaggtcaggagtttgagaccaacctggccaatgtggcaaaactctgtctctactaaaaatggaaaaattagctgggtgtggtggcagcacctgtaatcccaactacttgggaggctgaggcaggagaatcccttgaacccaggaggtggaggttgcagtgagccgacattgtgccactgtactccagcctgcgcaacagagtgaaactccatctcaaaaaaaaaaaaaaaagaaaagaaagaaaagttgagtgctgctacccagctcctctgagcaactgtgacttgactccttgctaagtagccagaaatgtaattaaatacttgaggcttgaaattatttaatcccagacaatttcttttaatgccagattgaagaacctcaaggagtgaagattcttagattttccagtcctattttctatggcaatgtcgatggttttaaaaaatgtatcaagtccacagtaagtattttatccctagaaatttgttttctaacctcttttgagacttcattcattctacaagtatttactggggtccaatcaggaataggccctagaccctcttccctttgtgtagggcaatgagaattaaaatataacatccttgccttcaaataatttacagtctatttggggattaaaaaaacacatatgttaaaacccaggtagtaataactatgccagacaaaaacattagagatgtttctaggcagaatttgagtaagtttcaaataagtagtatagacagttgagagttcatagaaggaagagatccaggttagttggactaacaggctttgtgcggcagttggctgaaggaaagaaatcatgtcaagtgtttgagaactatgtgagctgagagacagtggaaacatgtattggaggtagggttgcccagttgcagcaaaaagtaaaatatactagtggaataaataagtagaaaatcctggcaccaaatgccatgctatgctttgaacttggagtttgcctctaaatcagtgtttttctaagtgagttccagggcaaactgattcagaggaatgttcacagattttgcaataacaaaagggctctgtgataccaagggaatttggaaaacccagacttaaactaaagagggtattttggggtgtgtgtgtgtttctgttttgtcttttactgtcttggagcctttgatatgctactgtcttctcagagcttctgggtaggagtagggtagcctgggagtagacagagatctactccatcagaccttacaatttcttttttggcaggatagctcaaggaattataccctttgagaaatagcctttccagataacagttgccattaataagctttaggtgccaggcattttaagtaacttgacatttatttccaaaggttggatttgatgccattagagtatataataagaggctgaaagcgctgaggaaaatacagaaactaataaaaagtggacaattaagagcaacaaaggtgagatgacatctttcttttcccccttaaattatttcctttccctgatgagagcagttagagggtctaaaattaaatctatcctctttagtatccagatgtgaatgaacaaatgacatgtacgtatcaaagaacaactgagctattcttataggcaagcgggagtggaagggaatgaaataaaatcagcagcgctgcctggaaacacaggatgtgatttttttccccaccatgaacagtgtctgcattattttctatactttatttttactgaaaggaacaaaataagtgcaaattcaatgccagaaaactacccaccatagaaggcagtctaagtccataaacttttggagctgttaacttttactcagattcttctggccagtgtatttcttggcaaagttccacaatcatccagaaaacaaaagtttcctggctcctctgttctcccagttttcttcctagacaacatcaaagtttgggctgaggtgaaacccatccttaaaaattcatctccttgatgtcttgcttaccaaggaacagtgtgtaggtcttttggataatttgatatgaatggttgaaagatttcaaatctttgacaattaagttgacagtgttttcttcgtttagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtggtaaggttctggttttctgaattatacatttggagctttggcaatagtaaaatgatgtgggttgtccagtattgcaacagggcaaatacatgggctttgtaatttttctaggtgaatgcttttgtaaaaaagtgtaatattttaaagcataggctctggagccagactacctgggggagatactggcttcaccacttactagcagtacgaccctgggcaagttgcttaatctgtctatatctcagtttcttcatctgtaatatggaggtaatgatggtatctaccttcacaggttgttacaaggattaaataagctaatagatataaggtgtttagaagagtgtctggttcaggctgggcatggtggctcacgcctgtaatcccagcacttcgggaggctgaggcaggtggatcatgaggtcaggagttcaagaccagcctggccaatatggtgaaaccccgtctctaccaaaaatacaaaaattagctgggcatggtggcgcacacctgtagtcccagctcctaggaggctgtggcaggagaatcgcttgaacccgggaggtggaggttgcagctgagattgtgccactgtactccagcctgggtgacagagtgagacttcatctcaaaaaaaaaaaaaaaaaagaatatctggttcaaagatactccccagcaaattaattcacatttattatgttttatctttctgagaatgtataacaagtggtatatgaaaagaaaaaaatgggcaaaagtttattaagtattacatttctatttgttatgttaacagcaacaggataaggaataccaggtgtatgttaggactggaaaagccaggcattattaagaggttagagtaggaagcaggtcagacatctggaaggtcaaagcaagagttgaggagtatgcaggatagaaacattaatgatatcagaaaccagttatgcaagctgaagttcattaattctgtcaacaaatagatgtgaaatgcctaatgtgtactgagcactctgccaggcacaagagactagtgatgaagtagacacagtttctacctacgtgggacttagcagtctggaggagaaggcaaacattaagtcatcttataaataacaattgtagtaagtgctccaaagaagatgaataggacatcacctgaagcatcaagttcagtgaagcttcaggtaggtatcaaaagggtcaggcagaaaagtaagcaggtcaagacaccaacaatcagatgtaataatataaagctgactcctaactgaggttccacttagctaaggcttctttgctacctctataaggggaggaaatgattccaggatgtgggtactaggtaggtagggtagagaagtgattgtgcttggcaaaagagtatgagaatatgctgacaggttctgttctagttctcttagcagattttgttattagctcatttgaaatcactcttgttctttgttgtacatttcactctaagcattggctgagatccattgtagactatattggccagagagttcaatgaattacttttggggacagtcttaacatatttaatgccagaatatttcccacacaacgaatatttactttcaaatattataatgacagttatgatagctaaagaagactccaaactttatacttgaacagaggtcttgatttgttttcctccataattctcaaattctcttttattattgactatgggtcttgtgtcttgactttttaaaattaaatgtttattttgacataattgtaaattcacatgtagttgtaaggaataatatttagatcctatataccccttacccagtttctcccaatgagtaacatcttgtaaaactgtagcataaaatcacagtaggacactgacattcaaaatagaggccttgacttttaggtcaatagtaacattttgaacccgataagcatagccacacccatatctcttctccaatcagcagggcctatattaagaagcctcctgtactctcttctttccacaaatgcttatttagcacctccacgctatcaagtgctgtgctattgagcgctggatgttgccatctcttgagatataatcctaattataccattactgtagtttgaaaacctccatggttttgcaataataacctttccttaaagtcctgattaaccatgaaagtaataatgtttctcctgagcaagtaactgaatgctactgaattatgggcagataaggttgttaattgttacaaactctccttttttatttttagattgtcaaagaattccaaagaattgatgtgaatgtgtattttgcatcacttcaaggtaaatacatatatctacatatctacctgtaagactttcccgtaagccctttctcctatctgggactgtggtcacattatgtctgaaggccttttttttttttcttttaaagatctcaattgtcattatttgcagttctggaatctggcactgcttcattccataaaacagaataagtgttccaaggaactaagcaggagttcagtattatacacagaaaaggccaaagacagcaaaagcaagcaacaaagagtatattagtccattcaaagttactttcttgtaagtcagagacagtgagacagaacaataggaaaataatggattagttaacatgaggtcatgtcaggatacttttttgtatgaggattattgcagaggaagctttattattatgccaactggaaatttaaactgtcctgttttaggaaatttgctattacctctctctcctgatttcttcaagtatcagataacaatttagtttaggtttgataacttgaaactttagcatgagtgactccattttgatttttagtcttgtctgttgtcgtctagtgcgggagcttagtctaaaacaatggccttttatttttattttttattttactatgtgcctgtgccttgcatgaccccaggatgggcacagctgcttggcccctgagccaccgaaggctgctctactccttcgttctggctactaaaagctgcaaagctctgggagggcaaagctcaagaagactaaaacaatggcctttaccatttttgtttgatgattttccccgttttgatcaggctctcacctaggtaagagtgaacaaaatgtaggagatcagtgctactctgttaccatcattttgggtttctggtctcaggaggtcatgtgtagcttatggtgccctcctcattatcatgtatgtctctgagtttttgttgtttcagagagagaccattcgatgtctgacaggtggctttttggaaacatttaaaactttgagagggtataatgtaccaggaagactgctattatgactatcagggagataataccaagggtttatagtatgctccttagccaggattctcatgaatcaaaccaactaaaattgaatagcctgacaaggagactacctgttttaaccaagaagtcttctctgtagtacctgatgtatttatatatgtggaacaagaagtgtcacccaactgcacagatgcttccttgttgagttagcaggtaatctagcattccatgactggttaaattaaagcagaaagtgtaagttacccaaagaagctactcattgtgaagttttaactacagcactatcctgccaagtgaaagaggtaggcacaagtaagggaaaattaaaagggataagcatcttatgatagggagtcttgttctgacagtcttggagctgtctacagcatgaagttgacaacttcttgttttggtttgtagttttattgtctctagttgtggcatccagcattctggcgaactctctatgtggcccacagtttaagcatgagattcatctcttgaaatttacactgagttgctcagcttcagcttacagagcttcaggaactgagtagttcttggtcttcattggagtgttgtagccagatattagtgaaagctaaaagaattaagaatccagctcagtctacaggtagataataaaaactcataaatattgaacagggctacagtctaagaacagttttactatgcttttcttttgaaccatatgtttttctttctatagtcacctccatttctatcaaagacgatcatggtagaccaatttgtttacaaaataagtttggtctcaaacttggcctgattgattatttacacagtacagcaagaataactacataggtgactcctttcaaatttgctttgccaagcctggcgaggtattgcttgcctgtgagcccagctacttgggaggctgaggtgagaggattgcttgagcccaggaattcaaggctgcagtgcactatgattgtgcctgtgaatagccactgcactatagcctggacaacacagtgagaccatgtctccaaaaaataattgctttgatagaaattttgacaaggaatctcagattggactttttaaaacgtcttgatgctatgaagtcaaaccaaggcagacattaggctttgcctgatgtatctaatatcttgaagttactgggcctcccaggaaggaacgactttttattcactcattgtaaggctagcagcccttgaagccaggaattctgtgcacattttcaaatatgatattctattcaaagccttgataatataaccaatgttttccaattgtattctatttaaaagaacagattctattgaactttcatgtaaataatcatattgccataaaaataagaatactcacaaagagtttccaaattctggaaggatcaggtagagaggaaaagcaaatgtttcaatttttgtttatgaaagtatgcttaacaaggctgggtgcggtggctcacatctgtaatcccagcactttgggaagccaaggcggacaattgctttgagctcagagtttgagaccagcctggcaacatggcaaaacgccgttactacaaaaaatacaaaaaattagctgggcatggagcctgaggctgagaatcacttgagctggggaagtggaggtagcagtgagcccagatggtgccactgcactccaacctgggtgacagagtgagaccctgtctcaaaaaataaaaaaaaagtatgcttaaccaagtggctgtaaactgcagatagctttaaagaaaaattttctttagatctggaaaacaaatattaaaagaaccagcaatgtttcaaataaaaaagccataaaacctgtaattcttctccatcagttcattcagtctcatgtaattaattcttgctctgtttgatcttggctggtagtttaattccaacgaatggtatgaattcaaagttattagaaacctgtatttgtcagagttcttttcattcttcccatataactctttgaagtcacagcactttagaattataatggcttacaaagagctttcagaaaaagtatcagaacaaaacaattaactgtggacaacaagacttaaagtggctatatttaaagatctgatgtgagttacccaattgacaaggatatttggatatttctgtggcacacaacaatttaaaataaccaaaattatgactggtagcatttataccaagacctatcacatttctaggaatgttatataattttggaacatattaataacatatctataaaaatatagcacaaagaaagttaaacatcatttcttattttaacagtgcttcccatataatttaacctatcagataaggccatttggtttaacatctctcttttacagattctttaagaaattccagggtcctctggaacatcccaaagttagttcaaggtcaaaaagacttaattttgatttttgagaagtttgtcaaataccaaaggtttaaaacatttgatcaaaatcggatcataggtcactatgaaataaaaccaaagtgaaaaaagagttcaaaggcaaaaagcacaagaagagttatattgatgaaacatgaaatctctgttttctaggccagttacctggaagagaaaatcctctcacaattttctattaagagtaaaccaatcctctgagaaaactctattgttccaacacataggcccacactttagccttccatcagtgtactttaatattaatgctcaatttttagaaaaacttataaataattcccttctacttttagccaactcaatcacataaaatttttcatgatatttatcttctacaaaccttctacaacttgcttaaaccttcatttggtcctatacttccttttttaaaattggcattgtaccttaggacaaagatttacttttcttttctccttatcattttgaccatataaggttatctcctatacaaaagaaaaaattactctcttttcaattttctttatctcttcatacttgtaaatttcttctcacatctttcctacctagtggttccttcctgccttgttttgatttccttcataagtccatatttagaaacaacctttaaataacctctgattgccaagctagagttaagttttaaataaataaataggccggtcatggcggctcacatctgtaatcccagcactttgggaggcaaaggtcagaggatcacttcaggccaggagtttgagaccagcctgagcaacatggtgagaccccgtctctattattttttttttaaaaaacaaaaagcataaaaactctgagaacattttgaacccagaaagatattacttccaatttgtgccacagagctggtaatgtatggaaatgtgtatcttgacagtgggtgggggtggggtgaatattgacaatggggggatgttgttatgaggagagcaaagctggagattacttagattccttagaacaggaggactctcaagggtgtgagtctctgtgtgtttctttatgaatgtatgtgtgtaggaaagtgctaaggcttaggttgaattctttagcagcagagcccaagatggggattcttgcacaagcgatttattgagaatacatctgaggaaatttatgaagcagtgaggggagcaggatagggaaggggaagaagctgatcaaagacatggtttctgaagtccagcgtagatccctgatttcacagggagcaccaccacaggaagagtaccagaaggttatgcctccttgaggcaagaggacctgctttctcccccagtaatgagtcagtcagtggctgcaggccagccatgtgtggggatgaaacctcccagttctctttatttacttatttatttgagacagggtcttgctctgtcacccaggctggagtgcagtggtgcaactgtggctcactgcagcctcaaactcctgggctcaagtgatcctcccatctcagcctcctgagtagctgagactataggtgcacaaccacacctggctaatttttgtatttttttataaagatggggtttcaccatattgcccagaagttctgggttcaaagtgatctgccctcctcggcctctcaaagtgctggaattacagacccagtcctctttagatgacacagctcccaacgggcaagggaaagtctccagagaagggcacagctgtgaaacattggcagcctggcccacagcagcaggaggatggatacactggcttggccaaagggacgtgatcgtccacaaggttgactacgaccagttatgggataaccattctatatactactggaaaagaaactgtcatttcaaatctgggtcacattttggataagagaaaaataaataaataaaaggaggagctacaaaaacttagtataggttgatgctttaaaatttcttttcttagctgggcatggtagggtgtgccctgtagtcctagctaattgggagggtgaggtggggggatcacttgaacttgggacgcggaggttgcagtgagcaatgatgccactgcactccagcctgggcaatagaatgagactctgtctcaaaaacaaacaaaaatttcttttcctaggaactaacaaaacattgtgtctttcttttgaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacggtaaatattcaacctttctacagatgtatcttttctaaactatcatgatttctataaatggcaaacattacacaagtctagtctagctgttgaattttaagctacctatataacttcatggagcctcagttttttcatcagtaaaatggaagtaaaaacattaaccttgctaggtagatatgaagactaaataagatagtttataggaaattacctggtagtacccagtactgaatagttctacaatgtgtaggtttattaataaaagtgggcattagctataatgccttttaagaatattgaatcttggatcttttgtgatctgggatttgtgtacataaattccacttaaattctcagcaatctggaaaaccttgaattataaatgtctttaaacaggatccaccaggccataagtgatctttgaaaaagaaatttaaaaatcaagccacaaagaagcccagggttaatttttccctacaaaatagaataggccctgatgggaggcctcatggcacaacagtagcgcatctggctccagaataggccctgatttcccatgggtattattttgttaatttttctgtcaaatacccagaagaggtgagcattctggtctggttcattgggcttggcctaatggtgctcctggtggtacaagaatctcatttctcatagtgaatttaggaaactaaagcaaatacctatcaagatataataacagtaatgattccagtgacatcgtggatattttaaattgcttcccatttttctaaaagtagaataagtagatccttattgtagattggcagttgattgaagaaaactaacttaggaaatgatattatattattctttttaggcactggtagtggagagattgttctatttgatgttccttggtaaaatactagtcctaactaccaagctgtggatcttaaatataaaaaaggaaaaaaaaaaaagagtaaagctacaaatctaaagaaaaaaaaactgtttatcattgaaaattcataaactttttctatccaacttaaaattctctaacccataggtgataagaaagttggatcatatgaagtatcctttaagagatccacattaaaagtaaaacagaaccaaggtatcaagttctttctcaacatgaaagcagttattttatttgtttatacccattcttactaagattttaggctatgaatgtcataaaaaccactaaccaagagtgtatctactaggttagactcactcttgtttgcaatcagagaggtattgtgagtggctgggatggatttttttggaccttaaacaaatagactaagcataacggaatgcttgtattacatacttactggtccactaaaagtgggttggacattcctggtggcctgtgagatcaagaggcttatatttctgtgtcttctcagagtctagcacaaagctttatatgtgataagtaggtgaatgatgataaactccttcaagagggagtccctctgtatcaaagatattgtgactgcaatgacacaaccttcttcctatccctttattttaccatcacaggactatctcaggacacaacttgtagttagacttatcctctacctttcccattatgtcataccatttgcacagggacttaaccctctaattcattctcatactagctaagagaattgggctatttgtgagttgaaaagtagctaagtagcatttcaaaatgttattttagcctggatatatatttggagttgctttgaaaatgtccttttccatgcaaaacacaaagccaaaattgtcgttggtttcctctgtgtagaagattaaattacatgccacctctaaacagtagagcttttctgaataaccaacttggtccatagacattggtttccatctccaatagaattaatttccacccaattccatttgtggctgttttttgtcatcagtgacaagctcttcactgtgcattcattgcacactcaacgctgtgctaagtgctcttagcttagccattgagagatgcactattgactgctgaatcatttaggcagagggggtgacttgttaagaggcatacactcaagaggttggggaagagagctgaaaaggagtttacacaatggagaactataccagttcacctttcaatgtgcaaaaaaatgatatacaaaaaattttagttgggaaatataaaagaacaatacagctgaagaggattctgaagtatgtaaagacagatgagaagcaccaggaaagcttcaaatcattttcagtggagcatcaggtgggttgatgctattctatttctaccctgtgttctctttttcaagatcactctcattcaggattgtaaagatacccttgaattaatagaaacagagctgacggaagaagaacttgatgtccaggatgaggtatgatcattttcttctgaagaaaatatttgaattacattttgaataattagagtaatacaaatagtgaatatatctgattaagaactgtcagggaacataattcccccaaatgcagaaaagatggcttcatagcagggaaaaagagaaaataagaattctcccttgaagacattaagtacttggggatcttttgtcccaggcatttggggatgatcagcaactggcaagagccaacacactgctcctgatttgatacctacacctctgcatactgctcttctagagatgtaaggccatggggtaggaaaaaaatgaatagaggaaacttctaagctaggaactttataagcctcaggaatagtatgtgaggaacagaaaccctatgaccttccttcatatgactgataaaggctttaaaaacacactctccccaccctagactgattctgtgtatttcagatgctcttgtgacatcagactattcctaggaaacttgtctgaacttgtagcatctgagtaaattttgcagcttcgccactggataagaggtgaacactgaaggaagatcacagaattgtaaaaagttgaaagatttcttagaatcaagtagtctaacattctagcccatgcagaaatccttcctacaccatccttgatgaatgttcattcagcttttatgtgaacatttccagacattggccactagcggcttctcaagggagtttgttacatggttggaagtctctaaatgttacttgtgcttacagaactcaatttggcctttctgtagtaaccacctagtgaactgagatctgccttccttagcaacacagaccttcccactgaacagcccttcaaatatttgagaatagttctcatgtctccagcagactctcttccagttgagactcttctagttccttctattccttacaaacaagcagtcagtgcttctctggtcacgatggggacacaaaaccctaaatgcggccaggccagcatggagattattcaaccatgacctaccttgatctcatcaagatattcctattactacagcttaaaaatgtgtttcctttcataatagcaacatcagaattttagcttctattgagcaggggccaactaaagactggggcattttcatacaagctatggtctagtagagttccctagttatatttatttaattgagctttgaccctaatacagaactacacctatttctgctggatttcatcttgttggttcttgtttactgacataattttgtattattattagtattattattattattattatttgtgactgagtcttactctgtcacctggactggagtgcagtggtacaatctcagctcactgcaatccctccctcccaggttcaagagattctcacgtgtgcctcagcttcccaagtagttgggattacaggcgctcgccaccacgtccagataattttttttttttgtattcttagtagagacagggttttgccatgttgtccaggctggtcttgaactcctgacctcaagtaatccacctgccttgccctcccagattgctgggattacaggcatgagccaccacattggccagttttgtgttttgaatctaacatccaggatattgtctaccccagtacccgttacctggtgtcataatcaaatctgataagcctgccttctctgcctttttctaggtcttgaaaaaatgttgcaaagaaaacattcaaccccctatagtgaaatcaggcagtaaggatgacaggcagagatttctgtggaggaaatacattgctagagtgaaaatcacagtacctgcatcttggcccaccagcttagtgagcccaggaaagtcattatcgacctgagaagtccttttagctccagtagtttatgactctatggatatagggtgccttagtttgagtaaaaactcatatgtcaacattaaaacttaagactgctatattataggatatttattggttgccacgtaaaataatcattcagggagggtttctatagatggtaaaacaatatagcacaatagtttcaagggcatggactttagaacaagacagacctgggtgctagtcctcattctgctacttactggctgtgcaccccagggcaaattacttatcctgtcttcatattagtatcttcatgtaaagtagtaataataccacagagttgtgaaaattaaaggataaatgtaaagtaacaagagcagtgccttgtacatagtaagtgctcaataaatagtaatcattgctcttaaataaatccaattaaagtgtgatgaatacaggccaggctcagtgactcaagcctgtaatcccagcactttgggaggctgaggcagggggatcacttgaggtcaggagtttgagaccagcctgactaacatggtaaccccccgaccatctctactaaaaaaaaaaaaaaaaaaattagctgggtgtggtggcaggcacctgtaatcccagctactcgggaggctgaggcaggagaattgcttgaacctgggaggcagaggttgcagtgagctgagatcacgccacagcactccagcctgggcaacagtgagtgagattcagtctccaaaaaaaaaaaaaagtataatgaatacgaaatgaagtttttaccctatttctattgtgatgatatacacctaagatgagtagcagtaagcaatcaatactataaaaacatatttataaaaaagataatgcagacttaaggagaattcagttgtatcaacactttgttttccccttgcttccacaggctatgcgtacacttgcatcctgaaagtgggttcgggaggtctctatgagcaaggaatacaagacaaaacttcctcaatgcattgactatttcttcagactcaaaacactcattcttttttctattaagccattgaaagagaagcactaagactgcttctaggctttatttataaaataaacaccttatccctaacatgggcaaaatggctagaattattcagacgatttggcagcgtccagggtaagctggtgttataatacgctgctgatctacatcacagatttgctaataatgttcacgtgggccctggcatatctctgttcagttagagtgagtgctgacccaacagcctctgtggtcaagcgagtcacgaatgattaatcataaagaaaaatcagtttttgactgacctggatatccatgagctgcactgatcaccatgtaaggtcacatttagtaaatgctgaaataaaatgattaatgcatttatcaataaaagcctttgaaaatactttggataataaattggagttttaaaaatgcaaatttgcttagtatctaataatgaagtgttattacatatagccggaattgaggatctctttgatcctggaaatggtttacctaaaagctacagaaccaggccaatatattttgaaatattgatgcagacaaatgaaataataaagagattttcatggtttataaaaatcttttttgatatgataataatcatgatcacaactgagatcaaaaaaatatatgacagattattttgtttaaaaatgcagttttaattatcttagtctatagaaatgatcattgcatggaggcatgtataggtatgatctgtgtaaaatctgacataaaaacagtgctattctgagtgaaaatttttttgatgtgcttacataaccatggtgattaaaatgagtttatattttttctcaaaaattttagcagtgtgtaaagtaagtaatctttaactgaactctgaccacttaaaaaaaaatctaaaaattgaactacctatagtagtctgtgtttaaagtgaatttttaaagacaaagcattctaaatgaactcaatataaaaacattcatttggaatgtacatactgaaaaatacaggtttttttgaccaaaagtttttatatcttttctttttatttatttttttcctaagtgccaacaattttctagatattatatacaacacaggctttgatcttggggacttttcccatatatttcacactggagtgaatgaagttgtacttcatttctagagaaaagttatacccaggtccccaattgagaatgtcttgcttgattgaaaacgacatcatcccttggtatactccagggattggtttcaggacccctgcatttaccaaaatttgtgcacactcaagtcctgcagtcacccctgcctaaagatagaatggcttctctgtttttcttctgaaatacaaccagaaacaatgtgtctatttctgaaagaataggattaatgatcatacaaatgggttaatcctgaattctggttgtaaatctggttacagcataactaggattataatgctgcctcattttcacagcactacttgcttatattgacaacaaatcatctcgctaaagagtgaatgtaggccaggcgcggtggctcatgcctgtaatcccagcactttgggaggccgaggcgggtggatcacgaggtcaggagatcgagaccatcctggctaacatggtaaaaccccgtctctactaaaaatagaaaaaaagaaattagcctagcgtggtggctggcgggcgcctgtagtcccagctatttgggaggctaaggcaggagaatggcgtgaacccgggaggcggagcttgcagtgagccgaggtcgtgccactgcactccagcctgggcgacagagcaagactccgtctcaaaaaaaaaaaaaaaaaaaaaaaaagagtgaatgtaatagtcttgcagaaaatgaatgaatacctttgttcaataaaggaaatatgcactgctcacttttttgaaggaaatgccaaagttacgttttacaacaaggctagagtttgtaaattctgggttcatttgtgatgacataagtcagcaaactgcgggaatactgtctcttctatgtattttgtgaatagtaagcataattttagttttgtattatcaatgaaaatttcacttgaaattaaagctgccttttgttatatttttaacctataggataagattccagtattgtatatgagttttaacaaattaaaaaatcaaatcatgtacatttgaaaatatttgcacacatttaaaaataaatgtaaagttgtcttttaaactactcggatgtgtcctttctgaacaaaactattaaatataataaagaatgtgccaggagcagtggtgcatgcccttgtagtctcagctacttggaagcctgaggtggaagggtcccttgagcccaggagttagagtctcagcctgggcaacacagcaagaccctgtttctataaataaataaataaaatataataaaaatacttcattctaagtttgtcattgctcttatcagtcatcattatgaggcattctttatagcaccacttctgtttataattcctttgtgtagatttatcagtgcaataaacaacattactcacaaatatcacagttaaaccttgtgattcttatccaaatatttccagtttattttgtgcaatggttattattattttaagcaaacttttcattctggcacaacatatgtaaaaaaggatacaaatcacagtgtactgcttgaagaattttcacaaaacaagcacacaagggtggcctgtgcccagatcaagcagcagaatgccaccggcaccacataaactcttcatgctccctcttagttggttgccccaaagggcaattactgtcctgacttttaaaatctgttttttttgaattgcatagaaatggaatcaaagattatttcttttttccccctttcttttgtgtctgtcctctttcactcaacattataatcaagagattcatccatcttattcaatatagcaatcattcattcattctccctgctgtatagtattcattccattgtagaatttaccaccacttatgtattcatcctacagtttgtggatatttgtgtcgtttccagttttgggctctcatgaatagtgctactatgaacattcttgtgaggcatccttgcacattccagaaatatttggtggggtggggttcctctatctggggcttagatctgtgaggtgagaatcaatgaaattagggttattttccatttgcatcaggttaggtatctgaagaaagtttggtttcagttagagtagcatttatcatgcaaacaatttaacagtgtaacaaaaaccacacaaactttagttaaatcacatgaagttcagttctgtaagaaagtcacataatgacaaatacctagaaaaagagtggaagagagaaaaagtagaaacaatgaggggagaaggaaaggaagttcagaggggaaagtaagggagaaaaaggtaagagaaccacagagaaaaagaatgacaattagggaaggaagagaaataagaaagggaaaattaacggaagaaagaaaagacgaaggtaaaataaaaggtggaaggaagagaaagactaagaagaagaaaggaatggtggcgtagagaatttcataatgggaaaataaaatccaaatagaggattgatttcaatataatcatatccttcgagaactcagagaagtgagcaacactccctcccccacatgccacaccatctcctgaattcttggtcccccttacccagctcttttcattaatatcagcttctagtatgctgtgtaattcacttattactgtttctcttcctgccgcctcgcttatactagaacataagcttcaccaaggcagggttctttattttgttcctggacatataccaagtattcagaagagtaaagagtgtcagacacacggtagaggtgcaataaatactctcagatgaatgcattaagctaaagtcagtgatgctgagtaaggttaacataatttgagaatggcagtcattaagtacataagacattcagagtttcctgagcttgcaggcctattccaaagacttactgtgacagctcacctgcttgcctgtctgccttacctattggtctccagaaaatggcctgggaattcacttgtatgttagcagagcttcagggaattaaaggccctttaatgtctatatccattttgaatatgccaaccaagtcagcccacttcatccttttcttcaggaacatttttgggcgtgaacattgaggtgctgtttttgcataa

SEQ ID NO: 4 - DNA, 4928 bp, Homo sapiens
ctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtcatggcagcgccaggcggcaggtcggagccgccgcagctccccgagtacagctgcagctacatggtgtcgcggccggtctactcggagctagctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcctaaaagtgggttcgggaggtctctatgagcaaggaatacaagacaaaacttcctcaatgcattgactatttcttcagactcaaaacactcattcttttttctattaagccattgaaagagaagcactaagactgcttctaggctttatttataaaataaacaccttatccctaacatgggcaaaatggctagaattattcagacgatttggcagcgtccagggtaagctggtgttataatacgctgctgatctacatcacagatttgctaataatgttcacgtgggccctggcatatctctgttcagttagagtgagtgctgacccaacagcctctgtggtcaagcgagtcacgaatgattaatcataaagaaaaatcagtttttgactgacctggatatccatgagctgcactgatcaccatgtaaggtcacatttagtaaatgctgaaataaaatgattaatgcatttatcaataaaagcctttgaaaatactttggataataaattggagttttaaaaatgcaaatttgcttagtatctaataatgaagtgttattacatatagccggaattgaggatctctttgatcctggaaatggtttacctaaaagctacagaaccaggccaatatattttgaaatattgatgcagacaaatgaaataataaagagattttcatggtttataaaaatcttttttgatatgataataatcatgatcacaactgagatcaaaaaaatatatgacagattattttgtttaaaaatgcagttttaattatcttagtctatagaaatgatcattgcatggaggcatgtataggtatgatctgtgtaaaatctgacataaaaacagtgctattctgagtgaaaatttttttgatgtgcttacataaccatggtgattaaaatgagtttatattttttctcaaaaattttagcagtgtgtaaagtaagtaatctttaactgaactctgaccacttaaaaaaaaatctaaaaattgaactacctatagtagtctgtgtttaaagtgaatttttaaagacaaagcattctaaatgaactcaatataaaaacattcatttggaatgtacatactgaaaaatacaggtttttttgaccaaaagtttttatatcttttctttttatttatttttttcctaagtgccaacaattttctagatattatatacaacacaggctttgatcttggggacttttcccatatatttcacactggagtgaatgaagttgtacttcatttctagagaaaagttatacccaggtccccaattgagaatgtcttgcttgattgaaaacgacatcatcccttggtatactccagggattggtttcaggacccctgcatttaccaaaatttgtgcacactcaagtcctgcagtcacccctgcctaaagatagaatggcttctctgtttttcttctgaaatacaaccagaaacaatgtgtctatttctgaaagaataggattaatgatcatacaaatgggttaatcctgaattctggttgtaaatctggttacagcataactaggattataatgctgcctcattttcacagcactacttgcttatattgacaacaaatcatctcgctaaagagtgaatgtaggccaggcgcggtggctcatgcctgtaatcccagcactttgggaggccgaggcgggtggatcacgaggtcaggagatcgagaccatcctggctaacatggtaaaaccccgtctctactaaaaatagaaaaaaagaaattagcctagcgtggtggctggcgggcgcctgtagtcccagctatttgggaggctaaggcaggagaatggcgtgaacccgggaggcggagcttgcagtgagccgaggtcgtgccactgcactccagcctgggcgacagagcaagactccgtctcaaaaaaaaaaaaaaaaaaaaaaaaagagtgaatgtaatagtcttgcagaaaatgaatgaatacctttgttcaataaaggaaatatgcactgctcacttttttgaaggaaatgccaaagttacgttttacaacaaggctagagtttgtaaattctgggttcatttgtgatgacataagtcagcaaactgcgggaatactgtctcttctatgtattttgtgaatagtaagcataattttagttttgtattatcaatgaaaatttcacttgaaattaaagctgccttttgttatatttttaacctataggataagattccagtattgtatatgagttttaacaaattaaaaaatcaaatcatgtacatttgaaaatatttgcacacatttaaaaataaatgtaaagttgtcttttaaactactcggatgtgtcctttctgaacaa

SEQ ID NO: 5 - DNA, 4928 bp, Homo sapiens
ctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcctgaaagtgggttcgggaggtctctatgagcaaggaatacaagacaaaacttcctcaatgcattgactatttcttcagactcaaaacactcattcttttttctattaagccattgaaagagaagcactaagactgcttctaggctttatttataaaataaacaccttatccctaacatgggcaaaatggctagaattattcagacgatttggcagcgtccagggtaagctggtgttataatacgctgctgatctacatcacagatttgctaataatgttcacgtgggccctggcatatctctgttcagttagagtgagtgctgacccaacagcctctgtggtcaagcgagtcacgaatgattaatcataaagaaaaatcagtttttgactgacctggatatccatgagctgcactgatcaccatgtaaggtcacatttagtaaatgctgaaataaaatgattaatgcatttatcaataaaagcctttgaaaatactttggataataaattggagttttaaaaatgcaaatttgcttagtatctaataatgaagtgttattacatatagccggaattgaggatctctttgatcctggaaatggtttacctaaaagctacagaaccaggccaatatattttgaaatattgatgcagacaaatgaaataataaagagattttcatggtttataaaaatcttttttgatatgataataatcatgatcacaactgagatcaaaaaaatatatgacagattattttgtttaaaaatgcagttttaattatcttagtctatagaaatgatcattgcatggaggcatgtataggtatgatctgtgtaaaatctgacataaaaacagtgctattctgagtgaaaatttttttgatgtgcttacataaccatggtgattaaaatgagtttatattttttctcaaaaattttagcagtgtgtaaagtaagtaatctttaactgaactctgaccacttaaaaaaaaatctaaaaattgaactacctatagtagtctgtgtttaaagtgaatttttaaagacaaagcattctaaatgaactcaatataaaaacattcatttggaatgtacatactgaaaaatacaggtttttttgaccaaaagtttttatatcttttctttttatttatttttttcctaagtgccaacaattttctagatattatatacaacacaggctttgatcttggggacttttcccatatatttcacactggagtgaatgaagttgtacttcatttctagagaaaagttatacccaggtccccaattgagaatgtcttgcttgattgaaaacgacatcatcccttggtatactccagggattggtttcaggacccctgcatttaccaaaatttgtgcacactcaagtcctgcagtcacccctgcctaaagatagaatggcttctctgtttttcttctgaaatacaaccagaaacaatgtgtctatttctgaaagaataggattaatgatcatacaaatgggttaatcctgaattctggttgtaaatctggttacagcataactaggattataatgctgcctcattttcacagcactacttgcttatattgacaacaaatcatctcgctaaagagtgaatgtaggccaggcgcggtggctcatgcctgtaatcccagcactttgggaggccgaggcgggtggatcacgaggtcaggagatcgagaccatcctggctaacatggtaaaaccccgtctctactaaaaatagaaaaaaagaaattagcctagcgtggtggctggcgggcgcctgtagtcccagctatttgggaggctaaggcaggagaatggcgtgaacccgggaggcggagcttgcagtgagccgaggtcgtgccactgcactccagcctgggcgacagagcaagactccgtctcaaaaaaaaaaaaaaaaaaaaaaaaagagtgaatgtaatagtcttgcagaaaatgaatgaatacctttgttcaataaaggaaatatgcactgctcacttttttgaaggaaatgccaaagttacgttttacaacaaggctagagtttgtaaattctgggttcatttgtgatgacataagtcagcaaactgcgggaatactgtctcttctatgtattttgtgaatagtaagcataattttagttttgtattatcaatgaaaatttcacttgaaattaaagctgccttttgttatatttttaacctataggataagattccagtattgtatatgagttttaacaaattaaaaaatcaaatcatgtacatttgaaaatatttgcacacatttaaaaataaatgtaaagttgtcttttaaactactcggatgtgtcctttctgaacaa

SEQ ID NO: 6 - protein, 780 aa, Homo sapiens
MAAPGGRSEPPQLPEYSCSYMVSRPVYSELAFQQQHERRLQERKTLRESLAKCCSCSRKRAFGVLKTLVPILEWLPKYRVKEWLLSDVISGVSTGLVATLQGMAYALLAAVPVGYGLYSAFFPILTYFIFGTSRHISVGPFPVVSLMVGSVVLSMAPDEHFLVSSSNGTVLNTTMIDTAARDTARVLIASALTLLVGIIQLIFGGLQIGFIVRYLADPLVGGFTTAAAFQVLVSQLKIVLNVSTKNYNGVLSIIYTLVEIFQNIGDTNLADFTAGLLTIVVCMAVKELNDRFRHKIPVPIPIEVIVTIIATAISYGANLEKNYNAGIVKSIPRGFLPPELPPVSLFSEMLAASFSIAVVAYAIAVSVGKVYATKYDYTIDGNQEFIAFGISNIFSGFFSCFVATTALSRTAVQESTGGKTQVAGIISAAIVMIAILALGKLLEPLQKSVLAAVVIANLKGMFMQLCDIPRLWRQNKIDAVIWVFTCIVSIILGLDLGLLAGLIFGLLTVVLRVQFPSWNGLGSIPSTDIYKSTKNYKNIEEPQGVKILRFSSPIFYGNVDGFKKCIKSTVGFDAIRVYNKRLKALRKIQKLIKSGQLRATKNGIISDAVSTNNAFEPDEDIEDLEELDIPTKEIEIQVDWNSELPVKVNVPKVPIHSLVLDCGAISFLDVVGVRSLRVIVKEFQRIDVNVYFASLQDYVIEKLEQCGFFDDNIRKDTFFLTVHDAILYLQNQVKSQEGQGSILETITLIQDCKDTLELIETELTEEELDVQDEAMRTLAS

SEQ ID NO: 7 - protein, 806 aa, Artificial Sequence
  source: /note=“Description of Artificial Sequence Synthetic polypeptide”
MAAPGGRSEPPQLPEYSCSYMVSRPVYSELAFQQQHERRLQERKTLRESLAKCCSCSRKRAFGVLKTLVPILEWLPKYRVKEWLLSDVISGVSTGLVATLQGMAYALLAAVPVGYGLYSAFFPILTYFIFGTSRHISVGPFPVVSLMVGSVVLSMAPDEHFLVSSSNGTVLNTTMIDTAARDTARVLIASALTLLVGIIQLIFGGLQIGFIVRYLADPLVGGFTTAAAFQVLVSQLKIVLNVSTKNYNGVLSIIYTLVEIFQNIGDTNLADFTAGLLTIVVCMAVKELNDRFRHKIPVPIPIEVIVTIIATAISYGANLEKNYNAGIVKSIPRGFLPPELPPVSLFSEMLAASFSIAVVAYAIAVSVGKVYATKYDYTIDGNQEFIAFGISNIFSGFFSCFVATTALSRTAVQESTGGKTQVAGIISAAIVMIAILALGKLLEPLQKSVLAAVVIANLKGMFMQLCDIPRLWRQNKIDAVIWVFTCIVSIILGLDLGLLAGLIFGLLTVVLRVQFPSWNGLGSIPSTDIYKSTKNYKNIEEPQGVKILRFSSPIFYGNVDGFKKCIKSTVGFDAIRVYNKRLKALRKIQKLIKSGQLRATKNGIISDAVSTNNAFEPDEDIEDLEELDIPTKEIEIQVDWNSELPVKVNVPKVPIHSLVLDCGAISFLDVVGVRSLRVIVKEFQRIDVNVYFASLQDYVIEKLEQCGFFDDNIRKDTFFLTVHDAILYLQNQVKSQEGQGSILETITLIQDCKDTLELIETELTEEELDVQDEAMRTLASGSRADYKDHDGDYKDHDIDYKDDDDK

SEQ ID NO: 8 - DNA, 2208 bp, Artificial Sequence
  source: /note=“Description of Artificial Sequence Synthetic polynucleotide”
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 9 - protein, 736 aa, Artificial Sequence
  source: /note=“Description of Artificial Sequence Synthetic polypeptide”
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 10 - DNA, 145 bp, Artificial Sequence
  source: /note=“Description of Artificial Sequence Synthetic polynucleotide”
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 145 bp, Artificial Sequence
  source: /note=“Description of Artificial Sequence Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 12 - DNA, 119 bp, Artificial Sequence
  source: /note=“Description of Artificial Sequence Synthetic polynucleotide”
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 13 - DNA, 130 bp, Artificial Sequence
  source: /note=“Description of Artificial Sequence Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 279 bp, Artificial Sequence
  source: /note=“Description of Artificial Sequence Synthetic polynucleotide”
gtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 15 - DNA, 277 bp, Artificial Sequence
  source: /note=“Description of Artificial Sequence Synthetic polynucleotide”
gtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 16 - DNA, 660 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 17 - DNA, 658 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 18 - DNA, 1725 bp, Homo sapiens
ctatggagtttgcataacaaacgtttggcagctcgctctcttacactccattaacaagctgtaacatatagctgcaggttgctataatctcattaatattttggaaacttgaatattgagtatttctgagtgctcattccccatatgccagccacttctgccatgctgactggttcctttctctccattattagcaattagcttcttaccttccaaagtcagatccaaggtatccaagatactagcaaaggaatcaactatgtgtgcaagttaagcatgcttaatatcacccaaacaaacaaagaggcagcatttcttaaagtaatgaagatagataaatcgggttagtcctttgcgacactgctggtgctttctagagttttatatattttaagcagcttgctttatattctgtctttgcctcccaccccaccagcacttttatttgtggagggttttggctcgccacactttgggaaacttatttgatttcacggagagctgaaggaagatcatttttggcaacagacaagtttaaacacgatttctatgggacattgctaactggggcccctaaggagaaaggggaaactgagcggagaatgggttaaatccttggaagcaggggagaggcaggggaggagagaagtcggaggagtataaagaaaaggacaggaaccaagaagcgtgggggtggtttgccgtaatgtgagtgtttcttaattagagaacggttgacaatagagggtctggcagaggctcctggccgcggtgcggagcgtctggagcggagcacgcgctgtcagctggtgagcgcactctcctttcaggcagctccccggggagctgtgcggccacatttaacaccatcatcacccctccccggcctcctcaacctcggcctcctcctcgtcgacagccttccttggcccccaccagcagagctcacagtagcgagcgtctctcgccgtctcccgcactcggccggggcctctctcctcccccagctgcgcagcgggagccgccactgcccactgcacctcccagcaaccagcccagcacgcaaagaagctgcgcaaagttaaagccaagcaatgccaaggggaggggaagctggaggcgggctttgagtggcttctgggcgcctggcgggtccagaatcgcccagagccgcccgcggtcgtgcacatctgacccgagtcagcttgggcaccagccgagagccggctccgcaccgctcccgcaccccagccgccggggtggtgacacacaccggagtcgaattacagccctgcaattaacatatgaatctgacgaatttaaaagaaggaaaaaaaaaaaaaaacctgagcaggcttgggagtcctctgcacacaagaacttttctcggggtgtaaaaactctttgattggctgctcgcacgcgcctgcccgcgccctccattggctgagaagacacgcgaccggcgcgaggagggggttgggagaggagcggggggagactgagtggcgcgtgccgctttttaaaggggcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaa

SEQ ID NO: 19 - DNA, 381 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 20 - DNA, 1013 bp, Artificial Sequence
  source: /note=“Description of Artificial Sequence Synthetic polynucleotide”
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 21 - DNA, 224 bp, Homo sapiens
ctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtc

SEQ ID NO: 22 - DNA, 2361 bp, Homo sapiens
aagtgggttcgggaggtctctatgagcaaggaatacaagacaaaacttcctcaatgcattgactatttcttcagactcaaaacactcattcttttttctattaagccattgaaagagaagcactaagactgcttctaggctttatttataaaataaacaccttatccctaacatgggcaaaatggctagaattattcagacgatttggcagcgtccagggtaagctggtgttataatacgctgctgatctacatcacagatttgctaataatgttcacgtgggccctggcatatctctgttcagttagagtgagtgctgacccaacagcctctgtggtcaagcgagtcacgaatgattaatcataaagaaaaatcagtttttgactgacctggatatccatgagctgcactgatcaccatgtaaggtcacatttagtaaatgctgaaataaaatgattaatgcatttatcaataaaagcctttgaaaatactttggataataaattggagttttaaaaatgcaaatttgcttagtatctaataatgaagtgttattacatatagccggaattgaggatctctttgatcctggaaatggtttacctaaaagctacagaaccaggccaatatattttgaaatattgatgcagacaaatgaaataataaagagattttcatggtttataaaaatcttttttgatatgataataatcatgatcacaactgagatcaaaaaaatatatgacagattattttgtttaaaaatgcagttttaattatcttagtctatagaaatgatcattgcatggaggcatgtataggtatgatctgtgtaaaatctgacataaaaacagtgctattctgagtgaaaatttttttgatgtgcttacataaccatggtgattaaaatgagtttatattttttctcaaaaattttagcagtgtgtaaagtaagtaatctttaactgaactctgaccacttaaaaaaaaatctaaaaattgaactacctatagtagtctgtgtttaaagtgaatttttaaagacaaagcattctaaatgaactcaatataaaaacattcatttggaatgtacatactgaaaaatacaggtttttttgaccaaaagtttttatatcttttctttttatttatttttttcctaagtgccaacaattttctagatattatatacaacacaggctttgatcttggggacttttcccatatatttcacactggagtgaatgaagttgtacttcatttctagagaaaagttatacccaggtccccaattgagaatgtcttgcttgattgaaaacgacatcatcccttggtatactccagggattggtttcaggacccctgcatttaccaaaatttgtgcacactcaagtcctgcagtcacccctgcctaaagatagaatggcttctctgtttttcttctgaaatacaaccagaaacaatgtgtctatttctgaaagaataggattaatgatcatacaaatgggttaatcctgaattctggttgtaaatctggttacagcataactaggattataatgctgcctcattttcacagcactacttgcttatattgacaacaaatcatctcgctaaagagtgaatgtaggccaggcgcggtggctcatgcctgtaatcccagcactttgggaggccgaggcgggtggatcacgaggtcaggagatcgagaccatcctggctaacatggtaaaaccccgtctctactaaaaatagaaaaaaagaaattagcctagcgtggtggctggcgggcgcctgtagtcccagctatttgggaggctaaggcaggagaatggcgtgaacccgggaggcggagcttgcagtgagccgaggtcgtgccactgcactccagcctgggcgacagagcaagactccgtctcaaaaaaaaaaaaaaaaaaaaaaaaagagtgaatgtaatagtcttgcagaaaatgaatgaatacctttgttcaataaaggaaatatgcactgctcacttttttgaaggaaatgccaaagttacgttttacaacaaggctagagtttgtaaattctgggttcatttgtgatgacataagtcagcaaactgcgggaatactgtctcttctatgtattttgtgaatagtaagcataattttagttttgtattatcaatgaaaatttcacttgaaattaaagctgccttttgttatatttttaacctataggataagattccagtattgtatatgagttttaacaaattaaaaaatcaaatcatgtacatttgaaaatatttgcacacatttaaaaataaatgtaaagttgtcttttaaactactcggatgtgtcctttctgaacaa

SEQ ID NO: 23 - DNA, 17 bp, Unknown
  source: /note=“Description of Unknown Poly(A) signal sequence”
aaataaaatacgaaatg

SEQ ID NO: 24 - DNA, 225 bp, Artificial Sequence
  source: /note=“Description of Artificial Sequence Synthetic polynucleotide”
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 25 - DNA, 122 bp, Artificial Sequence
  source: /note=“Description of Artificial Sequence Synthetic polynucleotide”
aacttgtttattgcagcttataatggttacaaataaagcaatagcatcacaaatttcacaaataaagcatttttttcactgcattctagttgtggtttgtccaaactcatcaatgtatctta

SEQ ID NO: 26 - DNA, 22 bp, Artificial Sequence
  source: /note=“Description of Artificial Sequence Synthetic oligonucleotide”
ttgtcgacgcggccgcacgcgt

SEQ ID NO: 27 - DNA, 44 bp, Artificial Sequence
  source: /note=“Description of Artificial Sequence Synthetic oligonucleotide”
ctcctgggcaacgtgctggttattgtgaccggtcgctagccacc

SEQ ID NO: 28 - DNA, 24 bp, Artificial Sequence
  source: /note=“Description of Artificial Sequence Synthetic oligonucleotide”
taagagctcgctgatcagcctcga

SEQ ID NO: 29 - DNA, 39 bp, Artificial Sequence
  source: /note=“Description of Artificial Sequence Synthetic oligonucleotide”
aagcttgaattcagctgacgtgcctcggaccgtcctagg

SEQ ID NO: 30 - DNA, 14 bp, Artificial Sequence
  source: /note=“Description of Artificial Sequence Synthetic oligonucleotide”
gcggccgcacgcgt

SEQ ID NO: 31 - DNA, 39 bp, Artificial Sequence
  source: /note=“Description of Artificial Sequence Synthetic oligonucleotide”
ctcctgggcaacgtgctggttattgtgaccggtgccacc

SEQ ID NO: 32 - DNA, 24 bp, Artificial Sequence
  source: /note=“Description of Artificial Sequence Synthetic oligonucleotide”
taagagctcgctgatcagcctcga

SEQ ID NO: 33 - DNA, 34 bp, Artificial Sequence
  source: /note=“Description of Artificial Sequence Synthetic oligonucleotide”
aagcttgaattcagctgacgtgcctcggaccgct

SEQ ID NO: 34 - protein, 159 aa, Artificial Sequence
  source: /note=“Description of Artificial Sequence Synthetic polypeptide”
MISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 35 - DNA, 483 bp, Artificial Sequence
  source: /note=“Description of Artificial Sequence Synthetic polynucleotide”
ggtaccatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataa

SEQ ID NO: 36 - DNA, 474 bp, Artificial Sequence
  source: /note=“Description of Artificial Sequence Synthetic polynucleotide”
atcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcga

SEQ ID NO: 37 - protein, 112 aa, Artificial Sequence
  source: /note=“Description of Artificial Sequence Synthetic polypeptide”
MGVEKQVIRPGNGPKPAPGQTVTVHCTGFGKDGDLSQKFWSTKDEGQKPFSFQIGKGAVIKGWDEGVIGMQIGEVARLRCSSDYAYGAGGFPAWGIQPNSVLDFEIEVLSVQ

SEQ ID NO: 38 - DNA, 78 bp, Artificial Sequence
  source: /note=“Description of Artificial Sequence Synthetic oligonucleotide”
ggatcccgggctgactacaaagaccatgacggtgattataaagatcatgacatcgactacaaggatgacgatgacaag

SEQ ID NO: 39 - DNA, 4657 bp, Artificial Sequence
  source: /note=“Description of Artificial Sequence Synthetic polynucleotide”
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtcgctagccaccatggcagcgccaggcggcaggtcggagccgccgcagctccccgagtacagctgcagctacatggtgtcgcggccggtctactcggagctagctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatcctaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 40 - DNA, 4674 bp, Artificial Sequence
  source: /note=“Description of Artificial Sequence Synthetic polynucleotide”
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggcagcgccaggcggcaggtcggagccgccgcagctccccgagtacagctgcagctacatggtgtcgcggccggtctactcggagctagctttccagcaacagcacgagcggcgcctgcaggagcgcaagacgctgcgggagagcctggccaagtgctgcagttgttcaagaaagagagcctttggtgtgctaaagactcttgtgcccatcttggagtggctccccaaataccgagtcaaggaatggctgcttagtgacgtcatttcgggagttagtactgggctagtggccacgctgcaagggatggcatatgccctactagctgcagttcctgtcggatatggtctctactctgcttttttccctatcctgacatactttatctttggaacatcaagacatatctcagttggaccttttccagtggtgagtttaatggtgggatctgttgttctgagcatggcccccgacgaacactttctcgtatccagcagcaatggaactgtattaaatactactatgatagacactgcagctagagatacagctagagtcctgattgccagtgccctgactctgctggttggaattatacagttgatatttggtggcttgcagattggattcatagtgaggtacttggcagatcctttggttggtggcttcacaacagctgctgccttccaagtgctggtctcacagctaaagattgtcctcaatgtttcaaccaaaaactacaatggagttctctctattatctatacgctggttgagatttttcaaaatattggtgataccaatcttgctgatttcactgctggattgctcaccattgtcgtctgtatggcagttaaggaattaaatgatcggtttagacacaaaatcccagtccctattcctatagaagtaattgtgacgataattgctactgccatttcatatggagccaacctggaaaaaaattacaatgctggcattgttaaatccatcccaagggggtttttgcctcctgaacttccacctgtgagcttgttctcggagatgctggctgcatcattttccatcgctgtggtggcttatgctattgcagtgtcagtaggaaaagtatatgccaccaagtatgattacaccatcgatgggaaccaggaattcattgcctttgggatcagcaacatcttctcaggattcttctcttgttttgtggccaccactgctctttcccgcacggccgtccaggagagcactggaggaaagacacaggttgctggcatcatctctgctgcgattgtgatgatcgccattcttgccctggggaagcttctggaacccttgcagaagtcggtcttggcagctgttgtaattgccaacctgaaagggatgtttatgcagctgtgtgacattcctcgtctgtggagacagaataagattgatgctgttatctgggtgtttacgtgtatagtgtccatcattctggggctggatctcggtttactagctggccttatatttggactgttgactgtggtcctgagagttcagtttccttcttggaatggccttggaagcatccctagcacagatatctacaaaagtaccaagaattacaaaaacattgaagaacctcaaggagtgaagattcttagattttccagtcctattttctatggcaatgtcgatggttttaaaaaatgtatcaagtccacagttggatttgatgccattagagtatataataagaggctgaaagcgctgaggaaaatacagaaactaataaaaagtggacaattaagagcaacaaagaatggcatcataagtgatgctgtttcaacaaataatgcttttgagcctgatgaggatattgaagatctggaggaacttgatatcccaaccaaggaaatagagattcaagtggattggaactctgagcttccagtcaaagtgaacgttcccaaagtgccaatccatagccttgtgcttgactgtggagctatatctttcctggacgttgttggagtgagatcactgcgggtgattgtcaaagaattccaaagaattgatgtgaatgtgtattttgcatcacttcaagattatgtgatagaaaagctggagcaatgcgggttctttgacgacaacattagaaaggacacattctttttgacggtccatgatgctatactctatctacagaaccaagtgaaatctcaagagggtcaaggttccattttagaaacgatcactctcattcaggattgtaaagatacccttgaattaatagaaacagagctgacggaagaagaacttgatgtccaggatgaggctatgcgtacacttgcatccggatcccgggctgactacaaagaccatgacggtgattataaagatcatgacatcgactacaaggatgacgatgacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 82 bp, Artificial Sequence
  source: /note=“Description of Artificial Sequence Synthetic oligonucleotide”
gtaagtatcaaggttacaagacaggtttaaggagaccaatagaaactgggcttgtcgagacagagaagactcttgcgtttct

SEQ ID NO: 42 - DNA, 51 bp, Artificial Sequence
  source: /note=“Description of Artificial Sequence Synthetic oligonucleotide”
gataggcacctattggtcttactgacatccactttgcctttctctccacag

SEQ ID NO: 43 - DNA, 1673 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 44 - DNA, 1671 bp, Artificial Sequence
  source: /note=“Description of Artificial Sequence Synthetic polynucleotide”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 45 - DNA, 60 bp, Homo sapiens
ctgccttctgagagcgctataaaggcagcggaagggtagtccgcggggcattccgggcgg

SEQ ID NO: 46 - DNA, 60 bp, Homo sapiens
ctctaggcgggctctgctcttctttaaggagtcccacagggcctggcccgcccctgacct

SEQ ID NO: 47 - DNA, 2000 bp, Homo sapiens
taaagagttgtgagttgtgtaggtgagttgccatggagctacaaatatgagttgatattctgaaatcctagacagccatctccaaggttaagaaaaatccttatgcactcacttgcaaagatatccacagcatgctcttaatggagaaaaacaaagccttagatcaaatatgtaaagtaatttttagttttttgaaaaggtatgtttgggctatagataaatctgttcaaaaaacatgagagaagataataatggttgaaaggagacacagtgcttgccctcaagaagtttttgtctagtgagggagagagaacttgtatgtaaataaaattgtgttactaaggtagatagtgagaagtaacttaagagaggatcagataaggtattaagagaatacagaaaagggtctggattaattctgaacagcatcaaagaatgttcttgcaagagatagtgttttcaccagatcttgaaggtatggatgagggtatacagagtgagtatattcagattctactttaaaacaaatactttcctctgttgtagtggagttgagctatacatccaacaataatgaaaaaatacacgcatatatacatatatggagagagatacatattttagtacatgtagcaattgattaataaatgtacagtttaagtcgcatgcaaaaccttggagtgatagcaaacttcattgtaggatgtttagcagcatctctggtctctactcactagatcccaatagcatctccctaggtgtgacaaccaaaaatgtctccaggcattgacctctggaggcaaaaaaagccctttattaagaaccagtggtatacataagtaaaacatacacaagagattcctcccctcttctctgtatgtgaataaaaattgcaaagttcatgacctggattttccttttaggtttcttctttagtggttcttaacttcattgggtgaagtaagcctttgaagatctgttgaaagctgttgactcattcacttctcaggaaaacgcacatgctgactaccatttcagagaatttgcatcagggttctctggggaggagttctgagttctgtttccaggagctcgtagaattgtcatggtctgcatatgcaaggcaggtggattacggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggc

SEQ ID NO: 48 - DNA, 1400 bp, Homo sapiens
ggctgctcggaaaacaggacgaggggagagacttgctcaataagctgaaagttctgcccccgagagggctgcgacagctgctggaatgtgcctgcagcgtccgcctcttggggacccgcggagcgcgccctgacggttccacgcctggcccgggggtctgcacctctcctccagtgcgcacctggagctgcgtcccgggtcaggtgcggggagggagggaatctcagtgtccccttccagccttgcaagcgcctttggcccctgccccagcccctcggtttgggggagatttcagaacgcggacagcgccctggctgcgggccataggggactgggtggaactcgggaagcccccagagcaggggcttactcgcttcaagtttggggaaccccgggcagcgggtgcaggccacgagacccgaaggttctcaggtgcccccctgcaggctggccgtgcgcgccgtggggcgcttgtcgcgagcgccgagggctgcaggacgcggaccagactcgcggtgcaggggggcctggctgcagctaacaggtgatcccgttctttctgttcctcgctcttcccctccgatcgtcctcgcttaccgcgtgtcctccctcctcgctgtcctctggctcgcaggtcatggcagcgccaggcggcaggtcggagccgccgcagctccccgagtacagctgcagctacatggtgtcgcggccggtctacagcgagctcgctttccagcaacagcacgagcggcgcctgcaggagcgcaagacgctgcgggagagcctggccaagtgctgcaggtagcggccgcgcgggcctgcgtagagagaagcggagcggggcgtccacgccttggggagggaagggcgtccccagcgggcgagagtggggtgcgggcggcggagcccctgggcgccagctgcttctcccagaggcccgactttcggtctccggtcctccacgccgcccttctggtgggagggtggctccatcagtctcgggcccgaaatgaacttacctgggaaactcgcctttggggagagtgggttctaggagccccgtctctctttttcctctctgaaggaaacttggagtgcctcttggggtacagtgggtccctgttgccttcttgggagcttgtttaaatgaaatgaatagggaaacccagctcttgaccaggaggagtccttgaaacactcaagctaagtaggcgggctaccattcagttagagaccaggatgcaagctagaacccaggggagcgcggggtgtgccaagtacttcatcagcaggctgtgggacccctggggaaagccaccctcagtctctaaacccaaacatgccgtaactagatgtcacaaacataaagaaattagagtttctaaaacctttcattatag

SEQ ID NO: 49 - DNA, 16 bp, Artificial Sequence
  source: /note=“Description of Artificial Sequence Synthetic probe”
ccagcagagtcagggc

SEQ ID NO: 50 - DNA, 900 bp, Homo sapiens
cggaaggttgatgtacagaggtctgtattttggagcctcttctgtatttacttcagaacactaacaatcaggcgagaatgttctggtttatcaaacccttccttctgcctttcatcttaaccatgcattagttttaacaaagttcatcccaacagaagacaaaacactgatgaggtaggatagctccagctcctcctccctctcttctagtcttgatttccatgtagtccagtttattccttccctgattgtccaggagaatgagaaaaagaaaaaacagagtctagtgggtaagaaagggccacctggacggcttgatttggattgtgaaataaaacacacacacatgcacacgtagaataagtggctaaaatctgagtaaatcgtgaactctctgtatcctccacccattgaatactcctaaaagactttctagaaattcaaggacttattaatatagaaacctggccattgttcctcttctcctccccatgtggtatgagagcacctgtggcaggctcccagagaccacggacctcttcctctaggcgggctctgctcttctttaaggagtcccacagggcctggcccgcccctgacctcgcaacccttgagattagtaacgggatgagtgaggatccgggtggcccctgcgtggcagccagtaagagtctcagccttcccggttcgggaaaggggaagaatgcaggaggggtaggatttctttcctgataggatcggttgggaaagaccgcagcctgtgtgtgtctttcccttcgaccaaggtgtctgttgctccgtaaataaaacgtcccactgccttctgagagcgctataaaggcagcggaagggtagtccgcggggcattccgggcggggcgcgagcagagacaggtgagtt

SEQ ID NO: 51 - DNA, 1269 bp, Homo sapiens
agggctatttgtacctcaacgagggcttctctccaagaaagccctgaatccttttcctcctttttcctgcagattcactataggacactttttgaagcaagagcatgcattttccccctggcgctctgcagcggttctcagagcccagtgtcactcacataggtgggactgctctcagttcagagagcgctgggacacttaagatgaaaagtccctggaagttagcaaacagccatctgtcactctggcatcgatttactaaaagtgacttctagggtattctaaaccacttttaaaaaacaaatgagtcacttcgacttcctcaccccgcaagagataggaaggcagcagtggagtgctcgctcaggagctgtatttgtttagcgattagcctagagctttgattttagggcaaaagcgagccagacagtgcggcagacgtaaggatcaaaaaggccacctatcattcgccggggacgcctgcctccttaccctgataacgtaactatttctctgcataggattttagtttttgtgtttttgttttgttttattctgtttaatcacttcaagtatctcatccattatttgaagcgggctcggaggaaacgtgccgcatcctccagtccttgtgcgtctgtttaggtctctccgaagcaggtccctctcgactcttagatctgggtctccagcacgcatgaaggggtaagggtgggggggtcccctattccggcgcgcggcgttgagcactgaatcttccaggcggaggctcagtgggagcgccgagaactcgccagtaccgcgcgctgcctgctgcctgctgcctcccagcccaggacttgggaaaggagggaggggacaagtggagggaaagtggggccgggcggggggtgcctgggaagccaggctgcgctgacgtcactgggcgcgcaattcgggctggagcgctttaaaaaacgagcgtgcaagcagagatgctgctccacaccgctcaggccgcgagcagcagcaaggcgcaccgccactgtcgccgctgcagccagggctgctccgaaggccggcgtggcggcaaccggcacctctgtccccgccgcgcttctcctcgccgcccacgccgtggggtcaggaacgcggcgtctggcgctgcagacgcccgctgagttgcagaagcccacggagcggcgcccggcgcgccacggcccgtagcagtccggtgctgctctccgcccgcgtccggctcgtggccccctacttcgggcaccgaccggt

SEQ ID NO: 52 - DNA, 463 bp, Homo sapiens
tgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 53 - DNA, 2204 bp, Homo sapiens
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 54 - DNA, 23 bp, Artificial Sequence
  source: /note=“Description of Artificial Sequence Synthetic primer”
gatacagctagagtcctgattgc

SEQ ID NO: 55 - DNA, 23 bp, Artificial Sequence
  source: /note=“Description of Artificial Sequence Synthetic primer”
gatctgccaagtacctcactatg

SEQ ID NO: 56 - protein, 780 aa, Mus sp.
MAARGGRSEPPQLAEYSCSYTVSRPVYSELAFQQQRERRLPERRTLRDSLARSCSCSRKRAFGVVKTLLPILDWLPKYRVKEWLLSDIISGVSTGLVGTLQGMAYALLAAVPVQFGLYSAFFPILTYFVFGTSRHISVGPFPVVSLMVGSVVLSMAPDDHFLVPSGNGSALNSTTLDTGTRDAARVLLASTLTLLVGIIQLVFGGLQIGFIVRYLADPLVGGFTTAAAFQVLVSQLKIVLNVSTKNYNGILSIIYTLIEIFQNIGDTNIADFIAGLLTIIVCMAVKELNDRFKHRIPVPIPIEVIVTIIATAISYGANLEKNYNAGIVKSIPSGFLPPVLPSVGLFSDMLAASFSIAVVAYAIAVSVGKVYATKHDYVIDGNQEFIAFGISNVFSGFFSCFVATTALSRTAVQESTGGKTQVAGLISAVIVMVAIVALGRLLEPLQKSVLAAVVIANLKGMFMQVCDVPRLWKQNKTDAVIWVFTCIMSIILGLDLGLLAGLLFALLTVVLRVQFPSWNGLGSVPSTDIYKSITHYKNLEEPEGVKILRFSSPIFYGNVDGFKKCINSTVGFDAIRVYNKRLKALRRIQKLIKKGQLRATKNGIISDIGSSNNAFEPDEDVEEPEELNIPTKEIEIQVDWNSELPVKVNVPKVPIHSLVLDCGAVSFLDVVGVRSLRMIVKEFQRIDVNVYFALLQDDVLEKMEQCGFFDDNIRKDRFFLTVHDAILHLQNQVKSREGQDSLLETVARIRDCKDPLDLMEAEMNAEELDVQDEAMRRLAS

SEQ ID NO: 57 - protein, 780 aa, Artificial Sequence
  source: /note=“Description of Artificial Sequence Synthetic polypeptide”
MAARGGRSEPPQLAEYSCSYTVSRPVYSELAFQQQRERRLPERRTLRDSLARSCSCSRKRAFGVVKTLLPILDWLPKYRVKEWLLSDIISGVSTGLVGTLQGMAYALLAAVPVQFGLYSAFFPILTYFVFGTSRHISVGPFPVVSLMVGSVVLSMAPDDHFLVPSGNGSALNSTTLDTGTRDAARVLLASTLTLLVGIIQLVFGGLQIGFIVRYLADPLVGGFTTAAAFQVLVSQPKIVLNVSTKNYNGILSIIYTLIEIFQNIGDTNIADFIAGLLTIIVCMAVKELNDRFKHRIPVPIPIEVIVTIIATAISYGANLEKNYNAGIVKSIPSGFLPPVLPSVGLFSDMLAASFSIAVVAYAIAVSVGKVYATKHDYVIDGNQEFIAFGISNVFSGFFSCFVATTALSRTAVQESTGGKTQVAGLISAVIVMVAIVALGRLLEPLQKSVLAAVVIANLKGMFMQVCDVPRLWKQNKTDAVIWVFTCIMSIILGLDLGLLAGLLFALLTVVLRVQFPSWNGLGSVPSTDIYKSITHYKNLEEPEGVKILRFSSPIFYGNVDGFKKCINSTVGFDAIRVYNKRLKALRRIQKLIKKGQLRATKNGIISDIGSSNNAFEPDEDVEEPEELNIPTKEIEIQVDWNSELPVKVNVPKVPIHSLVLDCGAVSFLDVVGVRSLRMIVKEFQRIDVNVYFALLQDDVLEKMEQCGFFDDNIRKDRFFLTVHDAILHLQNQVKSREGQDSLLETVARIRDCKDPLDLMEAEMNAEELDVQDEAMRRLAS

SEQ ID NO: 58 - RNA, 5000 nt, Artificial Sequence
  source: /note=“Description of Artificial Sequence Synthetic polynucleotide”
  misc_feature (1)..(5000): /note=“This sequence may encompass 50, 60, 70, 100, 200, 500, 1000, 2000, 3000, 4000, or 5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0201372 A1
(43) Pub. Date: Jun. 29, 2023

(54) COMPOSITIONS AND METHODS FOR TREATING SLC26A4-ASSOCIATED HEARING LOSS

(71) Applicants: Akouos, Inc., Boston, MA (US); Taipei Veterans General Hospital, Taipei (TW)

(72) Inventors: Emmanuel John Simons, Brookline, MA (US); Robert Ng, Newton, MA (US); Yen-Fu Cheng, Taipei (TW)

(21) Appl. No.: 17/924,933

(22) PCT Filed: May 12, 2021
(86) PCT No.: PCT/US21/31983

§ 371 Date: Nov. 11, 2022

Related U.S. Application Data
(60) Provisional application No. 63/024,466, filed on May 13, 2020.

(51) Int. Cl. A61K 48/00; A61K 38/17; C07K 14/705; C12N 15/86; A61P 27/16

(52) U.S. Cl. CPC A61K 48/005; A61K 38/1709; C07K 14/705; C12N 15/86; A61P 27/16; A61K 48/0075; C12N 2750/14143

(57) ABSTRACT

The present disclosure provides constructs comprising a coding sequence operably linked to a promoter, wherein the coding sequence encodes a pendrin protein. Exemplary constructs include AAV constructs. Also provided are methods of using disclosed constructs for the treatment of hearing loss and/or deafness.

### CROSS REFERENCE TO RELATED APPLICATIONS

This application claims the benefit of U.S. Provisional Application No. 63/024,466, filed May 13, 2020, the contents of which is hereby incorporated in its entirety.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present disclosure provides the recognition that diseases or conditions associated with hearing loss can be treated via, e.g., the replacement or addition of certain gene products. The present disclosure further provides that gene products involved in the development, function, and/or maintenance of inner ear cells can be useful for treatment of diseases or conditions associated with hair cell and/or supporting cell loss. The present disclosure thus provides for the administration of compositions that result in expression of gene products involved in the development, function, and/or maintenance of inner ear cells including supporting cells and hair cells, and/or the use of such compositions in the treatment of hearing loss, or diseases or conditions associated with hearing loss. In some embodiments, a gene product can be encoded by an SLC26A4 gene or a characteristic portion thereof. In some embodiments, a gene product can be pendrin protein or a characteristic portion thereof.

The present disclosure further provides that AAV particles can be useful for administration of compositions that result in expression of gene products involved in the development, function, and/or maintenance of inner ear cells, and/or the treatment of hearing loss, or diseases or conditions associated with hearing loss. As described herein, AAV particles comprise (i) a AAV polynucleotide construct (e.g., a recombinant AAV polynucleotide construct), and (ii) a capsid comprising capsid proteins. In some embodiments, an AAV polynucleotide construct comprises an SLC26A4 gene or a characteristic portion thereof.

The present disclosure further provides compositions comprising polynucleotide constructs comprising an SLC26A4 gene or a characteristic portion thereof. In some embodiments, a construct may further include regulatory elements operably attached to a coding sequence. In certain embodiments, included regulatory elements facilitate tissue specific expression at physiologically suitable levels.

The present disclosure also provides a genetically modified mouse whose genome comprises a modified Slc26a4 gene. In some embodiments, the genetically modified mouse comprises a modified Slc26a4 gene that encodes a polypeptide according to SEQ ID NO: 57. In some embodiments, the genetically modified mouse is of a mouse strain suitable for use in audiological analysis experiments. In some embodiments, the genetically modified mouse is of a mouse strain suitable for use in coordination analysis experiments. In some embodiments, the genetically modified mouse is not of CBA/CaJ or CBA/J strain. In some embodiments, the genetically modified mouse suitable for use in audiological analysis experiments is of FVB strain. In some embodiments, the genetically modified mouse suitable for use in audiological analysis experiments is of FVB, 129/Sv-+p+Tyr-c+Mgf-SIJ/J, A/HeJ, AKR/J, BALB/cByJ, BALB/cJ, BDP/J, BXSB/MpJ, C3H/HeJ, C3H/HeOuJ, C3HeB/FeJ, C57BL/10J, C57BL/10SnJ, C57BL/6ByJ, CASA/RK, CAST/Ei, CBA/J, CZECH II/Ei, DBA/2HaSmn, FVB/NJ, HRS/J hrl+, MOLD/Rk, MOLF/Ei, MOLG/Dn, NON/LtJ, NZB/B1NJ, NZO/NIJ, NZW/LacJ, PERA/camEi, PERC/Ei, PL/J, RBA/Dn, RBF/DnJ, RF/J, RHJ/Le hrrh-J/+, RIIIS/J, SEC/1ReJ, SENCARC/PtJ, SF/CamEi, SHR/GnEi, SJL/J, SM/J, SPRET/Ei, ST/bJ, or SWR/J strain. In some embodiments, a genetically modified mouse is treated with AAV particles, constructs, or compositions described herein.

Also provided herein are methods of administering constructs and compositions described herein. In certain embodiments, administration involves surgical intervention and the delivery of rAAV particles comprising therapeutic constructs. In certain embodiments, AAV particles may be delivered to the inner ear of a subject in need thereof by surgical introduction through the round window membrane. In some embodiments, a purpose of an intervention is to treat hearing loss in a subject. In some embodiments, efficacy of an intervention is determined through established tests, and measurements are compared to known control measurements.

### Definitions

The scope of the present disclosure is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., certain embodiments or aspects “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a composition to a subject or system to achieve delivery of an agent to a subject or system. In some embodiments, an agent is, or is included in, a composition; in some embodiments, an agent is generated through metabolism of a composition or one or more components thereof. Those of ordinary skill in the art will be aware of a variety of routes that may, in appropriate circumstances, be utilized for administration to a subject, for example a human. For example, in some embodiments, administration may be systematic or local. In some embodiments, a systematic administration can be intravenous. In some embodiments, administration can be local. Local administration can involve delivery to cochlear perilymph via, e.g., injection through a round-window membrane or into scala-tympani, a scala-media injection through endolymph, perilymph and/or endolymph following canalostomy. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some embodiments, an amino acid has a general structure, e.g., H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some embodiments, an amino acid, including a carboxy- and/or amino-terminal amino acid in a polypeptide, can contain a structural modification as compared with general structure as shown above. For example, in some embodiments, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some embodiments, such modification may, for example, alter circulating half-life of a polypeptide containing a modified amino acid as compared with one containing an otherwise identical unmodified amino acid. In some embodiments, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some embodiments, the term “approximately” or “about” refers to a range of values that fall within ±10% (greater than or less than) of a stated reference value unless otherwise stated or otherwise evident from context (except where such number would exceed 100% of a possible value). For example, in some embodiments, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some embodiments, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some embodiments, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral construct, and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral construct, etc.) that is capable of replicating when associated with proper control elements. Thus, in some embodiments, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein.

CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid |
| | | (Kauer, U.S. Pat. No. 4,511,390) |
| Serine | S | D-Ser, Thr, D-Thr, allo-Thr, Met, D-Met, |
| | | Met (O), D-Met (O), L-Cys, D-Cys |
| Threonine | T | D-Thr, Ser, D-Ser, allo-Thr, Met, D-Met, |
| | | Met (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some embodiments, a control is a historical control (e.g., of a test or assay performed previously, or an amount or result that is previously known). In some embodiments, a control is or comprises a printed or otherwise saved record. In some embodiments, a control is a positive control. In some embodiments, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some embodiments, “Assaying for the presence of” can be determining an amount of something present and/or determining whether or not it is present or absent.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” describes something that exists in a form in which it exhibits a property and/or activity by which it is characterized. For example, in some embodiments, a “functional” biological molecule is a biological molecule in a form in which it exhibits a property and/or activity by which it is characterized. In some such embodiments, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some embodiments, a gene includes coding sequence (i.e., sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof, the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some embodiments, a gene may encode a polypeptide, but that polypeptide may not be functional, e.g., a gene variant may encode a polypeptide that does not function in the same way, or at all, relative to the wild-type gene. In some embodiments, a gene may encode a transcript which, in some embodiments, may be toxic beyond a threshold level. In some embodiments, a gene may encode a polypeptide, but that polypeptide may not be functional and/or may be toxic beyond a threshold level.

Hearing loss: As used herein, the term “hearing loss” may be used to a partial or total inability of a living organism to hear. In some embodiments, hearing loss may be acquired. In some embodiments, hearing loss may be hereditary. In some embodiments, hearing loss may be genetic. In some embodiments, hearing loss may be as a result of disease or trauma (e.g., physical trauma, treatment with one or more agents resulting in hearing loss, etc.). In some embodiments, hearing loss may be due to one or more known genetic causes and/or syndromes. In some embodiments, hearing loss may be of unknown etiology. In some embodiments, hearing loss may or may not be mitigated by use of hearing aids or other treatments.

Heterologous: As used herein, the term “heterologous” may be used in reference to one or more regions of a particular molecule as compared to another region and/or another molecule. For example, in some embodiments, heterologous polypeptide domains, refers to the fact that polypeptide domains do not naturally occur together (e.g., in the same polypeptide). For example, in fusion proteins generated by the hand of man, a polypeptide domain from one polypeptide may be fused to a polypeptide domain from a different polypeptide. In such a fusion protein, two polypeptide domains would be considered “heterologous” with respect to each other, as they do not naturally occur together.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some embodiments, a value is statistically significantly difference that a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, an appropriate reference is a negative reference; in some embodiments, an appropriate reference is a positive reference.

Nucleic acid: As used herein, the term “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments, a nucleic acid has a nucleotide sequence comprising at least one element that encodes, or is complementary to a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some embodiments, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may include one or more pendant groups or other modifications, e.g., modifying or attached to one or more amino acid side chains, at a polypeptide's N-terminus, at a polypeptide's C-terminus, or any combination thereof. In some embodiments, such pendant groups or modifications may be acetylation, amidation, lipidation, methylation, pegylation, etc., including combinations thereof. In some embodiments, polypeptides may contain L-amino acids, D-amino acids, or both and may contain any of a variety of amino acid modifications or analogs known in the art. In some embodiments, useful modifications may be or include, e.g., terminal acetylation, amidation, methylation, etc. In some embodiments, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, a protein is antibodies, antibody fragments, biologically active portions thereof, and/or characteristic portions thereof.

Polynucleotide: As used herein, the term “polynucleotide” refers to any polymeric chain of nucleic acids. In some embodiments, a polynucleotide is or comprises RNA; in some embodiments, a polynucleotide is or comprises DNA. In some embodiments, a polynucleotide is, comprises, or consists of one or more natural nucleic acid residues. In some embodiments, a polynucleotide is, comprises, or consists of one or more nucleic acid analogs. In some embodiments, a polynucleotide analog differs from a nucleic acid in that it does not utilize a phosphodiester backbone. Alternatively or additionally, in some embodiments, a polynucleotide has one or more phosphorothioate and/or 5′-N-phosphoramidite linkages rather than phosphodiester bonds. In some embodiments, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a polynucleotide comprises one or more modified sugars (e.g., 2′-fluororibose, ribose, 2′-deoxyribose, arabinose, and hexose) as compared with those in natural nucleic acids. In some embodiments, a polynucleotide has a nucleotide sequence that encodes a functional gene product such as an RNA or protein. In some embodiments, a polynucleotide includes one or more introns. In some embodiments, a polynucleotide is prepared by one or more of isolation from a natural source, enzymatic synthesis by polymerization based on a complementary template (in vivo or in vitro), reproduction in a recombinant cell or system, and chemical synthesis. In some embodiments, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a polynucleotide is partly or wholly single stranded; in some embodiments, a polynucleotide is partly or wholly double stranded. In some embodiments, a polynucleotide has a nucleotide sequence comprising at least one element that encodes, or is the complement of a sequence that encodes, a polypeptide. In some embodiments, a polynucleotide has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some embodiments, a reference is a negative control reference; in some embodiments, a reference is a positive control reference.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe (e.g., virus), a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some embodiments, a variant has a reduction or elimination in activity or functionality or a change in activity that results in a negative outcome (e.g., increased electrical activity resulting in chronic depolarization that leads to cell death). Such a variant is referred to herein as a loss-of-function variant. For example, in some embodiments, a SLC26A4 gene sequence is a wild-type sequence, which encodes a functional protein and exists in a majority of members of species with genomes containing the SLC26A4 gene. In some such embodiments, a gain-of-function variant can be a gene sequence of SLC26A4 that contains one or more nucleotide differences relative to a wild-type SLC26A4 gene sequence. In some embodiments, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some embodiments, a loss-of-function variant has one or more changes that result in a transcript or polypeptide that is defective in some way (e.g., decreased function, non-functioning) relative to the wild type transcript and/or polypeptide. For example, in some embodiments, a mutation in a SLC26A4 sequence results in a non-functional or otherwise defective pendrin protein.

### BRIEF DESCRIPTION OF THE DRAWING

FIG. 1 panel (A) depicts a simplified endogenous AAV genome; panel (B) depicts a simplified recombinant AAV (rAAV) construct capable of expressing an SLC26A4 gene.

FIG. 2 depicts an exemplary nucleotide construct sequence map.

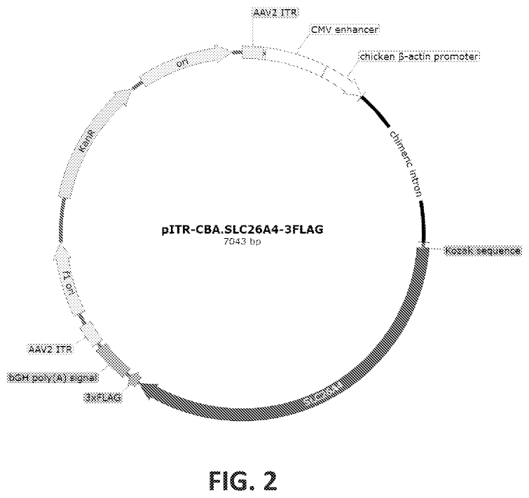

FIG. 3 depicts an exemplary rAAV construct comprising an SLC26A4 gene.

FIG. 4 depicts an exemplary rAAV construct comprising an SLC26A4 gene.

FIG. 5 depicts pendrin protein expression in HEK293FT cells that have been transfected with exemplary rAAV constructs.

FIG. 6 depicts SLC26A4 mRNA expression in HEK293FT cells and wild type neonatal CD1 explants that have been transduced with exemplary rAAV constructs.

FIG. 7 depicts inner ear morphology of wild type neonatal CD1 explants that have been transduced with exemplary rAAV constructs.

FIG. 8 panel (A) depicts an inner ear morphology of P21 day old C57BL/6J mice; panel (B) depicts an inner ear morphology of P21 day old C57BL/6J Slc26a4^{tm1Dontuh/tm1Dontuh} mice that underwent unilateral intracochlear injection at day P3 of compositions comprising exemplary rAAV constructs.

FIG. 9 panel (A) depicts control hearing levels in C57BL/6J heterozygous Slc26a4^{tm1Dontuh/+} mice; panel (B) depicts auditory brainstem response (ABR) results from P21 day old C57BL/6J Slc26a4^{tm1Dontuh/tm1Dontuh} mice that underwent unilateral intracochlear injection at day P0 with compositions comprising exemplary rAAV constructs; panel (C) is a graphical representation of ABR data from control, and test mice injected at day P0 or day P3 with compositions comprising exemplary rAAV constructs; panel (D) depicts auditory brainstem response results from P21 day old C57BL/6J Slc26a4^{tm1Dontuh/tm1Dontuh} mice that underwent unilateral intracochlear injection at day P3 with compositions comprising exemplary rAAV constructs.

FIG. 10 depicts eGFP protein expression in HEK293T cells under the power of various exemplary promoters, cells were sorted and quantified 72 hours after transfection.

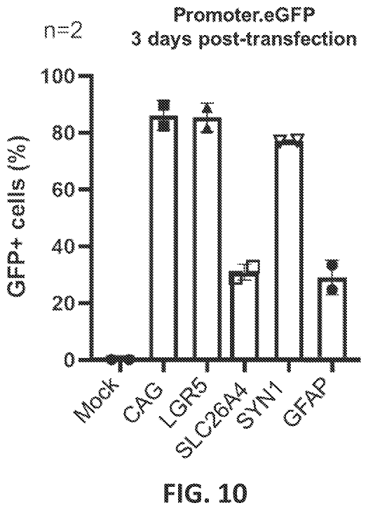

FIG. 11 depicts ABR results from a control homozygous Slc26a4 mutant mouse (S1c26a4^{L236P/L236P}) a control WT mouse (Slc26a4^{WT/WT}), and a homozygous S1c26a4^{L236P/L236P} mouse provided with a construct as represented in FIG. 4 through a round window membrane (RWM) injection at day P2. On the Y axis is ABR threshold in dB SPL in response to click stimuli, while the X axis represents age at time of measurement, ranging from P30 to P180, the injected ear is noted as a “treated”, while the non-injected ear is “contralateral”.

FIG. 12 depicts ABR results from homozygous S1c26a4^{L236P/L236P} mutant mice provided with a construct as represented in FIG. 4 through a RWM injection at day P2 (N=4). On the Y axis is ABR threshold in dB SPL, while the X axis represents noise stimuli provided (clicks, or pure tone at noted frequency). Measurements occurred at P30, the injected ear is noted as a “treated”, while the non-injected ear is “contralateral”.

FIG. 13A depicts ABR results from homozygous S1c26a4^{L236P/L236P} mutant mouse provided with a construct as represented in FIG. 4 through a RWM injection with posterior semicircular canal (PSCC) fenestration at day P23. On the Y axis is ABR threshold in dB SPL, while the X axis represents noise stimuli provided (clicks, or pure tone at noted frequency). Pre-injection measurements occurred at P22, and post-injection measurements occurred at P50, the injected ear is noted as a “treated”, while the non-injected ear is “contralateral”.

FIG. 13B depicts ABR results from homozygous S1c26a4^{L236P/L236P} mutant mouse provided with a construct as represented in FIG. 4 through a RWM injection with PSCC fenestration at day P23. On the Y axis is ABR threshold in dB SPL, while the X axis represents noise stimuli provided (clicks, or pure tone at noted frequency). Pre-injection measurements occurred at P22, and post-injection measurements occurred at P50, the injected ear is noted as “treated”, while the non-injected ear is “contralateral”.

FIG. 14A depicts ABR results from four groups of untreated homozygous S1c26a4^{L236P/L236P} mutant mice over time (P30-P150). On the Y axis is ABR threshold in dB SPL in response to click stimuli, while the X axis represents age over time. Mice are grouped based upon hearing levels. Phenotypes such as circling were observed in groups of mice with degenerative hearing or stable levels of poor hearing.

FIG. 14B depicts ABR results from one group of untreated homozygous S1c26a4^{L236P/L236P} mutant mice over time (P21-P150). On the Y axis is ABR threshold in dB SPL, while the X axis represents noise stimuli provided (clicks, or pure tone at noted frequency). Animals in this group had stable levels of severely poor hearing over time and displayed a circling behavior.

FIG. 14C depicts ABR results from one group of untreated homozygous S1c26a4^{L236P/L236P} mutant mice over time (P21-P150). On the Y axis is ABR threshold in dB SPL, while the X axis represents noise stimuli provided (clicks, or pure tone at noted frequency). Animals in this group had stable levels of poor hearing over time and displayed a circling behavior.

FIG. 14D depicts ABR results from one group of untreated homozygous S1c26a4^{L236P/L236P} mutant mice over time (P30-P150). On the Y axis is ABR threshold in dB SPL, while the X axis represents noise stimuli provided (clicks, or pure tone at noted frequency). Animals in this group had hearing degenerate to poor by P60, at which point hearing stabilized at a poor level and animals displayed a circling behavior.

FIG. 14E depicts ABR results from one group of untreated homozygous S1c26a4^{L236P/L236P} mutant mice over time (P30-P150). On the Y axis is ABR threshold in dB SPL, while the X axis represents noise stimuli provided (clicks, or pure tone at noted frequency). Animals in this group had stable hearing and displayed no circling behavior.

FIG. 15 illustrates a perspective of a device for delivering fluid to an inner ear, according to aspects of the present disclosure.

FIG. 16 illustrates a sideview of a bent needle sub-assembly, according to aspects of the present disclosure.

FIG. 17 illustrates a perspective view of a device for delivering fluid to an inner ear, according to aspects of the present disclosure.

FIG. 18 illustrates a perspective view of a bent needle sub-assembly coupled to the distal end of a device, according to aspects of the present disclosure.

### DETAILED DESCRIPTION OF CERTAIN EMBODIMENTS

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some embodiments, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB4, and Pendred syndrome, respectively). Environmental causes of hearing impairment or loss may include, e.g., certain medications, specific infections before or after birth, and/or exposure to loud noise over an extended period. In some embodiments, hearing loss can result from noise, ototoxic agents, presbycusis, disease, infection or cancers that affect specific parts of the ear. In some embodiments, ischemic damage can cause hearing loss via pathophysiological mechanisms. In some embodiments, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some embodiments, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and PCT filing application US2019/060328, each of which is incorporated in its entirety herein by reference).

In some embodiments, nonsyndromic hearing loss and/or deafness is not associated with other signs and symptoms. In some embodiments, syndromic hearing loss and/or deafness occurs in conjunction with abnormalities in other parts of the body. Approximately 70 percent to 80 percent of genetic hearing loss and/or deafness cases are nonsyndromic; remaining cases are often caused by specific genetic syndromes. Nonsyndromic deafness and/or hearing loss can have different patterns of inheritance, and can occur at any age. Types of nonsyndromic deafness and/or hearing loss are generally named according to their inheritance patterns. For example, autosomal dominant forms are designated DFNA, autosomal recessive forms are DFNB, and X-linked forms are DFN. Each type is also numbered in the order in which it was first described. For example, DFNA1 was the first described autosomal dominant type of nonsyndromic deafness. Between 75 percent and 80 percent of genetically causative hearing loss and/or deafness cases are inherited in an autosomal recessive pattern, which means both copies of the gene in each cell have mutations. Usually, each parent of an individual with autosomal recessive hearing loss and/or deafness is a carrier of one copy of the mutated gene, but is not affected by this form of hearing loss. Another 20 percent to 25 percent of nonsyndromic hearing loss and/or deafness cases are autosomal dominant, which means one copy of the altered gene in each cell is sufficient to result in deafness and/or hearing loss. People with autosomal dominant deafness and/or hearing loss most often inherit an altered copy of the gene from a parent who is deaf and/or has hearing loss. Between 1 to 2 percent of cases of deafness and/or hearing loss show an X-linked pattern of inheritance, which means the mutated gene responsible for the condition is located on the X chromosome (one of the two sex chromosomes). Males with X-linked nonsyndromic hearing loss and/or deafness tend to develop more severe hearing loss earlier in life than females who inherit a copy of the same gene mutation. A characteristic of X-linked inheritance is that fathers cannot pass X-linked traits to their sons. Mitochondrial nonsyndromic deafness, which results from changes to mitochondrial DNA, occurs in less than one percent of cases in the United States. The altered mitochondrial DNA is passed from a mother to all of her sons and daughters. This type of deafness is not inherited from fathers. The causes of syndromic and nonsyndromic deafness and/or hearing loss are complex. Researchers have identified more than 30 genes that, when altered, are associated with syndromic and/or nonsyndromic deafness and/or hearing loss; however, some of these genes have not been fully characterized. Different mutations in the same gene can be associated with different types of deafness and/or hearing loss, and some genes are associated with both syndromic and nonsyndromic deafness and/or hearing loss.

In some embodiments, deafness and/or hearing loss can be conductive (arising from the ear canal or middle ear), sensorineural (arising from the inner ear or auditory nerve), or mixed. In some embodiments, nonsyndromic deafness and/or hearing loss is associated with permanent hearing loss caused by damage to structures in the inner ear (sensorineural deafness). In some embodiments, sensorineural hearing loss can be due to poor hair cell function. In some embodiments, sensorineural hearing impairments involve the eighth cranial nerve (the vestibulocochlear nerve) or the auditory portions of the brain. In some such embodiments, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some embodiments, hair cells may be abnormal at birth, or damaged during the lifetime of an individual. In some embodiments, outer hair cells may be able to regenerate. In some embodiments, inner hair cells are not capable of regeneration after illness or injury. In some embodiments, sensorineural hearing loss is due to abnormalities in hair cells.

As is known to those of skill in the art, hair cells do not occur in isolation, and their function is supported by a wide variety of cells which can collectively be referred to as supporting cells. Supporting cells may fulfil numerous functions, and include a number of cell types, including but not limited to Hensen's cells, Deiters' cells, pillar cells, Claudius cells, inner phalangeal cells, and border cells. In some embodiments, sensorineural hearing loss is due to abnormalities in supporting cells. In some embodiments, supporting cells may be abnormal at birth, or damaged during the lifetime of an individual. In some embodiments, supporting cells may be able to regenerate. In some embodiments, certain supporting cells may not be capable of regeneration.

### Solute Carrier Family 26 Member 4 (SLC26A4)

The SLC26A4 gene is highly conserved and encodes pendrin protein. The human SLC26A4 gene is located on chromosome 7q22. It contains 21 exons encompassing about 57 kilobases (kb) (NCBI Accession No. NG_008489.1). Full-length wild type pendrin protein expressed from the human SLC26A4 gene is approximately 780 amino acids in length.

Pendrin is an anion exchange protein, which functions as a sodium-independent chloride-iodide exchanger, as well as an exchanger of formate and bicarbonate. Pendrin has homology to sulfate transporters. In the inner ear, pendrin is thought to function as an ion exchanger, specifically as a chloride and bicarbonate exchanger, where it helps control the pH of endolymphatic fluid. The lack of appropriately functioning pendrin protein can result in an imbalance of particular ions. The resultant ion imbalance can disrupt the development and/or function of the thyroid gland and structures in the inner ear. In the inner ear of mammals, loss of appropriately functioning pendrin results in endolymph acidification, severe degeneration of sensory cells in the organ of Corti and vestibular maculae, and malformation of the otoconia and otoconial membrane.

Pendrin protein has a complex tertiary structure and is considered difficult to fold. It is thought that some of the mutations in SLC26A4 and/or pendrin lead to misfolding/defective trafficking and subsequent degradation. Although some pendrin variants do make it to the plasma membrane and show impaired transport function.

SLC26A4 protein is expressed in multiple non-sensory cell populations of the cochlea, vestibular labyrinth, and endolymphatic sac and duct. While not being limited by current theory, in the inner ear, pendrin is thought to be expressed in the epithelium of the endolymphatic sac and duct, on the apical membrane of transitional cells in the saccule, utricle, ampulla, and in a variety of diverse cell types in the cochlea (inner and outer hair cells, Deiter's cells, Claudius cells, spiral ligament, spiral ganglion, spiral prominence, external sulcus cells), and in marginal, intermediate, and basal cells.

Mutations in SLC26A4 gene have been associated with hearing loss and deafness (Albert et al., Eur. J. Hum. Genet. 14:773-779, 2006; and Qing et al., Genet. Test Mol. Biomarkers 19(1):52-58, 2015, each of which is incorporated in its entirety herein by reference). Mutations in the SLC26A4 gene alter the structure or function of pendrin, disrupting its endogenous function. There are over 200 reported mutations in SLC26A4 associated with hearing loss. For example, point mutations E29Q, V138F, L236P, G209V, L236P, V239D, V250A, D266N, E303Q, F345S, N392Y, R409H, T410M, T416P, L445W, L597S, D697, K715N, H723R, and E737D have been reported in patients from around the world (e.g., at least Chinese, Taiwanese, Mongolian, Turkish, Pakistani, French, Spanish, Czech, Iranian, Dutch, German, British, and/or North American patients) and are associated with syndromic or nonsyndromic hearing loss (Dai et al., Physiol Genomics 38(3): 281-290, 2015, and Tsukada et al., Ann Otol Rhinol Laryngol. 2015 May; 124 Suppl 1:61S-76S., each of which is incorporated in its entirety herein by reference). Additional exemplary mutations in an SLC26A4 gene detected in subjects having nonsyndromic sensorineural hearing loss or syndromic sensorineural hearing loss, and methods of sequencing a nucleic acid encoding SLC26A4 are described in, e.g., Albert et al., Eur. J. Hum. Genet. 14: 773-779, 2006; and Qing et al., Genet. Test Mol. Biomarkers 19(1):52-58, 2015, each of which is incorporated in its entirety herein by reference. Methods of detecting mutations in a gene are well-known in the art. Non-limiting examples of such techniques include: real-time polymerase chain reaction (RT-PCR), PCR, Sanger sequencing, next-generation sequencing, Southern blotting, and Northern blotting.

Mutations in the SLC26A4 gene and encoded pendrin protein have been linked with Pendred Syndrome. Pendred syndrome is an autosomal recessive inherited disorder comprising congenital sensorineural hearing loss, cochlear abnormalities (enlarged vestibular aqueduct or Mondini dysplasia) and thyroid enlargement (goiter). It is estimated that about 8% of congenital hearing loss cases are due to Pendred syndrome, and that Pendred syndrome has a prevalence of ˜8-16 of every 100,000 live births in the US or EU5, resulting in population of about 40-80,000 impacted individuals. In most people with Pendred syndrome, severe to profound hearing loss caused by changes in the inner ear is evident prior to, at, or shortly after birth. Classically, the hearing loss is bilateral, severe to profound, and congenital (or prelingual). However, hearing loss may be later in onset and progressive, the progression can be rapid in early childhood and may be associated with head injury, or infection.

Mutations in SLC26A4 gene are also known to cause autosomal recessive deafness-4 (DFNB4) with enlarged vestibular aqueduct (EVA), another congenital cause of deafness. This disease state may sometimes also be referred to as nonsyndromic enlarged vestibular aqueduct (NSEVA). Certain mutations of SLC26A4 are more likely to cause DFNB4, while others are more linked with Pendred Syndrome (Azaiez, et al. (December 2007), Hum. Genet. 122 (5): 451-7, which is incorporated in its entirety herein by reference). Patients with DFNB4 generally lack the additional Pendred syndrome presentation of goiter.

A correlation has been reported between Pendred syndrome and the presence of two mutant SLC26A4 alleles, while DFNB4 has been reported to sometimes be associated with either one or even zero mutant SLC26A4 alleles. Rarely, DFNB4 can also occur through mutation of the FOXI1 gene, or through digenic inheritance of heterozygous mutations in the SLC26A4 gene and the interacting genes FOXI1 or KCNJ10. For individuals with DFNB4 hearing loss, the degree of hearing impairment and its presentation may vary. For example, it is common in people with DFNB4 to be born with normal hearing, and progressively become hearing impaired during childhood. The majority of persons with DFNB4 (about 80%) report fluctuations in hearing.

Certain mice models homozygous for S1c26A4 knockout (KO) were reported to show severe endolymphatic dilation after embryonic day 15, by the second postnatal week, severe degeneration of sensory cells in the Corti organ and vestibular maculae, and malformation of otoconia and otoconial membrane has occurred. In mice, loss of pendrin is associated with the acidification of the endolymph.

As discussed above, hundreds of SLC26A4 gene mutations have been identified, and in recent years, various mouse models have accelerated the understanding of the pathogenesis of DFNB4 and Pendred syndrome associated with such gene mutations (see e.g., A. Nishio, et al., Slc26a4 expression prevents fluctuation of hearing in a mouse model of large vestibular aqueduct syndrome, Neuroscience 329 (2016) 74e82; T. Ito, et al., Progressive irreversible hearing loss is caused by stria vascularis degeneration in an Slc26a4-insufficient mouse model of large vestibular aqueduct syndrome, Neuroscience 310 (2015) 188e197; Y. C. Lu, et al., Differences in the pathogenicity of the p.H723R mutation of the common deafness-associated SLC26A4 gene in humans and mice, PLoS One 8 (6) (2014), e64906; T. Ito, et al., Slc26a4-insufficiency causes fluctuating hearing loss and stria vascularis dysfunction, Neurobiol. Dis. 66 (2014) 53e65; P. Wangemann, Mouse models for pendrin-associated loss of cochlear and vestibular function, Cell. Physiol. Biochem. 32 (7) (2013) 157e165; and X. Li, et al., SLC26A4 targeted to the endolymphatic sac rescues hearing and balance in SLC26A4 mutant mice, PLoS Genet. 9 (7) (2013), e1003641; each of which is incorporated herein by reference in its entirety).

In some situations, patients with different SLC26A4 mutations are associated with different clinical phenotypes (see e.g., H. Azaiez, et al., Genotype-phenotype correlations for SLC26A4-related deafness, Hum. Genet. 122 (5) (2007) 451e457; which is incorporated herein by reference in its entirety). Similar to humans, mice with different mutations are reported to have different phenotypes. For example, pds−/− mice are completely deaf and also display signs of vestibular dysfunction, severe degeneration of hair cells occur in both the organ of corti and vestibular, and malformation of otoconia and otoconial membranes was observed in vestibular. Although the Slc26a4^{loop/loop} mice with the p.S408F mutation are profoundly deaf and present abnormal vestibular behavior and malformation of otoconia, the morphology of the vestibular hair cells is normal. However the Slc26a4^{tm2Dontuh/tm2Dontuh} mice with the p.H723R mutation may display normal audio and vestibular phenotypes and inner ear morphology. Cell line studies have shown that certain SLC26A4 mutations only partially impair the function of pendrin (see e.g., B. Y. Choi, et al., Hypo-functional SLC26A4 variants associated with nonsyndromic hearing loss and enlargement of the vestibular aqueduct: genotype-phenotype correlation or coincidental polymorphisms? Hum. Mutat. 30 (4) (2009) 599e608; which is incorporated herein by reference in its entirety), indicating that in some situations, pathology associated with each of the different mutation is different. In addition, patients typically exhibit moderate to profound sensorineural hearing impairment (see e.g., Y. Yuan, et al., Molecular epidemiology and functional assessment of novel allelic variants of SLC26A4 in non-syndromic hearing loss patients with enlarged vestibular aqueduct in China, PLoS One 7 (11) (2012), e49984; which is incorporated herein by reference in its entirety), however, in almost all cases, existing Slc26a4 mutant mouse strains (e.g., Slc26a4^{tm2Dontuh/tm2Dontuh}) have a profound loss of hearing and vestibular function, and severe inner ear malformations that do not necessarily mimic human phenotypes.

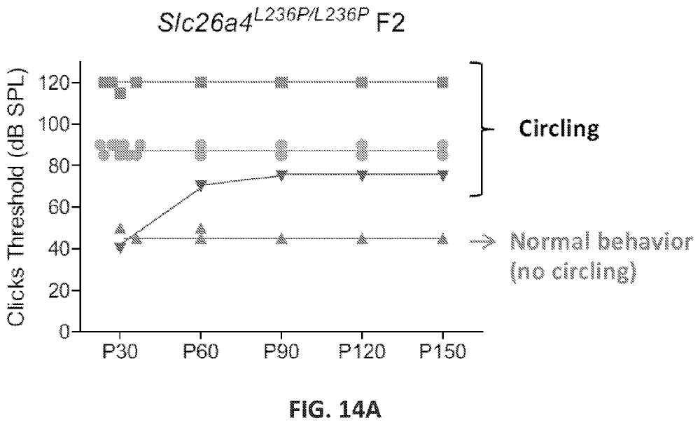

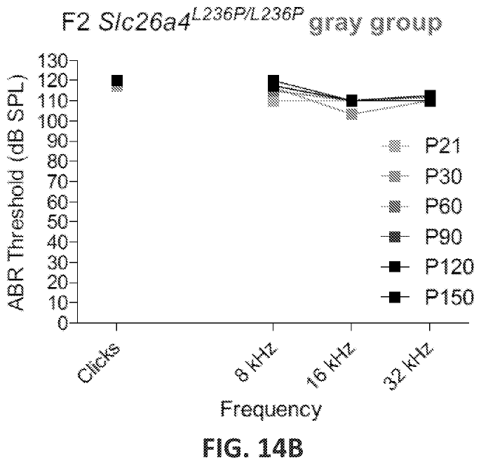

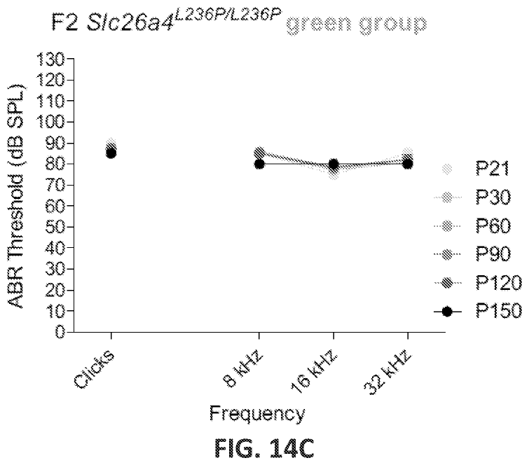

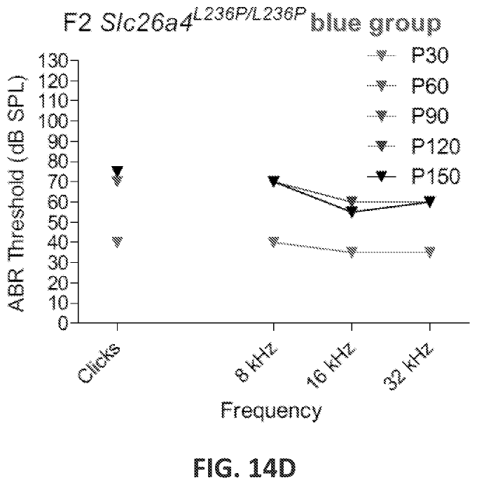

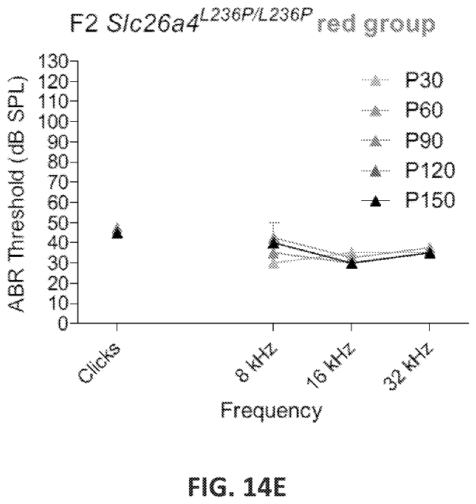

In some embodiments described herein, CRISPR/Cas technology was utilized to create S1c26a4^{L236P/L236P} mice to mimic the most common SLC26A4 mutation in Caucasians (see e.g., J. S. Yoon, et al., Heterogeneity in the processing defect of SLC26A4 mutants, J. Med. Genet. 45 (7) (2008) 4Ile419; which is incorporated herein by reference in its entirety). Mutant S1c26a4^{L236P/L236P} mice have variable phenotypic profiles, mimicking the human spectrum of disease manifestation. In some embodiments, the L236P mice exhibited moderate to profound hearing loss (see e.g., FIG. 14A). The Slc26a4^{L236P/L236P} mutant mice can mimic human disease states, and provide a useful tool for describing pathogenesis of Pendred syndrome & efficacy of potential gene therapy methods for alleviating symptoms associated with Pendred syndrome. In some embodiments, results generated in such a model may more accurately mimic human disease states and the results of potential therapeutic interventions when compared to previously described mouse models.

### SLC26A4 Polynucleotides

Among other things, the present disclosure provides polynucleotides, e.g., polynucleotides comprising an SLC26A4 gene or characteristic portion thereof, as well as compositions including such polynucleotides and methods utilizing such polynucleotides and/or compositions.

In some embodiments, a polynucleotide comprising an SLC26A4 gene or characteristic portion thereof can be DNA or RNA. In some embodiments, DNA can be genomic DNA or cDNA. In some embodiments, RNA can be an mRNA. In some embodiments, a polynucleotide comprises exons and/or introns of an SLC26A4 gene.

In some embodiments, a gene product is expressed from a polynucleotide comprising an SLC26A4 gene or characteristic portion thereof. In some embodiments, expression of such a polynucleotide can utilize one or more control elements (e.g., promoters, enhancers, splice sites, poly-adenylation sites, translation initiation sites, etc.). Thus, in some embodiments, a polynucleotide provided herein can include one or more control elements.

In some embodiments, an SLC26A4 gene is a mammalian SLC26A4 gene. In some embodiments, an Slc26a4 gene is a murine Slc26a4 gene. In some embodiments, an SLC26A4 gene is a primate SLC26A4 gene. In some embodiments, a SLC26A4 gene is a human SLC26A4 gene. An exemplary human SLC26A4 cDNA sequence is or includes the sequence of SEQ ID NO: 1 or SEQ ID NO: 2. An exemplary human SLC26A4 genomic DNA sequence can be found in SEQ ID NO: 3. An exemplary human SLC26A4 cDNA sequence including untranslated regions is or includes the sequence of SEQ ID NO: 4 or 5.

Exemplary Human SLC26A4 cDNA coding Sequence
(SEQ ID NO: 1)
ATGGCAGCGCCAGGCGGCAGGTCGGAGCCGCCGCAGCTCCCCGAGTACAGCTGCAGCTACATGG
TGTCGCGGCCGGTCTACTCGGAGCTAGCTTTCCAGCAACAGCACGAGCGGCGCCTGCAGGAGCG
CAAGACGCTGCGGGAGAGCCTGGCCAAGTGCTGCAGTTGTTCAAGAAAGAGAGCCTTTGGTGTG
CTAAAGACTCTTGTGCCCATCTTGGAGTGGCTCCCCAAATACCGAGTCAAGGAATGGCTGCTTA
GTGACGTCATTTCGGGAGTTAGTACTGGGCTAGTGGCCACGCTGCAAGGGATGGCATATGCCCT
ACTAGCTGCAGTTCCTGTCGGATATGGTCTCTACTCTGCTTTTTTCCCTATCCTGACATACTTT
ATCTTTGGAACATCAAGACATATCTCAGTTGGACCTTTTCCAGTGGTGAGTTTAATGGTGGGAT
CTGTTGTTCTGAGCATGGCCCCCGACGAACACTTTCTCGTATCCAGCAGCAATGGAACTGTATT
AAATACTACTATGATAGACACTGCAGCTAGAGATACAGCTAGAGTCCTGATTGCCAGTGCCCTG
ACTCTGCTGGTTGGAATTATACAGTTGATATTTGGTGGCTTGCAGATTGGATTCATAGTGAGGT
ACTTGGCAGATCCTTTGGTTGGTGGCTTCACAACAGCTGCTGCCTTCCAAGTGCTGGTCTCACA
GCTAAAGATTGTCCTCAATGTTTCAACCAAAAACTACAATGGAGTTCTCTCTATTATCTATACG
CTGGTTGAGATTTTTCAAAATATTGGTGATACCAATCTTGCTGATTTCACTGCTGGATTGCTCA
CCATTGTCGTCTGTATGGCAGTTAAGGAATTAAATGATCGGTTTAGACACAAAATCCCAGTCCC
TATTCCTATAGAAGTAATTGTGACGATAATTGCTACTGCCATTTCATATGGAGCCAACCTGGAA
AAAAATTACAATGCTGGCATTGTTAAATCCATCCCAAGGGGGTTTTTGCCTCCTGAACTTCCAC
CTGTGAGCTTGTTCTCGGAGATGCTGGCTGCATCATTTTCCATCGCTGTGGTGGCTTATGCTAT
TGCAGTGTCAGTAGGAAAAGTATATGCCACCAAGTATGATTACACCATCGATGGGAACCAGGAA
TTCATTGCCTTTGGGATCAGCAACATCTTCTCAGGATTCTTCTCTTGTTTTGTGGCCACCACTG
CTCTTTCCCGCACGGCCGTCCAGGAGAGCACTGGAGGAAAGACACAGGTTGCTGGCATCATCTC
TGCTGCGATTGTGATGATCGCCATTCTTGCCCTGGGGAAGCTTCTGGAACCCTTGCAGAAGTCG
GTCTTGGCAGCTGTTGTAATTGCCAACCTGAAAGGGATGTTTATGCAGCTGTGTGACATTCCTC
GTCTGTGGAGACAGAATAAGATTGATGCTGTTATCTGGGTGTTTACGTGTATAGTGTCCATCAT
TCTGGGGCTGGATCTCGGTTTACTAGCTGGCCTTATATTTGGACTGTTGACTGTGGTCCTGAGA
GTTCAGTTTCCTTCTTGGAATGGCCTTGGAAGCATCCCTAGCACAGATATCTACAAAAGTACCA
AGAATTACAAAAACATTGAAGAACCTCAAGGAGTGAAGATTCTTAGATTTTCCAGTCCTATTTT
CTATGGCAATGTCGATGGTTTTAAAAAATGTATCAAGTCCACAGTTGGATTTGATGCCATTAGA
GTATATAATAAGAGGCTGAAAGCGCTGAGGAAAATACAGAAACTAATAAAAAGTGGACAATTAA
GAGCAACAAAGAATGGCATCATAAGTGATGCTGTTTCAACAAATAATGCTTTTGAGCCTGATGA
GGATATTGAAGATCTGGAGGAACTTGATATCCCAACCAAGGAAATAGAGATTCAAGTGGATTGG
AACTCTGAGCTTCCAGTCAAAGTGAACGTTCCCAAAGTGCCAATCCATAGCCTTGTGCTTGACT
GTGGAGCTATATCTTTCCTGGACGTTGTTGGAGTGAGATCACTGCGGGTGATTGTCAAAGAATT
CCAAAGAATTGATGTGAATGTGTATTTTGCATCACTTCAAGATTATGTGATAGAAAAGCTGGAG
CAATGCGGGTTCTTTGACGACAACATTAGAAAGGACACATTCTTTTTGACGGTCCATGATGCTA
TACTCTATCTACAGAACCAAGTGAAATCTCAAGAGGGTCAAGGTTCCATTTTAGAAACGATCAC
TCTCATTCAGGATTGTAAAGATACCCTTGAATTAATAGAAACAGAGCTGACGGAAGAAGAACTT
GATGTCCAGGATGAGGCTATGCGTACACTTGCATCCTAA
Exemplary Human SLC26A4 cDNA coding Sequence
(SEQ ID NO: 2)
ATGGCAGCGCCAGGCGGCAGGTCGGAGCCGCCGCAGCTCCCCGAGTACAGCTGCAGCTACATGG
TGTCGCGGCCGGTCTACAGCGAGCTCGCTTTCCAGCAACAGCACGAGCGGCGCCTGCAGGAGCG
CAAGACGCTGCGGGAGAGCCTGGCCAAGTGCTGCAGTTGTTCAAGAAAGAGAGCCTTTGGTGTG
CTAAAGACTCTTGTGCCCATCTTGGAGTGGCTCCCCAAATACCGAGTCAAGGAATGGCTGCTTA
GTGACGTCATTTCGGGAGTTAGTACTGGGCTAGTGGCCACGCTGCAAGGGATGGCATATGCCCT
ACTAGCTGCAGTTCCTGTCGGATATGGTCTCTACTCTGCTTTTTTCCCTATCCTGACATACTTT
ATCTTTGGAACATCAAGACATATCTCAGTTGGACCTTTTCCAGTGGTGAGTTTAATGGTGGGAT
CTGTTGTTCTGAGCATGGCCCCCGACGAACACTTTCTCGTATCCAGCAGCAATGGAACTGTATT
AAATACTACTATGATAGACACTGCAGCTAGAGATACAGCTAGAGTCCTGATTGCCAGTGCCCTG
ACTCTGCTGGTTGGAATTATACAGTTGATATTTGGTGGCTTGCAGATTGGATTCATAGTGAGGT
ACTTGGCAGATCCTTTGGTTGGTGGCTTCACAACAGCTGCTGCCTTCCAAGTGCTGGTCTCACA
GCTAAAGATTGTCCTCAATGTTTCAACCAAAAACTACAATGGAGTTCTCTCTATTATCTATACG
CTGGTTGAGATTTTTCAAAATATTGGTGATACCAATCTTGCTGATTTCACTGCTGGATTGCTCA
CCATTGTCGTCTGTATGGCAGTTAAGGAATTAAATGATCGGTTTAGACACAAAATCCCAGTCCC
TATTCCTATAGAAGTAATTGTGACGATAATTGCTACTGCCATTTCATATGGAGCCAACCTGGAA
AAAAATTACAATGCTGGCATTGTTAAATCCATCCCAAGGGGGTTTTTGCCTCCTGAACTTCCAC
CTGTGAGCTTGTTCTCGGAGATGCTGGCTGCATCATTTTCCATCGCTGTGGTGGCTTATGCTAT
TGCAGTGTCAGTAGGAAAAGTATATGCCACCAAGTATGATTACACCATCGATGGGAACCAGGAA
TTCATTGCCTTTGGGATCAGCAACATCTTCTCAGGATTCTTCTCTTGTTTTGTGGCCACCACTG
CTCTTTCCCGCACGGCCGTCCAGGAGAGCACTGGAGGAAAGACACAGGTTGCTGGCATCATCTC
TGCTGCGATTGTGATGATCGCCATTCTTGCCCTGGGGAAGCTTCTGGAACCCTTGCAGAAGTCG
GTCTTGGCAGCTGTTGTAATTGCCAACCTGAAAGGGATGTTTATGCAGCTGTGTGACATTCCTC
GTCTGTGGAGACAGAATAAGATTGATGCTGTTATCTGGGTGTTTACGTGTATAGTGTCCATCAT
TCTGGGGCTGGATCTCGGTTTACTAGCTGGCCTTATATTTGGACTGTTGACTGTGGTCCTGAGA
GTTCAGTTTCCTTCTTGGAATGGCCTTGGAAGCATCCCTAGCACAGATATCTACAAAAGTACCA
AGAATTACAAAAACATTGAAGAACCTCAAGGAGTGAAGATTCTTAGATTTTCCAGTCCTATTTT
CTATGGCAATGTCGATGGTTTTAAAAAATGTATCAAGTCCACAGTTGGATTTGATGCCATTAGA
GTATATAATAAGAGGCTGAAAGCGCTGAGGAAAATACAGAAACTAATAAAAAGTGGACAATTAA
GAGCAACAAAGAATGGCATCATAAGTGATGCTGTTTCAACAAATAATGCTTTTGAGCCTGATGA
GGATATTGAAGATCTGGAGGAACTTGATATCCCAACCAAGGAAATAGAGATTCAAGTGGATTGG
AACTCTGAGCTTCCAGTCAAAGTGAACGTTCCCAAAGTGCCAATCCATAGCCTTGTGCTTGACT
GTGGAGCTATATCTTTCCTGGACGTTGTTGGAGTGAGATCACTGCGGGTGATTGTCAAAGAATT
CCAAAGAATTGATGTGAATGTGTATTTTGCATCACTTCAAGATTATGTGATAGAAAAGCTGGAG
CAATGCGGGTTCTTTGACGACAACATTAGAAAGGACACATTCTTTTTGACGGTCCATGATGCTA
TACTCTATCTACAGAACCAAGTGAAATCTCAAGAGGGTCAAGGTTCCATTTTAGAAACGATCAC
TCTCATTCAGGATTGTAAAGATACCCTTGAATTAATAGAAACAGAGCTGACGGAAGAAGAACTT
GATGTCCAGGATGAGGCTATGCGTACACTTGCATCCTGA
Exemplary Human SLC26A4 Genomic DNA Sequence
(SEQ ID NO: 3)
CGAATGAATGGAATACTTAAACATCCAACTCGTGGTAACACAGCACATCAGTGTTTACTTGAAA
CAATCCTCAAGCTTCCCTTTTCTCCCTCCATTAACAAACCACAGATTTCATGAGATAAGATATG
CAAGGATAACTTTAAAAGTGCAATACACTTAAATAATAAGAAATTTGGAAGGATCCTGAAATTA
TTCTTTGCTTAGAAAACTATGCCATTATTGGCTCGACATAGTGGCACAGGCCTGTAATGCCAGC
TACTTCAGGGACTGAGGTGAGACCCCCATCTCTAAAAAAAAGAAAGAAAACATCCAGGCCAAGT
GCAGTTGGCTCCTGCCTGTAATGTCATTTTATAATCAGCATTTTGGAAGGCTGAGGTGGTAGGA
TTATTTGAGCCCAGGAGTTTGAGACCAGCCTGGGTAACAAAGCGAGACCCCATCTCTTGAAAAA
AAAAAATCATCATTAAAACAAACATTAAAAGAAACCCACCTGGGTGTGGTGGCTCACACATGTA
ATCCCAGCACCCTGGGAGGATGAAGCAGGTGGATCTCTTGAGCACAGGAGTTTGAGACCAGCCT
GAGCAAGATGGCAAAACCCTGTCTCTACAAAAAATACAAAAATTAGCCGGGTGTGGTGGCACAT
GCCTATAGTCTCAACTACTTGGGAGGCTGAGGTGGAGAATGGCTTGAGCTTTGGAGGTGAAGGT
TGCAGTGAGCTGAGATAGTGTCACTGCACTTCAACCTGGGTGACAGAGCCAGACCCTGTTTAAA
ACAAACAAACAAACACACACACACACACACACACACAGAAAACCCCTCAAAACCCAAAACTATG
CCATCATTTCGTTCTAGCAATTTACTAAACCACATATCAAGTTTGCATTCATATTAAAGAAGAG
GCAGATGGGCTCTGCCTGAGAAATTTATTGAGTTATAGTTTTATTGACACTGTAAGTTGTTTAA
AATTGAATGGATTGCCACATAATATTTACAAATAATTTCATTTTTTGATGTTTTAATGGTTGGA
TTCTCTTCTTTTAAAACTAGTTTTTTTTCTACCCAAATATTTCTCAAAATTCCATTGTTAAACA
ATCAATTGATCAATAATTTAACAAGTCTGGTACCTATTATATTGTCATAATTTGTCAAGGTATT
GTGTAGGGAAATAAAGAGACTAGCATTATAATTTTACTTTAATAAAAATGTATACCTATGTCTG
CATAGAAAGAATAAATAAAAAAAGAAGCAAAACAGTGATTATCTGGATAATGAGATAAAATGTG
ATTACTATATTATACATTAATGTAGTTTCCAAATTATTTACTACAGGCACATCTTATTTTTGTA
ACACCATAAAAGACATATACTATTTATGTCCCTAGGGAGCATATCTTATAACTGGAGAAGAAAA
TAAAGTAGCATACAGTAAACAAATAGAAAACAATTGAGTGCAAGATAAAAATAACAACAAAAAA
CCAAGAAAACAATTGAGTGCAAAACTGTATGATTACTGAGAATAAGCCCAATAAGTTGCTCAGA
GAAGAAGAGATCAGAAATATAGAAAGAAAGTTCAGTTCAGTTCTCTTTTTGTATAAGTATGACA
AAAATTTAGCATTTGCTATCTTGAGAAAATTAACACGCATAATGAAATAAAAGCTCAGGATTAG
GCCTGGCTCAAGTGATCCTCCCGCCTCAGCTTCTCAAAGTGTTGGGATTACACTGTGAGCCACA
GCGCCCAGCCAACATGTTTGCATTCTTTTGCAATTCTTCTGATTTGAGAACATTTTAAATTGTA
AAAAAAAAAAAAAAAAGAAAAAATTTATACCACAATATTCGCTCTCAGACAAAAATAATGTAGC
AAGATTGACTGCTCTTAAGTTAAATAAACGAAGGATTCAATTAAATCATTCTTGTAGCACACGT
AGTCACTCAAAAGCATAGATACTCTGTCAGGTGTGGTGGTGTGCACCTATAGTCCCAGCTATTC
TGGAGACTGGGAGTTCAAGTCCAGCCTGGGCAAAATAGCAAGATCCCATCTCTAAAAAAGAAAA
AAAAGGCATAGATACACTATAAATTTATGTAAATATCTATATTTTATTAATTATAGAATTCTAT
TATAAAACTCATTTTATGAATCACAAAATACGCTATCAATTAAACTATAATATATAGTAGATCA
TCAATTATAACATGAACCCTGATAAAATGTGAAAAATCTGTATTTTATAATCAGTGAAATAAGA
TACACATTTTGTGACCATTTGATTAGTGTCTACTGCACCCATAAATATGAGGCCAGGGTATAAG
CTTTTGTTCATCATTGTACCCACAGCCAGCACAAATAATTTCCTAATAAATCCTTGAATGAATG
AATGAATGAACAGGTGAATGCGTAATCAAACAAGGGAGTTTGTCTTACACTGCAAATGCCTAAA
TGATAAATAAGAAAATGCCTTTTAAAAAAGTATTAACTGCTAAATAAGTACTGTAAAGTATCAA
TAATATTGTCTGATATTTATTTTTAAGTGATGCATCTATTTCTTTGGAAAGAAGTCCTGAATAT
TTTAAAAGTCTGATAAATAAGAACCACCCACCTCTGTGTGAAACAGGAGAAGGTTGCCTTATAA
AGCCCAAGTGAGTAATCTAAGGTGTTTAATATCACAATTAAATAGGGTGGAGAGAGCAAGCTGT
TATTTCCTCCTACTCTTCTGTTTTTAAAAAGCCAAATTCAAACGGTCTGCCTTTTACCTATGAG
AAAAGCAGTTCTATACCCTTATAGAATCCTGTAACAGAGATACCATTTTGTTCTGCCCCAGCTA
TTTCTGCTATGGAAAAGCATTGAACAGAAAAAAGCAATTATCTTCATCCAGATACATGCAGGAC
TTATTATATTGTGATTATTGAACCAAACTCTTATATAGAGAGATGCATTTCAAATAAGAATTCT
ATTAGCCAAGCTAAGTTACTCTTTTGCCTCCTGTTGTTACTCAAGTCTTTTCTCTTCTGTCCTT
CTGCCAGCCTTACCCCACTCCTTAATCCTCTGAACCAGCAAACCATTGCCAAGTTCTGATGCAA
AGTGGTTTATAGGCCTGACTGGACCAGACTAAAAGTGTTCAAAATAGCAAGCAACAAGGAGCAG
AAATCCATATTAGAATGGGATATGGACTATATTTATATTGGTACAGAATGCCTTCAATAAAGAG
TTGTGAGTTGTGTAGGTGAGTTGCCATGGAGCTACAAATATGAGTTGATATTCTGAAATCCTAG
ACAGCCATCTCCAAGGTTAAGAAAAATCCTTATGCACTCACTTGCAAAGATATCCACAGCATGC
TCTTAATGGAGAAAAACAAAGCCTTAGATCAAATATGTAAAGTAATTTTTAGTTTTTTGAAAAG
GTATGTTTGGGCTATAGATAAATCTGTTCAAAAAACATGAGAGAAGATAATAATGGTTGAAAGG
AGACACAGTGCTTGCCCTCAAGAAGTTTTTGTCTAGTGAGGGAGAGAGAACTTGTATGTAAATA
AAATTGTGTTACTAAGGTAGATAGTGAGAAGTAACTTAAGAGAGGATCAGATAAGGTATTAAGA
GAATACAGAAAAGGGTCTGGATTAATTCTGAACAGCATCAAAGAATGTTCTTGCAAGAGATAGT
GTTTTCACCAGATCTTGAAGGTATGGATGAGGGTATACAGAGTGAGTATATTCAGATTCTACTT
TAAAACAAATACTTTCCTCTGTTGTAGTGGAGTTGAGCTATACATCCAACAATAATGAAAAAAT
ACACGCATATATACATATATGGAGAGAGATACATATTTTAGTACATGTAGCAATTGATTAATAA
ATGTACAGTTTAAGTCGCATGCAAAACCTTGGAGTGATAGCAAACTTCATTGTAGGATGTTTAG
CAGCATCTCTGGTCTCTACTCACTAGATCCCAATAGCATCTCCCTAGGTGTGACAACCAAAAAT
GTCTCCAGGCATTGACCTCTGGAGGCAAAAAAAGCCCTTTATTAAGAACCAGTGGTATACATAA
GTAAAACATACACAAGAGATTCCTCCCCTCTTCTCTGTATGTGAATAAAAATTGCAAAGTTCAT
GACCTGGATTTTCCTTTTAGGTTTCTTCTTTAGTGGTTCTTAACTTCATTGGGTGAAGTAAGCC
TTTGAAGATCTGTTGAAAGCTGTTGACTCATTCACTTCTCAGGAAAACGCACATGCTGACTACC
ATTTCAGAGAATTTGCATCAGGGTTCTCTGGGGAGGAGTTCTGAGTTCTGTTTCCAGGAGCTCG
TAGAATTGTCATGGTCTGCATATGCAAGGCAGGTGGATTACGGAAGGTTGATGTACAGAGGTCT
GTATTTTGGAGCCTCTTCTGTATTTACTTCAGAACACTAACAATCAGGCGAGAATGTTCTGGTT
TATCAAACCCTTCCTTCTGCCTTTCATCTTAACCATGCATTAGTTTTAACAAAGTTCATCCCAA
CAGAAGACAAAACACTGATGAGGTAGGATAGCTCCAGCTCCTCCTCCCTCTCTTCTAGTCTTGA
TTTCCATGTAGTCGAGTTTATTCCTTCCCTGATTGTCGAGGAGAATGAGAAAAAGAAAAAACAG
AGTCTAGTGGGTAAGAAAGGGCCACCTGGACGGCTTGATTTGGATTGTGAAATAAAACACACAC
ACATGCACACGTAGAATAAGTGGCTAAAATCTGAGTAAATCGTGAACTCTCTGTATCCTCCACC
CATTGAATACTCCTAAAAGACTTTCTAGAAATTCAAGGACTTATTAATATAGAAACCTGGCCAT
TGTTCCTCTTCTCCTCCCCATGTGGTATGAGAGCACCTGTGGCAGGCTCCCAGAGACCACGGAC
CTCTTCCTCTAGGCGGGCTCTGCTCTTCTTTAAGGAGTCCCACAGGGCCTGGCCCGCCCCTGAC
CTCGCAACCCTTGAGATTAGTAACGGGATGAGTGAGGATCCGGGTGGCCCCTGCGTGGCAGCCA
GTAAGAGTCTCAGCCTTCCCGGTTCGGGAAAGGGGAAGAATGCAGGAGGGGTAGGATTTCTTTC
CTGATAGGATCGGTTGGGAAAGACCGCAGCCTGTGTGTGTCTTTCCCTTCGACCAAGGTGTCTG
TTGCTCCGTAAATAAAACGTCCCACTGCCTTCTGAGAGCGCTATAAAGGCAGCGGAAGGGTAGT
CCGCGGGGCATTCCGGGCGGGGCGCGAGCAGAGACAGGTGAGTTCGCCCTGAAGATGCCCACAC
CGCCCGGCCCGGGCTCCACTCCCGGGGAGGCCTCGAGGGTTGCGGATGGGACTCTTAAGTGGTC
ACGGATCAGGTGGGCAGGGGGCAGTACAGCTTTCTTTCTGAGACGCCGAGAGCGAACAGGCTGC
TCGGAAAACAGGACGAGGGGAGAGACTTGCTCAATAAGCTGAAAGTTCTGCCCCCGAGAGGGCT
GCGACAGCTGCTGGAATGTGCCTGCAGCGTCCGCCTCTTGGGGACCCGCGGAGCGCGCCCTGAC
GGTTCCACGCCTGGCCCGGGGGTCTGCACCTCTCCTCCAGTGCGCACCTGGAGCTGCGTCCCGG
GTCAGGTGCGGGGAGGGAGGGAATCTCAGTGTCCCCTTCCAGCCTTGCAAGCGCCTTTGGCCCC
TGCCCCAGCCCCTCGGTTTGGGGGAGATTTCAGAACGCGGACAGCGCCCTGGCTGCGGGCCATA
GGGGACTGGGTGGAACTCGGGAAGCCCCCAGAGCAGGGGCTTACTCGCTTCAAGTTTGGGGAAC
CCCGGGCAGCGGGTGCAGGCCACGAGACCCGAAGGTTCTCAGGTGCCCCCCTGCAGGCTGGCCG
TGCGCGCCGTGGGGCGCTTGTCGCGAGCGCCGAGGGCTGCAGGACGCGGACCAGACTCGCGGTG
CAGGGGGGCCTGGCTGCAGCTAACAGGTGATCCCGTTCTTTCTGTTCCTCGCTCTTCCCCTCCG
ATCGTCCTCGCTTACCGCGTGTCCTCCCTCCTCGCTGTCCTCTGGCTCGCAGGTCATGGCAGCG
CCAGGCGGCAGGTCGGAGCCGCCGCAGCTCCCCGAGTACAGCTGCAGCTACATGGTGTCGCGGC
CGGTCTACAGCGAGCTCGCTTTCCAGCAACAGCACGAGCGGCGCCTGCAGGAGCGCAAGACGCT
GCGGGAGAGCCTGGCCAAGTGCTGCAGGTAGCGGCCGCGCGGGCCTGCGTAGAGAGAAGCGGAG
CGGGGCGTCCACGCCTTGGGGAGGGAAGGGCGTCCCCAGCGGGCGAGAGTGGGGTGCGGGCGGC
GGAGCCCCTGGGCGCCAGCTGCTTCTCCCAGAGGCCCGACTTTCGGTCTCCGGTCCTCCACGCC
GCCCTTCTGGTGGGAGGGTGGCTCCATCAGTCTCGGGCCCGAAATGAACTTACCTGGGAAACTC
GCCTTTGGGGAGAGTGGGTTCTAGGAGCCCCGTCTCTCTTTTTCCTCTCTGAAGGAAACTTGGA
GTGCCTCTTGGGGTACAGTGGGTCCCTGTTGCCTTCTTGGGAGCTTGTTTAAATGAAATGAATA
GGGAAACCCAGCTCTTGACCAGGAGGAGTCCTTGAAACACTCAAGCTAAGTAGGCGGGCTACCA
TTCAGTTAGAGACCAGGATGCAAGCTAGAACCCAGGGGAGCGCGGGGTGTGCCAAGTACTTCAT
CAGCAGGCTGTGGGACCCCTGGGGAAAGCCACCCTCAGTCTCTAAACCCAAACATGCCGTAACT
AGATGTCACAAACATAAAGAAATTAGAGTTTCTAAAACCTTTCATTATAGAACATTTCAAATAT
ATGCATAAGTAGACGTAGTAGTATAATGAACTCCCCACCCCCACCGTTTTTAACCCATCACTCG
GCCTCAACTGTGATCAAATCCAAGTTACTCTTGTTTCTTGTATCTACACCTACTTGCTCCCTCC
AGTATTATTTTTAAGCAAATCTCATATAGCATACTGTCTTTATGTATTTTAGTGCATATCTTTA
AAGACTCTTAAAAAATATAACCACATTTCATGATCACACCTTAATATCTAAAAATAACCTCCTA
ATATCAAAAATTGAGTCATTATTAAAATGTCCAATCGTCCCATAAATGCCTTAATTAAAAAACA
ATTTATTCAAATCAGGAGTTATACCACCTACTTTTGATATTTTTCTTTCATCTGTAGGTCACTG
AATTTAAAAACTACCAAGTAGCAAGGTATAAGGTATACATTCCTCATGCTAAGATTTTTGTAAA
AACTAGCTCCAGGCTTGTATTGCCAAAATATACTCAGTGTGTTTATCTTCTTTAAAGAAAATAA
TAATAATAAGGCGCCTGGATTAGGAGTCTGAAAAGTAATCTCCATTTAGAGACTCCACATCCAT
CAGTCTCTGGCTGGACCAGAAAATAGGTTTTTGTGTAGGAATATTTTTTCAGAGGATTAAAAAT
GTGAGCGAGGGGTGGGACACTTAATCCTGTGTTCTCTAGAAGAGTGCATTTAAAAGGATAGATA
CAGTTCTTGGCAAAAGCATGGTAAGCACTTCAGGGTTATTATTTTCCAGGAAATACTTATCCTT
TTTCCAAATAGTTATAAACATGAGCAGAATCGAGTTCATAACTTTGTGATTTGCAAATTGGTTG
TGACTGAGATTGGATTGAAAACCCAGTTTTCTTGCTTTTTGACAGTTGTTCAAGAAAGAGAGCC
TTTGGTGTGCTAAAGACTCTTGTGCCCATCTTGGAGTGGCTCCCCAAATACCGAGTCAAGGAAT
GGCTGCTTAGTGACGTCATTTCGGGAGTTAGTACTGGGCTAGTGGCCACGCTGCAAGGTAAGAT
GTTGGCAGATTGAGAGTTCTGGTCTCCAGCAGGAGTTTAACACTTCTCCCCAGCTACCATAGGT
CTGTGACAGATGGTTGCTTACCCTTCAAGGCCTGTATCTTTCCTGTAGAGCCCCTTAGTGGAGA
GAGTCACCTCTCTTCTCCCCTTCCTTAGAGTTCTCTTCCTGGGAAACTGCTGCCCCACTAGGTG
CAGAGGTCCAATTTAGAGGCATATACTAGGCAGTGGCTTCTCAATTTTTTTAAATTTTATTTTA
TTTGAGACGGGGGCTCGCTCTGTCACCTAGGCTGGAGTGCAGTGGCGCAATCCTGGCTCACTGC
AAGCTCCGTCTCCCGGGTTCACGCCATTCTCCCGCCTCAGCCTCCCCAGTAGCTGGGACTACAG
GCGCCCGCCACTACGCCCGGCTAATTTTTTGTATTTTTAGTAGAGACGGGGTTTCACCGGGTTA
GCCAGGATGGTATCGACCTCCTGACCTCGTGATCCGCCTGCCTTGGCCTCCCAAAATGCTGGGA
TTACAGGCGTGAGCCACTGCACCCAGCCAGCTTCTCTATTTTCATTGACCACAATTCAATAAGA
AATGTGTAAAGAGTTTCAAGTCAAGATTTAAAAAAAAAAAAGAAATGTGTTACATCCTGATATA
TAGATATATATCTAAAGTTCCTGTGAAATATTTATTATAACAATGTGCTAATCTTTCACTTTGT
TCTATTATGCTTAGGTTTTTTAAATGATGATTGCAGCACATTAAATTATTTTATATCCACACGT
GGATCTTTTTTGTTGGTTTGTTTTGCTTTGCTTTTTAGAGATACAGTCTTGCTCTGTCACCCAG
GCTGGTGTGCAGTGGCACGATCTTGGCTCAATGCAACCTCTCTGCCTCTCGGGTTCAAGTGATT
CTTGTGCCTCAGCCTTCTGAGTAGCTGGGGCTACAGGCATGTGCCACCATGTCCAGCTAATTAC
TGTATTTTTAGTAGAGATGGGGTTTCGCCATGTTGGCCAGGGTGGTCTGGAACTCCTAACCTCA
AGTGATCCGCCCTCCTTGGTATCCCAAAGTACTGTGATTACAGGCATGAGCCACCATGCCCAGC
CTCCCACACGTGGATCTTGAGGTGTAGTTTGAAACTGGCTTAAGGAAAAGTGAAGGGAGCTTCC
TAGCCTGGACTCCACATGAATGTTTTGCCTGCCCCTCATCCCTTTTTAAAAAAATTCATCCTCT
TATTTTAATGTGTGGGAAAATGTGCCAACATAGGTGGTTATTTGTTCTTTTATAAACTGAGTTC
ATACCATAATATTCCCATAAGCATCTTAGAAATCTGTTGTATAGTTGGTGCCTAGCAGCATGGC
ATTCTCCTCTGTCCCCTATCCCCACCATCTCGTTTCTGCCCCACCCCCAACACCCTGACCAATT
TCCCAAAGACCCTGAATCATGAAAACAGAAATATCGTTATTTATTATAATTTGTGAAACACCTA
ATATGTTCCAGGCACTAGGTATGCTGACACGAATAAGATGCATCCTCTGCTTCCAAGGAACTCA
TAGTCCAATGGAGAAAGAGAAAACAATTCAATGTGATAAGGGCTGTGGCAGGGGAGTGCAAATT
ACTTGCTAAGGGAGTGCAAATGAGGTTCACTCAAAGCGGTCTTAGGGGATCTTGAATAGCTTCC
TGGAAGTTGAATGTCAAAGCTGAGTTTCCCTGATGGCTAAGTCTGAGTTTGCCAAGTGATGGGA
AGTAGGGTGGGGTGGGGATGGCTTGAAAATAGACTATTTCAGAAGCTAGTAATGAATGTTGAGC
AATATTGTGAGCTAAAATTTGGCTATGGAGCTTTCAAGATTGCTCAGATCAGGCCAGGTAGATG
CCCGAAGAGAAGGAGGATGGCCTATGAACCCAGGGTGGAGCCTCAGCCTGATCAAGCATGTTCT
TAATCACAAAGGTGGACGGGAGAGAGACTAGGATGGGCTGTGTGGTTCCTAAGTTAAACAATGC
CAAGTATTCTACACTGTGCTCTAAGGGCTGTGACATGGTGACACCCTGTGACTCCTGGGGGACA
GGAACCATGCCTCATTCATGTTTGTTCAGCAGGTGCCTAGCACAGAGACAGGCACATAATAGAT
GGTATATTTAGTGAAAAAGATTGAATTGCATTCCTGATAATGAATCTTCTTTATATATAAAAAA
TGGTGGTTCATGCTCCATGTCTCCCCAGTTTTCATAGACAAGTATCTTATAAGAATTCATTTTG
TTTATAAGCATGAGACACTATGCTAAGTAAGAATGTTGCCAGCTATCTCATTTAATCCTCATAA
CAATCCTGCTAGGTAGGTCTATTATTATTTCATTTTCAGTAGGTGAAAAAATCTATTAAATCTT
GCCCAGGGTCACGTGGCTGGTAAGTGGTACAGCTGGGATTTTAATCCGGTGTCTATTGTGTAGT
TCCTTACTTTATGGGTAAGATGCTTTTCTAGAAACATTATCTCTATAAAGTTTTCTGTGTAAGG
TGAATTTCTGGGAGGTTAATCTGGCTGAGTTAGACATCCATTTATCATTCATTCACTTAATAAA
TATTTATAAACTAATTCTGTAGACCAGGTATCCAGCCATGTGCACTCAGTGGTTCATTAACAGG
CACAGGTGCTGACTTCATGGAGCTTTGTAAAATTATTTTGAGGGGTAGGGATGGGAATTAAATA
AGTAGATAAATAGAAAAATAATTATAGATTGCAATATCTATTGTGGTGGATTAAATATACTGCA
ATAGGGATTAATTTGAAAGGTGGATCTTTATTTTTATATTTTTTTGAGACAGGGTCTTGCTCTG
TTGCGCAGGCTGGAGTGCAGTGGTGAGATCACAGCTCACCACAGCCTTGGCCTCCTGGGCCCAG
GTGATCCTCCCATCTCAGCCTCGCCAGTAGCTGGAACTACAGGCATGTGCCACCATGCTTGGCT
AATTTTTGTATTTTTTGTAGAGACAAGGTTTCACCATGTTGCCCAGGCTGGTCTGGAACTCCTA
GGCTCAAGTTATCTGCCTGCCTCGGCCTCCCAAAGTGCTGGGATTAGAGGTGTGAGCCATTGTG
CCCAGCCAGGAAGGGAGGTCTATTTTAGATATAATGGTCAAGGAAGAACTTACTGAGAAGGTGA
AATTTAGTAAAAGACAAGAGAATGAGAAGGAGACAGCCATGTGATAAGCAGAAAAAAAGAGTGT
ACCAGGTAAAGGTGCAAAGGCCCTGAGGCCAGAAAACTTAGCATTTTCCAGGGAGGAAGAGGAA
GAATTGTAGTGTGGATGAGTGCAGTGGGGGAGGGTGAGTAAACCATGAAATAAACTGAGGCAGG
ACTGGGATTAGGATGAGGTAAGGGAGGCACTCACTCTCAGGATGCCTCTTTAAATATTGTACCC
TAGGAAACTCATTTGCCTCACCCTATTCCTGGCCCTGTGGGGATATTGCTGTGAATTAAAGGGA
GAATGGGTGTGTCAGCTTGAACCAGATGATGCAGGCTATGAAGAGAGCTGCAAGATAGGAATAT
ATTGTGCAGCAAGAATCACTGACACATAATGATGAGTGTGGATGAAAATCACTGATGCCTCCAA
GGTTGTTGGCTTGAGCAACCGGATAGATCATTACATCATTTATCTAAATGGCAAAGACAGGCTG
AGGAGCAGGTTTGGGATAGGAAATCAAGTTTCCTTTTGGATATGCTAAGTTTGAGACACAGGGT
GAGACATCTAAGTGGAGATGCCACCTAGAGAAGTGGGAGCTACTGCAAGTGTGAAGGTTAGAGG
AGAGGTCTAGGCTAGACATAAAAATCAGGAAAGCATTGCTGAAGAGGTGATATTTACAAGAGTG
GGAATGCCTGTGATTACCTAGGAAGAAAGGGCAGGTGTAGAAGAAAAGAGAGCCAGAGTACTTA
GAAGTTAGGAAGGCAAGGAGAGGGGCAGCCAAGAACCTGAGAAAGCCTGGCGCAGGAAGTTGAA
GACAAAGAACAACCAAAGGAATGTGGGATCAGAAGCCAAGAGAGAAGGTTTAAAAAAAAAAAAA
AAAAAAAAAAAGCATAGAGGGGCTGAGAGTGTTGAATTTGCCTGAGGGGTGGGGTATGATGAAG
GATTACAGACTGCTTTTAAGAAGTTTTGCCATGAATCAGAGGGAGATAAGAGATGAAGACAGAG
TGAAGGGAGAATTTGTTTTTCTTAATATGGGAGATACTAGAACGTATTTGTGAACCGATAGGAA
TGACCTAGTAGAAAGAAAGAGATCTGTGGTGCAAGAAGTACAAGAGAAATAACTAGAAGCAAAG
TAGTAAAAAGGGGCAAACGTGGAGGGGTTTGAGAATGGAACTAGGAGAACTACTCCTTAAAGGC
AGGAGGGATATTTCTTCCATTTTCAAAGGAAGATAGCCCAAATGGTGCAGATGCAGACGGGTTT
GAAATTTTGATGAAAAGTAACTTGGGAGCATCTCATGCATTCTGTGTTTTCGATTAAATGCAGA
ATATGTCCCTTAACTTTGAGTCTGGTGGAGAAGGGTTTGGGCAGATTGAGGTAAGTAGGGGAGG
TTTGAATAGTTGTCTCAAGGAGAGGAAAGGCACACCCCCTGAGGAAATGTAACTGGGTCCCCAG
GCAGTGTTGAGTGCCCATCTAGCACTTGTGGTCACACATTTTACATGAAATTGGTTAGCCCAGA
TGTAGATGCCTTCAGTCCAGTTTATCTAGCTGCTCAAAGGCTCTCATGGATAAGGTAGATGTTT
GAGGTCAGCCAAATTGGGGTTTTGCCAGACAAGAAAAGGAAGGAAAGAGAGAGTGAAAAAAGGG
AGTTAAGAATATTTGCAAAATTCCATGGAATCTGTGTGGGCAAGGGTGGAAGTGAAGACAGGAA
GGTGTGGAGGATAGAGAGAAAGTGGATCGGTTAATTAATTAGAAGCCTCGTGAGCACAAATAAA
TGTTGCAATAAGGATTCTGCAGTAGATGAGCTGGAAGGTTTGGAGGCTGTAGCTGGAGAGAGGC
TCAAAATTCAGATTTTGGAGGTGGTGGTGTGGTTTCCAGTGATGACAATGTCCATGCCACAACC
ACTGGCTGAGCAGCTGAGTAGTAGAACAACTCCGAGAAGACCAGCTCAGGTTACTGGGAAGCCA
GGAAGTTGCTAAGGATGATGGGATCTTTGTGATTTTTGTTATTAAAACAAATTCCCATCAGACA
GCAAAAGATCGATGCAACCCTTGTAGTGCTATTTTTATTCACAACTTTAAGGAGTCTGCTGTCT
GCTTTCTCCTTTTGACTTTTATGTATTTGTTTGTTTAAACAACAGAAATTTATTTCTGCAACAT
GAAGGTGCCTGTAGATTCAGTTTCTGGTGACAGCCCGTCACCTTGCTGCGTCCTCACATGGTGG
AAGGCTTTACCTTTAGAAGAAATTTAAATTTAATATAAATTTCAACTGTTTATTAGCACCTGCT
TTGCCAGTATTTGTCACCATGCTATAAATCATGACTAGCAACAGAGAAATTCTATCATTTAAAT
CAATGGCTCAGATTTCTTCTAATTTTTTTTCTTTTTTGAGACTCTTTCTCACACAGGCTGGAGT
GTAGTGGTATGACCTTGGCTCACTGCAACCACTGCCTCCTGGGTTCAAACTATCCTTGTGCCTC
AGCCTCCTCAGTAGCTGAAATTACAGGCATGTACCAACACCCCCAGCTAATTTGTGTATTTTTA
GTAGAGACGAGATTTCACCATGTTGGCCGGGCTGGTCTTGAACTCCTGGCCTCAGTGATCTTCC
CACCTCAGCCTCCCGAAATGCTAGGATTACAATCATGAGCCATCACACCTAGCCCCAGATTTCT
TTACAAATTGTGTAATTTTAAAACTGTTGTACCAAGTTTCTTTATAATAAAGATGATTCCAGTA
TATTTGAGTAGTAGTGGTCCCTAAATGGGACACTCTGTATGAAAACTGAAAAGTTCCCCTTTTC
CAGGCATATTAAGAACTGCAGAGCCCAGTTTGGGGAAATGGAAGTTTGAGAAGGACTTGAAAAA
TGTCCATATGAAATAGACAAGTTGTGCTTTCATCCCCTGCTCCTTCTTTTCTTATCTTCCTTCT
GTGGAGTCACCTTCCTATTTTATTCCATGCTGTTGGGACTACTGACTATTGCAATTCCTGTTTG
TAATTTAGATATTAAAACAATTGTACTAATAGATATATTTTTCTAGAATACAAACTCCACTAGG
GCAGAGACTTTGTTTTAACAGTGCCTGACACATGGTAGGTGCATAATCAACTGTTGTTGAATGA
ATGAATGCTTATTGTAGAAAACTAGAAAATACATATAAGGAACATGAAAAAATACCTCCACTAG
AAACTAGAAGTCTCCTCTGATCCTACCATACAGATAACCCATTGATATTTCAGTAAATATCTTA
CTAGGCACATAAATACTTATCTATATAAAAGTATAATGAAATGTCTGGAAATAATAAGCACCAA
ATTCAGGATAATTGTTGCCTTCTGAGGGTAGAGGGAGGAAAGAGGATGTGATTGAGGAAGAGTA
TACTGAGAGTTTCCGCTGCATTGGCAGTAATTTATTTCTTAAGCTAGATGATGGATACATGAAA
GTTTATTATATTATTCATTGTGTATTGTGTTTCTTGTATATGTAAATTACTTCATATTTTATTC
AAAATACCTCCAGATTTTTTTTTTTTTTTGAGATGGAGTCTTGCTCTGTTGCCCAGGCTGGAAT
GCAGTGGCGCAATCTCGGCTCACTGAAACCTCCGCCTCCTGGGTTCAAGCAGTTCTCTGCCTCA
GCCTCCTGAGTAGCTGGGATTACAGGTGCCTACCCCCATGCCTGGCTAATTTTTGTATTTTTAG
TAGAGACGGGGTTTCACCATCTTGGCCAGGGTGGTCTTGAGCTCCTGACCTCCTGATCCACCCA
CTTCGGCCTCCCAAAGTGCTGGGATTACAGGTGTGACTCACCGCGCCCGGTCCCTCCGGCTTTT
TTTAATGCGTACACTCCCACAGGCTGGAGTGGGAGTGTATTTTTTACAAAAGAGGAAGCATACA
TACCTATCAGTTTTGAATCTTGATTTTACTCACTTTTTTCCCTCTTACTTCACATCTCTCCACG
TTACTGAATGTACTTCTGAGCATCACATCAATTTCATTTCATAGAATTAGGAAGTATGAAGCAT
AACTTAGTTAACCACTTCCCTGCTTGGGGACATTCATCTGATAAGTAATATTGAGTGTGTGGGG
GCCAGCATATTCAGGCTGTTGCCATTTTTAATAAACTACTATAAATGTAATTCCTTATTAATGA
CTCAAGAGTGAAAAGGCTTGATTTTCTCTCATTGTGAGTGAGTGAAGGGTGAGGAGGTAAAACG
GAGTAAGATATGCTGGCTACCTAAGTATTTAACCAAGGAAAAGAATGTCATTATCCTCTTCTAA
CCCTCATGTAAACTAGAATGTTGATTTCTCTATAGCCAGGCATTAATGGGTCTGGGGGCTGCTG
CCATTGCCTAGTGGAGGAGTTGGTCAACTTCATCAGGGTAGAGTTGTCTTGTCTGCTGCTGAGT
ATAATTATCATATTTTTTCTTTGGGTCTAACTTTTCTTCCCCACCAAAAAAGGAGAAAGGGCAG
TGTAACAATGACTGTGTCCCTCAGAACGCAGAGAAATATTGAGCTCAGACACAACTGCCTCTGT
GCAAAACTGGTCTGAGCCTTTGGAGTGAATTGACCAAAACTTTTTTTTTTCTTTTTTAGACATA
AGGTCTTGCTCTGTTGCCCAGGCTGGAGTGCAGTGGCACGATCATAGCTTGCTGTAACCTCAAA
CTCCTGGGCTCAAACAATCCTCCTGCCTCAGCCTCCTTAATAACTAGGACTACAGGCGTGAACC
AATATGCTGGGCTCATTCATTTTATTGTAGAGATGGAGTCTTGCTATGTTTTCCAGGCTGGTCT
CAAACTCCTGGGCTCAACTGATACTCCTGCCTATGGCTTCCCAAAGCACTGGGATTATAGGTGT
AGGACACCAGGCCTGACCAAGTTTTTAATCAACATTTGACTTGATTGTTTTTTCCCACTGATCT
GATTGGTGGAAGACTGAATTACTGATCAACTGAGTCCATTCATTTGAGCAGGTGATGTTTGTAG
ATGCAATCATTGTTCCAGGTGATTTGGATCCTGTTCTGCATCCTAGCATAGCCAGTCTGTATGC
AAGATAAGGAGACCAACTCAGGACAGGCTGATCTGTTGAAACTGTAAATTGAGAGTATCTGTGT
AAAGGCCCTTCTTTCTGCAGTAGAAAGACAGATGCTCCTCTACTTCTCATGAGTTACATCCTAA
TAACCCATTGCAAGTTGAACATATTTTAAGTCAAAAATGCACTTAATACACCTAACCTATGGAA
CATCATCGCTTAGGCTATCCTACCTGAAATGTGCTCAGAACACTTACAATAACCTACAGTTGGG
CAAAATAATCTAACGCAAGCCTATTTTATAATAAAGTATTGAATATCTCATGTAGTTTACTGAA
TAATGTACTGAAAGTGAAAAACAGAATGGTTGTATGGTTACTTAAAGTATGGTTTCTACTGAAT
GCATATTGCTTTTGCATCATCATAAAGGCAAAGTCATAAGTGGAACCATTGTAAGTTGAGGACT
TTCTGCATACTGTAACTTTGGTTTGTGAATGTAATCACTTTGCATGTGCTTTCAGGGATGGCAT
ATGCCCTACTAGCTGCAGTTCCTGTCGGATATGGTCTCTACTCTGCTTTTTTCCCTATCCTGAC
ATACTTTATCTTTGGAACATCAAGACATATCTCAGTTGGTAATTATAAGTATATTTTAGAATTA
TATTTGCTCATGTTTAAAGTGTTTTGGCTATATTAAGTGCATTATACCTCTATTAGGTTGGTGC
AAAAGTAATTGCGGTTTTCACAATTATACTTTTAATTGTGAAAACCGCAATTACTTTTGCACCA
ACCTAATATATCTGTGTTAATGTTGTCAGGGAAATGGGATTTCAGTGTTTTGCCTGCTTTTTCT
ATTCACTGATGTTAGGTAACTTTTTTAATGAAGTGGAAAAATAAAAAAACTGAAAATGACAGCC
TACTTTAACATTTTAGCATGTTTTGCTTTTTAAAAACGTGATCAATTTGACTCCATTTTTGGAG
TCATAATGACAGGGAAAAAATGACACAAGGTCTTTGAACCTACCATTTTACTGGATTAACTTAG
AAATTACTAAGTATGTTCATCAAATACATGTTCATGATTTAATTAAAGAGCATACTTATTTTAA
ATCACTAATATTAGAAGACTGGCAAAATCTTACCAAAACAATTTAGAAGCTAAGCTTGGCTTTC
TTTCTTGGTATGGCTTATAATAAGTTTTCCTGCTATTCCTAAAATTTGAATTCTCGTTTGTTAG
AATGTCCTAGTAAATGAGTGTTTTAATGCTATTTTGTGACTTTGACATTTATACTAGAGAAAAT
TTTGCTCCTACATCCATCTGCTGGCTAGACATGTCTACCTCGATGATCCTCTTGAAATAAATGC
AACCATCTACTTTATCCCCAACCCATGTTTTTGTCCTTAGTTGTATTTCCTTAGAAGGAGATCA
GAAAGAGGTTTTGATTGAAAGAACTTCCACACCTTGGAGAATCAATCAGTGTTGAGTGCTTGAC
TAGATGAGGAACATGGAAGCTGGTTTTTCTCTTTGGTATTCTATACATTGAGATATTCCAGGTG
ACTGGGGTTGGGTTTTGAGACCTCAAAACCATAAAGCCTTCCATGATGAGAGGGTAGAATCATC
ATCATCGTCATGGATAAAAATAACAGTAGTGAACGGAGCAATTGCTGAAAAAAAAATATTTTTT
AAAATATAAAATCTTACAGATTGACATTTGATATGAAAAAATGTTTTGTCTTACAAAAAGAGAA
AGAAACTTCATACTCCCTTTGCTGTTTTTTTAATCCTAACTTCGACCCTGTGATATTGATCAAG
GTACTCAGATCCACAGAGATCAGGACATTGGACACTCTAGGAAATTAATGAGATTATATGAAAT
AAAAGTTGAGCGGAACAAGTAAACACTCAATGTATGTGCTACCTTTGCCAGAAAACCTTTCCTG
ACAACTCTCATCCCTGTCCCCACCACCCATACACATGAGGATCACACACAGACAGGTTAGACGA
CCTTTTGATATACTCACGAAGCATCCTGAATGTTGTATTCTAATAACTACCTACGTGTCTCTCT
CGCCTATTTCTAGGTCATGAATGCCTCGAGATATCTGCGTTTTTAGCTTCTTTTTGTTGTTGTT
GAGACAGAATCTGGCTCTGTCACCTAGGCTGGAGTGCGGTGGCGTGATCTCAGCTCACTGCAGC
CTCCACCTCCCCAGCTCAAGCAATCCCCCCACCTCAGCCTCCCGAGTAGCTGGGACCATAGGCA
CGCACCACCACACCCAGCTAATTTTTGTCTTATTTTGGTAGAGATGGGGTTTTACTATGTTGCT
TAGGCTGGTCTGGAACTCCTGGGCTCAAGCGATCTTCCCACCTCAGTCTCCCAAAGTGCTGCGG
TTACAGATGTGAGCCACTGGGTCCGGCTCAGCTTCTTTCGTGAACAAACAATATTTTCCTAGTC
ACAGCTAAATCTTTTATACATTTTTTAAACCCTATGCAGACACATTGAACATTTGTGATTAATA
ACTGATTAATTGTTAGAGACTTTTTTTCCCCAGGACCTTTTCCAGTGGTGAGTTTAATGGTGGG
ATCTGTTGTTCTGAGCATGGCCCCCGACGAACACTTTCTCGTATCCAGCAGCAATGGAACTGTA
TTAAATACTACTATGATAGACACTGCAGCTAGAGATACAGCTAGAGTCCTGATTGCCAGTGCCC
TGACTCTGCTGGTTGGAATTATACAGGTAATGAACTTACAAGTAAAATATAGATGGATGTAATT
TTTATTTGAAATTAACTTTAAAGCATATAGACTTAAAGATTCTACTAAAAACAAAACAAAGTAA
TTTCCTGGAACCCAAAATTATTTTCTAAATTACGTTGTTTTAGGTCAGGTGCTAAAATAGTAAG
CAAGACCCCACTTATTAAGGCTCACTTATCATCAAAGTCAGAGACAGAAAAAAGCACAGAAGAA
CATGTGTGATTCAATTGAGGATGAGAGAAGGGAAATATACGGGAATCCATAAAGGAGAAAAAGT
GTTCTGGTGAGCGGAGACACAGGACCGAAAGCCACATAAATAAGCTCTGGGTTTTTGCTTTTCT
AAGTACATGTATAGAAATACTTCAAGGTTTTTATTACACTTAGTTTTCAAATTTTAGAGTGGTG
GAGGAAGGGGAGTGATAGGGTATTAAGAAATTCATATTTTTTTCTACCAGTATTTTTGTGCTAT
AGGCAGGCTACTAGTGTTTTCATTGGTATTAAGCTTGATGTAATATTTCCAGAGAGTAGGTTTC
TATCTCAGGCAAACATTTAATTTTTCTTTCCTTTTCCTTATCGTAGTTGATATTTGGTGGCTTG
CAGATTGGATTCATAGTGAGGTACTTGGCAGATCCTTTGGTTGGTGGCTTCACAACAGCTGCTG
CCTTCCAAGTGCTGGTCTCACAGCTAAAGATTGTCCTCAATGTTTCAACCAAAAACTACAATGG
AGTTCTCTCTATTATCTATGTAAGTGTTGCTTCTTGCTCCAGGGATGGGTCACTGTTCATTCCA
GAAACAATTGTATTCATTCTCTGAGTCTGGGCCAGGCGTGGTGGCTCACACCTGTAATCCCAGC
ACTTTGGAAGGCCGAGGTGGGCAGATTGCTTGAGCCCAGGAGTTTGAGACGTGAGACCTCATCT
CTTAAAAAAAAAAAAAAAAAAAAAGAAAGAAAGAAAAGAAAAGAAAAAGAAAGAAAAAATCCAA
AAATCCGAAAATTTGCTGGGTGTGGTGGTGCACACTTGTAGTCTCAGCTACTTGGGAGGCTGAG
GTGGGAAGATCTCCTGAGCCCTGGAGGTTGAGGCTGCAGTGAGCTGTGATCGCCCCACTGCACT
CCACCCTGGGTGACAGAGCAAGAACCTGTTTCAAGCAAAAACAAAATCAAAACAAAACAAAACT
TGAGTCTGGGAGCACCCACATTTCTTTCTTTCTTTCTTTTTTTTTTTTTTTTTGAGACAGAGTC
TTACTCTGTCACCCAGGCTGGAGTGCAGTGGCATGATATTGGCTCACCACAACCTCCACCTCCC
GGGTTCAAGCGATTCTCCTGCCTCAGCCTCCTGAGTAGCTGGGATTACAGGCGCCCGCCACCAC
ACCCAGCTAATTTTTGTATATTTAGTAGAGACAGGGTTTCACCATTTGGGCCAGGCTGGTCTTG
AACTCCTGACCTTGTGATCCACCCACATCGGCCTCCCAAAGTGCTGGGATTACAGGCGTGAGCC
ACCGCGCCCGGCCTGGGAGCACCCACATTTCTCTATGCATACTTTGGGAGCCATTGAAAACATT
TGGTTGCTTCTGTCAGAAATACTATTGTACTTAAAAACATTAATGAAAACAAAGTGGTCTAATG
AAGTAAAGGCTGCTCTGGAATGTAACAGACTTGATTTCTAGCCTCAGCTTTGTCTCTAACAGGT
AAGTGACCCTGGATACATCATGTAGGGTGCTAATTGAAAGCTCAGTTTTAGAACTGGATGGACC
TTGATTCATATTCTGGCTCTACTGTCTACTAGCCATGTGAGCCAATCACCATGCCTCAGTTTCC
TTCTCTGCAAAATTGGGTTAGTAATAGTATATGCCTCATTGGATTGTTAAATAAGAATAGATAA
GCACTTGGCCCAGGGGCTGGTACATAACAAGTACTCTAAATAAAGGAGTTATTTTGAAATGATT
ATTTCAAGCCATCTTTTCTTTTTTATTGGTGAAATGAAATGATTGGAGATGTATCTCTAAAAGC
TTTTTCTAACCATAAGAGTCCGTAAAATGCATAATGTAAATGTCTCAACAATTATAAATGAAAA
GGAACATTAGATTCAGAGATGATTCACCATGCAAAAGAAATGCAAAAGCAGGATGTAGATCACA
CTAATTAGATTTGAAAAAGGTTTTGGATCTAAAAATGTGTTCTACATAAAGCATGTTATTGATG
CTTGAAAAATGGTGATAATAGTAACATGATGTCATCCTAGCTTGGGGGGAATGATACCATACAA
AAATAACGTGTCAACATTAAGAATGGAGTTTGAGTCTTTAAAGACCCACCTATTAGCTTCTAAA
ACAATGTAAGGCCATGTACACATATCTAAGAATTGGTGAGTATGTCAATAATTGCTCAATTTTA
TAACGGTGCCATAATCCCATACTATTACACTCCATCCATTCAACAAATATTACCAAGTGCTTAC
TACAGGCCAGGTACTATGCTAAGCAGTGAGAATAGACAGAGGAGGCAGAATAACATAGTGAATA
AGATCATGAGGCCAGGTGTGGTGGCTCATGCCTGTAATCCCAGCAGTTTGGGAGACTGACACAG
GTGGATCACTTGATCCCATGAGTTCAAGATCAGCTTGGGCAACATGACAAAACCTTGTCCCTAC
TAATAATACAAAAATAATTAGACATGCATTGTGGCACGCACCTGTAGTCCCAGCTACTTGGGAG
GCTGAGGTGAGAGGATCACCTGAGCTCAGGAAGTTGAGGCTGCAGTGAGCCGAGATCATGCCAC
TGCCTTCCAGCCTGGGTGATGGGAGTGAGACCCTGTCTCAAACAACAATAACAGCAACAACAAA
CAAAAAACCAAAAACAAACAAAAAAGATCATGGACTTTGAACTTCAACAGACTTGAGTTGGCAT
CTTGGTTCTCCACCCACTACCCATGATGTTTATACTCAACTTCCCTAAGCCTCAGTGTCCTCAA
TTGTAAAATGGGAGTAGTAATAGTAGCTACCTCTTAGAGCAGTGAGGATTGAAAAAGAAAATAC
GTGTAAGGCACTTAGCACAATATCTGTCACATAGTAAGAATTGAGTTTATTATTATTATTATCA
CAGATGAATAGACCTCATGAAGCTCGCCGTTCATTTCCTCGGGACCCAGCCTATTAGGAAACAT
TAGCAAAAGTTTTTGATTCAGCATTATATGGGTCATTCTTGATATGTTTTTTCCTGTCAAATCC
TCAGTTTTCTATTAAAAAAGAATTTTTTTTTTTTTTTGAGACAGGGTCTTGCCCCATTGCCTGG
GCTGGAGTACAGTAGCACAGTCATAGCTCACTGCGACCTCCATCTCCCTGTGTCAAGCGATTCT
TCCCCCTCAGCCTCATGAGTAGCTGGAACTACAGGTGTGCACCACCACACCTGGCTAATTTTCT
TTTTCTTTGTTGAATTTTAGTAGAGGCAATGTCTTGCTATGTTGCCCAGGTTAGTCTCGAACTC
CTGAGTTCAAGTGATCCTCTCACTTTGGCCTCCCAAAGTTCTGGGATTAAAGGAGTGAGCCACC
ATGTCCAGCCCATATTTTAAAATTAAAATTAAAATTTTAAGATTTTATTTTGGTAGAGACAGGG
GTTTTGCAATGTCGCCCAGGCTGGTCTTGAACCCCTGGCCTCAAGCAGTCATCTTACTGCATGC
CTCCTAATGTGTTGGGATTATAGGTGTGGGCCACTGCACCTGGCCAATCCTTAGTTCTTTTTGA
GACCTCTGACTGCCTTATTTTGACAGGCTCTGTAACTTTTGCAGCTCCTGGCACATCTTGCTTT
TACTTACTTTTACATTTTCTCCATCAAATGTTCACTCATTTATTCCTTCATTCTCATTCATTCA
GTTTATCTACTCAATAAACACATATTGAATACTTACCATGTGCCAGGCACTGTTCACTCCCTAT
ACAAACCCAGTGACTCATTAACCTACCAAGACATCTCTTACTGAGTATTCTCACCTCACTGGGC
AAACATTGCTAGGCCTTCATCTACACAGATCGCAGGAGGTCCAAACTAATTGTTGACCTGGGTC
CATGGCCATTTCAGGACCCCTGCAGAACAGGAGAAATAGTTCTTCTGGTCTCATAGGATTATGT
TTAAGAAGAAACTTTCTGCAAATGATTTCACTTTGACAGGGACAAAAGTGAGCAGGAAACACTA
GACCAAGGTCAAAAATAGTTTTTAAACCAAGTGCAGTCATTATGTAACTTAGTTCCTGTTGAAT
ATTTTTCTTGAAATCTCAGTTTAGCATACAGCATAGAATTTTGCAAGAAACTCCTCACAGGCCA
CCCAAGTTGGAAAAGGTATTTCAGTCATCCCTATATGGTGGTGAATATAAGTTTGCTCTTCTGA
AAAAAAAAAAAAATCAGCAGGTCTGAAAACGATTGCATAGGGTTAAATGAGCCGCTTAGTCTCT
GAACAGAGAATTTTGCTTATGACATTAGCATTGCAATTATACATGAAAGAATCCTTCAATAAAT
GCAGAGTAGTAGAGGATTCTCCCAGAAAAGACTGTAAGATTGTTGTAAAATACTTTACCTGGAG
TGTTAACTTATCTTTGTTCTAAGGTTGCACTTTGGTTTGTCCTGCCCTCTCCCCCAAAAAGGTG
CTGACAACTAGAAGTAACAAATGCAAAGAAATAGGGGAGAAAATATTAGAAGAAAAAGAAATCA
GTTAATTTACAAATGTCAGAGATACTGGATGTAGGAGGCAGAAGGGAGAATGACAGGGTATTGG
CAGCAATTTAGAATTAGTCAATACCCTTGATGCTACCATGTAGCTACTCAGTTATCTAAGTAAA
TCCCAGTCCACTTAGGGGTTGATTTCCCACCACTCAATTTGAATGGTTTGTCTCAGTAGACAAG
GGTGAGAGCTTTGAAAGCCACCACTAAGCTTGATTAGATTTGTTACTTTGTACCTATTTCAGTA
TGTAAAATTTATACATTGTATTAAAGTAACACTTTAAGGATTGATCTATACATGTATACACAAA
AGATATGGAAGGAAATGCCTGACAGCAAAAGGTGGTCGTGAAGCATTTGTGATATTCGTAGAGA
TAAAATAAACAAAAATAAATTCAGTTCCAATAGTAAACTGATCAATTTTCTCATACACATTATT
GGGCATAATTTTTTCTTACGGTTTCATAAACACAGGTAATCCTGGGCAAGAACAAAGAAATTAT
CTAAATGAAATTGTCCTAAAATTTTCCAAACACAGCAGGAAATATAATAAAATATTATCTATTA
CCAAAAAGTTCATTATCATAATTTGAATTATTTGGAATACTAAGCTAAAATACAGAATTATATG
TGTATCCATACTATTAACATACTTTATGGTATTTTAACAGTAAACTAAATTAGAAGTACTGTTC
AGTGTTTTTGTAGAGTCTAATTTCTATGTCGTTAAATATAATCTTATATTATTATATAATATAA
TCTTATATTATTATATAATCTTATATTATTATATTATATAATCTTATATTATATAATCTTATCT
TATATAATCTTATATTATATGCTCTTATATAATATAATCTTATTATATAATCTTATCTTATATA
ATATAATCTTATTATATAATATAATCTTATTATATAATATAATCTTATCTTATTATATAATATA
ATCTTATTATATAATATAATCTTATCTTATTATATAATATAATCTTATTATATAATATATTCTT
ATCTTATTATATAATCTTATCTTATTATATAATATAATCTTATCTTATTATATAATATAATCTT
ATCTTATTATATAATATAATCTTATCTTATTATATAATATAATCTTATCTTATTATATAATATA
ATCTTATAATATAATCTTAACTTATTATATAATATAATCTTATTATATAATCTTATCTTATTAT
ATAATATAATCTTATCTTATTATATAATATAATCTTATATTATTATATAATCTTATCTTATTAT
ATAATGTTATATTATTATATAATCTTATCTTATTATATAATGTTATATTATTATATAATCTTAT
CTTATTATATAATCTTATATTATTATATAAGTATAATCTTTTATTATATTATTAGATAATATAT
AAGTATAATCTTATATTATTATATAATATTATTATTATACAATAATAATGTAAACTGACTATTG
CAGTTTACATGGTGAGTTTTATTTCTGCACAGATTAAACTTTCTATCATAGAGCTCTTCTTAAA
AGATTCTGAATGACTAGTCAATATGTAGGCAACTTATTTACATACTCAGATACCTTGCATGATG
CAATTCTGTAATATAAACTGATTTTGGTTTTTGAACCTCGTGTTACATTTCTAATCCACCAGAC
ATACTTTTTATTCAAAGGTCTAATTGATTCTCATGGATTAAACTGGAATCCTGTAGTGATTATT
TTTGTAAGGTAAAAATGTTTGTGTGATTGGGATAATGATTCCAAATGTTTTGTTTTTCAAGTTT
TAATTTTTGCATCTTGAGTTGAAAAGTAGACCTTTAATGAATAGACAATTCATCAAAATGTTAT
ACCTAGCTCCTGAATTAATTAGGGTCATTCATCAGAAAACTGATTATGTAAAGGACCAAACAGC
AATGATAACCTTCAAGCCTCTGGGATAGGTAGGGCTGTTTGCTCCTGTGCTAAATGCCCTAAGT
TCTTGTTATCGTGTCTGCTGGTGGTAGTTATCCCATTTTCTTTTTCACTCTGCTGTCCCTGGCA
TCATCTTGTTTCTGTCAGTGTAAATGCCTTTAGCAATTTTCAGTGACTCTGAAAACCAGACAAA
GTGAATTTGTTAGTACTGGATTTCAAGAAAGAGCAAAAGTACCCTCTGAGTGAAAGTTCAAGAG
TCTTCCTACAAGTTAAATGCCTGAACTCTAGTCCAAGTGTTTGGTGGGAGGTATTTTGCATGCA
TAATAGCTTCTCCCCCATTTCCTTACTCTTCTTGAATGTGTTCCAGAGAGCTGAGGTTGCTGGC
AAAGTGACTCCTAGATTACTTAAGGTTCATTAAGTACTTAAAGTTCATTAAGCTCATACATAAT
TGTCACCAGATTATCATTATAATTATTTTAAAAGTAGTTTATCTGTGAAAATAAGTAAAATACA
TTTTATAAATGTTATAAACTATAAGATGCTCCATTATTTTATCTTCCTATAAAATATGTCTATA
AAAAAGCTCATTTAAAGAAAAGTTACCTTCACTGTGAGATTGTCTCAAAGAAATGTCTCAAAGA
TACCTTCTTAAATCCTGCCTTCTACAGGACTGAAGTTAATATAACAAAGATATTAGTGTTCTTC
CTTGGGATTCTTCCTTGCTAATTTCCTTGAAAACACCCTTGATTCCCAGAAGTCTCCTATTTAC
AAAACAAAACTAGTTTTTCTTGAATTGATTCATCTGTCTACTTAAAGATTTTTTTTGGCCAGTT
GTGGTAGCTCATGCCTGTAACCCCAACACTTTGAGAGGTTGGGCAGGAGGATTTCTTGAGGCCA
GGAGTTCAAGACCAGTCTGGGCAACAAAGTGAGACACCATCCCCTGCCCCCACCTCCTCCCGCC
CCAATCTCTACAAAAAATAAAATAGCCTATTTATTGTACTCTCTAGAATAAATAAAATAAATAT
AGATGCCTGCCTGTAGCTCCAGCTACTCTGGAGGGTGATGTGGAAGGATTGCTTGAGCTCAGGA
GTTCGAGGCCACTGTACTCCAGCCTGGGCAAGAGAGCTAAAAAAAAAAAAAAAAAAAAAAAAAA
TTTTTTTTATGTTATCTTAGTTCAAGTTTGAGAAATAAAATTAAGTACCAAATTTATTCCCAAA
AATGTATGCTTGTCAGGAGATATACCTAATGTTAAATGACGAGTTAATGGGTGCAGTACACCAA
CATGGCACACGTATACATATGTAACAAACCTGCACGTTGTGCACATGTACCCTAAAACTTAAAG
TATAATTTAAAAACAAAAAACAGAAAAAAACTACAGATTCAATTCAGCTTTAGGAATTAAAAAA
AAGATTTGATGTAATTATATCATTATAAAATCAAATTTTTTAAATAAAACATGAAATTGTTTTA
ATTTAAAAAAAAGTATGCTTGGCCTATATGTCAATTCAATAGCTTTTCTTTGCCAGAAAGAATG
AAGTCAGTCTACCTGTATCCACATATGAAGTATTTTAGGATTTTTTGTAATTTTTTGTTATTAT
CTATATCATTCAGGTTTAGGAAAGAACTTTACCTAAAATACTGGCTAATTGTGCCCCAAAAAAA
AGAAAGCTAGAAAGAAAGAACACACACACACACACATACACACATATACACACACAAAAAAAAC
AACCAACTGATCAACTGGGAGTTTCAGGTTTATCAGCCTTTAGTCTCAATGCTGTATCACTGTA
GTAAAAATCAGGCTACTTGATAGCACCTAGAAACCCATTTTAAAATATAAACTTGTAAAGCATT
TCTGTAAGTAAAAGTTAGTATGTTTATAATGACATTTTTTCTTTTAAGACATCTTTATTATTAT
CATAAAAAATACAGAGAAGCAAAAATAAGAATGTAGAAATCACCCAGTTTTTCCTTTCCAAGTA
AACAATCATAAAAAACTTGGTGTATATATATATTGCAGCAATTGTATGTATGTAATGGTCTCTG
TATCAACCAACACATTTTTATCATTTTACTGAAACTTTTGAGTGTTGTTTGATGCTGATATCAT
GGTTTTTCATGTGGGAAGATTCATATGAGAATTGATTGTGTGTGTGTGCGTGTGTGTGTGCTCG
TGTGCGTGTAGCAGCAGGAAGTATATAAAATTATTTTCTTTTTATAGACGCTGGTTGAGATTTT
TCAAAATATTGGTGATACCAATCTTGCTGATTTCACTGCTGGATTGCTCACCATTGTCGTCTGT
ATGGCAGTTAAGGAATTAAATGATCGGTTTAGACACAAAATCCCAGTCCCTATTCCTATAGAAG
TAATTGTGGTAAGTAGAATATGTAGTTAGAAAGTTCAGCATTATTTGGTTGACAAACAAGGAAT
TATTAAAACCAATGGAGTTTTTAACATCTTTTGTTTTATTTCAGACGATAATTGCTACTGCCAT
TTCATATGGAGCCAACCTGGAAAAAAATTACAATGCTGGCATTGTTAAATCCATCCCAAGGGGG
TGAGTGTGGTGTTCCTCTTAGTACTAATACATTAAGTCAGTAAGTCAGTCTTTTTTATTTAAAT
AAAACCTTTTATTACAAGCTTCATTTCACTGATACTCCTTCAATAGTCCTATTTGTGTGTGATC
TGGAAGAAACAACCATAAGACAAACAGTATGTGTGTAGATAAACGTCAAGCCATTTGTTTAAAA
GCAGCCCGTATATGCTCTTTTTTCTTCATTTTCTTAAAACATGGCAGAACATGTACGGTTAACA
CCAGAGTCCTGATTTAAACTTATACATAACATTATAATATAATATAAATAGAATCATTTCTCAG
TAACATCTATTGCACTGTGAACAATCTGCTTAAGGAATCAGGGACGTCTGGCCTAAATTCTCTA
TCATTTAGTTTAATCAATTTTGGATAATAATTATATCCTCGTACCCATAAGTAAAATGCATTAT
CTTCAAGGGAAGTTTTAGAAACATTTGGTAGTTTGGTGAATTAGAAGAGTAAAAGATCAAGGTC
CTTCATTCATCTCTGTCACCTTCCAGTTTTGTGACCTGAGGAGATTCTCCTATATAAGACAAGG
ATAATGAAAACAGCCGAAGCCTCAACTCAAGCTTATTATGAAACACCAGTGAGATCTCTCATGA
AATGAAGAGTAGTCAAGACACCCTACACCATAATTTTCTCATATGGGGTTACAAAGGAGGCCTA
GACGTTTATTATATTTGTAACATACACTCATAACATTTAAGACTAGACTTGCTGTTAATCAAGA
AGTACTAGTTTTCCCTAAGACTGGAAGAGTTTGGTACCGGTGTTTCTACCAGGAAAGGGAGTGG
AGGGGAGGGAAGGGCACGGAGCCACTGTTTGGTACCAGTGTTTGTTTTCCATGCTCTTAATAGA
AACTTCCTCAGGCAAGCTTATTCCCAGAGCTTATTCTACAGGGAACTGCTGTATTCCCATTTGA
TGAATCTTATAATTTCCTCTAATATCTGTTCCAAACTATTTGAAAAAGCACAGCTGGCTACATG
GGACTTAATTTGCATGCTATCCATTACAGTGTTTCTCAAACTTACCCTTTTGTAAGAATCACAT
GGGATCCTTTTTCACACAGATTCCTGGTTCCCACCCCAAACCTATTGAATCAGGCTTTCCAGAG
GTGGGGCTCAGGAATTAATATTTTTCAAAAGTGCCTCAAGTGATTCTTGCCCTCAAGGAAACTG
GAGAAACTCTTTAAGAGAGGATCAACAAATCTAATCTAACAACTAGATAATTTCATTAGTTAGA
AACAATTACAGAAAAAGATACTTTAGAGATCTCTTTTGTCAGGCTTCCTAGGATGTATCAAGTT
ATAGGTGGTCCACAGAGCAGGCCCTGTCCCATCCATCCGTGTTCCATTAGTTGGTTTAGGGAAC
TCATTTCTTCTGCCCTAAATTATCCCTCCAGGTCCTCTGCAGAGCTTTCCTGTGCTCTCCTCCT
GAGAACAGATTTCTAGCCTCTCGCCCTTGAGTTTAATGAAGCTGAATCCTTTGACAGACCACCT
GGTCAGCTTGGTCAGCTAATTTAACCTGCCTGGCCCCAGGGACTTTAAGCTACTCCATGAGGGC
AGTGACCCCATTGGTGTTGTTCAGCTCTGTCCCAGAGCACAGAGCTCATCTCACAGTAAGCTCT
CAGAAAACACTTGTTGAAGGAATAAAATACCCAAGCCTATGTCATTATAAACAAGCCACAACAA
AGGCCTTGGTTATCTGATCTCACTTGGTAGCACAGAAGCAATTCTGATGATATTAACAAACCTG
AAAAAGAAAGGGAGTGGAGAGATTGGAAAAAAGCAAGCAGAGTTTTAAAGTGGCCCTGAGGTGA
GCACAGTGAAAGGAAGAGGGACAGAGAGATTGTGATATGCCCCCTTTTGATCTCCTCCCTGCTT
CAATCCTCCATTAAGGCTGCTGCTCGTAGTTCCCTTTATCCTATTGAGGGTCCATAAGTTAGGA
TCACTCCTAACCTAGGACCATACGTTAGGACCATAAGTTAGTCCCCAATAACAATGCCCCATTG
GACAGCCAAAAACAGTGGCTCCATGCCCTTCCCTCTACTCCCTTTCCTGGTAGAAACATACAAC
TTTTCCTTGTCCAGTCAACACCTAAGCGCCCCACTAGGATTCACTCATTCTCCCATCCTGGGTG
CTTTCTTAACCAGCAGACACTATGAGGGCCAGTCATGTGCCTCTCTTCTCATGCAACTGGTCCT
TATCTGCAACTCGTAATCTGCCCAGTTCCTCACTCATCCTGCCCAGGCTTCTCTTTTACCTCTT
CTGTTATAGTGGTGTGTGTGTGTCTGTGTGTATGTGTGTGTGTGTGCTCACGCACGTGTATGTG
TAAACCCATAACAAGATTGACAGCTCTGTCCTCTAAATTTTAATCCCTATTCACTCTCTTGCCT
CCTGTCCTTGCCAGTTCCAGAGGTCCCCTTGTTGTCATGTTTGTTACTCTCTTTTTTCTTTTCT
TTTCTTTAACAGAGTCTCACTCTGTCACCCAGCCTGGAATGCAGTGGCATGATCTCTGTGGTGT
GTGTGTGTGGGTGTGTGTGTGTGTGTATAAACCCATAACAAGATTGACGGCTCTGTCCTCTAAA
TTTTAATCCCTATTCACTGTCTTGCCTCCTGTCTTCGTCAGTTCCAGAGGTCCTCTTGTTGTCA
TGTTTGTTACTCTTTCTTTCATTCTCTTTCTTTCTTCCCTTCCTTCCCTCCCTTCCCTCCCTTT
CCTACCTTCCCTCCCTTCCCTCCCTTCCCTCCCTTTCCTACCTGCACTCCCTTTCCTCCCCTTC
CTACCTTCCCTCCCTTTCCTCCCCTTCCTTCCTTCCTTCCTCTCTCTCTTTTTTCTTTCTTTCT
TTCTTTCTTTCTTTCTTTCTTTCTTTCTTTCTTTCTTTTCTTTCTTTCCTTCTTTCTTTCTTTT
TCTTTTCCTTTCTTTCCTTTTCTGAAACAGAACCTCACTCTGTCACCCAGCATGGAATGCAGTG
GCATGATCTCGGCTCACTGCAACCTCTGCCTCCCAGGTTCAAGCAATTCTCCTGCCTCAGCCTC
CTGAGTAGCTGGGACAACAGGCACACACTGCCATGCCTGGCTAATTTTTAGTAGAGACGGGGTT
TCACCATGTTGGCCAGGCTGGCCTCAAACTCCTGACCTCAGGCGATCTGCCTGCCTCAGCCTCC
CAAAGTGCTGGGATTACAGGCACTGTTATAGACCCCTTTTGTCAGGTTTCCTAGGATGTATCAA
GTTATAGGTGGCCCGCAGAACATGCCCTGTCCCATCAATCTGGGTTCCATTCATTGGTTTGGGG
AACTCAGGCCTACTGTTGTTTTTAAAGTATGTGCTTTGCAGTTGTACTTGAAAGTGTGGCACAG
GGGCAAAGCCAACAAAACCACAGGGTTTTTTTCCTGGCTGCTTTCTGAGGGCTACTCTTCTCCT
CTAGCTGGAGTGGTGGTGCATTTGCCAAGTGTTCAGACCCAGTCTGAAGTACTCTGGTATTCTG
TTTGGTATTCTGTTTATGACGCTGGCCTGTCGTTTGTGGAATTTTGGGCCCTTCAGGGTTCACT
TCTCCCGGTCTTCATCTTTTCAGATAAGTGTGTGTTCACATGCTCACTCCCTTGGGCTCTTCAT
AGTGTTGTCTTATGATCAGGCCTGACATGCTTGGCTGGGGCCAAACAGGCCCACTGAGCAAAAA
ATTTGGAGAGTTATATCCACAGAAGGAGGGCAAGGGCAAGGGCAAGGACAAAAGGCTTAGTTAG
AGGTCATTAGAAGATATCCAGACGGGACTTCAATTGGTTTGGGGCAAATAATTGGCACACTTAG
GGTTCAGCAAGACAGCAGACATGAGTGGCAGGAGTATGACAGACTTAGATTCTCCCAATATATA
CCAAGAACCTACTATATGTGTAGACACTAATTACTTACTTACACAAGTGATTCCTCATTGAATC
ATATGAGAAGAATATATAGTCCCCATTTGACAGCTGAGGAAACTGGGATGTGGTGGGTTTAAAT
AACATGCCTAAAGTCATTTGAGAGTGATTCTGGTCCTTTGACTTAAGTCTTTGCACTTTACACT
TTGCACACCTGGATAATCAAGGATCAGGTCATATTTGGAGAACTAGGCAGCCAGGCAGGCAAAG
TGCATTAGGCTGCTATCAAAACTAAGCTGTATGTGAAACTGGAAAGTTATCCTGACAAGTCCGA
GTGACCTGAAGTAGGGTTGACTGGGTCTCAGGAGCAAGTCAGGAGCCTGTTTTGAGAAGTGTGG
TCCCAAGTCACATCCCAACTAGCAGGTGATGTCATGCTGAGTCGCCCAACTGTTGGATCAGGGT
TTCCAACATCCTGTCTGTCTACAGATGGGGTTCATTCCACAGTCTCATTGTGGGGTAGGATGGG
ATATAAGGAGAAAAGGAGTCTGAAAATTAACCTCACTGGTAGGGAGAGTAAGGCTACAAGGGCA
GAGAACCAGACAGATCTTGTTAGGTAGGAAATCGTCTAATATCTAACATTTAGACCTGGACCCA
CTAACTGACATCAAGCAAATCACTTCACGTTTCCAGGTCTAAGTTCCTTTATCAACAAGACAAG
ACAAGAGTGGTGGATTCAACAATACCTAGGATGCTTTCTCTATGGTTTTCTCTAAGAACCTTAT
TTCTATTTTATTTTTACCACTGTTCTCTAATTATAGGGGAAAATGTAGTTTCCTTATTAAAGAA
ACTTTAGGCATAAGTGATGTAGGTCGGGGAGACCTTTCATGGCTGGGGGCCTCTGGAATTATAC
CATATGGATAGTCATCAAGATATAGGTCCATCTCCCAAATTTTCATATTTGAGAGAACCTCGTG
GTATAATGTGTAGGTTAATTTTTTTATATGGTTTGTAGGGTAGCTCTGGTTTTGTCAAACAGTA
GTCATTGGACCCCACAATCACTGAGATATTTTAGTCACTATGGAGCGTTGTGATAATTAGATTA
ATTTAATCTTTTTTGCCTTAAGAAGATTTTAAATGTCCTGGCTCTCAAAGGATTTTCCACTGAG
CTCTTTTAGCTTCATACATTCTTTGTCTCTTTAACAGAGCTGATACCTCTTCAAACACAGTAAA
TCTCATATTATGGCTAGAATCTCTTTTAAATTCAAAGAACTTTAAGATGGAATTGTGGAAGGAG
AAACACTAACATGTGAAATGGCATGGATGGGGCTGTATGATATTAAAGAATCAGTAACAGCAGA
TTTATTCATGTGAGAGATAGAGAAGAGTCAGCATGGCTAGGCTCAAAAATAGAGGACAAAGAAA
TCAGCCAGTAAGATAACACCAACTTCTGATTAAACATTATAAAAGCTGCTACTATCATGTATGT
ATTTCGTGTGCTTTTTGTTCTTTTGGATCAAGTACTTTCATGTCTAATATGTGACTGAGCAGAT
ATAGCATTTGATGAGATGGGGAAAAAGGATGGTGGTCAAATCTTCACAGCATTTTTCACTTAAA
AACTCACTAGGTTTTTGCCTCCTGAACTTCCACCTGTGAGCTTGTTCTCGGAGATGCTGGCTGC
ATCATTTTCCATCGCTGTGGTGGCTTATGCTATTGCAGTGTCAGTAGGAAAAGTATATGCCACC
AAGTATGATTACACCATCGATGGGAACCAGGTATGGGTGCCCTTTTGCTGAACTGGTTTTATAG
GGCTGGAAACAGGAAAAAAACATAAATGGAAAAGATTTTGGTGTCAGCTAAAGAAGGGGTTGGA
TTCTTTCACCAGACCTTATTGGGTTGGTTTTCCTCTTGTGTTTGCTAATTAGAAATTATTTTGG
AATAGACACACTGCATCACTTTGCTTTTTTAAGAAATAAGTTGAAATAACACTAATTTTAAAAG
AGCTAATTCAGTCAGGTCATAAATAACATTCAAAGTGAACATATTAGGTCAGAAAGAGAATTCT
TCATGTAACCCAGTACCTCTTGGCAATTATTTTTAACGGCAGTTATAATGTGACAAAACAGTGC
AACAAACATTAATAATAGTTGTGACCACTTTAAATTGAGGTGGTTCACTGTGCAGTTAATATAT
ACAGGCAATTTTTGTAGGCATGTGATACATTGCCAGGTAAGCTGATTTCACACTGCTTGCAAAT
TTGCAAGTTCTGTATCTATATGTTTCTGAAAATACAGTATAGCAATAACAGTAGTTAAATGGTT
AGTAATCAAGCAGAATAACAGCACTGCTAGTCATAATATGCTTCATAGTGAATATTCAACCACA
GAAAAACACTTTATGTGAGAGCTGCAGACATTCCATTCACTAGGCAGTGAAAAAGCTTAGCAAA
TGATCAGGTGCTATTTCTTGTCAACTTTAAAAATTATCGAGAGCAATGAGACCTCTCTCAGATG
GTATGGCGTCCAAACTCCTGATGTCGTACAAGGACCCCAAGTACCTATCACGGTAAAAATTAAA
TTGGACCACCACGCAGAGTAGGCATGGGAGTTTTCATTCTTAATGTACTTCCTGAAATACTCAG
CGAAGGTCTTGCAAAGATTCAATTTGTAGGATCGTTGTCATCCAGTCTCTTCCTTAGGAATTCA
TTGCCTTTGGGATCAGCAACATCTTCTCAGGATTCTTCTCTTGTTTTGTGGCCACCACTGCTCT
TTCCCGCACGGCCGTCCAGGAGAGCACTGGAGGAAAGACACAGGTAGGAACAACAGCCTTATGA
TATCCATCTCAGAGAACAAGTCGAGGAATGGCAACAGAGGAAGGCTCGCACCGAGCTTAGCAGG
ACAATTTGCCTTTCAGACTTGTACTTCCTAATCTGATTCACCTCAGGCCTATTCCTCTTGTTCC
ACTCCCTCACCTGAAATCTCTTAAAAAACAACATGTATGGTTTTCTGATACAGTGATTCTCAAA
TCTATTTGTCAGTGCTTACCTGTCATGTAGCTACACTTACCTGCTGTGGTCAATAACAACTGAC
AAGGAATCAGTAATGAAGGATGCTGATTTTTGTTAATTTGTTACCTGGGGAGACAGATGGCCTG
GTAAAAGCGCTTCCTGGCTTTAGGAGCTATTCTATTTTCAGGAAAGTGAAAAGCTCTATTCACA
TTTCCTTTAGAAGGATCAGGAACTTCCTCCAGGGGCGTTTGGTCCCACAGAACAACAGTTTGCT
AGCCAAGAATATTTTTGTTGTAACATTTTTGGCATTTCTTCCTGAGTAAGATTTATGTTGTTGA
AGTTCTGCATGTGGGTTTAATCTTATCTTTTAAATGTGGTTTGCAGTTGTTGCCTTTACAAGGT
GGCCAAAAAGCCAGCATCCTAGTTAATCTCTGGATAATCCCTCCTTCAGTGTTCAGAAAGCTCA
GGAGCCATACTCAAAGGCCACTCTTTCCAGCAGGACACAGTCGAAGAGACCACACCAAGCAGAT
GGGCACAAGAGACAGACTCAGGAATTTTGCTTCTGTCTCTTATGCCTTGAGGTCCTTATCTTCC
CAACACAGAAAGAACTATTATTTAGGAGTAAGAAGTGCATTGAGACTCCAAAGAACAACAAACC
CAAACTACATAGTGCAATACACACACACACACACACACACACACACACACACATGCACACACAC
ATGCACAGTCTTCACAGTCTTCAAATGGGTTTTACTAAGGCGGGGACACTTAAAAAATAAATAT
AAGTCGGGCACAGTGGCTCATGCTTGTAATCCCAGCACTTTGGGAGGCTGAGGTGGGCGGATCA
ATTGAGGTCAGGAGTTCGAGACTAACATGGCCAACATGGTGAAACCCCTTCTCTACTAAAAATG
CAAAAATTAGCCAGGTGTGGTGGTAGATGCCTGTAATCCCAGCTACTTGGGAGGCTGAGGCAGG
AGAATCACTTGAACCTGGGAGGTGGAGGTTGCAGTGAGCCAATATCGTGCCACTACATTCCAGC
CTGGGCGACAAGAGCTAGACTCTGTGTCAAATATATATATACACACACACACACACACACACAC
ACACACAAACATATATATATATATATATTCTCATATATATATACGAGAATAACTGAAAAAACTA
ATGACTCCAACAGTGACTTGCCCAGGGAGGGCTTTAATTGTCCCCAATATGCATGTGATGCTCC
TTTCCTATTTATAATGGATCTTGTGACAGACTTGGAAGAATTGGCCTCTTCCAGTTTTTTGGCT
GCTTTTCAACAAAAAATTTTATTTTCAGAATTTTTCTTAATTTTTTATTTTTTTTGCAAATATT
TCCAGCTCTGAGGGAGCAGGTAAAATAATACAGCTCATATCCGGAGGCATAGCCAGAGATCTGT
GGTCAAGGGGAAGAGGTCAGAGCCAATTTCCAAAGCTGTGCACATTTCAGGCTGCAGCTGTCAG
CAGCAGGAACTTCTGCTGCTTTCATTTGAGACGGAGCACTGACAAGCTCTCCAGAGCACATGGT
CTTCAGCGGGTGCAGGCAAATCTCAAACTGCTGATGTCCATTCTCCTGATGTTACCTCCAGGCT
CAAATGAGGCATGGGCTGGCTGTCCCAGTAAGTATGCCCATGTAAAGTGACCTCCTTGGCACAG
GGTTTGGGGGATAATGGTGCTCTGGTAAGCTAATACCCCCTGCCACATACTGGCATGTATTATA
TTTAGGAATGAAGGATGCCAGGATTTCAGCATTAAAGAATGAAACGAGTAAAGAAATGGCTTCA
TGTTTAATTATTGAAATGTATACTAAATTCTGGGGTTAAAAGTCTAAGTGCCTGAACTGAAATC
CAGGGACTGATGGACAAATAAACTGGCAGGGCTTCTCTTGTGTGGCTTCTCGTGTTTTCTCTTA
TGTTGGAACTTCAGTTTCAGCATCTACAAAATGGAAATGATAATTGCATCCATTTCACAGGGAT
ATTGTGAGGATTAGGCAAGAAAATGTAAAGCCTTAGCACATACTTGTAAATGCTCATTGTTTCA
CTCCTTTAGGCAAGAATAGCATCTTAGCATCTATGATACATATATTGTGCCTGGCACATAAGAA
GCACCTGGCATATATTTGCTGAATGAATGAATGGAAGAATGAGTATTGGTGGACACGAACTGTT
TTTAGCACATCTACAGATTGTAGAAGAATTAAACATTGGAACCTTTTCTTTCCCTCACCCCCAT
CTGACTTCCACTGACTCACAGAAAAGTAATTCTGATTACAAACCACTCTTTAGCTCTGATTACC
CCCTCCCTTAACAGAATAATTTTTTAGTAACTGAATCTGGTTGTAGATATAAAGTCTACAGAGT
TTCTCACAAACAAGCCTTATCAAGTAAGTAGAAATAATTGATCACTCACCAATTTTAATATAGT
GGCCAAAATGAGCCTTGGCCTCCCATAATTGGAGACTCATTATGACATATAATTGGCAAGTAGG
TGGCCACTCTCCTGGCATGAGTTTGGCAGCTGACTGTGGTCCTTGTCCCCAAATCTCCATATCT
TTCTCCAGTTCACAGAGCTCCCCATCCACAGTGAGATGCAAGTGGCTCGGTAAATACTGGTTGA
GTAATTGCATGCAAGGAAGGAAGGGAGGGATAAGGGAAGGAAAGAAAGAAGAAAATGGAAGGAA
GGACAAGGGAGAGGAAGGGAAGGGATATGAAGGAAAGCGAAGAGAAGGGAGGGAAGTAGGATAT
CAGCTTCTCTGCTTAGTCAGGAGAAGGGAAAGAGCACCAGGGTACTGGGAATCTAGGATGGGGA
TAGGAAGAGCATAGAAAAGCCAGCCAGACATACAAAGCACGCTTTTTAGTACCTCATACACATC
ATATTGCCTCAGGCTGACCCAACTATAATCTAAATATAAATGATCAATTTGCCAGGTAGAGCAA
GTGCTTTCTAATTTTCTGTTTATATTTGCTGTGTCTGTCTCCCGTAACTCAGACCTTTTGCACT
AGACAGTACTTGTGCTCAACAAAATCTTCCCAGTTGTTTCCTCTCTGATTGCTTTGCTTGTAGT
TCCCTGTCTCCTTTTCTTCTTTGTTTCAGAGCTTCTAATCTTCATCTTGGCTCTGCCCCAGCTG
TCTCAACAGAGAATTCATTGGTCATCTAATCAAACGAGAGAGTAGTCCCCACCCCCTGAGCCTC
TCTCCTAACTAGTATGGATCTTAGGAGAGCTATTTTTGCCAGCGAAGGTAAAGTTTCTGAAGCT
TCCTTTTACCCCTGCTATTGCCCCGTTACTCCATAGTCACTGTTTCAGGCTGCCTTGGGGCAAA
GCTCTGAGCCTGCCTCCGTGGAACTGTCAGAGGAGGCAGGAGACACAATCCCACCTCTCAATGT
GCAAATGTGGCATTTCGAATTTGCAATAGGATGAGTAAATTTATTTTCTCTGAGACTTTAATGA
GACCATGTGCTACAAGTACGAAGTGTTATCAGAGTTGCTATTATAGCTGCAGCTTCAATAATGC
AGAAAAATGAGCTATGTGCCATGAGAATTTATTTCCCAAGAATACGACAATGATCTCTGTTGTT
GGGAAAGGGGGGATTGGTCCATGTTTCCTGCCATGGTAAATAACTAGAAGCTTGGCCTGAATGG
ACGCCGAAACCGCAGGTGTGTACTATCACCAAATAAAATCCCTGTCAAACTGGCTTTAATTTCA
TAGGATCAGTCCCCATATTTTCTTTAACTTCTCTGCCATAGCATATATAGATGCCATTTTTGTT
CAGTTTTGTGGCTTGAGCAAATAACTATCACTTTTCTCGACAGTATTGAGCAGAAGGGGGAGAC
AGGGAAGTATGAAGTGTGTCTGTGAACAGGCTGTCTCATACACACATCCAGTGAGCTGGAAGAC
ACAAGGGAGAAGGACGAATCCTTTTCATAGGAGGTGTGTGTCTTCCAGGTTGCTGGCATCATCT
CTGCTGCGATTGTGATGATCGCCATTCTTGCCCTGGGGAAGCTTCTGGAACCCTTGCAGAAGGT
ATAACCCTGCTTCTCTGCATACCGATTGCATAATTTCCCTTCACTACTCTGCTACCAGATAAAT
AACAGGAGATTTAACAATCATCACATGGAAAACCATTCCCTGAATAACACAGCCTTCTCTGTCT
CTCTTGGCAGTCGGTCTTGGCAGCTGTTGTAATTGCCAACCTGAAAGGGATGTTTATGCAGCTG
TGTGACATTCCTCGTCTGTGGAGACAGAATAAGATTGATGCTGTAAGTCACCTACCACCTATAT
TTATCTGAAATAAGATTTGGTTCTTATATGCTTCCTGCCATATCACTATATTCCCCCCATCCCC
TAAGTCTCACTTGTGCTTTGGGAACTCCAGAGGAGAAATTAGAATTGTGGGGATAAATCAAAGC
CATGAAGTCTTCCATGTGAACTGCATTTTGTGAAGATTCTGTATCTCTGGGAAGGTCAATTTCC
AGCAAGAATCCCATATGAGGCTCATTCATACACCTTTGGCCTTCTAAACACAAAAAGCAAAAAA
ATGTTTAAACACCCTAGTTTAGAAGGACAAACAACATACAAAACAATGCAAAAACAAAATACAA
AATGTAAAAACCCACGCTGACTTTAACTCTGACTAACATAACCTTTGCAACTATTCTGAGAAAC
TAAAAATCAGAAGCTAAATCCTTCAGAGTTCCTCTCTAATTTAAACCATAAAAAGAATGAGAAG
ACTTCCTAGATATTGATATGGAAAATTTTCCAAGACAAATCGTTAAGTGAAAAAGAGACCAGAA
TAGAAATGCTGGGGAAAAGCACAGACACAGGCAGAAATAAAAGCGGATACAAGGAGGGAGGGAA
GTTGGGAATGGGTGGAGAAGGGGGAGGGTTGTGAGCAAGAGTTTTCCCCTTTTTAATGATAATT
CCCTTATGAACCATACAAATATATTAGCTATTCAAAAATTTAGAAATAAATAAAATGGGACAGT
TGGTCTTAATTGAGAAGCCCAGTTTCTAGAGCTACTTAAATATAAAATGAATGAAGTCACAATA
TTTTTAAAAAGCTGTGATTTGGCCAGGCACAGTGGCTCAGGCCTGTAATCCCAGCAATTTGGGA
GGCAGGAGGATCACTTGAGCTCAGGAATTTGAGACCAGCCTGCGTAACACAGTGAGACCTTGTC
TCTACTACAAATAAAAGATTAAAAATTAGCCGGGTTTGGTGGTATGTACTTGTGGGTAGTCCCA
GCTACTTGGGAGGCTGAGGCGGGAGGATCACTTGAATCCAGAAGATGGAGGCTGCAGTGAACTA
TCATGGTGCCACTGCACTCCAGCCTGGGCAATAGAGTGTGACCCTATCTCAAAAGAAAAAAAAA
AATGTAATTTGTTTGTGGATCATTGATCTTATTTTTATAGGTAGTTATCACATGATGGTACCTG
ATACATTAATATAATTCTTTTCATTTCTATTTTTTTCCCTAGGTTATCTGGGTGTTTACGTGTA
TAGTGTCCATCATTCTGGGGCTGGATCTCGGTTTACTAGCTGGCCTTATATTTGGACTGTTGAC
TGTGGTCCTGAGAGTTGAGTTGTGAGTAACGTAAAACCCAGATTTCCTATAAACAGAACAACAC
ACTCTGAGCTTCCTTATACCATTTTGATAAATATAGTGAAGCCACTTTCTTTCGTTATAGTTAC
TGTATATTGAGTGCTTCTATGCATTAAGCAGACAGTGTTTTACAGACATACTTAATCTTCAAAA
ATAGCCTATGAATAGTTTTGATTAGTCTCATTCTACAGGTGAGAAAAGAGAAATTCAGAAAAGC
TAAGAAGCATCCCTAAGATCACACAGCTAGTACGTGGCAGAGCTAAGATTTGAACCTATGTACC
TACTCATGTCCTCTACTGCTGTGCTCTCCTTTTAGTTGTGGTAAGACTAAAAAGTACATAGTTT
AGCCAAAGGCTGAAGTTGCTTTGTAGTTTCTTCTTTAAATGAGATGAGGAATGTAAGGCGCTCG
GCCCCAGGCTTGGGACAAGACAGCTGTTATATATTATAGTTCATTTTCTTTTTTAAAGCTAGTA
ACCAAAAGTTACATAACCCAGAACAGGAGGACTTTAAAATAGCTGATGACAGCCAAAATTCATT
CAAATCTCTCATATAACCAATAAGGCATATAACCAATAAACCACACAATTATGTGAAGGAAACA
ATTAGCTCATATGCAGAGTGAAGGCAGCAAAGACATGATTTAAATAATGGAAAACAGCCTGTGA
GGGAAGGTGAGGAGCCTGAAGGTGGTTTTTTGGTTGGTTTTGTTTTGATTCTAGAGAGGCCTTA
GTTAAGTGCCCTCCTGGGCCTAGAGGGGAACAGAGTCTTTCTCTCCCTCCTCAATGTGTCCATC
GTCTTTCCAGACTTCTGGCCCCTGCTCTGCCTGTGCTTGGGCCGTGAAAGAAGAAGACACCTGG
AAAAACATCAAGATAGATGTCTTCAGCTTAGCATCCGCTCACCTCTCCTGTCCCACCAGCCAGT
TCTCCTTCCTGCCCTCCTTCCCTACCTTGGCATATTGGTGCCTACCCAAAAGGCTTGTGTTTTC
TACCTCAGCATCCTAAATGATTGCCCAAGGGCCTGAGAAGGTTTGCAGGCTCCCAAAGTTCTCA
ATTTGCTTCTAGGACACGTCTCAGAAACGCTCCTTAGAAGATGCTGTCTCTCATGCTGTATATG
CCTCAGTGTCTATTTTTTCTTGTGTGGATGTGGTTGGAAAAGAAGGGTTTTACAGAGAGAGATT
AGCTTATAAACTTTTAAGTTAAAAATCAGCACAAACCTGTAAGCCAAGGAATAACTGGTGGCAC
ATTTTAGCTGCTAGTTTGGTATCTAGGCTTTTAGCCAACAAAAAGCAGTATCCTCCACAGTATT
TAATTTCCAGTAATTATCATGCTGATCCAGAGAGCACAATGCTGAAAGTACACTAAACAGATAC
TCTTTCTTCAGGAATTGTATTTAGCATGTCTCTAGTCACACATGAAGAGTCACGGAATTTATCT
GATTGCATCAGGAACAAGAAATCAATAGTCTGGTTGACAGGAAGGACTTATTTCTGGTCCTCCA
GTCTCCTATTTCTATAAAAAATGACTAAGTCACAAAGAAGCGCCAGGCACTGGAAGACCCAGGG
GTTAAAGACAGAGGCTCTGCTGCCCTTTACCTCCAGGCCTTTTCTCACTTCTCTTAAGAAGTTT
AGTTAACAATAAAAATCAGGATAATACTGATGACCTTTCAGGGTTATGGCAGGGCTTAAATAAG
ATTTCACATGCGTGAAAGCTCTATGCTTATCCTATAACGTGTTGTGCACGTGCAAGTATGATGC
TCATTATTTCTCTCAGCCATCAGAAGAGAGGCACAGTTCTCCCCTCCCCTGCCGATTCCACACA
AACACCAGCTGTTCATTTCAGAGTTAGCTACAGGAAAATGTCATCTGCAATAAAGACAGAGTCC
AAAACACCAGAATGATGGGCTCTTTAGTAGCTGTTGTTTTTAACTTTTTATTCCAAAATACGGC
TGTTCCAAAAAATCTTGACCTTGATATTTTTTCTTCTAGTCCTTCTTGGAATGGCCTTGGAAGC
ATCCCTAGCACAGATATCTACAAAAGTACCAAGAATTACAAAAACGTAAGTACCTTTGTGAGAC
ATTTGCTGGACTTGGGTTTACTAGCCTGAAGTTTCAGCAGCTCCATTTTACGTACAAGGTAGCC
AAAGGGAGAAAATGCCTATTGGGAAAGTCTGTTAGTCCACAGGGAGTGTCATGAAAACTTTTGA
TCCAGTGCACCTTCTGACACCCATGGCTTATGTGAATTTTGTCTATGCTAGCTGAATGTCTTTT
TTTTTTTTTCTTTTTAGATGGAGTCTCACTCTTCACCCAGGCTGGAGTGCAGAGGCAGGATCTC
AGCTCACTGCAACCTCTGCCTCCTGGGTTCAAGTGATTCTCTTGTCTCAGCCTCCGAGTGGCTG
GGATTACAGGCAGGTGCCACCACGCCTGGCTAATTTTTGTATTTTTAGTAGAGATGGGGTTGCA
CCATGTTGCCCAGGCTGGTCTCAAACTCCTGAGCTCAAGTGATTTGCCCGTCTTGGCCTCCCAA
AGAGTTGGGATTACAGGCGTGAGCCACTGCACCTGGCTAGCTGAATGTCATTTTAATATAAAAC
TGTTGTAAGAGGAATTTAAAAATAATAGGCAATTTCTAGATGTTCATCCAAAATTTTGTTTATC
TGGTATATAATTCTTATCTTCTGTAAGATTTTCTTTATTCAGTTCTAACAAGAGATTATCAAGT
TCTCTATGACCAAGACCTTATTATTTGACACTTTGCTTTATCCTTGGTATTAGAAGAAATAAAA
TATGACACAGGAAATGAAAAACAAAGAAATTAAACATCCCATTGATGGACTTGTGTCACCTGCA
AATAAAATTGCTTAGTGGTGACTAGGGGAAACAAAGATAAAAAAGCAGAGAAAGTAAATCAAGA
AAATACAAATTTAGCAAATAATAATAATAATAATATCAACCCTAGGAAGTTGTAGTCTAATTTT
ACCTTAACATTAAAAAAAGTTGGACCTTGAAACTGGGATTTCAGTAGCTGCTACTTAATTTATT
TCCGTGATCTTTCTGTTATGTTGAGCCAAGTGCCATTAAGAGCTAAAATGATTTAGAGATTTTT
TTGGCTTTGGGGAGGGAGCTCTAAGCTGAGTTAGGGAATTCTGACTTCTAGGCTCCATCTTGGT
GAGACTTTGCCCAAGTCATTCAGCCTCTCAGGACCTCAGTTTTCTCATCTGAAGCAGCTAGACT
AAAATTGCTGATTCCTGAAGACTAGAAAAGTCTATGATTCTGTGATTCCTGGGTTCTATTTTGG
GTGTGGCCATTGTATGTCAGGGTGAGAACAGATGATATCATAGGCCTCAGTTCTGCAAATCCCA
TTGATTTTGCTTTTTTTCTTTGCTACTTTTAAAGAATGTTAGCAAAATCAGGAGGGTCAGTGGT
ATGAACTTCAGGCAAATCATGGCACTGTTCTAAGCCTCAGTTTCTTTTCTGGAAATCAGAGGGT
TGAATAAATTGCTTTCTAAGGCCCTTTCTAACTTTGACCTTCTATGAGACTGTAGAGGTCCAAG
AGTTTTTGTAACCTGTTGTCTCCCTATTCAAGAAAAGTTGTAGAGGCCGGGCACGGTGGTTCAC
ACCTGTAATCCCAGCACTTTGGGAGGCTGAGGCGGGTGAATCACTTGAGGTCAGGAGTTTGAGA
CCAACCTGGCCAATGTGGCAAAACTCTGTCTCTACTAAAAATGGAAAAATTAGCTGGGTGTGGT
GGCAGCACCTGTAATCCCAACTACTTGGGAGGCTGAGGCAGGAGAATCCCTTGAACCCAGGAGG
TGGAGGTTGCAGTGAGCCGACATTGTGCCACTGTACTCCAGCCTGCGCAACAGAGTGAAACTCC
ATCTCAAAAAAAAAAAAAAAAGAAAAGAAAGAAAAGTTGAGTGCTGCTACCCAGCTCCTCTGAG
CAACTGTGACTTGACTCCTTGCTAAGTAGCCAGAAATGTAATTAAATACTTGAGGCTTGAAATT
ATTTAATCCCAGACAATTTCTTTTAATGCCAGATTGAAGAACCTCAAGGAGTGAAGATTCTTAG
ATTTTCCAGTCCTATTTTCTATGGCAATGTCGATGGTTTTAAAAAATGTATCAAGTCCACAGTA
AGTATTTTATCCCTAGAAATTTGTTTTCTAACCTCTTTTGAGACTTCATTCATTCTACAAGTAT
TTACTGGGGTCCAATCAGGAATAGGCCCTAGACCCTCTTCCCTTTGTGTAGGGCAATGAGAATT
AAAATATAACATCCTTGCCTTCAAATAATTTACAGTCTATTTGGGGATTAAAAAAACACATATG
TTAAAACCCAGGTAGTAATAACTATGCCAGACAAAAACATTAGAGATGTTTCTAGGCAGAATTT
GAGTAAGTTTCAAATAAGTAGTATAGACAGTTGAGAGTTCATAGAAGGAAGAGATCCAGGTTAG
TTGGACTAACAGGCTTTGTGCGGCAGTTGGCTGAAGGAAAGAAATCATGTCAAGTGTTTGAGAA
CTATGTGAGCTGAGAGACAGTGGAAACATGTATTGGAGGTAGGGTTGCCCAGTTGCAGCAAAAA
GTAAAATATACTAGTGGAATAAATAAGTAGAAAATCCTGGCACCAAATGCCATGCTATGCTTTG
AACTTGGAGTTTGCCTCTAAATCAGTGTTTTTCTAAGTGAGTTCCAGGGCAAACTGATTCAGAG
GAATGTTCACAGATTTTGCAATAACAAAAGGGCTCTGTGATACCAAGGGAATTTGGAAAACCCA
GACTTAAACTAAAGAGGGTATTTTGGGGTGTGTGTGTGTTTCTGTTTTGTCTTTTACTGTCTTG
GAGCCTTTGATATGCTACTGTCTTCTCAGAGCTTCTGGGTAGGAGTAGGGTAGCCTGGGAGTAG
ACAGAGATCTACTCCATCAGACCTTACAATTTCTTTTTTGGCAGGATAGCTCAAGGAATTATAC
CCTTTGAGAAATAGCCTTTCCAGATAACAGTTGCCATTAATAAGCTTTAGGTGCCAGGCATTTT
AAGTAACTTGACATTTATTTCCAAAGGTTGGATTTGATGCCATTAGAGTATATAATAAGAGGCT
GAAAGCGCTGAGGAAAATACAGAAACTAATAAAAAGTGGACAATTAAGAGCAACAAAGGTGAGA
TGACATCTTTCTTTTCCCCCTTAAATTATTTCCTTTCCCTGATGAGAGCAGTTAGAGGGTCTAA
AATTAAATCTATCCTCTTTAGTATCCAGATGTGAATGAACAAATGACATGTACGTATCAAAGAA
CAACTGAGCTATTCTTATAGGCAAGCGGGAGTGGAAGGGAATGAAATAAAATCAGCAGCGCTGC
CTGGAAACACAGGATGTGATTTTTTTCCCCACCATGAACAGTGTCTGCATTATTTTCTATACTT
TATTTTTACTGAAAGGAACAAAATAAGTGCAAATTCAATGCCAGAAAACTACCCACCATAGAAG
GCAGTCTAAGTCCATAAACTTTTGGAGCTGTTAACTTTTACTCAGATTCTTCTGGCCAGTGTAT
TTCTTGGCAAAGTTCCACAATCATCCAGAAAACAAAAGTTTCCTGGCTCCTCTGTTCTCCCAGT
TTTCTTCCTAGACAACATCAAAGTTTGGGCTGAGGTGAAACCCATCCTTAAAAATTCATCTCCT
TGATGTCTTGCTTACCAAGGAACAGTGTGTAGGTCTTTTGGATAATTTGATATGAATGGTTGAA
AGATTTCAAATCTTTGACAATTAAGTTGACAGTGTTTTCTTCGTTTAGAATGGCATCATAAGTG
ATGCTGTTTCAACAAATAATGCTTTTGAGCCTGATGAGGATATTGAAGATCTGGAGGAACTTGA
TATCCCAACCAAGGAAATAGAGATTCAAGTGGATTGGAACTCTGAGCTTCCAGTCAAAGTGAAC
GTTCCCAAAGTGCCAATCCATAGCCTTGTGCTTGACTGTGGAGCTATATCTTTCCTGGACGTTG
TTGGAGTGAGATCACTGCGGGTGGTAAGGTTCTGGTTTTCTGAATTATACATTTGGAGCTTTGG
CAATAGTAAAATGATGTGGGTTGTCCAGTATTGCAACAGGGCAAATACATGGGCTTTGTAATTT
TTCTAGGTGAATGCTTTTGTAAAAAAGTGTAATATTTTAAAGCATAGGCTCTGGAGCCAGACTA
CCTGGGGGAGATACTGGCTTCACCACTTACTAGCAGTACGACCCTGGGCAAGTTGCTTAATCTG
TCTATATCTCAGTTTCTTCATCTGTAATATGGAGGTAATGATGGTATCTACCTTCACAGGTTGT
TACAAGGATTAAATAAGCTAATAGATATAAGGTGTTTAGAAGAGTGTCTGGTTCAGGCTGGGCA
TGGTGGCTCACGCCTGTAATCCCAGCACTTCGGGAGGCTGAGGCAGGTGGATCATGAGGTCAGG
AGTTCAAGACCAGCCTGGCCAATATGGTGAAACCCCGTCTCTACCAAAAATACAAAAATTAGCT
GGGCATGGTGGCGCACACCTGTAGTCCCAGCTCCTAGGAGGCTGTGGCAGGAGAATCGCTTGAA
CCCGGGAGGTGGAGGTTGCAGCTGAGATTGTGCCACTGTACTCCAGCCTGGGTGACAGAGTGAG
ACTTCATCTCAAAAAAAAAAAAAAAAAAGAATATCTGGTTCAAAGATACTCCCCAGCAAATTAA
TTCACATTTATTATGTTTTATCTTTCTGAGAATGTATAACAAGTGGTATATGAAAAGAAAAAAA
TGGGCAAAAGTTTATTAAGTATTACATTTCTATTTGTTATGTTAACAGCAACAGGATAAGGAAT
ACCAGGTGTATGTTAGGACTGGAAAAGCCAGGCATTATTAAGAGGTTAGAGTAGGAAGCAGGTC
AGACATCTGGAAGGTCAAAGCAAGAGTTGAGGAGTATGCAGGATAGAAACATTAATGATATCAG
AAACCAGTTATGCAAGCTGAAGTTCATTAATTCTGTCAACAAATAGATGTGAAATGCCTAATGT
GTACTGAGCACTCTGCCAGGCACAAGAGACTAGTGATGAAGTAGACACAGTTTCTACCTACGTG
GGACTTAGCAGTCTGGAGGAGAAGGCAAACATTAAGTCATCTTATAAATAACAATTGTAGTAAG
TGCTCCAAAGAAGATGAATAGGACATCACCTGAAGCATCAAGTTCAGTGAAGCTTCAGGTAGGT
ATCAAAAGGGTCAGGCAGAAAAGTAAGCAGGTCAAGACACCAACAATCAGATGTAATAATATAA
AGCTGACTCCTAACTGAGGTTCCACTTAGCTAAGGCTTCTTTGCTACCTCTATAAGGGGAGGAA
ATGATTCCAGGATGTGGGTACTAGGTAGGTAGGGTAGAGAAGTGATTGTGCTTGGCAAAAGAGT
ATGAGAATATGCTGACAGGTTCTGTTCTAGTTCTCTTAGCAGATTTTGTTATTAGCTCATTTGA
AATCACTCTTGTTCTTTGTTGTACATTTCACTCTAAGCATTGGCTGAGATCCATTGTAGACTAT
ATTGGCCAGAGAGTTCAATGAATTAGTTTTGGGGACAGTCTTAACATATTTAATGCCAGAATAT
TTCCCACACAACGAATATTTACTTTCAAATATTATAATGACAGTTATGATAGCTAAAGAAGACT
CCAAACTTTATACTTGAACAGAGGTCTTGATTTGTTTTCCTCCATAATTCTCAAATTCTCTTTT
ATTATTGACTATGGGTCTTGTGTCTTGACTTTTTAAAATTAAATGTTTATTTTGACATAATTGT
AAATTCACATGTAGTTGTAAGGAATAATATTTAGATCCTATATACCCCTTACCCAGTTTCTCCC
AATGAGTAACATCTTGTAAAACTGTAGCATAAAATCACAGTAGGACACTGACATTCAAAATAGA
GGCCTTGACTTTTAGGTCAATAGTAACATTTTGAACCCGATAAGCATAGCCACACCCATATCTC
TTCTCCAATCAGCAGGGCCTATATTAAGAAGCCTCCTGTACTCTCTTCTTTCCACAAATGCTTA
TTTAGCACCTCCACGCTATCAAGTGCTGTGCTATTGAGCGCTGGATGTTGCCATCTCTTGAGAT
ATAATCCTAATTATACCATTACTGTAGTTTGAAAACCTCCATGGTTTTGCAATAATAACCTTTC
CTTAAAGTCCTGATTAACCATGAAAGTAATAATGTTTCTCCTGAGCAAGTAACTGAATGCTAGT
GAATTATGGGCAGATAAGGTTGTTAATTGTTACAAACTCTCCTTTTTTATTTTTAGATTGTCAA
AGAATTCCAAAGAATTGATGTGAATGTGTATTTTGCATCACTTCAAGGTAAATACATATATCTA
CATATCTACCTGTAAGACTTTCCCGTAAGCCCTTTCTCCTATCTGGGACTGTGGTCACATTATG
TCTGAAGGCCTTTTTTTTTTTTCTTTTAAAGATCTCAATTGTCATTATTTGCAGTTCTGGAATC
TGGCACTGCTTCATTCCATAAAACAGAATAAGTGTTCCAAGGAACTAAGCAGGAGTTCAGTATT
ATACACAGAAAAGGCCAAAGACAGCAAAAGCAAGCAACAAAGAGTATATTAGTCCATTCAAAGT
TACTTTCTTGTAAGTCAGAGACAGTGAGACAGAACAATAGGAAAATAATGGATTAGTTAACATG
AGGTCATGTCAGGATACTTTTTTGTATGAGGATTATTGCAGAGGAAGCTTTATTATTATGCCAA
CTGGAAATTTAAACTGTCCTGTTTTAGGAAATTTGCTATTACCTCTCTCTCCTGATTTCTTCAA
GTATCAGATAACAATTTAGTTTAGGTTTGATAACTTGAAACTTTAGCATGAGTGACTCCATTTT
GATTTTTAGTCTTGTCTGTTGTCGTCTAGTGCGGGAGCTTAGTCTAAAACAATGGCCTTTTATT
TTTATTTTTTATTTTACTATGTGCCTGTGCCTTGCATGACCCCAGGATGGGCACAGCTGCTTGG
CCCCTGAGCCACCGAAGGCTGCTCTACTCCTTCGTTCTGGCTACTAAAAGCTGCAAAGCTCTGG
GAGGGCAAAGCTCAAGAAGACTAAAACAATGGCCTTTACCATTTTTGTTTGATGATTTTCCCCG
TTTTGATCAGGCTCTCACCTAGGTAAGAGTGAACAAAATGTAGGAGATCAGTGCTACTCTGTTA
CCATCATTTTGGGTTTCTGGTCTCAGGAGGTCATGTGTAGCTTATGGTGCCCTCCTCATTATCA
TGTATGTCTCTGAGTTTTTGTTGTTTCAGAGAGAGACCATTCGATGTCTGACAGGTGGCTTTTT
GGAAACATTTAAAACTTTGAGAGGGTATAATGTACCAGGAAGACTGCTATTATGACTATCAGGG
AGATAATACCAAGGGTTTATAGTATGCTCCTTAGCCAGGATTCTCATGAATCAAACCAACTAAA
ATTGAATAGCCTGAGAAGGAGACTACCTGTTTTAACCAAGAAGTCTTCTCTGTAGTACCTGATG
TATTTATATATGTGGAACAAGAAGTGTCACCCAACTGCACAGATGCTTCCTTGTTGAGTTAGCA
GGTAATCTAGCATTCCATGACTGGTTAAATTAAAGCAGAAAGTGTAAGTTACCCAAAGAAGCTA
CTCATTGTGAAGTTTTAACTACAGCACTATCCTGCCAAGTGAAAGAGGTAGGCACAAGTAAGGG
AAAATTAAAAGGGATAAGCATCTTATGATAGGGAGTCTTGTTCTGACAGTCTTGGAGCTGTCTA
CAGCATGAAGTTGACAACTTCTTGTTTTGGTTTGTAGTTTTATTGTCTCTAGTTGTGGCATCCA
GCATTCTGGCGAACTCTCTATGTGGCCCACAGTTTAAGCATGAGATTCATCTCTTGAAATTTAC
ACTGAGTTGCTCAGCTTCAGCTTACAGAGCTTCAGGAACTGAGTAGTTCTTGGTCTTCATTGGA
GTGTTGTAGCCAGATATTAGTGAAAGCTAAAAGAATTAAGAATCCAGCTCAGTCTACAGGTAGA
TAATAAAAACTCATAAATATTGAACAGGGCTACAGTCTAAGAACAGTTTTACTATGCTTTTCTT
TTGAACCATATGTTTTTCTTTCTATAGTCACCTCCATTTCTATCAAAGACGATCATGGTAGACC
AATTTGTTTACAAAATAAGTTTGGTCTCAAACTTGGCCTGATTGATTATTTACACAGTACAGCA
AGAATAACTACATAGGTGACTCCTTTCAAATTTGCTTTGCCAAGCCTGGCGAGGTATTGCTTGC
CTGTGAGCCCAGCTACTTGGGAGGCTGAGGTGAGAGGATTGCTTGAGCCCAGGAATTCAAGGCT
GCAGTGCACTATGATTGTGCCTGTGAATAGCCACTGCACTATAGCCTGGACAACACAGTGAGAC
CATGTCTCCAAAAAATAATTGCTTTGATAGAAATTTTGACAAGGAATCTCAGATTGGACTTTTT
AAAACGTCTTGATGCTATGAAGTCAAACCAAGGCAGACATTAGGCTTTGCCTGATGTATCTAAT
ATCTTGAAGTTACTGGGCCTCCCAGGAAGGAACGACTTTTTATTCACTCATTGTAAGGCTAGCA
GCCCTTGAAGCCAGGAATTCTGTGCACATTTTCAAATATGATATTCTATTCAAAGCCTTGATAA
TATAACCAATGTTTTCCAATTGTATTCTATTTAAAAGAACAGATTCTATTGAACTTTCATGTAA
ATAATCATATTGCCATAAAAATAAGAATACTCACAAAGAGTTTCCAAATTCTGGAAGGATCAGG
TAGAGAGGAAAAGCAAATGTTTCAATTTTTGTTTATGAAAGTATGCTTAACAAGGCTGGGTGCG
GTGGCTCACATCTGTAATCCCAGCACTTTGGGAAGCCAAGGCGGACAATTGCTTTGAGCTCAGA
GTTTGAGACCAGCCTGGCAACATGGCAAAACGCCGTTACTACAAAAAATACAAAAAATTAGCTG
GGCATGGAGCCTGAGGCTGAGAATCACTTGAGCTGGGGAAGTGGAGGTAGCAGTGAGCCCAGAT
GGTGCCACTGCACTCCAACCTGGGTGACAGAGTGAGACCCTGTCTCAAAAAATAAAAAAAAAGT
ATGCTTAACCAAGTGGCTGTAAACTGCAGATAGCTTTAAAGAAAAATTTTCTTTAGATCTGGAA
AACAAATATTAAAAGAACCAGCAATGTTTCAAATAAAAAAGCCATAAAACCTGTAATTCTTCTC
CATCAGTTCATTCAGTCTCATGTAATTAATTCTTGCTCTGTTTGATCTTGGCTGGTAGTTTAAT
TCCAACGAATGGTATGAATTCAAAGTTATTAGAAACCTGTATTTGTCAGAGTTCTTTTCATTCT
TCCCATATAACTCTTTGAAGTCACAGCACTTTAGAATTATAATGGCTTACAAAGAGCTTTCAGA
AAAAGTATCAGAACAAAACAATTAACTGTGGACAACAAGACTTAAAGTGGCTATATTTAAAGAT
CTGATGTGAGTTACCCAATTGACAAGGATATTTGGATATTTCTGTGGCACACAACAATTTAAAA
TAACCAAAATTATGACTGGTAGCATTTATACCAAGACCTATCACATTTCTAGGAATGTTATATA
ATTTTGGAACATATTAATAACATATCTATAAAAATATAGCACAAAGAAAGTTAAACATCATTTC
TTATTTTAACAGTGCTTCCCATATAATTTAACCTATCAGATAAGGCCATTTGGTTTAACATCTC
TCTTTTACAGATTCTTTAAGAAATTCCAGGGTCCTCTGGAACATCCCAAAGTTAGTTCAAGGTC
AAAAAGACTTAATTTTGATTTTTGAGAAGTTTGTCAAATACCAAAGGTTTAAAACATTTGATCA
AAATCGGATCATAGGTCACTATGAAATAAAACCAAAGTGAAAAAAGAGTTCAAAGGCAAAAAGC
ACAAGAAGAGTTATATTGATGAAACATGAAATCTCTGTTTTCTAGGCGAGTTACCTGGAAGAGA
AAATCCTCTCACAATTTTCTATTAAGAGTAAACCAATCCTCTGAGAAAACTCTATTGTTCCAAC
ACATAGGCCCACACTTTAGCCTTCCATCAGTGTACTTTAATATTAATGCTCAATTTTTAGAAAA
ACTTATAAATAATTCCCTTCTACTTTTAGCCAACTCAATCACATAAAATTTTTCATGATATTTA
TCTTCTACAAACCTTCTACAACTTGCTTAAACCTTCATTTGGTCCTATACTTCCTTTTTTAAAA
TTGGCATTGTACCTTAGGACAAAGATTTACTTTTCTTTTCTCCTTATCATTTTGACCATATAAG
GTTATCTCCTATACAAAAGAAAAAATTACTCTCTTTTCAATTTTCTTTATCTCTTCATACTTGT
AAATTTCTTCTCACATCTTTCCTACCTAGTGGTTCCTTCCTGCCTTGTTTTGATTTCCTTCATA
AGTCCATATTTAGAAACAACCTTTAAATAACCTCTGATTGCCAAGCTAGAGTTAAGTTTTAAAT
AAATAAATAGGCCGGTCATGGCGGCTCACATCTGTAATCCCAGCACTTTGGGAGGCAAAGGTCA
GAGGATCACTTCAGGCCAGGAGTTTGAGACCAGCCTGAGCAACATGGTGAGACCCCGTCTCTAT
TATTTTTTTTTTAAAAAACAAAAAGCATAAAAACTCTGAGAACATTTTGAACCCAGAAAGATAT
TACTTCCAATTTGTGCCACAGAGCTGGTAATGTATGGAAATGTGTATCTTGACAGTGGGTGGGG
GTGGGGTGAATATTGACAATGGGGGGATGTTGTTATGAGGAGAGCAAAGCTGGAGATTACTTAG
ATTCCTTAGAACAGGAGGACTCTCAAGGGTGTGAGTCTCTGTGTGTTTCTTTATGAATGTATGT
GTGTAGGAAAGTGCTAAGGCTTAGGTTGAATTCTTTAGCAGCAGAGCCCAAGATGGGGATTCTT
GCACAAGCGATTTATTGAGAATACATCTGAGGAAATTTATGAAGCAGTGAGGGGAGCAGGATAG
GGAAGGGGAAGAAGCTGATCAAAGACATGGTTTCTGAAGTCCAGCGTAGATCCCTGATTTCACA
GGGAGCACCACCACAGGAAGAGTACCAGAAGGTTATGCCTCCTTGAGGCAAGAGGACCTGCTTT
CTCCCCCAGTAATGAGTCAGTCAGTGGCTGCAGGCCAGCCATGTGTGGGGATGAAACCTCCCAG
TTCTCTTTATTTACTTATTTATTTGAGACAGGGTCTTGCTCTGTCACCCAGGCTGGAGTGCAGT
GGTGCAACTGTGGCTCACTGCAGCCTCAAACTCCTGGGCTCAAGTGATCCTCCCATCTCAGCCT
CCTGAGTAGCTGAGACTATAGGTGCACAACCACACCTGGCTAATTTTTGTATTTTTTTATAAAG
ATGGGGTTTCACCATATTGCCCAGAAGTTCTGGGTTCAAAGTGATCTGCCCTCCTCGGCCTCTC
AAAGTGCTGGAATTACAGACCCAGTCCTCTTTAGATGACACAGCTCCCAACGGGCAAGGGAAAG
TCTCCAGAGAAGGGCACAGCTGTGAAACATTGGCAGCCTGGCCCACAGCAGCAGGAGGATGGAT
ACACTGGCTTGGCCAAAGGGACGTGATCGTCCACAAGGTTGACTACGACCAGTTATGGGATAAC
CATTCTATATACTAGTGGAAAAGAAACTGTCATTTCAAATCTGGGTCACATTTTGGATAAGAGA
AAAATAAATAAATAAAAGGAGGAGCTACAAAAACTTAGTATAGGTTGATGCTTTAAAATTTCTT
TTCTTAGCTGGGCATGGTAGGGTGTGCCCTGTAGTCCTAGCTAATTGGGAGGGTGAGGTGGGGG
GATCACTTGAACTTGGGACGCGGAGGTTGCAGTGAGCAATGATGCCACTGCACTCCAGCCTGGG
CAATAGAATGAGACTCTGTCTCAAAAACAAACAAAAATTTCTTTTCCTAGGAACTAACAAAACA
TTGTGTCTTTCTTTTGAAGATTATGTGATAGAAAAGCTGGAGCAATGCGGGTTCTTTGACGACA
ACATTAGAAAGGACACATTCTTTTTGACGGTCCATGATGCTATACTCTATCTACAGAACCAAGT
GAAATCTCAAGAGGGTCAAGGTTCCATTTTAGAAACGGTAAATATTCAACCTTTCTACAGATGT
ATCTTTTCTAAACTATCATGATTTCTATAAATGGCAAACATTACACAAGTCTAGTCTAGCTGTT
GAATTTTAAGCTACCTATATAACTTCATGGAGCCTCAGTTTTTTCATCAGTAAAATGGAAGTAA
AAACATTAACCTTGCTAGGTAGATATGAAGACTAAATAAGATAGTTTATAGGAAATTACCTGGT
AGTACCCAGTACTGAATAGTTCTACAATGTGTAGGTTTATTAATAAAAGTGGGCATTAGCTATA
ATGCCTTTTAAGAATATTGAATCTTGGATCTTTTGTGATCTGGGATTTGTGTACATAAATTCCA
CTTAAATTCTCAGCAATCTGGAAAACCTTGAATTATAAATGTCTTTAAACAGGATCCACCAGGC
CATAAGTGATCTTTGAAAAAGAAATTTAAAAATCAAGCCACAAAGAAGCCCAGGGTTAATTTTT
CCCTACAAAATAGAATAGGCCCTGATGGGAGGCCTCATGGCACAACAGTAGCGCATCTGGCTCC
AGAATAGGCCCTGATTTCCCATGGGTATTATTTTGTTAATTTTTCTGTCAAATACCCAGAAGAG
GTGAGCATTCTGGTCTGGTTCATTGGGCTTGGCCTAATGGTGCTCCTGGTGGTACAAGAATCTC
ATTTCTCATAGTGAATTTAGGAAACTAAAGCAAATACCTATCAAGATATAATAACAGTAATGAT
TCCAGTGACATCGTGGATATTTTAAATTGCTTCCCATTTTTCTAAAAGTAGAATAAGTAGATCC
TTATTGTAGATTGGCAGTTGATTGAAGAAAACTAACTTAGGAAATGATATTATATTATTCTTTT
TAGGCACTGGTAGTGGAGAGATTGTTCTATTTGATGTTCCTTGGTAAAATACTAGTCCTAACTA
CCAAGCTGTGGATCTTAAATATAAAAAAGGAAAAAAAAAAAAGAGTAAAGCTACAAATCTAAAG
AAAAAAAAACTGTTTATCATTGAAAATTCATAAACTTTTTCTATCCAACTTAAAATTCTCTAAC
CCATAGGTGATAAGAAAGTTGGATCATATGAAGTATCCTTTAAGAGATCCACATTAAAAGTAAA
ACAGAACCAAGGTATCAAGTTCTTTCTCAACATGAAAGCAGTTATTTTATTTGTTTATACCCAT
TCTTACTAAGATTTTAGGCTATGAATGTCATAAAAACCACTAACCAAGAGTGTATCTACTAGGT
TAGACTCACTCTTGTTTGCAATCAGAGAGGTATTGTGAGTGGCTGGGATGGATTTTTTTGGACC
TTAAACAAATAGACTAAGCATAACGGAATGCTTGTATTAGATACTTACTGGTCCACTAAAAGTG
GGTTGGACATTCCTGGTGGCCTGTGAGATCAAGAGGCTTATATTTCTGTGTCTTCTCAGAGTCT
AGCACAAAGCTTTATATGTGATAAGTAGGTGAATGATGATAAACTCCTTCAAGAGGGAGTCCCT
CTGTATCAAAGATATTGTGACTGCAATGACACAACCTTCTTCCTATCCCTTTATTTTACCATCA
CAGGACTATCTCAGGACACAACTTGTAGTTAGACTTATCCTCTACCTTTCCCATTATGTCATAC
CATTTGCACAGGGACTTAACCCTCTAATTCATTCTCATACTAGCTAAGAGAATTGGGCTATTTG
TGAGTTGAAAAGTAGCTAAGTAGCATTTCAAAATGTTATTTTAGCCTGGATATATATTTGGAGT
TGCTTTGAAAATGTCCTTTTCCATGCAAAACACAAAGCCAAAATTGTCGTTGGTTTCCTCTGTG
TAGAAGATTAAATTACATGCCACCTCTAAACAGTAGAGCTTTTCTGAATAACCAACTTGGTCCA
TAGACATTGGTTTCCATCTCCAATAGAATTAATTTCCACCCAATTCCATTTGTGGCTGTTTTTT
GTCATCAGTGACAAGCTCTTCACTGTGCATTCATTGCACACTCAACGCTGTGCTAAGTGCTCTT
AGCTTAGCCATTGAGAGATGCACTATTGACTGCTGAATCATTTAGGCAGAGGGGGTGACTTGTT
AAGAGGCATACACTCAAGAGGTTGGGGAAGAGAGCTGAAAAGGAGTTTACACAATGGAGAACTA
TACCAGTTCACCTTTCAATGTGCAAAAAAATGATATACAAAAAATTTTAGTTGGGAAATATAAA
AGAACAATACAGCTGAAGAGGATTCTGAAGTATGTAAAGACAGATGAGAAGCACCAGGAAAGCT
TCAAATCATTTTCAGTGGAGCATCAGGTGGGTTGATGCTATTCTATTTCTACCCTGTGTTCTCT
TTTTCAAGATCACTCTCATTCAGGATTGTAAAGATACCCTTGAATTAATAGAAACAGAGCTGAC
GGAAGAAGAACTTGATGTCCAGGATGAGGTATGATCATTTTCTTCTGAAGAAAATATTTGAATT
ACATTTTGAATAATTAGAGTAATACAAATAGTGAATATATCTGATTAAGAACTGTCAGGGAACA
TAATTCCCCCAAATGCAGAAAAGATGGCTTCATAGCAGGGAAAAAGAGAAAATAAGAATTCTCC
CTTGAAGACATTAAGTACTTGGGGATCTTTTGTCCCAGGCATTTGGGGATGATCAGCAACTGGC
AAGAGCCAACACACTGCTCCTGATTTGATACCTACACCTCTGCATACTGCTCTTCTAGAGATGT
AAGGCCATGGGGTAGGAAAAAAATGAATAGAGGAAACTTCTAAGCTAGGAACTTTATAAGCCTC
AGGAATAGTATGTGAGGAACAGAAACCCTATGACCTTCCTTCATATGACTGATAAAGGCTTTAA
AAACACACTCTCCCCACCCTAGACTGATTCTGTGTATTTCAGATGCTCTTGTGACATCAGACTA
TTCCTAGGAAACTTGTCTGAACTTGTAGCATCTGAGTAAATTTTGCAGCTTCGCCACTGGATAA
GAGGTGAACACTGAAGGAAGATCACAGAATTGTAAAAAGTTGAAAGATTTCTTAGAATCAAGTA
GTCTAACATTCTAGCCCATGCAGAAATCCTTCCTACACCATCCTTGATGAATGTTCATTCAGCT
TTTATGTGAACATTTCCAGACATTGGCCACTAGCGGCTTCTCAAGGGAGTTTGTTACATGGTTG
GAAGTCTCTAAATGTTACTTGTGCTTACAGAACTCAATTTGGCCTTTCTGTAGTAACCACCTAG
TGAACTGAGATCTGCCTTCCTTAGCAACACAGACCTTCCCACTGAACAGCCCTTCAAATATTTG
AGAATAGTTCTCATGTCTCCAGCAGACTCTCTTCCAGTTGAGACTCTTCTAGTTCCTTCTATTC
CTTACAAACAAGCAGTCAGTGCTTCTCTGGTCACGATGGGGACACAAAACCCTAAATGCGGCCA
GGCCAGCATGGAGATTATTCAACCATGACCTACCTTGATCTCATCAAGATATTCCTATTACTAC
AGCTTAAAAATGTGTTTCCTTTCATAATAGCAACATCAGAATTTTAGCTTCTATTGAGCAGGGG
CCAACTAAAGACTGGGGCATTTTCATACAAGCTATGGTCTAGTAGAGTTCCCTAGTTATATTTA
TTTAATTGAGCTTTGACCCTAATACAGAACTACACCTATTTCTGCTGGATTTCATCTTGTTGGT
TCTTGTTTACTGACATAATTTTGTATTATTATTAGTATTATTATTATTATTATTATTTGTGACT
GAGTCTTACTCTGTCACCTGGACTGGAGTGCAGTGGTACAATCTCAGCTCACTGCAATCCCTCC
CTCCCAGGTTCAAGAGATTCTCACGTGTGCCTCAGCTTCCCAAGTAGTTGGGATTACAGGCGCT
CGCCACCACGTCCAGATAATTTTTTTTTTTTGTATTCTTAGTAGAGACAGGGTTTTGCCATGTT
GTCCAGGCTGGTCTTGAACTCCTGACCTCAAGTAATCCACCTGCCTTGCCCTCCCAGATTGCTG
GGATTACAGGCATGAGCCACCACATTGGCCAGTTTTGTGTTTTGAATCTAACATCCAGGATATT
GTCTACCCCAGTACCCGTTACCTGGTGTCATAATCAAATCTGATAAGCCTGCCTTCTCTGCCTT
TTTCTAGGTCTTGAAAAAATGTTGCAAAGAAAACATTCAACCCCCTATAGTGAAATCAGGCAGT
AAGGATGACAGGCAGAGATTTCTGTGGAGGAAATACATTGCTAGAGTGAAAATCACAGTACCTG
CATCTTGGCCCACCAGCTTAGTGAGCCCAGGAAAGTCATTATCGACCTGAGAAGTCCTTTTAGC
TCCAGTAGTTTATGACTCTATGGATATAGGGTGCCTTAGTTTGAGTAAAAACTCATATGTCAAC
ATTAAAACTTAAGACTGCTATATTATAGGATATTTATTGGTTGCCACGTAAAATAATCATTCAG
GGAGGGTTTCTATAGATGGTAAAACAATATAGCACAATAGTTTCAAGGGCATGGACTTTAGAAC
AAGACAGACCTGGGTGCTAGTCCTCATTCTGCTACTTACTGGCTGTGCACCCCAGGGCAAATTA
CTTATCCTGTCTTCATATTAGTATCTTCATGTAAAGTAGTAATAATACCACAGAGTTGTGAAAA
TTAAAGGATAAATGTAAAGTAACAAGAGCAGTGCCTTGTAGATAGTAAGTGCTCAATAAATAGT
AATCATTGCTCTTAAATAAATCCAATTAAAGTGTGATGAATACAGGCCAGGCTCAGTGACTCAA
GCCTGTAATCCCAGCACTTTGGGAGGCTGAGGCAGGGGGATCACTTGAGGTCAGGAGTTTGAGA
CCAGCCTGACTAACATGGTAACCCCCCGACCATCTCTACTAAAAAAAAAAAAAAAAAAATTAGC
TGGGTGTGGTGGCAGGCACCTGTAATCCCAGCTACTCGGGAGGCTGAGGCAGGAGAATTGCTTG
AACCTGGGAGGCAGAGGTTGCAGTGAGCTGAGATCACGCCACAGCACTCCAGCCTGGGCAACAG
TGAGTGAGATTCAGTCTCCAAAAAAAAAAAAAAGTATAATGAATACGAAATGAAGTTTTTACCC
TATTTCTATTGTGATGATATACACCTAAGATGAGTAGCAGTAAGCAATCAATACTATAAAAACA
TATTTATAAAAAAGATAATGCAGACTTAAGGAGAATTCAGTTGTATCAACACTTTGTTTTCCCC
TTGCTTCCACAGGCTATGCGTACACTTGCATCCTGAAAGTGGGTTCGGGAGGTCTCTATGAGCA
AGGAATACAAGACAAAACTTCCTCAATGCATTGACTATTTCTTCAGACTCAAAACACTCATTCT
TTTTTCTATTAAGCCATTGAAAGAGAAGCACTAAGACTGCTTCTAGGCTTTATTTATAAAATAA
ACACCTTATCCCTAACATGGGCAAAATGGCTAGAATTATTCAGACGATTTGGCAGCGTCCAGGG
TAAGCTGGTGTTATAATACGCTGCTGATCTACATCACAGATTTGCTAATAATGTTCACGTGGGC
CCTGGCATATCTCTGTTCAGTTAGAGTGAGTGCTGACCCAACAGCCTCTGTGGTCAAGCGAGTC
ACGAATGATTAATCATAAAGAAAAATCAGTTTTTGACTGACCTGGATATCCATGAGCTGCACTG
ATCACCATGTAAGGTCACATTTAGTAAATGCTGAAATAAAATGATTAATGCATTTATCAATAAA
AGCCTTTGAAAATACTTTGGATAATAAATTGGAGTTTTAAAAATGCAAATTTGCTTAGTATCTA
ATAATGAAGTGTTATTACATATAGCCGGAATTGAGGATCTCTTTGATCCTGGAAATGGTTTACC
TAAAAGCTACAGAACCAGGCCAATATATTTTGAAATATTGATGCAGACAAATGAAATAATAAAG
AGATTTTCATGGTTTATAAAAATCTTTTTTGATATGATAATAATCATGATCACAACTGAGATCA
AAAAAATATATGACAGATTATTTTGTTTAAAAATGCAGTTTTAATTATCTTAGTCTATAGAAAT
GATCATTGCATGGAGGCATGTATAGGTATGATCTGTGTAAAATCTGACATAAAAACAGTGCTAT
TCTGAGTGAAAATTTTTTTGATGTGCTTACATAACCATGGTGATTAAAATGAGTTTATATTTTT
TCTCAAAAATTTTAGCAGTGTGTAAAGTAAGTAATCTTTAACTGAACTCTGACCACTTAAAAAA
AAATCTAAAAATTGAACTACCTATAGTAGTCTGTGTTTAAAGTGAATTTTTAAAGACAAAGCAT
TCTAAATGAACTCAATATAAAAACATTCATTTGGAATGTACATACTGAAAAATACAGGTTTTTT
TGACCAAAAGTTTTTATATCTTTTCTTTTTATTTATTTTTTTCCTAAGTGCCAACAATTTTCTA
GATATTATATACAACACAGGCTTTGATCTTGGGGACTTTTCCCATATATTTCACACTGGAGTGA
ATGAAGTTGTACTTCATTTCTAGAGAAAAGTTATACCCAGGTCCCCAATTGAGAATGTCTTGCT
TGATTGAAAACGACATCATCCCTTGGTATACTCCAGGGATTGGTTTCAGGACCCCTGCATTTAC
CAAAATTTGTGCACACTCAAGTCCTGCAGTCACCCCTGCCTAAAGATAGAATGGCTTCTCTGTT
TTTCTTCTGAAATACAACCAGAAACAATGTGTCTATTTCTGAAAGAATAGGATTAATGATCATA
CAAATGGGTTAATCCTGAATTCTGGTTGTAAATCTGGTTACAGCATAACTAGGATTATAATGCT
GCCTCATTTTCACAGCACTAGTTGCTTATATTGACAACAAATCATCTCGCTAAAGAGTGAATGT
AGGCCAGGCGCGGTGGCTCATGCCTGTAATCCCAGCACTTTGGGAGGCCGAGGCGGGTGGATCA
CGAGGTCAGGAGATCGAGACCATCCTGGCTAACATGGTAAAACCCCGTCTCTACTAAAAATAGA
AAAAAAGAAATTAGCCTAGCGTGGTGGCTGGCGGGCGCCTGTAGTCCCAGCTATTTGGGAGGCT
AAGGCAGGAGAATGGCGTGAACCCGGGAGGCGGAGCTTGCAGTGAGCCGAGGTCGTGCCACTGC
ACTCCAGCCTGGGCGACAGAGCAAGACTCCGTCTCAAAAAAAAAAAAAAAAAAAAAAAAAGAGT
GAATGTAATAGTCTTGCAGAAAATGAATGAATACCTTTGTTCAATAAAGGAAATATGCACTGCT
CACTTTTTTGAAGGAAATGCCAAAGTTACGTTTTACAACAAGGCTAGAGTTTGTAAATTCTGGG
TTCATTTGTGATGACATAAGTCAGCAAACTGCGGGAATACTGTCTCTTCTATGTATTTTGTGAA
TAGTAAGCATAATTTTAGTTTTGTATTATCAATGAAAATTTCACTTGAAATTAAAGCTGCCTTT
TGTTATATTTTTAACCTATAGGATAAGATTCCAGTATTGTATATGAGTTTTAACAAATTAAAAA
ATCAAATCATGTAGATTTGAAAATATTTGCACACATTTAAAAATAAATGTAAAGTTGTCTTTTA
AACTACTCGGATGTGTCCTTTCTGAACAAAACTATTAAATATAATAAAGAATGTGCCAGGAGCA
GTGGTGCATGCCCTTGTAGTCTCAGCTACTTGGAAGCCTGAGGTGGAAGGGTCCCTTGAGCCCA
GGAGTTAGAGTCTCAGCCTGGGCAACACAGCAAGACCCTGTTTCTATAAATAAATAAATAAAAT
ATAATAAAAATACTTCATTCTAAGTTTGTCATTGCTCTTATCAGTCATCATTATGAGGCATTCT
TTATAGCACCACTTCTGTTTATAATTCCTTTGTGTAGATTTATCAGTGCAATAAACAACATTAG
TCACAAATATCACAGTTAAACCTTGTGATTCTTATCCAAATATTTCCAGTTTATTTTGTGCAAT
GGTTATTATTATTTTAAGCAAACTTTTCATTCTGGCACAACATATGTAAAAAAGGATACAAATC
ACAGTGTACTGCTTGAAGAATTTTCACAAAACAAGCACACAAGGGTGGCCTGTGCCCAGATCAA
GCAGCAGAATGCCACCGGCACCACATAAACTCTTCATGCTCCCTCTTAGTTGGTTGCCCCAAAG
GGCAATTAGTGTCCTGACTTTTAAAATCTGTTTTTTTTGAATTGCATAGAAATGGAATCAAAGA
TTATTTCTTTTTTCCCCCTTTCTTTTGTGTCTGTCCTCTTTCACTCAACATTATAATCAAGAGA
TTCATCCATCTTATTCAATATAGCAATCATTCATTCATTCTCCCTGCTGTATAGTATTCATTCC
ATTGTAGAATTTACCACCACTTATGTATTCATCCTACAGTTTGTGGATATTTGTGTCGTTTCCA
GTTTTGGGCTCTCATGAATAGTGCTACTATGAACATTCTTGTGAGGCATCCTTGCACATTCCAG
AAATATTTGGTGGGGTGGGGTTCCTCTATCTGGGGCTTAGATCTGTGAGGTGAGAATCAATGAA
ATTAGGGTTATTTTCCATTTGCATCAGGTTAGGTATCTGAAGAAAGTTTGGTTTCAGTTAGAGT
AGCATTTATCATGCAAACAATTTAACAGTGTAACAAAAACCACACAAACTTTAGTTAAATCACA
TGAAGTTCAGTTCTGTAAGAAAGTCACATAATGACAAATACCTAGAAAAAGAGTGGAAGAGAGA
AAAAGTAGAAACAATGAGGGGAGAAGGAAAGGAAGTTCAGAGGGGAAAGTAAGGGAGAAAAAGG
TAAGAGAACCACAGAGAAAAAGAATGACAATTAGGGAAGGAAGAGAAATAAGAAAGGGAAAATT
AACGGAAGAAAGAAAAGACGAAGGTAAAATAAAAGGTGGAAGGAAGAGAAAGACTAAGAAGAAG
AAAGGAATGGTGGCGTAGAGAATTTCATAATGGGAAAATAAAATCCAAATAGAGGATTGATTTC
AATATAATCATATCCTTCGAGAACTCAGAGAAGTGAGCAACACTCCCTCCCCCACATGCCACAC
CATCTCCTGAATTCTTGGTCCCCCTTACCCAGCTCTTTTCATTAATATCAGCTTCTAGTATGCT
GTGTAATTCACTTATTACTGTTTCTCTTCCTGCCGCCTCGCTTATACTAGAACATAAGCTTCAC
CAAGGCAGGGTTCTTTATTTTGTTCCTGGACATATACCAAGTATTCAGAAGAGTAAAGAGTGTC
AGACACACGGTAGAGGTGCAATAAATACTCTCAGATGAATGCATTAAGCTAAAGTCAGTGATGC
TGAGTAAGGTTAACATAATTTGAGAATGGCAGTCATTAAGTACATAAGACATTCAGAGTTTCCT
GAGCTTGCAGGCCTATTCCAAAGACTTACTGTGACAGCTCACCTGCTTGCCTGTCTGCCTTACC
TATTGGTCTCCAGAAAATGGCCTGGGAATTCACTTGTATGTTAGCAGAGCTTCAGGGAATTAAA
GGCCCTTTAATGTCTATATCCATTTTGAATATGCCAACCAAGTCAGCCCACTTCATCCTTTTCT
TCAGGAACATTTTTGGGCGTGAACATTGAGGTGCTGTTTTTGCATAA

TABLE 1
Nucleotides Corresponding to Introns and Exons in SEQ ID NO: 3

| Element | Nucleotides | Element | Nucleotides |
|---|---|---|---|
| Exon 1 | 1-221 | Intron 1 | 222-1004 |
| Exon 2 | 1005-1171 | Intron 2 | 1172-2261 |
| Exon 3 | 2262-2801 | Intron 3 | 2802-11503 |
| Exon 4 | 11504-11614 | Intron 4 | 1615-13529 |
| Exon 5 | 13530-13714 | Intron 5 | 13715-14310 |
| Exon 6 | 14311-14475 | Intron 6 | 14476-22567 |
| Exon 7 | 22568-22720 | Intron 7 | 22721-22820 |
| Exon 8 | 22821-22903 | Intron 8 | 22904-28418 |
| Exon 9 | 28419-28566 | Intron 9 | 28567-29489 |
| Exon 10 | 29490-29603 | Intron 10 | 29604-33768 |
| Exon 11 | 33769-33846 | Intron 11 | 33847-33986 |
| Exon 12 | 33987-34082 | Intron 12 | 34083-35298 |
| Exon 13 | 35299-35405 | Intron 13 | 35406-37407 |
| Exon 14 | 37408-37477 | Intron 14 | 37478-39448 |
| Exon 15 | 39449-39541 | Intron 15 | 39542-40466 |
| Exon 16 | 40467-40562 | Intron 16 | 40563-41192 |
| Exon 17 | 41193-41423 | Intron 17 | 41424-43696 |
| Exon 18 | 43697-43751 | Intron 18 | 43752-49419 |
| Exon 19 | 49420-49565 | Intron 19 | 49566-51904 |
| Exon 20 | 51905-51988 | Intron 20 | 51989-56347 |
| Exon 21 | 56348-57175 | — | — |

Exemplary Human SLC26A4 cDNA sequence including untranslated regions
(SEQ ID NO: 4)
CTCAGCCTTCCCGGTTCGGGAAAGGGGAAGAATGCAGGAGGGGTAGGATTTCTTTCCTGATAGG
ATCGGTTGGGAAAGACCGCAGCCTGTGTGTGTCTTTCCCTTCGACCAAGGTGTCTGTTGCTCCG
TAAATAAAACGTCCCACTGCCTTCTGAGAGCGCTATAAAGGCAGCGGAAGGGTAGTCCGCGGGG
CATTCCGGGCGGGGCGCGAGCAGAGACAGGTCATGGCAGCGCCAGGCGGCAGGTCGGAGCCGCC
GCAGCTCCCCGAGTACAGCTGCAGCTACATGGTGTCGCGGCCGGTCTACTCGGAGCTAGCTTTC
CAGCAACAGCACGAGCGGCGCCTGCAGGAGCGCAAGACGCTGCGGGAGAGCCTGGCCAAGTGCT
GCAGTTGTTCAAGAAAGAGAGCCTTTGGTGTGCTAAAGACTCTTGTGCCCATCTTGGAGTGGCT
CCCCAAATACCGAGTCAAGGAATGGCTGCTTAGTGACGTCATTTCGGGAGTTAGTACTGGGCTA
GTGGCCACGCTGCAAGGGATGGCATATGCCCTACTAGCTGCAGTTCCTGTCGGATATGGTCTCT
ACTCTGCTTTTTTCCCTATCCTGACATACTTTATCTTTGGAACATCAAGACATATCTCAGTTGG
ACCTTTTCCAGTGGTGAGTTTAATGGTGGGATCTGTTGTTCTGAGCATGGCCCCCGACGAACAC
TTTCTCGTATCCAGCAGCAATGGAACTGTATTAAATACTAGTATGATAGACACTGCAGCTAGAG
ATACAGCTAGAGTCCTGATTGCCAGTGCCCTGACTCTGCTGGTTGGAATTATACAGTTGATATT
TGGTGGCTTGCAGATTGGATTCATAGTGAGGTACTTGGCAGATCCTTTGGTTGGTGGCTTCACA
ACAGCTGCTGCCTTCCAAGTGCTGGTCTCACAGCTAAAGATTGTCCTCAATGTTTCAACCAAAA
ACTACAATGGAGTTCTCTCTATTATCTATACGCTGGTTGAGATTTTTCAAAATATTGGTGATAC
CAATCTTGCTGATTTCACTGCTGGATTGCTCACCATTGTCGTCTGTATGGCAGTTAAGGAATTA
AATGATCGGTTTAGACACAAAATCCGAGTCCCTATTCCTATAGAAGTAATTGTGACGATAATTG
CTACTGCCATTTCATATGGAGCCAACCTGGAAAAAAATTACAATGCTGGCATTGTTAAATCCAT
CCCAAGGGGGTTTTTGCCTCCTGAACTTCCACCTGTGAGCTTGTTCTCGGAGATGCTGGCTGCA
TCATTTTCCATCGCTGTGGTGGCTTATGCTATTGCAGTGTCAGTAGGAAAAGTATATGCCACCA
AGTATGATTACACCATCGATGGGAACCAGGAATTCATTGCCTTTGGGATCAGCAACATCTTCTC
AGGATTCTTCTCTTGTTTTGTGGCCACCACTGCTCTTTCCCGCACGGCCGTCCAGGAGAGCACT
GGAGGAAAGACACAGGTTGCTGGCATCATCTCTGCTGCGATTGTGATGATCGCCATTCTTGCCC
TGGGGAAGCTTCTGGAACCCTTGCAGAAGTCGGTCTTGGCAGCTGTTGTAATTGCCAACCTGAA
AGGGATGTTTATGCAGCTGTGTGACATTCCTCGTCTGTGGAGACAGAATAAGATTGATGCTGTT
ATCTGGGTGTTTACGTGTATAGTGTCCATCATTCTGGGGCTGGATCTCGGTTTACTAGCTGGCC
TTATATTTGGACTGTTGACTGTGGTCCTGAGAGTTCAGTTTCCTTCTTGGAATGGCCTTGGAAG
CATCCCTAGCACAGATATCTACAAAAGTACCAAGAATTACAAAAACATTGAAGAACCTCAAGGA
GTGAAGATTCTTAGATTTTCCAGTCCTATTTTCTATGGCAATGTCGATGGTTTTAAAAAATGTA
TCAAGTCCACAGTTGGATTTGATGCCATTAGAGTATATAATAAGAGGCTGAAAGCGCTGAGGAA
AATACAGAAACTAATAAAAAGTGGACAATTAAGAGCAACAAAGAATGGCATCATAAGTGATGCT
GTTTCAACAAATAATGCTTTTGAGCCTGATGAGGATATTGAAGATCTGGAGGAACTTGATATCC
CAACCAAGGAAATAGAGATTCAAGTGGATTGGAACTCTGAGCTTCCAGTCAAAGTGAACGTTCC
CAAAGTGCCAATCCATAGCCTTGTGCTTGACTGTGGAGCTATATCTTTCCTGGACGTTGTTGGA
GTGAGATCACTGCGGGTGATTGTCAAAGAATTCCAAAGAATTGATGTGAATGTGTATTTTGCAT
CACTTCAAGATTATGTGATAGAAAAGCTGGAGCAATGCGGGTTCTTTGACGACAACATTAGAAA
GGACACATTCTTTTTGACGGTCCATGATGCTATACTCTATCTACAGAACCAAGTGAAATCTCAA
GAGGGTCAAGGTTCCATTTTAGAAACGATCACTCTCATTCAGGATTGTAAAGATACCCTTGAAT
TAATAGAAACAGAGCTGACGGAAGAAGAACTTGATGTCCAGGATGAGGCTATGCGTACACTTGC
ATCCTAAAAGTGGGTTCGGGAGGTCTCTATGAGCAAGGAATACAAGACAAAACTTCCTCAATGC
ATTGACTATTTCTTCAGACTCAAAACACTCATTCTTTTTTCTATTAAGCCATTGAAAGAGAAGC
ACTAAGACTGCTTCTAGGCTTTATTTATAAAATAAACACCTTATCCCTAACATGGGCAAAATGG
CTAGAATTATTCAGACGATTTGGCAGCGTCCAGGGTAAGCTGGTGTTATAATACGCTGCTGATC
TACATCACAGATTTGCTAATAATGTTCACGTGGGCCCTGGCATATCTCTGTTCAGTTAGAGTGA
GTGCTGACCCAACAGCCTCTGTGGTCAAGCGAGTCACGAATGATTAATCATAAAGAAAAATCAG
TTTTTGACTGACCTGGATATCCATGAGCTGCACTGATCACCATGTAAGGTCACATTTAGTAAAT
GCTGAAATAAAATGATTAATGCATTTATCAATAAAAGCCTTTGAAAATACTTTGGATAATAAAT
TGGAGTTTTAAAAATGCAAATTTGCTTAGTATCTAATAATGAAGTGTTATTACATATAGCCGGA
ATTGAGGATCTCTTTGATCCTGGAAATGGTTTACCTAAAAGCTACAGAACCAGGCCAATATATT
TTGAAATATTGATGCAGACAAATGAAATAATAAAGAGATTTTCATGGTTTATAAAAATCTTTTT
TGATATGATAATAATCATGATCACAACTGAGATCAAAAAAATATATGACAGATTATTTTGTTTA
AAAATGCAGTTTTAATTATCTTAGTCTATAGAAATGATCATTGCATGGAGGCATGTATAGGTAT
GATCTGTGTAAAATCTGACATAAAAACAGTGCTATTCTGAGTGAAAATTTTTTTGATGTGCTTA
CATAACCATGGTGATTAAAATGAGTTTATATTTTTTCTCAAAAATTTTAGCAGTGTGTAAAGTA
AGTAATCTTTAACTGAACTCTGACCACTTAAAAAAAAATCTAAAAATTGAACTACCTATAGTAG
TCTGTGTTTAAAGTGAATTTTTAAAGACAAAGCATTCTAAATGAACTCAATATAAAAACATTCA
TTTGGAATGTACATACTGAAAAATACAGGTTTTTTTGACCAAAAGTTTTTATATCTTTTCTTTT
TATTTATTTTTTTCCTAAGTGCCAACAATTTTCTAGATATTATATACAACACAGGCTTTGATCT
TGGGGACTTTTCCCATATATTTCACACTGGAGTGAATGAAGTTGTACTTCATTTCTAGAGAAAA
GTTATACCCAGGTCCCCAATTGAGAATGTCTTGCTTGATTGAAAACGACATCATCCCTTGGTAT
ACTCCAGGGATTGGTTTCAGGACCCCTGCATTTACCAAAATTTGTGCACACTCAAGTCCTGCAG
TCACCCCTGCCTAAAGATAGAATGGCTTCTCTGTTTTTCTTCTGAAATACAACCAGAAACAATG
TGTCTATTTCTGAAAGAATAGGATTAATGATCATACAAATGGGTTAATCCTGAATTCTGGTTGT
AAATCTGGTTACAGCATAACTAGGATTATAATGCTGCCTCATTTTCACAGCACTACTTGCTTAT
ATTGACAACAAATCATCTCGCTAAAGAGTGAATGTAGGCCAGGCGCGGTGGCTCATGCCTGTAA
TCCCAGCACTTTGGGAGGCCGAGGCGGGTGGATCACGAGGTCAGGAGATCGAGACCATCCTGGC
TAACATGGTAAAACCCCGTCTCTACTAAAAATAGAAAAAAAGAAATTAGCCTAGCGTGGTGGCT
GGCGGGCGCCTGTAGTCCCAGCTATTTGGGAGGCTAAGGCAGGAGAATGGCGTGAACCCGGGAG
GCGGAGCTTGCAGTGAGCCGAGGTCGTGCCACTGCACTCCAGCCTGGGCGACAGAGCAAGACTC
CGTCTCAAAAAAAAAAAAAAAAAAAAAAAAAGAGTGAATGTAATAGTCTTGCAGAAAATGAATG
AATACCTTTGTTCAATAAAGGAAATATGCACTGCTCACTTTTTTGAAGGAAATGCCAAAGTTAC
GTTTTACAACAAGGCTAGAGTTTGTAAATTCTGGGTTCATTTGTGATGACATAAGTCAGCAAAC
TGCGGGAATACTGTCTCTTCTATGTATTTTGTGAATAGTAAGCATAATTTTAGTTTTGTATTAT
CAATGAAAATTTCACTTGAAATTAAAGCTGCCTTTTGTTATATTTTTAACCTATAGGATAAGAT
TCCAGTATTGTATATGAGTTTTAACAAATTAAAAAATCAAATCATGTACATTTGAAAATATTTG
CACACATTTAAAAATAAATGTAAAGTTGTCTTTTAAACTACTCGGATGTGTCCTTTCTGAACAA
Exemplary Human SLC26A4 cDNA sequence including untranslated regions
(SEQ ID NO: 5)
CTCAGCCTTCCCGGTTCGGGAAAGGGGAAGAATGCAGGAGGGGTAGGATTTCTTTCCTGATAGG
ATCGGTTGGGAAAGACCGCAGCCTGTGTGTGTCTTTCCCTTCGACCAAGGTGTCTGTTGCTCCG
TAAATAAAACGTCCCACTGCCTTCTGAGAGCGCTATAAAGGCAGCGGAAGGGTAGTCCGCGGGG
CATTCCGGGCGGGGCGCGAGCAGAGACAGGTCATGGCAGCGCCAGGCGGCAGGTCGGAGCCGCC
GCAGCTCCCCGAGTACAGCTGCAGCTACATGGTGTCGCGGCCGGTCTACAGCGAGCTCGCTTTC
CAGCAACAGCACGAGCGGCGCCTGCAGGAGCGCAAGACGCTGCGGGAGAGCCTGGCCAAGTGCT
GCAGTTGTTCAAGAAAGAGAGCCTTTGGTGTGCTAAAGACTCTTGTGCCCATCTTGGAGTGGCT
CCCCAAATACCGAGTCAAGGAATGGCTGCTTAGTGACGTCATTTCGGGAGTTAGTACTGGGCTA
GTGGCCACGCTGCAAGGGATGGCATATGCCCTACTAGCTGCAGTTCCTGTCGGATATGGTCTCT
ACTCTGCTTTTTTCCCTATCCTGACATACTTTATCTTTGGAACATCAAGACATATCTCAGTTGG
ACCTTTTCCAGTGGTGAGTTTAATGGTGGGATCTGTTGTTCTGAGCATGGCCCCCGACGAACAC
TTTCTCGTATCCAGCAGCAATGGAACTGTATTAAATACTACTATGATAGACACTGCAGCTAGAG
ATACAGCTAGAGTCCTGATTGCCAGTGCCCTGACTCTGCTGGTTGGAATTATACAGTTGATATT
TGGTGGCTTGCAGATTGGATTCATAGTGAGGTACTTGGCAGATCCTTTGGTTGGTGGCTTCACA
ACAGCTGCTGCCTTCCAAGTGCTGGTCTCACAGCTAAAGATTGTCCTCAATGTTTCAACCAAAA
ACTACAATGGAGTTCTCTCTATTATCTATACGCTGGTTGAGATTTTTCAAAATATTGGTGATAC
CAATCTTGCTGATTTCACTGCTGGATTGCTCACCATTGTCGTCTGTATGGCAGTTAAGGAATTA
AATGATCGGTTTAGACACAAAATCCCAGTCCCTATTCCTATAGAAGTAATTGTGACGATAATTG
CTACTGCCATTTCATATGGAGCCAACCTGGAAAAAAATTACAATGCTGGCATTGTTAAATCCAT
CCCAAGGGGGTTTTTGCCTCCTGAACTTCCACCTGTGAGCTTGTTCTCGGAGATGCTGGCTGCA
TCATTTTCCATCGCTGTGGTGGCTTATGCTATTGCAGTGTCAGTAGGAAAAGTATATGCCACCA
AGTATGATTACACCATCGATGGGAACCAGGAATTCATTGCCTTTGGGATCAGCAACATCTTCTC
AGGATTCTTCTCTTGTTTTGTGGCCACCACTGCTCTTTCCCGCACGGCCGTCCAGGAGAGCACT
GGAGGAAAGACACAGGTTGCTGGCATCATCTCTGCTGCGATTGTGATGATCGCCATTCTTGCCC
TGGGGAAGCTTCTGGAACCCTTGCAGAAGTCGGTCTTGGCAGCTGTTGTAATTGCCAACCTGAA
AGGGATGTTTATGCAGCTGTGTGACATTCCTCGTCTGTGGAGACAGAATAAGATTGATGCTGTT
ATCTGGGTGTTTACGTGTATAGTGTCCATCATTCTGGGGCTGGATCTCGGTTTACTAGCTGGCC
TTATATTTGGACTGTTGACTGTGGTCCTGAGAGTTCAGTTTCCTTCTTGGAATGGCCTTGGAAG
CATCCCTAGCACAGATATCTACAAAAGTACCAAGAATTACAAAAACATTGAAGAACCTCAAGGA
GTGAAGATTCTTAGATTTTCCAGTCCTATTTTCTATGGCAATGTCGATGGTTTTAAAAAATGTA
TCAAGTCCACAGTTGGATTTGATGCCATTAGAGTATATAATAAGAGGCTGAAAGCGCTGAGGAA
AATACAGAAACTAATAAAAAGTGGACAATTAAGAGCAACAAAGAATGGCATCATAAGTGATGCT
GTTTCAACAAATAATGCTTTTGAGCCTGATGAGGATATTGAAGATCTGGAGGAACTTGATATCC
CAACCAAGGAAATAGAGATTCAAGTGGATTGGAACTCTGAGCTTCCAGTCAAAGTGAACGTTCC
CAAAGTGCCAATCCATAGCCTTGTGCTTGACTGTGGAGCTATATCTTTCCTGGACGTTGTTGGA
GTGAGATCACTGCGGGTGATTGTCAAAGAATTCCAAAGAATTGATGTGAATGTGTATTTTGCAT
CACTTCAAGATTATGTGATAGAAAAGCTGGAGCAATGCGGGTTCTTTGACGACAACATTAGAAA
GGACACATTCTTTTTGACGGTCCATGATGCTATACTCTATCTACAGAACCAAGTGAAATCTCAA
GAGGGTCAAGGTTCCATTTTAGAAACGATCACTCTCATTCAGGATTGTAAAGATACCCTTGAAT
TAATAGAAACAGAGCTGACGGAAGAAGAACTTGATGTCCAGGATGAGGCTATGCGTACACTTGC
ATCCTGAAAGTGGGTTCGGGAGGTCTCTATGAGCAAGGAATACAAGACAAAACTTCCTCAATGC
ATTGACTATTTCTTCAGACTCAAAACACTCATTCTTTTTTCTATTAAGCCATTGAAAGAGAAGC
ACTAAGACTGCTTCTAGGCTTTATTTATAAAATAAACACCTTATCCCTAACATGGGCAAAATGG
CTAGAATTATTCAGACGATTTGGCAGCGTCCAGGGTAAGCTGGTGTTATAATACGCTGCTGATC
TACATCACAGATTTGCTAATAATGTTCACGTGGGCCCTGGCATATCTCTGTTCAGTTAGAGTGA
GTGCTGACCCAACAGCCTCTGTGGTCAAGCGAGTCACGAATGATTAATCATAAAGAAAAATCAG
TTTTTGACTGACCTGGATATCCATGAGCTGCACTGATCACCATGTAAGGTCACATTTAGTAAAT
GCTGAAATAAAATGATTAATGCATTTATCAATAAAAGCCTTTGAAAATACTTTGGATAATAAAT
TGGAGTTTTAAAAATGCAAATTTGCTTAGTATCTAATAATGAAGTGTTATTACATATAGCCGGA
ATTGAGGATCTCTTTGATCCTGGAAATGGTTTACCTAAAAGCTACAGAACCAGGCCAATATATT
TTGAAATATTGATGCAGACAAATGAAATAATAAAGAGATTTTCATGGTTTATAAAAATCTTTTT
TGATATGATAATAATCATGATCACAACTGAGATCAAAAAAATATATGACAGATTATTTTGTTTA
AAAATGCAGTTTTAATTATCTTAGTCTATAGAAATGATCATTGCATGGAGGCATGTATAGGTAT
GATCTGTGTAAAATCTGACATAAAAACAGTGCTATTCTGAGTGAAAATTTTTTTGATGTGCTTA
CATAACCATGGTGATTAAAATGAGTTTATATTTTTTCTCAAAAATTTTAGCAGTGTGTAAAGTA
AGTAATCTTTAACTGAACTCTGACCACTTAAAAAAAAATCTAAAAATTGAACTACCTATAGTAG
TCTGTGTTTAAAGTGAATTTTTAAAGACAAAGCATTCTAAATGAACTCAATATAAAAACATTCA
TTTGGAATGTACATACTGAAAAATACAGGTTTTTTTGACCAAAAGTTTTTATATCTTTTCTTTT
TATTTATTTTTTTCCTAAGTGCCAACAATTTTCTAGATATTATATACAACACAGGCTTTGATCT
TGGGGACTTTTCCCATATATTTCACACTGGAGTGAATGAAGTTGTACTTCATTTCTAGAGAAAA
GTTATACCCAGGTCCCCAATTGAGAATGTCTTGCTTGATTGAAAACGACATCATCCCTTGGTAT
ACTCCAGGGATTGGTTTCAGGACCCCTGCATTTACCAAAATTTGTGCACACTCAAGTCCTGCAG
TCACCCCTGCCTAAAGATAGAATGGCTTCTCTGTTTTTCTTCTGAAATACAACCAGAAACAATG
TGTCTATTTCTGAAAGAATAGGATTAATGATCATACAAATGGGTTAATCCTGAATTCTGGTTGT
AAATCTGGTTACAGCATAACTAGGATTATAATGCTGCCTCATTTTCACAGCACTAGTTGCTTAT
ATTGACAACAAATCATCTCGCTAAAGAGTGAATGTAGGCCAGGCGCGGTGGCTCATGCCTGTAA
TCCCAGCACTTTGGGAGGCCGAGGCGGGTGGATCACGAGGTCAGGAGATCGAGACCATCCTGGC
TAACATGGTAAAACCCCGTCTCTACTAAAAATAGAAAAAAAGAAATTAGCCTAGCGTGGTGGCT
GGCGGGCGCCTGTAGTCCCAGCTATTTGGGAGGCTAAGGCAGGAGAATGGCGTGAACCCGGGAG
GCGGAGCTTGCAGTGAGCCGAGGTCGTGCCACTGCACTCCAGCCTGGGCGACAGAGCAAGACTC
CGTCTCAAAAAAAAAAAAAAAAAAAAAAAAAGAGTGAATGTAATAGTCTTGCAGAAAATGAATG
AATACCTTTGTTCAATAAAGGAAATATGCACTGCTCACTTTTTTGAAGGAAATGCCAAAGTTAG
GTTTTACAACAAGGCTAGAGTTTGTAAATTCTGGGTTCATTTGTGATGACATAAGTCAGCAAAC
TGCGGGAATACTGTCTCTTCTATGTATTTTGTGAATAGTAAGCATAATTTTAGTTTTGTATTAT
CAATGAAAATTTCACTTGAAATTAAAGCTGCCTTTTGTTATATTTTTAACCTATAGGATAAGAT
TCCAGTATTGTATATGAGTTTTAACAAATTAAAAAATCAAATCATGTAGATTTGAAAATATTTG
CACACATTTAAAAATAAATGTAAAGTTGTCTTTTAAACTACTCGGATGTGTCCTTTCTGAACAA

The present disclosure recognizes that certain changes to a polynucleotide sequence will not impact its expression or a protein encoded by said polynucleotide. In some embodiments, a polynucleotide comprises a SLC26A4 gene having one or more silent mutations. In some embodiments, the disclosure provides a polynucleotide that comprises an SLC26A4 gene having one or more silent mutations, e.g., an SLC26A4 gene having a sequence different from SEQ ID NO: 1, 2, 3, 4 or 5 but encoding the same amino acid sequence as a functional SLC26A4 gene. In some embodiments, the disclosure provides a polynucleotide that comprises an SLC26A4 gene having a sequence different from SEQ ID NO: 1, 2, 3, 4 or 5 that encodes an amino acid sequence including one or more mutations (e.g., a different amino acid sequence when compared to that produced from a functional SLC26A4 gene), where the one or more mutations are conservative amino acid substitutions. In some embodiments, the disclosure provides a polynucleotide that comprises an SLC26A4 gene having a sequence different from SEQ ID NO: 1, 2, 3, 4 or 5 that encodes an amino acid sequence including one or more mutations (e.g., a different amino acid sequence when compared to that produced from a functional SLC26A4 gene), where the one or more mutations are not within a characteristic portion of an SLC26A4 gene or an encoded pendrin protein. In some embodiments, a polynucleotide in accordance with the present disclosure comprises an SLC26A4 gene that is at least 85%, at least 90%, at least 95%, at least 98%, or at least 99% identical to a sequence of SEQ ID NO: 1, 2, 3, 4 or 5. In some embodiments, a polynucleotide in accordance with the present disclosure comprises an SLC26A4 gene that is identical to the sequence of SEQ ID NO: 1, 2, 3, 4 or 5. As can be appreciated in the art, SEQ ID NO: 1, 2, 3, 4 or 5 can be optimized (e.g., codon optimized) to achieve increased or optimal expression in an animal, e.g., a mammal, e.g., a human.

### Polypeptides Encoded by SLC26A4 Gene

Among other things, the present disclosure provides polypeptides encoded by an SLC26A4 gene or characteristic portion thereof. In some embodiments, an SLC26A4 gene is a mammalian SLC26A4 gene. In some embodiments, an Slc2a4 gene is a murine Slc2a4 gene. In some embodiments, an SLC26A4 gene is a primate SLC26A4 gene. In some embodiments, a SLC26A4 gene is a human SLC26A4 gene.

In some embodiments, a polypeptide comprises a pendrin protein or characteristic portion thereof. In some embodiments, a pendrin protein or characteristic portion thereof is mammalian pendrin protein or characteristic portion thereof, e.g., primate pendrin protein or characteristic portion thereof. In some embodiments, a pendrin protein or characteristic portion thereof is a human pendrin protein or characteristic portion thereof.

In some embodiments, a polypeptide provided herein comprises post-translational modifications. In some embodiments, a pendrin protein or characteristic portion thereof provided herein comprises post-translational modifications. In some embodiments, post-translational modifications can comprise but is not limited to glycosylation (e.g., N-linked glycosylation, O-linked glycosylation), phosphorylation, acetylation, amidation, hydroxylation, methylation, ubiquitylation, sulfation, and/or a combination thereof.

An exemplary human pendrin protein sequence is or includes the sequence of SEQ ID NO: 6. An exemplary human pendrin protein sequence with a c-terminal flag tag is or includes the sequence of SEQ ID NO: 7.

Exemplary Human Pendrin Protein Sequence
(SEQ ID NO: 6)
MAAPGGRSEPPQLPEYSCSYMVSRPVYSELAFQQQHERRLQERKTLRESLAKCCSCSRKRAFGV
LKTLVPILEWLPKYRVKEWLLSDVISGVSTGLVATLQGMAYALLAAVPVGYGLYSAFFPILTYF
IFGTSRHISVGPFPVVSLMVGSVVLSMAPDEHFLVSSSNGTVLNTTMIDTAARDTARVLIASAL
TLLVGIIQLIFGGLQIGFIVRYLADPLVGGFTTAAAFQVLVSQLKIVLNVSTKNYNGVLSIIYT
LVEIFQNIGDTNLADFTAGLLTIVVCMAVKELNDRFRHKIPVPIPIEVIVTIIATAISYGANLE
KNYNAGIVKSIPRGFLPPELPPVSLFSEMLAASFSIAVVAYAIAVSVGKVYATKYDYTIDGNQE
FIAFGISNIFSGFFSCFVATTALSRTAVQESTGGKTQVAGIISAAIVMIAILALGKLLEPLQKS
VLAAVVIANLKGMFMQLCDIPRLWRQNKIDAVIWVFTCIVSIILGLDLGLLAGLIFGLLTVVLR
VQFPSWNGLGSIPSTDIYKSTKNYKNIEEPQGVKILRFSSPIFYGNVDGFKKCIKSTVGFDAIR
VYNKRLKALRKIQKLIKSGQLRATKNGIISDAVSTNNAFEPDEDIEDLEELDIPTKEIEIQVDW
NSELPVKVNVPKVPIHSLVLDCGAISFLDVVGVRSLRVIVKEFQRIDVNVYFASLQDYVIEKLE
QCGFFDDNIRKDTFFLTVHDAILYLQNQVKSQEGQGSILETITLIQDCKDTLELIETELTEEEL
DVQDEAMRTLAS
Exemplary Human Pendrin Protein Sequence with C-terminal Flag Tag
(SEQ ID NO: 7)
MAAPGGRSEPPQLPEYSCSYMVSRPVYSELAFQQQHERRLQERKTLRESLAKCCSCSRKRAFGV
LKTLVPILEWLPKYRVKEWLLSDVISGVSTGLVATLQGMAYALLAAVPVGYGLYSAFFPILTYF
IFGTSRHISVGPFPVVSLMVGSVVLSMAPDEHFLVSSSNGTVLNTTMIDTAARDTARVLIASAL
TLLVGIIQLIFGGLQIGFIVRYLADPLVGGFTTAAAFQVLVSQLKIVLNVSTKNYNGVLSIIYT
LVEIFQNIGDTNLADFTAGLLTIVVCMAVKELNDRFRHKIPVPIPIEVIVTIIATAISYGANLE
KNYNAGIVKSIPRGFLPPELPPVSLFSEMLAASFSIAVVAYAIAVSVGKVYATKYDYTIDGNQE
FIAFGISNIFSGFFSCFVATTALSRTAVQESTGGKTQVAGIISAAIVMIAILALGKLLEPLQKS
VLAAVVIANLKGMFMQLCDIPRLWRQNKIDAVIWVFTCIVSIILGLDLGLLAGLIFGLLTVVLR
VQFPSWNGLGSIPSTDIYKSTKNYKNIEEPQGVKILRFSSPIFYGNVDGFKKCIKSTVGFDAIR
VYNKRLKALRKIQKLIKSGQLRATKNGIISDAVSTNNAFEPDEDIEDLEELDIPTKEIEIQVDW
NSELPVKVNVPKVPIHSLVLDCGAISFLDVVGVRSLRVIVKEFQRIDVNVYFASLQDYVIEKLE
QCGFFDDNIRKDTFFLTVHDAILYLQNQVKSQEGQGSILETITLIQDCKDTLELIETELTEEEL
DVQDEAMRTLASGSRADYKDHDGDYKDHDIDYKDDDDK
Exemplary Mouse Pendrin Protein Sequence
(SEQ ID NO: 56)
MAARGGRSEPPQLAEYSCSYTVSRPVYSELAFQQQRERRLPERRTLRDSLARSCSCSRKRAFGV
VKTLLPILDWLPKYRVKEWLLSDIISGVSTGLVGTLQGMAYALLAAVPVQFGLYSAFFPILTYF
VFGTSRHISVGPFPVVSLMVGSVVLSMAPDDHFLVPSGNGSALNSTTLDTGTRDAARVLLASTL
TLLVGIIQLVFGGLQIGFIVRYLADPLVGGFTTAAAFQVLVSQLKIVLNVSTKNYNGILSIIYT
LIEIFQNIGDTNIADFIAGLLTIIVCMAVKELNDRFKHRIPVPIPIEVIVTIIATAISYGANLE
KNYNAGIVKSIPSGFLPPVLPSVGLFSDMLAASFSIAVVAYAIAVSVGKVYATKHDYVIDGNQE
FIAFGISNVFSGFFSCFVATTALSRTAVQESTGGKTQVAGLISAVIVMVAIVALGRLLEPLQKS
VLAAVVIANLKGMFMQVCDVPRLWKQNKTDAVIWVFTCIMSIILGLDLGLLAGLLFALLTVVLR
VQFPSWNGLGSVPSTDIYKSITHYKNLEEPEGVKILRFSSPIFYGNVDGFKKCINSTVGFDAIR
VYNKRLKALRRIQKLIKKGQLRATKNGIISDIGSSNNAFEPDEDVEEPEELNIPTKEIEIQVDW
NSELPVKVNVPKVPIHSLVLDCGAVSFLDVVGVRSLRMIVKEFQRIDVNVYFALLQDDVLEKME
QCGFFDDNIRKDRFFLTVHDAILHLQNQVKSREGQDSLLETVARIRDCKDPLDLMEAEMNAEEL
DVQDEAMRRLAS
Exemplary Mouse Mutant Pendrin Protein Sequence
(SEQ ID NO: 57)
MAARGGRSEPPQLAEYSCSYTVSRPVYSELAFQQQRERRLPERRTLRDSLARSCSCSRKRAFGV
VKTLLPILDWLPKYRVKEWLLSDIISGVSTGLVGTLQGMAYALLAAVPVQFGLYSAFFPILTYF
VFGTSRHISVGPFPVVSLMVGSVVLSMAPDDHFLVPSGNGSALNSTTLDTGTRDAARVLLASTL
TLLVGIIQLVFGGLQIGFIVRYLADPLVGGFTTAAAFQVLVSQPKIVLNVSTKNYNGILSIIYT
LIEIFQNIGDTNIADFIAGLLTIIVCMAVKELNDRFKHRIPVPIPIEVIVTIIATAISYGANLE
KNYNAGIVKSIPSGFLPPVLPSVGLFSDMLAASFSIAVVAYAIAVSVGKVYATKHDYVIDGNQE
FIAFGISNVFSGFFSCFVATTALSRTAVQESTGGKTQVAGLISAVIVMVAIVALGRLLEPLQKS
VLAAVVIANLKGMFMQVCDVPRLWKQNKTDAVIWVFTCIMSIILGLDLGLLAGLLFALLTVVLR
VQFPSWNGLGSVPSTDIYKSITHYKNLEEPEGVKILRFSSPIFYGNVDGFKKCINSTVGFDAIR
VYNKRLKALRRIQKLIKKGQLRATKNGIISDIGSSNNAFEPDEDVEEPEELNIPTKEIEIQVDW
NSELPVKVNVPKVPIHSLVLDCGAVSFLDVVGVRSLRMIVKEFQRIDVNVYFALLQDDVLEKME
QCGFFDDNIRKDRFFLTVHDAILHLQNQVKSREGQDSLLETVARIRDCKDPLDLMEAEMNAEEL
DVQDEAMRRLAS

The present disclosure recognizes that certain mutations in an amino acid sequence of a polypeptide described herein (e.g., including pendrin or a characteristic portion thereof) will not impact the expression, folding, or activity of the polypeptide. In some embodiments, a polypeptide (e.g., including pendrin or a characteristic portion thereof) includes one or more mutations, where the one or more mutations are conservative amino acid substitutions. In some embodiments, a polypeptide in accordance with the present disclosure comprises a pendrin or a characteristic portion thereof that is at least 85%, at least 90%, at least 95%, at least 98%, or at least 99% identical to a sequence of SEQ ID NO: 6. In some embodiments, a polypeptide in accordance with the present disclosure comprises a pendrin or a characteristic portion thereof that is identical to the sequence of SEQ ID NO: 6. In some embodiments, a polypeptide in accordance with the present disclosure comprises a pendrin or a characteristic portion thereof that is at least 85%, at least 90%, at least 95%, at least 98%, or at least 99% identical to a sequence of SEQ ID NO: 7. In some embodiments, a polypeptide in accordance with the present disclosure comprises a pendrin protein or a characteristic portion thereof that is identical to the sequence of SEQ ID NO: 7.

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide comprising an SLC26A4 gene or characteristic portion thereof. Those of skill in the art will be capable of selecting suitable constructs, as well as cells, for making any of the polynucleotides described herein. In some embodiments, a construct is a plasmid (i.e., a circular DNA molecule that can autonomously replicate inside a cell). In some embodiments, a construct can be a cosmid (e.g., pWE or sCos series).

In some embodiments, a construct is a viral construct. In some embodiments, a viral construct is a lentivirus, retrovirus, adenovirus, or adeno-associated virus construct. In some embodiments, a construct is an adeno-associated virus (AAV) construct (see, e.g., Asokan et al., Mol. Ther. 20: 699-7080, 2012, which is incorporated in its entirety herein by reference). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some embodiments, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, a construct is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a construct is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein.

### AAV Particles

Among other things, the present disclosure provides AAV particles that comprise a construct encoding an SLC26A4 gene or characteristic portion thereof described herein, and a capsid described herein. In some embodiments, AAV particles can be described as having a serotype, which is a description of the construct strain and the capsid strain. For example, in some embodiments an AAV particle may be described as AAV2, wherein the particle has an AAV2 capsid and a construct that comprises characteristic AAV2 Inverted Terminal Repeats (ITRs). In some embodiments, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid.

### AAV Construct

The present disclosure provides polynucleotide constructs that comprise an SLC26A4 gene or characteristic portion thereof. In some embodiments described herein, a polynucleotide comprising an SLC26A4 gene or characteristic portion thereof can be included in an AAV particle.

In some embodiments, a polynucleotide construct comprises one or more components derived from or modified from a naturally occurring AAV genomic construct. In some embodiments, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, or AAV Anc80 construct. Additional exemplary AAV constructs that can be used herein are known in the art. See, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference.

In some embodiments, provided constructs comprise coding sequence, e.g., an SLC26A4 gene or a characteristic portion thereof, one or more regulatory and/or control sequences, and optionally 5′ and 3′ AAV derived inverted terminal repeats (ITRs). In some embodiments wherein a 5′ and 3′ AAV derived ITR is utilized, the polynucleotide construct may be referred to as a recombinant AAV (rAAV) construct. In some embodiments, provided rAAV constructs are packaged into an AAV capsid to form an AAV particle.

In some embodiments, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some embodiments, at least 80% of a typical ITR sequence (e.g., at least 85%, at least 90%, or at least 95%) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some embodiments, any of the coding sequences and/or constructs described herein are flanked by 5′ and 3′ AAV ITR sequences. The AAV ITR sequences may be obtained from any known AAV, including presently identified AAV types.

In some embodiments, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some embodiments, provided constructs can be packaged into a capsid to create an AAV particle. An AAV particle may be delivered to a selected target cell. In some embodiments, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some embodiments, a nucleic acid coding sequence is operatively linked to and/or control components in a manner that permits coding sequence transcription, translation, and/or expression in a cell of a target tissue.

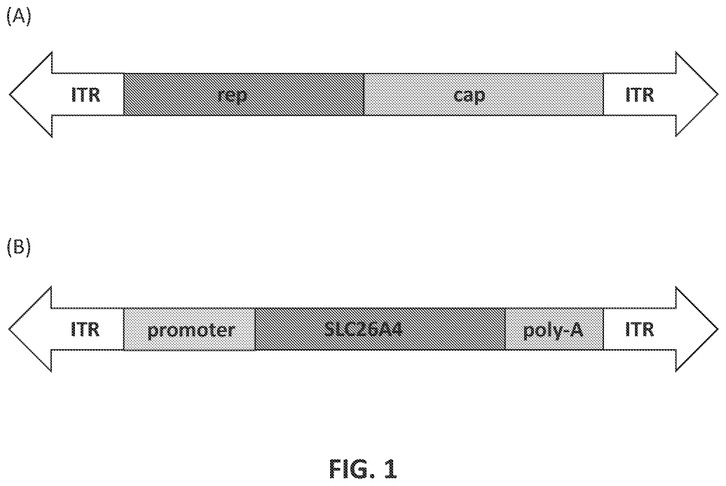

As shown in FIG. 1, panel (A), an unmodified AAV endogenous genome includes two open reading frames, “cap” and “rep,” which are flanked by ITRs. As shown in FIG. 1, panel (B), exemplary rAAV constructs similarly include ITRs flanking a coding region, e.g., a coding sequence (e.g., an SLC26A4 gene). In some embodiments, an rAAV construct also comprises conventional control elements that are operably linked to the coding sequence in a manner that permits its transcription, translation and/or expression in a cell transfected with the plasmid construct or infected with the virus produced by the disclosure. In some embodiments, an rAAV construct optionally comprises a promoter (shown in FIG. 1, panel (B)), an enhancer, an untranslated region (e.g., a 5′ UTR, 3′ UTR), a Kozak sequence, an internal ribosomal entry site (IRES), splicing sites (e.g., an acceptor site, a donor site), a polyadenylation site (shown in FIG. 1, panel (B)), or any combination thereof. Such additional elements are described further herein.

In some embodiments, a construct is an rAAV construct. In some embodiments, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some embodiments, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some embodiments, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of control sequences are described herein.

### Exemplary Construct Components

Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., SLC26A4 gene) and associated elements flanked by a 5′ and a 3′ AAV ITR sequences. In some embodiments, an ITR is or comprises about 145 nucleic acids. In some embodiments, all or substantially all of a sequence encoding an ITR is used. An AAV ITR sequence may be obtained from any known AAV, including presently identified mammalian AAV types. In some embodiments an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, an ITR (e.g., a 5′ ITR) can have a sequence according to SEQ ID NO: 10. In some embodiments, an ITR (e.g., a 3′ ITR) can have a sequence according to SEQ ID NO: 11. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides. In some embodiments, an ITR (e.g., a 5′ ITR) can have a sequence according to SEQ ID NO: 12. In some embodiments, an ITR (e.g., a 3′ ITR) can have a sequence according to SEQ ID NO: 13.

A non-limiting example of a 5′ AAV ITR sequence is SEQ ID NO: 10. A non-limiting example of a 3′ AAV ITR sequence is SEQ ID NO: 11. In some embodiments, rAAV constructs of the present disclosure comprise a 5′ AAV ITR and/or a 3′ AAV ITR. In some embodiments, a 5′ AAV ITR sequence is SEQ ID NO: 12. In some embodiments, a 3′ AAV ITR sequence is SEQ ID NO: 13. In some embodiments, the 5′ and a 3′ AAV ITRs (e.g., SEQ ID NOs: 10 and 11, or 12 and 13) flank a portion of a coding sequence, e.g., all or a portion of an SLC26A4 gene (e.g., SEQ ID NO: 1 or 2). The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 10 or 12. In some embodiments, a 3′ ITR sequence is at least 85%, 90%, 95%, 98% or 99% identical to a 3′ ITR sequence represented by SEQ ID NO: 11 or 13.

Exemplary 5′ AAV ITR
(SEQ ID NO: 10)
TTGGCCACTCCCTCTCTGCGCGCTCGCTCGCTCACTGAGGCCGCCCGGGCA
AAGCCCGGGCGTCGGGCGACCTTTGGTCGCCCGGCCTCAGTGAGCGAGCGA
GCGCGCAGAGAGGGAGTGGCCAACTCCATCACTAGGGGTTCCT
Exemplary 3′ AAV ITR
(SEQ ID NO: 11)
AGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGG
CGGCCTCAGTGAGCGAGCGAGCGCGCAGAGAGGGAGTGGCCAA
Exemplary 5′ AAV ITR
(SEQ ID NO: 12)
CTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTC
CATCACTAGGGGTTCCT
Exemplary 3′ AAV ITR
(SEQ ID NO: 13)
AGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGG
CGGCCTCAGTGAGCGAGCGAGCGCGCAG

Promoters

In some embodiments, a construct (e.g., an rAAV construct) comprises a promoter. The term “promoter” refers to a DNA sequence recognized by enzymes/proteins that can promote and/or initiate transcription of an operably linked gene (e.g., an SLC26A4 gene). For example, a promoter typically refers to, e.g., a nucleotide sequence to which an RNA polymerase and/or any associated factor binds and from which it can initiate transcription. Thus, in some embodiments, a construct (e.g., an rAAV construct) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EF1α, human cytomegalovirus (CMV) (U.S. Pat. No. 5,168,062, which is incorporated in its entirety herein by reference), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007, each of which is incorporated in its entirety herein by reference. In some embodiments, a promoter is the CMV immediate early promoter. In some embodiments, the promoter is a CAG promoter or a CAG/CBA promoter. In some embodiments, the promoter comprises or consists of SEQ ID NO: 14. In some embodiments, a promoter comprises or consists of SEQ ID NO: 15. In certain embodiments, a promoter comprises a CMV/CBA enhancer/promoter construct exemplified in SEQ ID NO: 16. In certain embodiments, a promoter comprises a CMV/CBA enhancer/promoter construct exemplified in SEQ ID NO: 17. In certain embodiments, a promoter comprises a CAG promoter or CMV/CBA/SV-40 enhancer/promoter construct exemplified in SEQ ID NO: 43. In certain embodiments, a promoter comprises a CAG promoter or CMV/CBA/SV-40 enhancer/promoter construct exemplified in SEQ ID NO: 44. In some embodiments, a promoter sequence is at least 85%, 90%, 95%, 98% or 99% identical to the promoter sequences represented by SEQ ID NO: 14 or 15. In some embodiments, an enhancer-promoter sequence is at least 85%, 90%, 95%, 98% or 99% identical to enhancer-promoter sequence represented by SEQ ID NO: 16, 17, 43, or 44.

The term “constitutive” promoter refers to a nucleotide sequence that, when operably linked with a nucleic acid encoding a protein (e.g., a pendrin protein), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985, which is incorporated in its entirety herein by reference), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088, which is incorporated in its entirety herein by reference); the ecdysone insect promoter (No et al, Proc. Natl. Acad Sci. US.A. 93:3346-3351, 1996, which is incorporated in its entirety herein by reference), the tetracycline-repressible system (Gossen et al, Proc. Natl. Acad Sci. USA. 89:5547-5551, 1992, which is incorporated in its entirety herein by reference), the tetracycline-inducible system (Gossen et al, Science 268:1766-1769, 1995, see also Harvey et al, Curr. Opin. Chem. Biol. 2:512-518, 1998, each of which is incorporated in their entirety herein by reference), the RU486-inducible system (Wang et al, Nat. Biotech. 15:239-243, 1997, and Wang et al, Gene Ther. 4:432-441, 1997, each of which is incorporated in their entirety herein by reference), and the rapamycin-inducible system (Magari et al. J Clin. Invest. 100:2865-2872, 1997, which is incorporated in its entirety herein by reference).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner.

In some embodiments, a tissue-specific promoter is a cochlea-specific promoter. In some embodiments, a tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a αl0ACHR promoter. In some embodiments, a promoter is a cochlear hair cell-specific promoter such as a PRESTIN promoter or an ONCOMOD promoter. See, e.g., Zheng et al., Nature 405:149-155, 2000; Tian et al. Dev. Dyn. 23 1: 199-203, 2004; and Ryan et al., Adv. Otorhinolaryngol. 66: 99-115, 2009, each of which is incorporated in their entirety herein by reference.

In some embodiments, a tissue-specific promoter is an ear cell specific promoter. In some embodiments, a tissue-specific promoter is an inner ear cell specific promoter. Non-limiting examples of inner ear non-sensory cell-specific promoters include but are not limited to: GJB2, GJB6, SLC26A4, TECTA, DFNA5, COCH, NDP, SYN1, GFAP, PLP, TAK1, or SOX21. In some embodiments, a cochlear non-sensory cell specific promoter may be an inner ear supporting cell specific promoter. Non-limiting examples of inner ear supporting cell specific promoters include but are not limited to: SOX2, FGFR3, PROX1, GLAST1, LGR5, HES1, HES5, NOTCH1, JAG1, CDKN1A, CDKNV1B, SOX10, P75, CD44, HEY2, LFNG, or S100b.

In some embodiments, provided AAV constructs comprise a promoter sequence selected from a CAG, a CBA, a CMV, or a CB7 promoter. In some embodiments of any of the therapeutic compositions described herein, the first or sole AAV construct further includes at least one promoter sequence selected from Cochlea and/or inner ear specific promoters.

Exemplary CBA promoter
(SEQ ID NO: 14)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCG
Exemplary CBA promoter
(SEQ ID NO: 15)
GTCGAGGTGAGCCCCACGTTCTGCTTCACTCTCCCCATCTCCCCCCCCTCCCCACCCCCAATTT
TGTATTTATTTATTTTTTAATTATTTTGTGCAGCGATGGGGGCGGGGGGGGGGGGGGCGCGCGC
CAGGCGGGGCGGGGCGGGGCGAGGGGCGGGGCGGGGCGAGGCGGAGAGGTGCGGCGGCAGCCAA
TCAGAGCGGCGCGCTCCGAAAGTTTCCTTTTATGGCGAGGCGGCGGCGGCGGCGGCCCTATAAA
AAGCGAAGCGCGCGGCGGGCG
Exemplary CMV/CBA enhancer/promoter
(SEQ ID NO: 16)
GAGATTGATTATTGAGTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCG
Exemplary CMV/CBA enhancer/promoter
(SEQ ID NO: 17)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCG
Exemplary CAG enhancer/promoter
(SEQ ID NO: 43)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCGGGAGTCGCTGCGTTGCCTTCGCCCCGTGCCCCGCTCCGCGCCGC
CTCGCGCCGCCCGCCCCGGCTCTGACTGACCGCGTTACTCCCACAGGTGAGCGGGCGGGACGGC
CCTTCTCCTCCGGGCTGTAATTAGCGCTTGGTTTAATGACGGCTCGTTTCTTTTCTGTGGCTGC
GTGAAAGCCTTAAAGGGCTCCGGGAGGGCCCTTTGTGCGGGGGGGAGCGGCTCGGGGGGTGCGT
GCGTGTGTGTGTGCGTGGGGAGCGCCGCGTGCGGCCCGCGCTGCCCGGCGGCTGTGAGCGCTGC
GGGCGCGGCGCGGGGCTTTGTGCGCTCCGCGTGTGCGCGAGGGGAGCGCGGCCGGGGGCGGTGC
CCCGCGGTGCGGGGGGGCTGCGAGGGGAACAAAGGCTGCGTGCGGGGTGTGTGCGTGGGGGGGT
GAGCAGGGGGTGTGGGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCGGGCCGGGGAGGGC
TCGGGGGAGGGGCGCGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGAAGCGGTGCGGCGCC
GGCAGGAAGGAAATGGGCGGGGAGGGCCTTCGTGCGTCGCCGCGCCGCCGTCCCCTTCTCCCTC
TCCAGCCTCGGGGCTGTCCGCGGGGGGACGGCTGCCTTCGGGGGGGACGGGGCAGGGCGGGGTT
CGGCTTCTGGCGTGTGACCGGCGGCTCTAGAGCCTCTGCTAACCATGTTCATGCCTTCTTCTTT
TTCCTAGAG
Exemplary CAG enhancer/promoter
(SEQ ID NO: 44)
GAGATTGATTATTGAGTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGAGGTCAATAATGAGGTATGTTCCCATAGTAACGCCAATAGGGAGTTTCCATTGAG
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGGGTC
GAGGTGAGCCCCACGTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGGGGGGCGCGCGCCAG
GCGGGGCGGGGCGGGGCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGGCGGCCCTATAAAAAG
CGAAGCGCGCGGCGGGCGGGAGTCGCTGCGTTGCCTTCGCCCCGTGCCCCGCTCCGCGCCGCCT
CGCGCCGCCCGCCCCGGCTCTGACTGACCGCGTTACTCCCACAGGTGAGCGGGCGGGACGGCCC
TTCTCCTCCGGGCTGTAATTAGCGCTTGGTTTAATGACGGCTCGTTTCTTTTCTGTGGCTGCGT
GAAAGCCTTAAAGGGCTCCGGGAGGGCCCTTTGTGCGGGGGGGAGCGGCTCGGGGGGTGCGTGC
GTGTGTGTGTGCGTGGGGAGCGCCGCGTGCGGCCCGCGCTGCCCGGCGGCTGTGAGCGCTGCGG
GCGCGGCGCGGGGCTTTGTGCGCTCCGCGTGTGCGCGAGGGGAGCGCGGCCGGGGGCGGTGCCC
CGCGGTGCGGGGGGGCTGCGAGGGGAACAAAGGCTGCGTGCGGGGTGTGTGCGTGGGGGGGTGA
GCAGGGGGTGTGGGCGCGGCGGTCGGGCTGTAACCCCCCCCTGCACCCCCCTCCCCGAGTTGCT
GAGCACGGCCCGGCTTCGGGTGCGGGGCTCCGTGCGGGGCGTGGCGCGGGGCTCGCCGTGCCGG
GCGGGGGGTGGCGGCAGGTGGGGGTGCCGGGCGGGGCGGGGCCGCCTCGGGCCGGGGAGGGCTC
GGGGGAGGGGCGCGGCGGCCCCCGGAGCGCCGGCGGCTGTCGAGGCGCGGCGAGCCGCAGCCAT
TGCCTTTTATGGTAATCGTGCGAGAGGGCGCAGGGACTTCCTTTGTCCCAAATCTGTGCGGAGC
CGAAATCTGGGAGGCGCCGCCGCACCCCCTCTAGCGGGCGCGGGGCGAAGCGGTGCGGCGCCGG
CAGGAAGGAAATGGGCGGGGAGGGCCTTCGTGCGTCGCCGCGCCGCCGTCCCCTTCTCCCTCTC
CAGCCTCGGGGCTGTCCGCGGGGGGACGGCTGCCTTCGGGGGGGACGGGGCAGGGCGGGGTTCG
GCTTCTGGCGTGTGACCGGCGGCTCTAGAGCCTCTGCTAACCATGTTCATGCCTTCTTCTTTTT
CCTACAG

In certain embodiments, a promoter is an endogenous human ATOH enhancer-promoter as set forth in SEQ ID NO: 18. In some embodiments, an enhancer-promoter sequence is at least 85%, 90%, 95%, 98% or 99% identical to enhancer-promoter sequence represented by SEQ ID NO: 18.

Exemplary Human ATOH1 enhancer-promoter
(SEQ ID NO: 18)
CTATGGAGTTTGCATAACAAACGTTTGGCAGCTCGCTCTCTTACACTCCATTAACAAGCTGTAA
CATATAGCTGCAGGTTGCTATAATCTCATTAATATTTTGGAAACTTGAATATTGAGTATTTCTG
AGTGCTCATTCCCCATATGCCAGCCACTTCTGCCATGCTGACTGGTTCCTTTCTCTCCATTATT
AGCAATTAGCTTCTTACCTTCCAAAGTCAGATCCAAGGTATCCAAGATACTAGCAAAGGAATCA
ACTATGTGTGCAAGTTAAGCATGCTTAATATCACCCAAACAAACAAAGAGGCAGCATTTCTTAA
AGTAATGAAGATAGATAAATCGGGTTAGTCCTTTGCGACACTGCTGGTGCTTTCTAGAGTTTTA
TATATTTTAAGCAGCTTGCTTTATATTCTGTCTTTGCCTCCCACCCCACCAGCACTTTTATTTG
TGGAGGGTTTTGGCTCGCCACACTTTGGGAAACTTATTTGATTTCACGGAGAGCTGAAGGAAGA
TCATTTTTGGCAACAGACAAGTTTAAACACGATTTCTATGGGACATTGCTAACTGGGGCCCCTA
AGGAGAAAGGGGAAACTGAGCGGAGAATGGGTTAAATCCTTGGAAGCAGGGGAGAGGCAGGGGA
GGAGAGAAGTCGGAGGAGTATAAAGAAAAGGACAGGAACCAAGAAGCGTGGGGGTGGTTTGCCG
TAATGTGAGTGTTTCTTAATTAGAGAACGGTTGACAATAGAGGGTCTGGCAGAGGCTCCTGGCC
GCGGTGCGGAGCGTCTGGAGCGGAGCACGCGCTGTCAGCTGGTGAGCGCACTCTCCTTTCAGGC
AGCTCCCCGGGGAGCTGTGCGGCCACATTTAACACCATCATCACCCCTCCCCGGCCTCCTCAAC
CTCGGCCTCCTCCTCGTCGACAGCCTTCCTTGGCCCCCACCAGCAGAGCTCACAGTAGCGAGCG
TCTCTCGCCGTCTCCCGCACTCGGCCGGGGCCTCTCTCCTCCCCCAGCTGCGCAGCGGGAGCCG
CCACTGCCCACTGCACCTCCCAGCAACCAGCCCAGCACGCAAAGAAGCTGCGCAAAGTTAAAGC
CAAGCAATGCCAAGGGGAGGGGAAGCTGGAGGCGGGCTTTGAGTGGCTTCTGGGCGCCTGGCGG
GTCCAGAATCGCCCAGAGCCGCCCGCGGTCGTGCACATCTGACCCGAGTCAGCTTGGGCACCAG
CCGAGAGCCGGCTCCGCACCGCTCCCGCACCCCAGCCGCCGGGGTGGTGACACACACCGGAGTC
GAATTACAGGCCTGCAATTAACATATGAATCTGAGGAATTTAAAAGAAGGAAAAAAAAAAAAAA
ACCTGAGCAGGCTTGGGAGTCCTCTGCACACAAGAACTTTTCTCGGGGTGTAAAAACTCTTTGA
TTGGCTGCTCGCACGCGCCTGCCCGCGCCCTCCATTGGCTGAGAAGACACGCGACCGGCGCGAG
GAGGGGGTTGGGAGAGGAGCGGGGGGAGACTGAGTGGCGCGTGCCGCTTTTTAAAGGGGCGCAG
CGCCTTCAGCAACCGGAGAAGCATAGTTGCACGCGACCTGGTGTGTGATCTCCGAGTGGGTGGG
GGAGGGTCGAGGAGGGAAAAAAAAATAAGACGTTGCAGAAGAGACCCGGAAAGGGCCTTTTTTT
TGGTTGAGCTGGTGTCCCAGTGCTGCCTCCGATCCTGAGCCTCCGAGCCTTTGCAGTGCAA

In certain embodiments, a promoter is an endogenous human SLC26A4 immediate promoter as set forth in SEQ ID NO: 45 or 46. In certain embodiments, a promoter is an endogenous human SLC26A4 enhancer-promoter as set forth in SEQ ID NO: 47, 48 or 50. In some embodiments, an enhancer-promoter sequence is at least 85%, 90%, 95%, 98% or 99% identical to a promoter or enhancer-promoter sequence represented by SEQ ID NO: 45, 46, 47, 48, or 50. In certain embodiments, a promoter is a human SLC26A4 endogenous enhancer-promoter sequence comprised within SEQ ID NO: 47, 48, or 50.

Exemplary Human SLC26A4 immediate promoter
(SEQ ID NO: 45)
CTGCCTTCTGAGAGCGCTATAAAGGCAGCGGAAGGGTAGTCCGCGGGGCATTCCGGGCGG
Exemplary Human SLC26A4 immediate promoter
(SEQ ID NO: 46)
CTCTAGGCGGGCTCTGCTCTTCTTTAAGGAGTCCCACAGGGCCTGGCCCGCCCCTGACCT
Exemplary Human SLC26A4 enhancer-promoter
(SEQ ID NO: 47)
TAAAGAGTTGTGAGTTGTGTAGGTGAGTTGCCATGGAGCTACAAATATGAGTTGATATTCTGAA
ATCCTAGACAGCCATCTCCAAGGTTAAGAAAAATCCTTATGGAGTGAGTTGCAAAGATATCGAG
AGCATGCTCTTAATGGAGAAAAACAAAGCCTTAGATCAAATATGTAAAGTAATTTTTAGTTTTT
TGAAAAGGTATGTTTGGGCTATAGATAAATCTGTTCAAAAAACATGAGAGAAGATAATAATGGT
TGAAAGGAGACACAGTGCTTGCCCTCAAGAAGTTTTTGTCTAGTGAGGGAGAGAGAACTTGTAT
GTAAATAAAATTGTGTTACTAAGGTAGATAGTGAGAAGTAACTTAAGAGAGGATCAGATAAGGT
ATTAAGAGAATACAGAAAAGGGTCTGGATTAATTCTGAACAGCATCAAAGAATGTTCTTGCAAG
AGATAGTGTTTTCACCAGATCTTGAAGGTATGGATGAGGGTATACAGAGTGAGTATATTCAGAT
TCTACTTTAAAACAAATACTTTCCTCTGTTGTAGTGGAGTTGAGCTATACATCCAACAATAATG
AAAAAATACACGCATATATACATATATGGAGAGAGATACATATTTTAGTAGATGTAGCAATTGA
TTAATAAATGTACAGTTTAAGTCGCATGCAAAACCTTGGAGTGATAGCAAACTTCATTGTAGGA
TGTTTAGCAGCATCTCTGGTCTCTACTCACTAGATCCCAATAGCATCTCCCTAGGTGTGACAAC
CAAAAATGTCTCCAGGCATTGACCTCTGGAGGCAAAAAAAGCCCTTTATTAAGAACCAGTGGTA
TAGATAAGTAAAACATACACAAGAGATTCCTCCCCTCTTCTCTGTATGTGAATAAAAATTGCAA
AGTTCATGACCTGGATTTTCCTTTTAGGTTTCTTCTTTAGTGGTTCTTAACTTCATTGGGTGAA
GTAAGCCTTTGAAGATCTGTTGAAAGCTGTTGACTCATTCACTTCTCAGGAAAACGCACATGCT
GACTACCATTTCAGAGAATTTGCATCAGGGTTCTCTGGGGAGGAGTTCTGAGTTCTGTTTCCAG
GAGCTCGTAGAATTGTCATGGTCTGCATATGCAAGGCAGGTGGATTACGGAAGGTTGATGTACA
GAGGTCTGTATTTTGGAGCCTCTTCTGTATTTACTTCAGAACACTAACAATCAGGCGAGAATGT
TCTGGTTTATCAAACCCTTCCTTCTGCCTTTCATCTTAACCATGCATTAGTTTTAACAAAGTTC
ATCCCAACAGAAGACAAAACACTGATGAGGTAGGATAGCTCCAGCTCCTCCTCCCTCTCTTCTA
GTCTTGATTTCCATGTAGTCCAGTTTATTCCTTCCCTGATTGTCCAGGAGAATGAGAAAAAGAA
AAAACAGAGTCTAGTGGGTAAGAAAGGGCCACCTGGACGGCTTGATTTGGATTGTGAAATAAAA
CACACACACATGCACACGTAGAATAAGTGGCTAAAATCTGAGTAAATCGTGAACTCTCTGTATC
CTCCACCCATTGAATACTCCTAAAAGACTTTCTAGAAATTCAAGGACTTATTAATATAGAAACC
TGGCCATTGTTCCTCTTCTCCTCCCCATGTGGTATGAGAGCACCTGTGGCAGGCTCCCAGAGAC
CACGGACCTCTTCCTCTAGGCGGGCTCTGCTCTTCTTTAAGGAGTCCCACAGGGCCTGGCCCGC
CCCTGACCTCGCAACCCTTGAGATTAGTAACGGGATGAGTGAGGATCCGGGTGGCCCCTGCGTG
GCAGCCAGTAAGAGTCTCAGCCTTCCCGGTTCGGGAAAGGGGAAGAATGCAGGAGGGGTAGGAT
TTCTTTCCTGATAGGATCGGTTGGGAAAGACCGCAGCCTGTGTGTGTCTTTCCCTTCGACCAAG
GTGTCTGTTGCTCCGTAAATAAAACGTCCCACTGCCTTCTGAGAGCGCTATAAAGGCAGCGGAA
GGGTAGTCCGCGGGGC
Exemplary Human SLC26A4 enhancer-promoter
(SEQ ID NO: 48)
GGCTGCTCGGAAAACAGGACGAGGGGAGAGACTTGCTCAATAAGCTGAAAGTTCTGCCCCCGAG
AGGGCTGCGACAGCTGCTGGAATGTGCCTGCAGCGTCCGCCTCTTGGGGACCCGCGGAGCGCGC
CCTGACGGTTCCACGCCTGGCCCGGGGGTCTGCACCTCTCCTCCAGTGCGCACCTGGAGCTGCG
TCCCGGGTCAGGTGCGGGGAGGGAGGGAATCTCAGTGTCCCCTTCCAGCCTTGCAAGCGCCTTT
GGCCCCTGCCCCAGCCCCTCGGTTTGGGGGAGATTTCAGAACGCGGACAGCGCCCTGGCTGCGG
GCCATAGGGGACTGGGTGGAACTCGGGAAGCCCCCAGAGCAGGGGCTTACTCGCTTCAAGTTTG
GGGAACCCCGGGCAGCGGGTGCAGGCCACGAGACCCGAAGGTTCTCAGGTGCCCCCCTGCAGGC
TGGCCGTGCGCGCCGTGGGGCGCTTGTCGCGAGCGCCGAGGGCTGCAGGACGCGGACCAGACTC
GCGGTGCAGGGGGGCCTGGCTGCAGCTAACAGGTGATCCCGTTCTTTCTGTTCCTCGCTCTTCC
CCTCCGATCGTCCTCGCTTACCGCGTGTCCTCCCTCCTCGCTGTCCTCTGGCTCGCAGGTCATG
GCAGCGCCAGGCGGCAGGTCGGAGCCGCCGCAGCTCCCCGAGTACAGCTGCAGCTACATGGTGT
CGCGGCCGGTCTACAGCGAGCTCGCTTTCCAGCAACAGCACGAGCGGCGCCTGCAGGAGCGCAA
GACGCTGCGGGAGAGCCTGGCCAAGTGCTGCAGGTAGCGGCCGCGCGGGCCTGCGTAGAGAGAA
GCGGAGCGGGGCGTCCACGCCTTGGGGAGGGAAGGGCGTCCCCAGCGGGCGAGAGTGGGGTGCG
GGCGGCGGAGCCCCTGGGCGCCAGCTGCTTCTCCCAGAGGCCCGACTTTCGGTCTCCGGTCCTC
CACGCCGCCCTTCTGGTGGGAGGGTGGCTCCATCAGTCTCGGGCCCGAAATGAACTTACCTGGG
AAACTCGCCTTTGGGGAGAGTGGGTTCTAGGAGCCCCGTCTCTCTTTTTCCTCTCTGAAGGAAA
CTTGGAGTGCCTCTTGGGGTACAGTGGGTCCCTGTTGCCTTCTTGGGAGCTTGTTTAAATGAAA
TGAATAGGGAAACCCAGCTCTTGACCAGGAGGAGTCCTTGAAACACTCAAGCTAAGTAGGCGGG
CTACCATTCAGTTAGAGACCAGGATGCAAGCTAGAACCCAGGGGAGCGCGGGGTGTGCCAAGTA
CTTCATCAGCAGGCTGTGGGACCCCTGGGGAAAGCCACCCTCAGTCTCTAAACCCAAACATGCC
GTAACTAGATGTCACAAACATAAAGAAATTAGAGTTTCTAAAACCTTTCATTATAG
Exemplary Human SLC26A4 enhancer-promoter
(SEQ ID NO: 50)
CGGAAGGTTGATGTACAGAGGTCTGTATTTTGGAGCCTCTTCTGTATTTACTTCAGAACACTAA
CAATCAGGCGAGAATGTTCTGGTTTATCAAACCCTTCCTTCTGCCTTTCATCTTAACCATGCAT
TAGTTTTAACAAAGTTCATCCCAACAGAAGACAAAACACTGATGAGGTAGGATAGCTCCAGCTC
CTCCTCCCTCTCTTCTAGTCTTGATTTCCATGTAGTCCAGTTTATTCCTTCCCTGATTGTCCAG
GAGAATGAGAAAAAGAAAAAACAGAGTCTAGTGGGTAAGAAAGGGCCACCTGGACGGCTTGATT
TGGATTGTGAAATAAAACACACACACATGCACACGTAGAATAAGTGGCTAAAATCTGAGTAAAT
CGTGAACTCTCTGTATCCTCCACCCATTGAATACTCCTAAAAGACTTTCTAGAAATTCAAGGAC
TTATTAATATAGAAACCTGGCCATTGTTCCTCTTCTCCTCCCCATGTGGTATGAGAGCACCTGT
GGCAGGCTCCCAGAGACCACGGACCTCTTCCTCTAGGCGGGCTCTGCTCTTCTTTAAGGAGTCC
CACAGGGCCTGGCCCGCCCCTGACCTCGCAACCCTTGAGATTAGTAACGGGATGAGTGAGGATC
CGGGTGGCCCCTGCGTGGCAGCCAGTAAGAGTCTCAGCCTTCCCGGTTCGGGAAAGGGGAAGAA
TGCAGGAGGGGTAGGATTTCTTTCCTGATAGGATCGGTTGGGAAAGACCGCAGCCTGTGTGTGT
CTTTCCCTTCGACCAAGGTGTCTGTTGCTCCGTAAATAAAACGTCCCACTGCCTTCTGAGAGCG
CTATAAAGGCAGCGGAAGGGTAGTCCGCGGGGCATTCCGGGCGGGGCGCGAGCAGAGACAGGTG
AGTT

In certain embodiments, a promoter is a human LGR5 enhancer-promoter as set forth in SEQ ID NO: 51. In some embodiments, an enhancer-promoter sequence is at least 85%, 90%, 95%, 98% or 99% identical to enhancer-promoter sequence represented by SEQ ID NO: 51. In some embodiments, a promoter is a human LGR5 endogenous enhancer-promoter sequence comprised within SEQ ID NO: 51.

Exemplary Human LGR5 enhancer-promoter
(SEQ ID NO: 51)
AGGGCTATTTGTACCTCAACGAGGGCTTCTCTCCAAGAAAGCCCTGAATCCTTTTCCTCCTTTT
TCCTGCAGATTCACTATAGGACACTTTTTGAAGCAAGAGCATGCATTTTCCCCCTGGCGCTCTG
CAGCGGTTCTCAGAGCCCAGTGTCACTCACATAGGTGGGACTGCTCTCAGTTCAGAGAGCGCTG
GGACACTTAAGATGAAAAGTCCCTGGAAGTTAGCAAACAGCCATCTGTCACTCTGGCATCGATT
TAGTAAAAGTGAGTTCTAGGGTATTCTAAACGAGTTTTAAAAAACAAATGAGTGAGTTCGAGTT
CCTCACCCCGCAAGAGATAGGAAGGCAGCAGTGGAGTGCTCGCTCAGGAGCTGTATTTGTTTAG
CGATTAGCCTAGAGCTTTGATTTTAGGGCAAAAGCGAGCCAGACAGTGCGGCAGACGTAAGGAT
CAAAAAGGCCACCTATCATTCGCCGGGGACGCCTGCCTCCTTACCCTGATAACGTAACTATTTC
TCTGCATAGGATTTTAGTTTTTGTGTTTTTGTTTTGTTTTATTCTGTTTAATCACTTCAAGTAT
CTCATCCATTATTTGAAGCGGGCTCGGAGGAAACGTGCCGCATCCTCCAGTCCTTGTGCGTCTG
TTTAGGTCTCTCCGAAGCAGGTCCCTCTCGACTCTTAGATCTGGGTCTCCAGCACGCATGAAGG
GGTAAGGGTGGGGGGGTCCCCTATTCCGGCGCGCGGCGTTGAGCACTGAATCTTCCAGGCGGAG
GCTCAGTGGGAGCGCCGAGAACTCGCCAGTACCGCGCGCTGCCTGCTGCCTGCTGCCTCCCAGC
CCAGGACTTGGGAAAGGAGGGAGGGGACAAGTGGAGGGAAAGTGGGGCCGGGCGGGGGGTGCCT
GGGAAGCCAGGCTGCGCTGACGTCACTGGGCGCGCAATTCGGGCTGGAGCGCTTTAAAAAACGA
GCGTGCAAGCAGAGATGCTGCTCCACACCGCTCAGGCCGCGAGCAGCAGCAAGGCGCACCGCCA
CTGTCGCCGCTGCAGCCAGGGCTGCTCCGAAGGCCGGCGTGGCGGCAACCGGCACCTCTGTCCC
CGCCGCGCTTCTCCTCGCCGCCCACGCCGTGGGGTCAGGAACGCGGCGTCTGGCGCTGCAGACG
CCCGCTGAGTTGCAGAAGCCCACGGAGCGGCGCCCGGCGCGCCACGGCCCGTAGCAGTCCGGTG
CTGCTCTCCGCCCGCGTCCGGCTCGTGGCCCCCTACTTCGGGCACCGACCGGT

In certain embodiments, a promoter is a human SYN1 enhancer-promoter as set forth in SEQ ID NO: 52. In some embodiments, an enhancer-promoter sequence is at least 85%, 90%, 95%, 98% or 99% identical to enhancer-promoter sequence represented by SEQ ID NO: 52. In some embodiments, a promoter is a human SYN1 endogenous enhancer-promoter sequence comprised within SEQ ID NO: 52.

Exemplary Human SYN1 enhancer-promoter
(SEQ ID NO: 52)
TGCGTATGAGTGCAAGTGGGTTTTAGGACCAGGATGAGGCGGGGTGGGG
GTGCCTACCTGACGACCGACCCCGACCCACTGGACAAGCACCCAACCCC
CATTCCCCAAATTGCGCATCCCCTATCAGAGAGGGGGAGGGGAAACAGG
ATGCGGCGAGGCGCGTGCGCACTGCCAGCTTCAGCACCGCGGACAGTGC
CTTCGCCCCCGCCTGGCGGCGCGCGCCACCGCCGCCTCAGCACTGAAGG
CGCGCTGACGTCACTCGCCGGTCCCCCGCAAACTCCCCTTCCCGGCCAC
CTTGGTCGCGTCCGCGCCGCCGCCGGCCCAGCCGGACCGCACCACGCGA
GGCGCGAGATAGGGGGGCACGGGCGCGACCATCTGCGCTGCGGCGCCGG
CGACTCAGCGCTGCCTCAGTCTGCGGTGGGCAGCGGAGGAGTCGTGTCG
TGCCTGAGAGCGCAGTCGAGAA

In certain embodiments, a promoter is a human GFAP enhancer-promoter as set forth in SEQ ID NO: 53. In some embodiments, an enhancer-promoter sequence is at least 85%, 90%, 95%, 98% or 99% identical to enhancer-promoter sequence represented by SEQ ID NO: 53. In some embodiments, a promoter is a human GFAP endogenous enhancer-promoter sequence comprised within SEQ ID NO: 53.

Exemplary Human GFAP enhancer-promoter
(SEQ ID NO: 53)
CCCACCTCCCTCTCTGTGCTGGGACTCACAGAGGGAGACCTCAGGAGGCAGTCTGTCCATCACA
TGTCCAAATGCAGAGCATACCCTGGGCTGGGCGCAGTGGCGCACAACTGTAATTCCAGCACTTT
GGGAGGCTGATGTGGAAGGATCACTTGAGCCCAGAAGTTCTAGACCAGCCTGGGCAACATGGCA
AGACCCTATCTCTACAAAAAAAGTTAAAAAATGAGCCACGTGTGGTGAGACACACCTGTAGTCC
CAGCTATTCAGGAGGCTGAGGTGAGGGGATCACTTAAGGCTGGGAGGTTGAGGCTGCAGTGAGT
CGTGGTTGCGCCACTGCACTCCAGCCTGGGCAACAGTGAGACCCTGTCTCAAAAGACAAAAAAA
AAAAAAAAAAAAAAAAGAACATATCCTGGTGTGGAGTAGGGGACGCTGCTCTGACAGAGGCTCG
GGGGCCTGAGCTGGCTCTGTGAGCTGGGGAGGAGGCAGACAGCCAGGCCTTGTCTGCAAGCAGA
CCTGGCAGCATTGGGCTGGCCGCCCCCCAGGGCCTCCTCTTCATGCCCAGTGAATGACTCACCT
TGGCACAGACACAATGTTCGGGGTGGGCACAGTGCCTGCTTCCCGCCGCACCCCAGCCCCCCTC
AAATGCCTTCCGAGAAGCCCATTGAGCAGGGGGCTTGCATTGCACCCCAGCCTGACAGCCTGGC
ATCTTGGGATAAAAGCAGCACAGCCCCCTAGGGGCTGCCCTTGCTGTGTGGCGCCACCGGCGGT
GGAGAACAAGGCTCTATTCAGCCTGTGCCCAGGAAAGGGGATCAGGGGATGCCCAGGCATGGAC
AGTGGGTGGCAGGGGGGGAGAGGAGGGCTGTCTGCTTCCCAGAAGTCCAAGGACACAAATGGGT
GAGGGGACTGGGCAGGGTTCTGACCCTGTGGGACCAGAGTGGAGGGCGTAGATGGACCTGAAGT
CTCCAGGGACAACAGGGCCCAGGTCTCAGGCTCCTAGTTGGGCCCAGTGGCTCCAGCGTTTCCA
AACCCATCCATCCCCAGAGGTTCTTCCCATCTCTCCAGGCTGATGTGTGGGAACTCGAGGAAAT
AAATCTCCAGTGGGAGACGGAGGGGTGGCCAGGGAAACGGGGCGCTGCAGGAATAAAGACGAGC
CAGCACAGCCAGCTCATGTGTAACGGCTTTGTGGAGCTGTCAAGGCCTGGTCTCTGGGAGAGAG
GCACAGGGAGGCCAGACAAGGAAGGGGTGACCTGGAGGGACAGATCCAGGGGCTAAAGTCCTGA
TAAGGCAAGAGAGTGCCGGCCCCCTCTTGCCCTATCAGGACCTCCACTGCCACATAGAGGCCAT
GATTGACCCTTAGACAAAGGGCTGGTGTCCAATCCCAGCCCCCAGCCCCAGAACTCCAGGGAAT
GAATGGGCAGAGAGCAGGAATGTGGGACATCTGTGTTCAAGGGAAGGACTCCAGGAGTCTGCTG
GGAATGAGGCCTAGTAGGAAATGAGGTGGCCCTTGAGGGTACAGAACAGGTTCATTCTTCGCCA
AATTCCCAGCACCTTGCAGGCACTTACAGCTGAGTGAGATAATGCCTGGGTTATGAAATCAAAA
AGTTGGAAAGCAGGTCAGAGGTCATCTGGTACAGCCCTTCCTTCCCTTTTTTTTTTTTTTTTTT
GTGAGACAAGGTCTCTCTCTGTTGCCCAGGCTGGAGTGGCGCAAACACAGCTCACTGCAGCCTC
AACCTACTGGGCTCAAGCAATCCTCCAGCCTCAGCCTCCCAAAGTGCTGGGATTACAAGCATGA
GCCACCCCACTCAGCCCTTTCCTTCCTTTTTAATTGATGCATAATAATTGTAAGTATTCATCAT
GGTCCAACCAACCCTTTCTTGACCCACCTTCCTAGAGAGAGGGTCCTCTTGCTTCAGCGGTCAG
GGCCCCAGACCCATGGTCTGGCTCCAGGTACCACCTGCCTCATGCAGGAGTTGGCGTGCCCAGG
AAGCTCTGCCTCTGGGCACAGTGACCTCAGTGGGGTGAGGGGAGCTCTCCCCATAGCTGGGCTG
CGGCCCAACCCCACCCCCTCAGGCTATGCCAGGGGGTGTTGCCAGGGGCACCCGGGCATCGCCA
GTCTAGCCCACTCCTTCATAAAGCCCTCGCATCCCAGGAGCGAGCAGAGCCAGAGCAGGTTGGA
GAGGAGACGCATCACCTCCGCTGCTCGC

Enhancers

In some instances, a construct can include an enhancer sequence. The term “enhancer” refers to a nucleotide sequence that can increase the level of transcription of a nucleic acid encoding a protein of interest (e.g., a pendrin protein). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some embodiments, a construct comprises a CMV enhancer exemplified by SEQ ID NO: 19. In some embodiments, an enhancer sequence is at least 85%, 90%, 95%, 98% or 99% identical to the enhancer sequence represented by SEQ ID NO: 19. In some embodiments, an SV-40 derived enhancer is the SV-40 T intron sequence, which is exemplified by SEQ ID NO: 20. In some embodiments, an enhancer sequence is at least 85%, 90%, 95%, 98% or 99% identical to the enhancer sequence represented by SEQ ID NO: 20.

Exemplary CMV enhancer
(SEQ ID NO: 19)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGACCCC
CGCCCATTGACGTCAATAATGACGTATGTTCCCATAGTAACGCCAATAGGGACTTTCCATTGAC
GTCAATGGGTGGACTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATG
ACCTTATGGGACTTTCCTACTTGGCAGTACATCTACGTATTAGTCATCGCTATTACCATGG
Exemplary SV-40 synthetic intron
(SEQ ID NO: 20)
GGAGTCGCTGCGTTGCCTTCGCCCCGTGCCCCGCTCCGCGCCGCCTCGCGCCGCCCGCCCCGGC
TCTGACTGACCGCGTTACTCCCACAGGTGAGCGGGCGGGACGGCCCTTCTCCTCCGGGCTGTAA
TTAGCGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGTGGGG
AGCGCCGCGTGCGGCCCGCGCTGCCCGGCGGCTGTGAGCGCTGCGGGCGCGGCGCGGGGCTTTG
TGCGCTCCGCGTGTGCGCGAGGGGAGCGCGGCCGGGGGCGGTGCCCCGCGGTGCGGGGGGGCTG
CGAGGGGAACAAAGGCTGCGTGCGGGGTGTGTGCGTGGGGGGGTGAGCAGGGGGTGTGGGCGCG
GCGGTCGGGCTGTAACCCCCCCCTGCACCCCCCTCCCCGAGTTGCTGAGCACGGCCCGGCTTCG
GGTGCGGGGCTCCGTGCGGGGCGTGGCGCGGGGCTCGCCGTGCCGGGCGGGGGGTGGCGGCAGG
TGGGGGTGCCGGGCGGGGCGGGGCCGCCTCGGGCCGGGGAGGGCTCGGGGGAGGGGCGCGGCGG
CCCCCGGAGCGCCGGCGGCTGTCGAGGCGCGGCGAGCCGCAGCCATTGCCTTTTATGGTAATCG
TGCGAGAGGGCGCAGGGACTTCCTTTGTCCCAAATCTGTGCGGAGCCGAAATCTGGGAGGCGCC
GCCGCACCCCCTCTAGCGGGCGCGGGGCGAAGCGGTGCGGCGCCGGCAGGAAGGAAATGGGCGG
GGAGGGCCTTCGTGCGTCGCCGCGCCGCCGTCCCCTTCTCCCTCTCCAGCCTCGGGGCTGTCCG
CGGGGGGACGGCTGCCTTCGGGGGGGACGGGGCAGGGCGGGGTTCGGCTTCTGGCGTGTGACCG
GCGGCTCTAGAGCCTCTGCTAACCATGTTCATGCCTTCTTCTTTTTCCTACAG

Flanking Untranslated Regions, 5′ UTRs and 3′ UTRs

In some embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a pendrin protein.

Natural 5′ UTRs include a sequence that plays a role in translation initiation. in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

In some embodiments, a 5′ UTR from an mRNA that is transcribed by a cell in the cochlea can be included in any of the constructs, compositions, kits, and methods described herein. In some embodiments, a 5′ UTR is derived from the endogenous SLC26A4 gene loci and may include all or part of the endogenous sequence exemplified by SEQ ID NO: 21. In some embodiments, a 5′ UTR sequence is at least 85%, 90%, 95%, 98% or 99% identical to the 5′ UTR sequence represented by SEQ ID NO: 21

3′ UTRs are found immediately 3′ to the stop codon of the gene of interest. In some embodiments, a 3′ UTR from an mRNA that is transcribed by a cell in the cochlea can be included in any of the constructs, compositions, kits, and methods described herein. In some embodiments, a 3′ UTR is derived from the endogenous SLC26A4 gene loci and may include all or part of the endogenous sequence exemplified by SEQ ID NO: 22. In some embodiments, a 3′ UTR sequence is at least 85%, 90%, 95%, 98% or 99% identical to the 3′ UTR sequence represented by SEQ ID NO: 22.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mal. Cell. Biol. 15:5777-5788, 1995; Chen et al., Mal. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a pendrin protein. In other embodiments, AREs can be removed or mutated to increase the intracellular stability and thus increase translation and production of a pendrin protein.

In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 21)
CTCAGCCTTCCCGGTTCGGGAAAGGGGAAGAATGCAGGAGGGGTAGGATTTCTTTCCTGATAGG
ATCGGTTGGGAAAGACCGCAGCCTGTGTGTGTCTTTCCCTTCGACCAAGGTGTCTGTTGCTCCG
TAAATAAAACGTCCCACTGCCTTCTGAGAGCGCTATAAAGGCAGCGGAAGGGTAGTCCGCGGGG
CATTCCGGGCGGGGCGCGAGCAGAGACAGGTC
Exemplary 3′ UTR Sequence
(SEQ ID NO: 22)
AAGTGGGTTCGGGAGGTCTCTATGAGCAAGGAATACAAGACAAAACTTCCTCAATGCATTGACT
ATTTCTTCAGACTCAAAACACTCATTCTTTTTTCTATTAAGCCATTGAAAGAGAAGCACTAAGA
CTGCTTCTAGGCTTTATTTATAAAATAAACACCTTATCCCTAACATGGGCAAAATGGCTAGAAT
TATTCAGACGATTTGGCAGCGTCCAGGGTAAGCTGGTGTTATAATACGCTGCTGATCTACATCA
CAGATTTGCTAATAATGTTCACGTGGGCCCTGGCATATCTCTGTTCAGTTAGAGTGAGTGCTGA
CCCAACAGCCTCTGTGGTCAAGCGAGTCACGAATGATTAATCATAAAGAAAAATCAGTTTTTGA
CTGACCTGGATATCCATGAGCTGCACTGATCACCATGTAAGGTCACATTTAGTAAATGCTGAAA
TAAAATGATTAATGCATTTATCAATAAAAGCCTTTGAAAATACTTTGGATAATAAATTGGAGTT
TTAAAAATGCAAATTTGCTTAGTATCTAATAATGAAGTGTTATTACATATAGCCGGAATTGAGG
ATCTCTTTGATCCTGGAAATGGTTTACCTAAAAGCTACAGAACCAGGCCAATATATTTTGAAAT
ATTGATGCAGACAAATGAAATAATAAAGAGATTTTCATGGTTTATAAAAATCTTTTTTGATATG
ATAATAATCATGATCACAACTGAGATCAAAAAAATATATGACAGATTATTTTGTTTAAAAATGC
AGTTTTAATTATCTTAGTCTATAGAAATGATCATTGCATGGAGGCATGTATAGGTATGATCTGT
GTAAAATCTGACATAAAAACAGTGCTATTCTGAGTGAAAATTTTTTTGATGTGCTTACATAACC
ATGGTGATTAAAATGAGTTTATATTTTTTCTCAAAAATTTTAGCAGTGTGTAAAGTAAGTAATC
TTTAACTGAACTCTGACCACTTAAAAAAAAATCTAAAAATTGAACTACCTATAGTAGTCTGTGT
TTAAAGTGAATTTTTAAAGACAAAGCATTCTAAATGAACTCAATATAAAAACATTCATTTGGAA
TGTACATACTGAAAAATACAGGTTTTTTTGACCAAAAGTTTTTATATCTTTTCTTTTTATTTAT
TTTTTTCCTAAGTGCCAACAATTTTCTAGATATTATATACAACACAGGCTTTGATCTTGGGGAC
TTTTCCCATATATTTCACACTGGAGTGAATGAAGTTGTACTTCATTTCTAGAGAAAAGTTATAC
CCAGGTCCCCAATTGAGAATGTCTTGCTTGATTGAAAACGACATCATCCCTTGGTATACTCCAG
GGATTGGTTTCAGGACCCCTGCATTTACCAAAATTTGTGCACACTCAAGTCCTGCAGTCACCCC
TGCCTAAAGATAGAATGGCTTCTCTGTTTTTCTTCTGAAATACAACCAGAAACAATGTGTCTAT
TTCTGAAAGAATAGGATTAATGATCATACAAATGGGTTAATCCTGAATTCTGGTTGTAAATCTG
GTTACAGCATAACTAGGATTATAATGCTGCCTCATTTTCACAGCACTACTTGCTTATATTGACA
ACAAATCATCTCGCTAAAGAGTGAATGTAGGCCAGGCGCGGTGGCTCATGCCTGTAATCCCAGC
ACTTTGGGAGGCCGAGGCGGGTGGATCACGAGGTCAGGAGATCGAGACCATCCTGGCTAACATG
GTAAAACCCCGTCTCTACTAAAAATAGAAAAAAAGAAATTAGCCTAGCGTGGTGGCTGGCGGGC
GCCTGTAGTCCCAGCTATTTGGGAGGCTAAGGCAGGAGAATGGCGTGAACCCGGGAGGCGGAGC
TTGCAGTGAGCCGAGGTCGTGCCACTGCACTCCAGCCTGGGCGACAGAGCAAGACTCCGTCTCA
AAAAAAAAAAAAAAAAAAAAAAAAGAGTGAATGTAATAGTCTTGCAGAAAATGAATGAATACCT
TTGTTCAATAAAGGAAATATGCACTGCTCACTTTTTTGAAGGAAATGCCAAAGTTACGTTTTAC
AACAAGGCTAGAGTTTGTAAATTCTGGGTTCATTTGTGATGACATAAGTCAGCAAACTGCGGGA
ATACTGTCTCTTCTATGTATTTTGTGAATAGTAAGCATAATTTTAGTTTTGTATTATCAATGAA
AATTTCACTTGAAATTAAAGCTGCCTTTTGTTATATTTTTAACCTATAGGATAAGATTCCAGTA
TTGTATATGAGTTTTAACAAATTAAAAAATCAAATCATGTACATTTGAAAATATTTGCACACAT
TTAAAAATAAATGTAAAGTTGTCTTTTAAACTACTCGGATGTGTCCTTTCTGAACAA

Internal Ribosome Entry Sites (IRES)

In some embodiments, a construct encoding a pendrin protein can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001, each of which is incorporated in its entirety herein by reference.

In some embodiments, the IRES sequence that is incorporated into a construct that encodes a pendrin protein, or a C-terminal portion of a pendrin protein is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11: 1921-1931, 2000; and Klump et al., Gene Therapy 8:811-817, 2001, each of which is incorporated in its entirety herein by reference).

An IRES can be utilized in an AAV construct. In some embodiments, a construct encoding the C-terminal portion of the pendrin protein can include a polynucleotide internal ribosome entry site (IRES). In some embodiments, an IRES can be part of a composition comprising more than one construct. In some embodiments, an IRES is used to produce more than one polypeptide from a single gene transcript.

Splice Sites

In some embodiments, any of the constructs provided herein can include splice donor and/or splice acceptor sequences, which are functional during RNA processing occurring during transcription. In some embodiments, splice sites are involved in trans-splicing.

Exemplary splice donor intron
(SEQ ID NO: SEQ ID NO: 41)
GTAAGTATCAAGGTTACAAGACAGGTTTAAGGAGACCAATAGAAACTGG
GCTTGTCGAGACAGAGAAGACTCTTGCGTTTCT
Exemplary splice acceptor intron
(SEQ ID NO: SEQ ID NO: 42)
GATAGGCACCTATTGGTCTTACTGACATCCACTTTGCCTTTCTCTCCAC
AG

Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. USA. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71(2):313-319, 1988, each of which is incorporated herein by reference in its entirety), human collagen, polyoma virus (Batt et al., Mal. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mal. Therapy 15(7):1340-1347, 2007, which is incorporated herein by reference in its entirety), the group consisting of SV40 poly(A) site, such as the SV40 late and early poly(A) site (Schek et al., Mal. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al, Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG (SEQ ID NO: 23)) (see, e.g., WO 05/073384, which is incorporated herein by reference in its entirety). In some embodiments, a poly(A) signal sequence comprises or consists of the SV40 poly(A) site. In some embodiments, a poly(A) signal comprises or consists of SEQ ID NO: 25. In some embodiments, a poly(A) signal sequence comprises or consists of bGHpA. In some embodiments, a poly(A) signal comprises or consists of SEQ ID NO: 24. Additional examples of poly(A) signal sequences are known in the art. In some embodiments, a poly(A) sequence is at least 85%, 90%, 95%, 98% or 99% identical to the poly(A) sequence represented by SEQ ID NO: 24 or 25.

Exemplary bGH poly(A) signal sequence
(SEQ ID NO: 24)
CTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAAT
GAGGAAATTGCATCGCATTGTCTGAGTAGGTGTCATTCTATTCTGGGGG
GTGGGGTGGGGCAGGACAGCAAGGGGGAGGATTGGGAAGACAATAGCAG
GCATGCTGGGGATGCGGTGGGCTCTATGG
Exemplary SV40 poly(A) signal sequence
(SEQ ID NO: 25)
AACTTGTTTATTGCAGCTTATAATGGTTACAAATAAAGCAATAGCATCA
CAAATTTCACAAATAAAGCATTTTTTTCACTGCATTCTAGTTGTGGTTT
GTCCAAACTCATCAATGTATCTTA

Additional Sequences

In some embodiments, constructs of the present disclosure may comprise a T2A element or sequence. In some embodiments, constructs of the present disclosure may include one or more cloning sites. In some such embodiments, cloning sites may not be fully removed prior to manufacturing for administration to a subject. In some embodiments, cloning sites may have functional roles including as linker sequences, or as portions of a Kozak site. As will be appreciated by those skilled in the art, cloning sites may vary significantly in primary sequence while retaining their desired function. In some embodiments, constructs may contain any combination of cloning sites, exemplary cloning sites are represented by SEQ ID NO: 26-33.

Exemplary cloning site A
(SEQ ID NO: 26)
TTGTCGACGCGGCCGCACGCGT
Exemplary cloning site B
(SEQ ID NO: 27)
CTCCTGGGCAACGTGCTGGTTATTGTGACCGGTCGCTAGCCACC
Exemplary cloning site C
(SEQ ID NO: 28)
TAAGAGCTCGCTGATCAGCCTCGA
Exemplary cloning site D
(SEQ ID NO: 29)
AAGCTTGAATTCAGCTGACGTGCCTCGGACCGTCCTAGG
Exemplary cloning site E
(SEQ ID NO: 30)
GCGGCCGCACGCGT
Exemplary cloning site F
(SEQ ID NO: 31)
CTCCTGGGCAACGTGCTGGTTATTGTGACCGGTGCCACC
Exemplary cloning site G
(SEQ ID NO: 32)
TAAGAGCTCGCTGATCAGCCTCGA
Exemplary cloning site H
(SEQ ID NO: 33)
AAGCTTGAATTCAGCTGACGTGCCTCGGACCGCT

Destabilization Domains

In some embodiments, any of the constructs provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506—and rapamycin-binding protein (FKBP12) sequence, and the stabilizing ligand is Shield-1 (Shld1) (Banaszynski et al. (2012) Cell 126(5): 995-1004, which is incorporated in its entirety herein by reference). In some embodiments, a destabilizing sequence is a DIFR sequence, and a stabilizing ligand is trimethoprim (TMP) (Iwamoto et al. (2010) Chem Biol 17:981-988, which is incorporated in its entirety herein by reference).

In some embodiments, a destabilizing sequence is a FKBP12 sequence, and a presence of an AAV construct carrying the FKBP12 gene in a subject cell (e.g., a supporting cochlear outer hair cell) is detected by western blotting. In some embodiments, a destabilizing sequence can be used to verify the temporally-specific activity of any of the AAV constructs described herein.

Exemplary DHFR destabilizing amino acid sequence
(SEQ ID NO: 34)
MISLIAALAVDYVIGMENAMPWNLPADLAWFKRNTLNKPVIMGRHTWESIGRPLPGRKNIILSS
QPSTDDRVTWVKSVDEAIAACGDVPEIMVIGGGRVIEQFLPKAQKLYLTHIDAEVEGDTHFPDY
EPDDWESVFSEFHDADAQNSHSYCFEILERR
Exemplary DHFR destabilizing nucleotide sequence
(SEQ ID NO: 35)
GGTACCATCAGTCTGATTGCGGCGTTAGCGGTAGATTACGTTATCGGCATGGAAAACGCCATGC
CGTGGAACCTGCCTGCCGATCTCGCCTGGTTTAAACGCAACACCTTAAATAAACCCGTGATTAT
GGGCCGCCATACCTGGGAATCAATCGGTCGTCCGTTGCCAGGACGCAAAAATATTATCCTCAGC
AGTCAACCGAGTACGGACGATCGCGTAACGTGGGTGAAGTCGGTGGATGAAGCCATCGCGGCGT
GTGGTGACGTACCAGAAATCATGGTGATTGGCGGCGGTCGCGTTATTGAACAGTTCTTGCCAAA
AGCGCAAAAACTGTATCTGACGCATATCGACGCAGAAGTGGAAGGCGACACCCATTTCCCGGAT
TACGAGCCGGATGACTGGGAATCGGTATTCAGCGAATTCCACGATGCTGATGCGCAGAACTCTC
ACAGCTATTGCTTTGAGATTCTGGAGCGGCGATAA
Exemplary destabilizing domain
(SEQ ID NO: 36)
ATCAGTCTGATTGCGGCGTTAGCGGTAGATTACGTTATCGGCATGGAAAACGCCATGCCGTGGA
ACCTGCCTGCCGATCTCGCCTGGTTTAAACGCAACACCTTAAATAAACCCGTGATTATGGGCCG
CCATACCTGGGAATCAATCGGTCGTCCGTTGCCAGGACGCAAAAATATTATCCTCAGCAGTCAA
CCGAGTACGGACGATCGCGTAACGTGGGTGAAGTCGGTGGATGAAGCCATCGCGGCGTGTGGTG
ACGTACCAGAAATCATGGTGATTGGCGGCGGTCGCGTTATTGAACAGTTCTTGCCAAAAGCGCA
AAAACTGTATCTGACGCATATCGACGCAGAAGTGGAAGGCGACACCCATTTCCCGGATTACGAG
CCGGATGACTGGGAATCGGTATTCAGCGAATTCCACGATGCTGATGCGCAGAACTCTCACAGCT
ATTGCTTTGAGATTCTGGAGCGGCGA
Exemplary FKBP12 destabilizing peptide amino acid sequence
(SEQ ID NO: 37)
MGVEKQVIRPGNGPKPAPGQTVTVHCTGFGKDGDLSQKFWSTKDEGQKPFSFQIGKGAVIKGWD
EGVIGMQIGEVARLRCSSDYAYGAGGFPAWGIQPNSVLDFEIEVLSVQ

Reporter Sequences or Elements

In some embodiments, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a mammalian cell (e.g., a cochlear hair cell) is detected by assays for beta-galactosidase activity. When the reporter is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3×FLAG tag), and the presence of a construct carrying the FLAG tag in a mammalian cell (e.g., an inner ear cell, e.g., a cochlear hair or supporting cell) is detected by protein binding or detection assays (e.g., Western blots, immunohistochemistry, radioimmunoassay (RIA), mass spectrometry). An exemplary 3×FLAG tag sequence is provided as SEQ ID NO: 38.

Exemplary 3xFLAG tag sequence
(SEQ ID NO: 38)
GGATCCCGGGCTGACTACAAAGACCATGACGGTGATTATAAAGATCAT
GACATCGACTACAAGGATGAGGATGAGAAG

AAV Capsids

The present disclosure provides one or more polynucleotide constructs packaged into an AAV capsid. In some embodiments, an AAV capsid is from or derived from an AAV capsid of an AAV2, 3, 4, 5, 6, 7, 8, 9, 10, rh8, rh10, rh39, rh43 or Anc80 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some embodiments, an AAV capsid is an Anc80 capsid (e.g., an Anc80L65 capsid). In some embodiments, an AAV capsid is created using a template nucleotide coding sequence comprising SEQ ID NO: 8. In some embodiments, the capsid comprises a polypeptide represented by SEQ ID NO: 9. In some embodiments, the capsid comprises a polypeptide with at least 85%, 90%, 95%, 98% or 99% sequence identity to the polypeptide represented by SEQ ID NO: 9.

As provided herein, any combination of AAV capsids and AAV constructs (e.g., comprising AAV ITRs) may be used in recombinant AAV (rAAV) particles of the present disclosure. For example, wild type or variant AAV2 ITRs and Anc80 capsid, wild type or variant AAV2 ITRs and AAV6 capsid, etc. In some embodiments of the present disclosure, an AAV particle is wholly comprised of AAV2 components (e.g., capsid and ITRs are AAV2 serotype). In some embodiments, an AAV particle is an AAV2/6, AAV2/8 or AAV2/9 particle (e.g., an AAV6, AAV8 or AAV9 capsid with an AAV construct having AAV2 ITRs). In some embodiments of the present disclosure, an AAV particle is an AAV2/Anc80 particle that comprises an Anc80 capsid (e.g., comprising a polypeptide of SEQ ID NO: 9) that encapsidates an AAV construct with AAV2 ITRs (e.g., SEQ ID NOs: 10 and 11) flanking a portion of a coding sequence, for example, an SLC26A4 gene or characteristic portion thereof (e.g., SEQ ID NO: 1, 2, 3, 4, or 5). Other AAV particles are known in the art and are described in, e.g., Sharma et al., Brain Res Bull. 2010 Feb. 15; 81(2-3): 273, which is incorporated in its entirety herein by reference. In some embodiments, a capsid sequence is at least 85%, 90%, 95%, 98% or 99% identical to a capsid nucleotide or amino acid sequence represented by SEQ ID NO: 8 or 9, respectively.

Exemplary AAV Anc80 Capsid DNA Sequence
(SEQ ID NO: 8)
ATGGCTGCCGATGGTTATCTTCCAGATTGGCTCGAGGACAACCTCTCTGAGGGCATTCGCGAGT
GGTGGGACTTGAAACCTGGAGCCCCGAAACCCAAAGCCAACCAGCAAAAGCAGGACGACGGCCG
GGGTCTGGTGCTTCCTGGCTACAAGTACCTCGGACCCTTCAACGGACTCGACAAGGGGGAGCCC
GTCAACGCGGCGGACGCAGCGGCCCTCGAGCACGACAAGGCCTACGACCAGCAGCTCAAAGCGG
GTGACAATCCGTACCTGCGGTATAACCACGCCGACGCCGAGTTTCAGGAGCGTCTGCAAGAAGA
TACGTCTTTTGGGGGCAACCTCGGGCGAGCAGTCTTCCAGGCCAAGAAGCGGGTTCTCGAACCT
CTCGGTCTGGTTGAGGAAGGCGCTAAGACGGCTCCTGGAAAGAAGAGACCGGTAGAGCAATCAC
CCCAGGAACCAGACTCCTCTTCGGGCATCGGCAAGAAAGGCCAGCAGCCCGCGAAGAAGAGACT
CAACTTTGGGCAGACAGGCGACTCAGAGTCAGTGCCCGACCCTCAACCACTCGGAGAACCCCCC
GCAGCCCCCTCTGGTGTGGGATCTAATACAATGGCAGCAGGCGGTGGCGCTCCAATGGCAGACA
ATAACGAAGGCGCCGACGGAGTGGGTAACGCCTCAGGAAATTGGCATTGCGATTCCACATGGCT
GGGCGACAGAGTCATCACCACCAGCACCCGAACCTGGGCCCTCCCCACCTACAACAACCACCTC
TACAAGCAAATCTCCAGCCAATCGGGAGCAAGCACCAACGACAACACCTACTTCGGCTACAGCA
CCCCCTGGGGGTATTTTGACTTTAACAGATTCCACTGCCACTTCTCACCACGTGACTGGCAGCG
ACTCATCAACAACAACTGGGGATTCCGGCCCAAGAGACTCAACTTCAAGCTCTTCAACATCCAG
GTCAAGGAGGTCACGACGAATGATGGCACCACGACCATCGCCAATAACCTTACCAGCACGGTTC
AGGTCTTTACGGACTCGGAATACCAGCTCCCGTACGTCCTCGGCTCTGCGCACCAGGGCTGCCT
GCCTCCGTTCCCGGCGGACGTCTTCATGATTCCTCAGTACGGGTACCTGACTCTGAACAATGGC
AGTCAGGCCGTGGGCCGTTCCTCCTTCTACTGCCTGGAGTACTTTCCTTCTCAAATGCTGAGAA
CGGGCAACAACTTTGAGTTCAGCTACACGTTTGAGGACGTGCCTTTTCACAGCAGCTACGCGCA
CAGCCAAAGCCTGGACCGGCTGATGAACCCCCTCATCGACCAGTACCTGTACTACCTGTCTCGG
ACTCAGACCACGAGTGGTACCGCAGGAAATCGGACGTTGCAATTTTCTCAGGCCGGGCCTAGTA
GCATGGCGAATCAGGCCAAAAACTGGCTACCCGGGCCCTGCTACCGGCAGCAACGCGTCTCCAA
GACAGCGAATCAAAATAACAACAGCAACTTTGCCTGGACCGGTGCCACCAAGTATCATCTGAAT
GGCAGAGACTCTCTGGTAAATCCCGGTCCCGCTATGGCAACCCACAAGGACGACGAAGACAAAT
TTTTTCCGATGAGCGGAGTCTTAATATTTGGGAAACAGGGAGCTGGAAATAGCAACGTGGACCT
TGACAACGTTATGATAACGAGTGAGGAAGAAATTAAAACCACCAACCGAGTGGCCACAGAACAG
TACGGCACGGTGGCCACTAACCTGCAATCGTCAAACACCGCTCCTGCTACAGGGACCGTCAACA
GTCAAGGAGCCTTACCTGGCATGGTCTGGCAGAACCGGGACGTGTACCTGCAGGGTCCTATCTG
GGCCAAGATTCCTCACACGGACGGACACTTTCATCCCTCGCCGCTGATGGGAGGCTTTGGACTG
AAACACCCGCCTCCTCAGATCCTGATTAAGAATACACCTGTTCCCGCGAATCCTCCAACTACCT
TCAGTCCAGCTAAGTTTGCGTCGTTCATCACGCAGTACAGCACCGGACAGGTCAGCGTGGAAAT
TGAATGGGAGCTGCAGAAAGAAAACAGCAAACGCTGGAACCCAGAGATTCAATACACTTCCAAC
TACAACAAATCTACAAATGTGGACTTTGCTGTTGACACAAATGGCGTTTATTCTGAGCCTCGCC
CCATCGGCACCCGTTACCTCACCCGTAATCTG
Exemplary AAV Anc80 Capsid Amino Acid Sequence
(SEQ ID NO: 9)
MAADGYLPDWLEDNLSEGIREWWDLKPGAPKPKANQQKQDDGRGLVLPGYKYLGPFNGLDKGEP
VNAADAAALEHDKAYDQQLKAGDNPYLRYNHADAEFQERLQEDTSFGGNLGRAVFQAKKRVLEP
LGLVEEGAKTAPGKKRPVEQSPQEPDSSSGIGKKGQQPAKKRLNFGQTGDSESVPDPQPLGEPP
AAPSGVGSNTMAAGGGAPMADNNEGADGVGNASGNWHCDSTWLGDRVITTSTRTWALPTYNNHL
YKQISSQSGASTNDNTYFGYSTPWGYFDFNRFHCHFSPRDWQRLINNNWGFRPKRLNFKLFNIQ
VKEVTTNDGTTTIANNLTSTVQVFTDSEYQLPYVLGSAHQGCLPPFPADVFMIPQYGYLTLNNG
SQAVGRSSFYCLEYFPSQMLRTGNNFEFSYTFEDVPFHSSYAHSQSLDRLMNPLIDQYLYYLSR
TQTTSGTAGNRTLQFSQAGPSSMANQAKNWLPGPCYRQQRVSKTANQNNNSNFAWTGATKYHLN
GRDSLVNPGPAMATHKDDEDKFFPMSGVLIFGKQGAGNSNVDLDNVMITSEEEIKTTNPVATEQ
YGTVATNLQSSNTAPATGTVNSQGALPGMVWQNRDVYLQGPIWAKIPHTDGHFHPSPLMGGFGL
KHPPPQILIKNTPVPANPPTTFSPAKFASFITQYSTGQVSVEIEWELQKENSKRWNPEIQYTSN
YNKSTNVDFAVDTNGVYSEPRPIGTRYLTRNL*

### Compositions

Among other things, the present disclosure provides compositions. In some embodiments, a composition comprises a construct as described herein. In some embodiments, a composition comprises one or more constructs as described herein. In some embodiments, a composition comprises a plurality of constructs as described herein. In some embodiments, when more than one construct is included in the composition, the constructs are each different.

In some embodiments, a composition comprises an AAV particle as described herein. In some embodiments, a composition comprises one or more AAV particles as described herein. In some embodiments, a composition comprises a plurality of AAV particles. In come embodiments, when more than one AAV particle is included in the composition, the AAV particles are each different.

In some embodiments, a composition comprises pendrin protein. In some embodiments, a composition comprises a cell.

In some embodiments, a composition is or comprises a pharmaceutical composition.

Single AAV Construct Compositions

In some embodiments, the present disclosure provides compositions or systems comprising AAV particles comprised of a single construct. In some such embodiments, a single construct may deliver a polynucleotide that encodes a functional (e.g., wild type or otherwise functional, e.g., codon optimized) copy of an SLC26A4 gene. In some embodiments, a construct is or comprises an rAAV construct. In some embodiments described herein, a single rAAV construct is capable of expressing a full-length SLC26A4 messenger RNA or a characteristic protein thereof in a target cell (e.g., an inner ear cell). In some embodiments, a single construct (e.g., any of the constructs described herein) can include a sequence encoding a functional pendrin protein (e.g., any construct that generates functional pendrin protein). In some embodiments, a single construct (e.g., any of the constructs described herein) can include a sequence encoding a functional pendrin protein (e.g., any construct that generates functional pendrin protein) and optionally additional polypeptide sequences (e.g., regulatory sequences, and/or reporter sequences).

In some embodiments, a single construct composition or system may comprise any or all of the exemplary construct components described herein. In some embodiments, an exemplary single construct is represented by SEQ ID NO: 39. In some embodiments, an exemplary single construct is represented by SEQ ID NO: 40. In some embodiments, an exemplary single construct is at least 85%, 90%, 95%, 98% or 99% identical to the sequence represented by SEQ ID NO: 39 or 40. One skilled in the art would recognize that constructs may undergo additional modifications including codon-optimization, introduction of novel but functionally equivalent (e.g., silent mutations), addition of reporter sequences, and/or other routine modification.

In some embodiments, an exemplary construct comprises: a 5′ ITR exemplified by SEQ ID NO: 10, optionally a cloning site exemplified by SEQ ID NO: 26, a CMV enhancer exemplified by SEQ ID NO: 19, a CBA promoter exemplified by SEQ ID NO: 14, a chimeric intron exemplified by SEQ ID NO: 20, optionally a cloning site exemplified by SEQ ID NO: 27, an SLC26A4 coding region exemplified by SEQ ID NO: 1, optionally a cloning site exemplified by SEQ ID NO: 28, a poly(A) site exemplified by SEQ ID NO: 24, optionally a cloning site exemplified by SEQ ID NO: 29, and a 3′ ITR exemplified by SEQ ID NO: 12.

In some embodiments, an exemplary construct comprises: a 5′ ITR exemplified by SEQ ID NO: 11, optionally a cloning site exemplified by SEQ ID NO: 30, a CMV enhancer exemplified by SEQ ID NO: 19, a CBA promoter exemplified by SEQ ID NO: 15, a chimeric intron exemplified by SEQ ID NO: 20, optionally a cloning site exemplified by SEQ ID NO: 31, an SLC26A4 coding region exemplified by SEQ ID NO: 1, optionally a reporter sequence exemplified by SEQ ID NO: 38, optionally a cloning site exemplified by SEQ ID NO: 32, a poly(A) site exemplified by SEQ ID NO: 24, optionally a cloning site exemplified by SEQ ID NO: 34, and a 3′ ITR exemplified by SEQ ID NO: 13.

Exemplary Single Construct sequence
(SEQ ID NO: 39)
TTGGCCACTCCCTCTCTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGAGGGAGTGGCCAACTC
CATCACTAGGGGTTCCTTTGTCGACGCGGCCGCACGCGTGACATTGATTATTGACTAGTTATTA
ATAGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGACTATTTACGGTA
AACTGCCCACTTGGCAGTACATCAAGTGTATCATATGCCAAGTACGCCCCCTATTGACGTCAAT
GACGGTAAATGGCCCGCCTGGCATTATGCCCAGTACATGACCTTATGGGACTTTCCTACTTGGC
AGTACATCTACGTATTAGTCATCGCTATTACCATGGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCGGGGCGAGGGG
CGGGGCGGGGCGAGGCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCGCGGCGGGCGGGAGT
CGCTGCGTTGCCTTCGCCCCGTGCCCCGCTCCGCGCCGCCTCGCGCCGCCCGCCCCGGCTCTGA
CTGACCGCGTTACTCCCACAGGTGAGCGGGCGGGACGGCCCTTCTCCTCCGGGCTGTAATTAGC
GCTTGGTTTAATGACGGCTCGTTTCTTTTCTGTGGCTGCGTGAAAGCCTTAAAGGGCTCCGGGA
GGGCCCTTTGTGCGGGGGGGAGCGGCTCGGGGGGTGCGTGCGTGTGTGTGTGCGTGGGGAGCGC
CGCGTGCGGCCCGCGCTGCCCGGCGGCTGTGAGCGCTGCGGGCGCGGCGCGGGGCTTTGTGCGC
TCCGCGTGTGCGCGAGGGGAGCGCGGCCGGGGGCGGTGCCCCGCGGTGCGGGGGGGCTGCGAGG
GGAACAAAGGCTGCGTGCGGGGTGTGTGCGTGGGGGGGTGAGCAGGGGGTGTGGGCGCGGCGGT
CGGGCTGTAACCCCCCCCTGCACCCCCCTCCCCGAGTTGCTGAGCACGGCCCGGCTTCGGGTGC
GGGGCTCCGTGCGGGGCGTGGCGCGGGGCTCGCCGTGCCGGGCGGGGGGTGGCGGCAGGTGGGG
GTGCCGGGCGGGGCGGGGCCGCCTCGGGCCGGGGAGGGCTCGGGGGAGGGGCGCGGCGGCCCCC
GGAGCGCCGGCGGCTGTCGAGGCGCGGCGAGCCGCAGCCATTGCCTTTTATGGTAATCGTGCGA
GAGGGCGCAGGGACTTCCTTTGTCCCAAATCTGTGCGGAGCCGAAATCTGGGAGGCGCCGCCGC
ACCCCCTCTAGCGGGCGCGGGGCGAAGCGGTGCGGCGCCGGCAGGAAGGAAATGGGCGGGGAGG
GCCTTCGTGCGTCGCCGCGCCGCCGTCCCCTTCTCCCTCTCCAGCCTCGGGGCTGTCCGCGGGG
GGACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGCTCCTGGGCAACGTGC
TGGTTATTGTGACCGGTCGCTAGCCACCATGGCAGCGCCAGGCGGCAGGTCGGAGCCGCCGCAG
CTCCCCGAGTACAGCTGCAGCTACATGGTGTCGCGGCCGGTCTACTCGGAGCTAGCTTTCCAGC
AACAGCACGAGCGGCGCCTGCAGGAGCGCAAGACGCTGCGGGAGAGCCTGGCCAAGTGCTGCAG
TTGTTCAAGAAAGAGAGCCTTTGGTGTGCTAAAGACTCTTGTGCCCATCTTGGAGTGGCTCCCC
AAATACCGAGTCAAGGAATGGCTGCTTAGTGACGTCATTTCGGGAGTTAGTACTGGGCTAGTGG
CCACGCTGCAAGGGATGGCATATGCCCTACTAGCTGCAGTTCCTGTCGGATATGGTCTCTACTC
TGCTTTTTTCCCTATCCTGACATACTTTATCTTTGGAACATCAAGACATATCTCAGTTGGACCT
TTTCCAGTGGTGAGTTTAATGGTGGGATCTGTTGTTCTGAGCATGGCCCCCGACGAACACTTTC
TCGTATCCAGCAGCAATGGAACTGTATTAAATACTAGTATGATAGACACTGCAGCTAGAGATAC
AGCTAGAGTCCTGATTGCCAGTGCCCTGACTCTGCTGGTTGGAATTATACAGTTGATATTTGGT
GGCTTGCAGATTGGATTCATAGTGAGGTACTTGGCAGATCCTTTGGTTGGTGGCTTCACAACAG
CTGCTGCCTTCCAAGTGCTGGTCTCACAGCTAAAGATTGTCCTCAATGTTTCAACCAAAAACTA
CAATGGAGTTCTCTCTATTATCTATACGCTGGTTGAGATTTTTCAAAATATTGGTGATACCAAT
CTTGCTGATTTCACTGCTGGATTGCTCACCATTGTCGTCTGTATGGCAGTTAAGGAATTAAATG
ATCGGTTTAGACACAAAATCCGAGTCCCTATTCCTATAGAAGTAATTGTGAGGATAATTGCTAC
TGCCATTTCATATGGAGCCAACCTGGAAAAAAATTACAATGCTGGCATTGTTAAATCCATCCCA
AGGGGGTTTTTGCCTCCTGAACTTCCACCTGTGAGCTTGTTCTCGGAGATGCTGGCTGCATCAT
TTTCCATCGCTGTGGTGGCTTATGCTATTGCAGTGTCAGTAGGAAAAGTATATGCCACCAAGTA
TGATTACACCATCGATGGGAACCAGGAATTCATTGCCTTTGGGATCAGCAACATCTTCTCAGGA
TTCTTCTCTTGTTTTGTGGCCACCACTGCTCTTTCCCGCACGGCCGTCCAGGAGAGCACTGGAG
GAAAGACACAGGTTGCTGGCATCATCTCTGCTGCGATTGTGATGATCGCCATTCTTGCCCTGGG
GAAGCTTCTGGAACCCTTGCAGAAGTCGGTCTTGGCAGCTGTTGTAATTGCCAACCTGAAAGGG
ATGTTTATGCAGCTGTGTGACATTCCTCGTCTGTGGAGACAGAATAAGATTGATGCTGTTATCT
GGGTGTTTACGTGTATAGTGTCCATCATTCTGGGGCTGGATCTCGGTTTACTAGCTGGCCTTAT
ATTTGGACTGTTGACTGTGGTCCTGAGAGTTCAGTTTCCTTCTTGGAATGGCCTTGGAAGCATC
CCTAGCACAGATATCTACAAAAGTACCAAGAATTACAAAAACATTGAAGAACCTCAAGGAGTGA
AGATTCTTAGATTTTCCAGTCCTATTTTCTATGGCAATGTCGATGGTTTTAAAAAATGTATCAA
GTCCACAGTTGGATTTGATGCCATTAGAGTATATAATAAGAGGCTGAAAGCGCTGAGGAAAATA
CAGAAACTAATAAAAAGTGGACAATTAAGAGCAACAAAGAATGGCATCATAAGTGATGCTGTTT
CAACAAATAATGCTTTTGAGCCTGATGAGGATATTGAAGATCTGGAGGAACTTGATATCCCAAC
CAAGGAAATAGAGATTCAAGTGGATTGGAACTCTGAGCTTCCAGTCAAAGTGAACGTTCCCAAA
GTGCCAATCCATAGCCTTGTGCTTGACTGTGGAGCTATATCTTTCCTGGACGTTGTTGGAGTGA
GATCACTGCGGGTGATTGTCAAAGAATTCCAAAGAATTGATGTGAATGTGTATTTTGCATCACT
TCAAGATTATGTGATAGAAAAGCTGGAGCAATGCGGGTTCTTTGACGACAACATTAGAAAGGAC
ACATTCTTTTTGACGGTCCATGATGCTATACTCTATCTACAGAACCAAGTGAAATCTCAAGAGG
GTCAAGGTTCCATTTTAGAAACGATCACTCTCATTCAGGATTGTAAAGATACCCTTGAATTAAT
AGAAACAGAGCTGACGGAAGAAGAACTTGATGTCCAGGATGAGGCTATGCGTACACTTGCATCC
TAAGAGCTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATGGAAGCTTG
AATTCAGCTGACGTGCCTCGGACCGTCCTAGGAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAGAGAGGGAGTGGCCAA
Exemplary Single Construct sequence with FLAG reporter
(SEQ ID NO: 40)
CTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCA
CGCGTGACATTGATTATTGACTAGTTATTAATAGTAATCAATTACGGGGTCATTAGTTCATAGC
CCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCA
TTGACGTCAATGGGTGGACTATTTACGGTAAACTGCCCACTTGGCAGTACATCAAGTGTATCAT
ATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCAT
GGGTCGAGGTGAGCCCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGGGGCGCGC
GCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATA
AAAAGCGAAGCGCGCGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTCTTTTCTGTGGC
TGCGTGAAAGCCTTAAAGGGCTCCGGGAGGGCCCTTTGTGCGGGGGGGAGCGGCTCGGGGGGTG
CGTGCGTGTGTGTGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGCGGCCGGGGGCGG
TGCCCCGCGGTGCGGGGGGGCTGCGAGGGGAACAAAGGCTGCGTGCGGGGTGTGTGCGTGGGGG
GGTGAGCAGGGGGTGTGGGCGCGGCGGTCGGGCTGTAACCCCCCCCTGCACCCCCCTCCCCGAG
TTGCTGAGCACGGCCCGGCTTCGGGTGCGGGGCTCCGTGCGGGGCGTGGCGCGGGGCTCGCCGT
GCCGGGCGGGGGGTGGCGGCAGGTGGGGGTGCCGGGCGGGGCGGGGCCGCCTCGGGCCGGGGAG
GGCTCGGGGGAGGGGCGCGGCGGCCCCCGGAGCGCCGGCGGCTGTCGAGGCGCGGCGAGCCGCA
GCCATTGCCTTTTATGGTAATCGTGCGAGAGGGCGCAGGGACTTCCTTTGTCCCAAATCTGTGC
GGAGCCGAAATCTGGGAGGCGCCGCCGCACCCCCTCTAGCGGGCGCGGGGCGAAGCGGTGCGGC
GCCGGCAGGAAGGAAATGGGCGGGGAGGGCCTTCGTGCGTCGCCGCGCCGCCGTCCCCTTCTCC
CTCTCCAGCCTCGGGGCTGTCCGCGGGGGGACGGCTGCCTTCGGGGGGGACGGGGCAGGGCGGG
GTTCGGCTTCTGGCGTGTGACCGGCGGCTCTAGAGCCTCTGCTAACCATGTTCATGCCTTCTTC
TTTTTCCTACAGCTCCTGGGCAACGTGCTGGTTATTGTGACCGGTGCCACCATGGCAGCGCCAG
GCGGCAGGTCGGAGCCGCCGCAGCTCCCCGAGTACAGCTGCAGCTACATGGTGTCGCGGCCGGT
CTACTCGGAGCTAGCTTTCCAGCAACAGCACGAGCGGCGCCTGCAGGAGCGCAAGACGCTGCGG
GAGAGCCTGGCCAAGTGCTGCAGTTGTTCAAGAAAGAGAGCCTTTGGTGTGCTAAAGACTCTTG
TGCCCATCTTGGAGTGGCTCCCCAAATACCGAGTCAAGGAATGGCTGCTTAGTGACGTCATTTC
GGGAGTTAGTACTGGGCTAGTGGCCACGCTGCAAGGGATGGCATATGCCCTACTAGCTGCAGTT
CCTGTCGGATATGGTCTCTACTCTGCTTTTTTCCCTATCCTGACATACTTTATCTTTGGAACAT
CAAGACATATCTCAGTTGGACCTTTTCCAGTGGTGAGTTTAATGGTGGGATCTGTTGTTCTGAG
CATGGCCCCCGACGAACACTTTCTCGTATCCAGCAGCAATGGAACTGTATTAAATACTACTATG
ATAGACACTGCAGCTAGAGATACAGCTAGAGTCCTGATTGCCAGTGCCCTGACTCTGCTGGTTG
GAATTATACAGTTGATATTTGGTGGCTTGCAGATTGGATTCATAGTGAGGTACTTGGCAGATCC
TTTGGTTGGTGGCTTCACAACAGCTGCTGCCTTCCAAGTGCTGGTCTCACAGCTAAAGATTGTC
CTCAATGTTTCAACCAAAAACTACAATGGAGTTCTCTCTATTATCTATACGCTGGTTGAGATTT
TTCAAAATATTGGTGATACCAATCTTGCTGATTTCACTGCTGGATTGCTCACCATTGTCGTCTG
TATGGGAGTTAAGGAATTAAATGATCGGTTTAGACACAAAATCCGAGTCCCTATTCCTATAGAA
GTAATTGTGACGATAATTGCTACTGCCATTTCATATGGAGCCAACCTGGAAAAAAATTACAATG
CTGGCATTGTTAAATCCATCCCAAGGGGGTTTTTGCCTCCTGAACTTCCACCTGTGAGCTTGTT
CTCGGAGATGCTGGCTGCATCATTTTCCATCGCTGTGGTGGCTTATGCTATTGCAGTGTCAGTA
GGAAAAGTATATGCCACCAAGTATGATTACACCATCGATGGGAACCAGGAATTCATTGCCTTTG
GGATCAGCAACATCTTCTCAGGATTCTTCTCTTGTTTTGTGGCCACCACTGCTCTTTCCCGCAC
GGCCGTCCAGGAGAGCACTGGAGGAAAGACACAGGTTGCTGGCATCATCTCTGCTGCGATTGTG
ATGATCGCCATTCTTGCCCTGGGGAAGCTTCTGGAACCCTTGCAGAAGTCGGTCTTGGCAGCTG
TTGTAATTGCCAACCTGAAAGGGATGTTTATGCAGCTGTGTGACATTCCTCGTCTGTGGAGACA
GAATAAGATTGATGCTGTTATCTGGGTGTTTACGTGTATAGTGTCCATCATTCTGGGGCTGGAT
CTCGGTTTACTAGCTGGCCTTATATTTGGACTGTTGACTGTGGTCCTGAGAGTTCAGTTTCCTT
CTTGGAATGGCCTTGGAAGCATCCCTAGCACAGATATCTACAAAAGTACCAAGAATTACAAAAA
CATTGAAGAACCTCAAGGAGTGAAGATTCTTAGATTTTCCAGTCCTATTTTCTATGGCAATGTC
GATGGTTTTAAAAAATGTATCAAGTCCACAGTTGGATTTGATGCCATTAGAGTATATAATAAGA
GGCTGAAAGCGCTGAGGAAAATACAGAAACTAATAAAAAGTGGACAATTAAGAGCAACAAAGAA
TGGCATCATAAGTGATGCTGTTTCAACAAATAATGCTTTTGAGCCTGATGAGGATATTGAAGAT
CTGGAGGAACTTGATATCCCAACCAAGGAAATAGAGATTCAAGTGGATTGGAACTCTGAGCTTC
CAGTCAAAGTGAACGTTCCCAAAGTGCCAATCCATAGCCTTGTGCTTGACTGTGGAGCTATATC
TTTCCTGGACGTTGTTGGAGTGAGATCACTGCGGGTGATTGTCAAAGAATTCCAAAGAATTGAT
GTGAATGTGTATTTTGCATCACTTCAAGATTATGTGATAGAAAAGCTGGAGCAATGCGGGTTCT
TTGACGACAACATTAGAAAGGACACATTCTTTTTGACGGTCCATGATGCTATACTCTATCTACA
GAACCAAGTGAAATCTCAAGAGGGTCAAGGTTCCATTTTAGAAACGATCACTCTCATTCAGGAT
TGTAAAGATACCCTTGAATTAATAGAAACAGAGCTGACGGAAGAAGAACTTGATGTCCAGGATG
AGGCTATGCGTACACTTGCATCCGGATCCCGGGCTGACTACAAAGACCATGACGGTGATTATAA
AGATCATGACATCGACTAGAAGGATGACGATGACAAGTAAGAGCTCGCTGATCAGCCTCGACTG
TGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCA
GGCATGCTGGGGATGCGGTGGGCTCTATGGAAGCTTGAATTCAGCTGACGTGCCTCGGACCGCT
AGGAACCCCTAGTGATGGAGTTGGCCACTCCCTCTCTGCGCGCTCGCTCGCTCACTGAGGCCGG
GCGACCAAAGGTCGCCCGACGCCCGGGCTTTGCCCGGGCGGCCTCAGTGAGCGAGCGAGCGCGC
AG

Multiple AAV Construct Compositions

The present disclosure recognizes that some coding sequences encoding a protein (e.g., pendrin protein) may be delivered by dividing the coding sequence into multiple portions, which are each included in a different construct. In some embodiments, provided herein are compositions or systems comprising at least two different constructs (e.g., two, three, four, five, or six). In some embodiments, each of the at least two different constructs includes a coding sequence that encodes a different portion of a coding region (e.g., encoding a target protein, e.g., an inner ear target protein, e.g., a pendrin protein), each of the encoded portions being at least 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) where the amino acid sequence of each of the encoded portions may optionally partially overlap with the amino acid sequence of a different one of the encoded portions; no single construct of the at least two different constructs encodes the active target protein; and, when introduced into a subject cell (e.g., an animal cell, e.g., a primate cell, e.g., a human cell), the at least two different constructs undergo homologous recombination with each other, where the recombined nucleic acid encodes an active target protein (e.g., a gene product encoded by an SLC26A4 gene or a characteristic portion thereof). In some embodiments, one of the nucleic acid constructs can include a coding sequence that encodes a portion of a target protein (e.g., an inner ear target protein, e.g., a pendrin protein), where the encoded portion is at most about 820 amino acids (e.g., at most about 10 amino acids, at most about 20 amino acids, at most about 30 amino acids, at most about 60 amino acids, at most about 70 amino acids, at most about 80 amino acids, at most about 90 amino acids, at most about 100 amino acids, at most about 110 amino acids, at most about 120 amino acids, at most about 130 amino acids, at most about 140 amino acids, at most about 150 amino acids, at most about 160 amino acids, at most about 170 amino acids, at most about 180 amino acids, at most about 190 amino acids, at most about 200 amino acids, at most about 210 amino acids, at most about 220 amino acids, at most about 230 amino acids, at most about 240 amino acids, at most about 250 amino acids, at most about 260 amino acids, at most about 270 amino acids, at most about 280 amino acids, at most about 290 amino acids, at most about 300 amino acids, at most about 310 amino acids, at most about 320 amino acids, at most about 330 amino acids, at most about 340 amino acids, at most about 350 amino acids, at most about 360 amino acids, at most about 370 amino acids, at most about 380 amino acids, at most about 390 amino acids, at most about 400 amino acids, at most about 410 amino acids, at most about 420 amino acids, at most about 430 amino acids, at most about 440 amino acids, at most about 450 amino acids, at most about 460 amino acids, at most about 470 amino acids, at most about 480 amino acids, at most about 490 amino acids, at most about 500 amino acids, at most about 510 amino acids, at most about 520 amino acids, at most about 530 amino acids, at most about 540 amino acids, at most about 550 amino acids, at most about 560 amino acids, at most about 570 amino acids, at most about 580 amino acids, at most about 590 amino acids, at most about 600 amino acids, at most about 610 amino acids, at most about 620 amino acids, at most about 630 amino acids, at most about 640 amino acids, at most about 650 amino acids, at most about 660 amino acids, at most about 670 amino acids, at most about 680 amino acids, at most about 690 amino acids, at most about 700 amino acids, at most about 710 amino acids, at most about 720 amino acids, at most about 730 amino acids, at most about 740 amino acids, at most about 750 amino acids, at most about 760 amino acids, at most about 770 amino acids, at most about 780 amino acids, at most about 790 amino acids, at most about 800 amino acids, at most about 810 amino acids, or at most about 820 amino acids).

In some embodiments, at least one of the constructs includes a nucleotide sequence spanning two neighboring exons of target genomic DNA (e.g., an inner ear target genomic DNA, e.g., SLC26A4 genomic DNA), and lacks the intronic sequence that naturally occurs between the two neighboring exons.

In some embodiments, an amino acid sequence of an encoded portion of each of the constructs does not overlap, even in part, with an amino acid sequence of a different one of the encoded portions. In some embodiments, an amino acid sequence of an encoded portion of a construct partially overlaps with an amino acid sequence of an encoded portion of a different construct. In some embodiments, an amino acid sequence of an encoded portion of each construct partially overlaps with an amino acid sequence of an encoded portion of at least one different construct. In some embodiments, an overlapping amino acid sequence is between about 10 amino acid residues to about 820 amino acids, or any of the subranges of this range (e.g., about 10 amino acids, about 20 amino acids, about 30 amino acids, about 60 amino acids, about 70 amino acids, about 80 amino acids, about 90 amino acids, about 100 amino acids, about 110 amino acids, about 120 amino acids, about 130 amino acids, about 140 amino acids, about 150 amino acids, about 160 amino acids, about 170 amino acids, about 180 amino acids, about 190 amino acids, about 200 amino acids, about 210 amino acids, about 220 amino acids, about 230 amino acids, about 240 amino acids, about 250 amino acids, about 260 amino acids, about 270 amino acids, about 280 amino acids, about 290 amino acids, about 300 amino acids, about 310 amino acids, about 320 amino acids, about 330 amino acids, about 340 amino acids, about 350 amino acids, about 360 amino acids, about 370 amino acids, about 380 amino acids, about 390 amino acids, about 400 amino acids, about 410 amino acids, about 420 amino acids, about 430 amino acids, about 440 amino acids, about 450 amino acids, about 460 amino acids, about 470 amino acids, about 480 amino acids, about 490 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, about 600 amino acids, about 610 amino acids, about 620 amino acids, about 630 amino acids, about 640 amino acids, about 650 amino acids, about 660 amino acids, about 670 amino acids, about 680 amino acids, about 690 amino acids, about 700 amino acids, about 710 amino acids, about 720 amino acids, about 730 amino acids, about 740 amino acids, about 750 amino acids, about 760 amino acids, about 770 amino acids, about 780 amino acids, about 790 amino acids, about 800 amino acids, about 810 amino acids, or about 820 amino acids in length).

In some examples, a desired gene product (e.g., a therapeutic gene product) is encoded by at least two different constructs. In some embodiments, each of at least two different constructs includes a different segment of an intron, where the intron includes a nucleotide sequence of an intron that is present in a target genomic DNA (e.g., an inner ear cell target genomic DNA (e.g., SLC26A4 genomic DNA) (e.g., any of the exemplary introns in SEQ ID NO: 3 described herein). In some embodiments, different intron segments overlap. In some embodiments, different intron segments overlap in sequence by at most about 12,000 nucleotides (e.g., at most about 100 nucleotides, at most about 200 nucleotides, at most about 300 nucleotides, at most about 600 nucleotides, at most about 700 nucleotides, at most about 800 nucleotides, at most about 900 nucleotides, at most about 1,000 nucleotides, at most about 1,100 nucleotides, at most about 1,200 nucleotides, at most about 1,300 nucleotides, at most about 1,400 nucleotides, at most about 1,500 nucleotides, at most about 1,600 nucleotides, at most about 1,700 nucleotides, at most about 1,800 nucleotides, at most about 1,900 nucleotides, at most about 2,000 nucleotides, at most about 2,100 nucleotides, at most about 2,200 nucleotides, at most about 2,300 nucleotides, at most about 2,400 nucleotides, at most about 2,500 nucleotides, at most about 2,600 nucleotides, at most about 2,700 nucleotides, at most about 2,800 nucleotides, at most about 2,900 nucleotides, at most about 3,000 nucleotides, at most about 3,100 nucleotides, at most about 3,200 nucleotides, at most about 3,300 nucleotides, at most about 3,400 nucleotides, at most about 3,500 nucleotides, at most about 3,600 nucleotides, at most about 3,700 nucleotides, at most about 3,800 nucleotides, at most about 3,900 nucleotides, at most about 4,000 nucleotides, at most about 4,100 nucleotides, at most about 4,200 nucleotides, at most about 4,300 nucleotides, at most about 4,400 nucleotides, at most about 4,500 nucleotides, at most about 4,600 nucleotides, at most about 4,700 nucleotides, at most about 4,800 nucleotides, at most about 4,900 nucleotides, at most about 5,000 nucleotides, at most about 5,100 nucleotides, at most about 5,200 nucleotides, at most about 5,300 nucleotides, at most about 5,400 nucleotides, at most about 5,500 nucleotides, at most about 5,600 nucleotides, at most about 5,700 nucleotides, at most about 5,800 nucleotides, at most about 5,900 nucleotides, at most about 6,000 nucleotides, at most about 6,100 nucleotides, at most about 6,200 nucleotides, at most about 6,300 nucleotides, at most about 6,400 nucleotides, at most about 6,500 nucleotides, at most about 6,600 nucleotides, at most about 6,700 nucleotides, at most about 6,800 nucleotides, at most about 6,900 nucleotides, at most about 7,000 nucleotides, at most about 7,100 nucleotides, at most about 7,200 nucleotides, at most about 7,300 nucleotides, at most about 7,400 nucleotides, at most about 7,500 nucleotides, at most about 7,600 nucleotides, at most about 7,700 nucleotides, at most about 7,800 nucleotides, at most about 7,900 nucleotides, at most about 8,000 nucleotides, at most about 8,100 nucleotides, at most about 8,200 nucleotides, at most about 8,300 nucleotides, at most about 8,400 nucleotides, at most about 8,500 nucleotides, at most about 8,600 nucleotides, at most about 8,700 nucleotides, at most about 8,800 nucleotides, at most about 8,900 nucleotides, at most about 9,000 nucleotides, at most about 9,100 nucleotides, at most about 9,200 nucleotides, at most about 9,300 nucleotides, at most about 9,400 nucleotides, at most about 9,500 nucleotides, at most about 9,600 nucleotides, at most about 9,700 nucleotides, at most about 9,800 nucleotides, at most about 9,900 nucleotides, at most about 10,000 nucleotides, at most about 10,100 nucleotides, at most about 10,200 nucleotides, at most about 10,300 nucleotides, at most about 10,400 nucleotides, at most about 10,500 nucleotides, at most about 10,600 nucleotides, at most about 10,700 nucleotides, at most about 10,800 nucleotides, at most about 10,900 nucleotides, at most about 11,000 nucleotides, at most about 11,100 nucleotides, at most about 11,200 nucleotides, at most about 11,300 nucleotides, at most about 11,400 nucleotides, at most about 11,500 nucleotides, at most about 11,600 nucleotides, at most about 11,700 nucleotides, at most about 11,800 nucleotides, at most about 11,900 nucleotides, or at most about 12,000 nucleotides) in length. In some embodiments, the overlapping nucleotide sequence in any two of the different constructs can include part or all of one or more exons of a target gene (e.g., an inner ear cell target gene (e.g., an SLC26A4 gene) (e.g., any one or more of the exemplary exons in SEQ ID NO: 3 described herein).

In some embodiments, a composition or system is or comprises two, three, four, or five different constructs. In compositions where the number of different constructs in the composition is two, the first of the two different constructs can include a coding sequence that encodes an N-terminal portion of a protein (e.g., pendrin protein), which may be referred to as a lead portion, a first construct, or a 5′ portion (e.g., an N-terminal portion of an inner ear cell protein, e.g., an N-terminal portion of a pendrin protein). In some examples, an N-terminal portion of the target gene is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) in length. In some examples, a first construct includes one or both of a promoter (e.g., any of the promoters described herein or known in the art) and a Kozak sequence (e.g., any of the exemplary Kozak sequences described herein or known in the art). In some examples, a first construct includes a promoter that is an inducible promoter, a constitutive promoter, or a tissue-specific promoter. In some examples, a second of the two different constructs includes a coding sequence that encodes a C-terminal portion of the protein, which may be referred to as a terminal portion, a second construct, or a 3′ portion (e.g., a C-terminal portion of an inner ear cell target protein, e.g., a C-terminal portion of a pendrin protein). In some examples, a C-terminal portion of the target protein is at least about 10 amino acids (e.g., at least about 10 amino acids, at least about 20 amino acids, at least about 30 amino acids, at least about 60 amino acids, at least about 70 amino acids, at least about 80 amino acids, at least about 90 amino acids, at least about 100 amino acids, at least about 110 amino acids, at least about 120 amino acids, at least about 130 amino acids, at least about 140 amino acids, at least about 150 amino acids, at least about 160 amino acids, at least about 170 amino acids, at least about 180 amino acids, at least about 190 amino acids, at least about 200 amino acids, at least about 210 amino acids, at least about 220 amino acids, at least about 230 amino acids, at least about 240 amino acids, at least about 250 amino acids, at least about 260 amino acids, at least about 270 amino acids, at least about 280 amino acids, at least about 290 amino acids, at least about 300 amino acids, at least about 310 amino acids, at least about 320 amino acids, at least about 330 amino acids, at least about 340 amino acids, at least about 350 amino acids, at least about 360 amino acids, at least about 370 amino acids, at least about 380 amino acids, at least about 390 amino acids, at least about 400 amino acids, at least about 410 amino acids, at least about 420 amino acids, at least about 430 amino acids, at least about 440 amino acids, at least about 450 amino acids, at least about 460 amino acids, at least about 470 amino acids, at least about 480 amino acids, at least about 490 amino acids, at least about 500 amino acids, at least about 510 amino acids, at least about 520 amino acids, at least about 530 amino acids, at least about 540 amino acids, at least about 550 amino acids, at least about 560 amino acids, at least about 570 amino acids, at least about 580 amino acids, at least about 590 amino acids, at least about 600 amino acids, at least about 610 amino acids, at least about 620 amino acids, at least about 630 amino acids, at least about 640 amino acids, at least about 650 amino acids, at least about 660 amino acids, at least about 670 amino acids, at least about 680 amino acids, at least about 690 amino acids, at least about 700 amino acids, at least about 710 amino acids, at least about 720 amino acids, at least about 730 amino acids, at least about 740 amino acids, at least about 750 amino acids, at least about 760 amino acids, at least about 770 amino acids, at least about 780 amino acids, at least about 790 amino acids, at least about 800 amino acids, at least about 810 amino acids, or at least about 820 amino acids) in length. In some examples, a second construct further includes a poly(A) sequence.

In some examples where the number of different constructs in the composition is two, an N-terminal portion encoded by one of the two constructs can include a portion including amino acid position 1 to about amino acid position 820, or any subrange of this range (e.g., amino acid 1 to at least about amino acid 10, amino acid 1 to at least about amino acid 20, amino acid 1 to at least about amino acid 30, amino acid 1 to at least about amino acid 60, amino acid 1 to at least about amino acid 70, amino acid 1 to at least about amino acid 80, amino acid 1 to at least about amino acid 90, amino acid 1 to at least about amino acid 100, amino acid 1 to at least about amino acid 110, amino acid 1 to at least about amino acid 120, amino acid 1 to at least about amino acid 130, amino acid 1 to at least about amino acid 140, amino acid 1 to at least about amino acid 150, amino acid 1 to at least about amino acid 160, amino acid 1 to at least about amino acid 170, amino acid 1 to at least about amino acid 180, amino acid 1 to at least about amino acid 190, amino acid 1 to at least about amino acid 200, amino acid 1 to at least about amino acid 210, amino acid 1 to at least about amino acid 220, amino acid 1 to at least about amino acid 230, amino acid 1 to at least about amino acid 240, amino acid 1 to at least about amino acid 250, amino acid 1 to at least about amino acid 260, amino acid 1 to at least about amino acid 270, amino acid 1 to at least about amino acid 280, amino acid 1 to at least about amino acid 290, amino acid 1 to at least about amino acid 300, amino acid 1 to at least about amino acid 310, amino acid 1 to at least about amino acid 320, amino acid 1 to at least about amino acid 330, amino acid 1 to at least about amino acid 340, amino acid 1 to at least about amino acid 350, amino acid 1 to at least about amino acid 360, amino acid 1 to at least about amino acid 370, amino acid 1 to at least about amino acid 380, amino acid 1 to at least about amino acid 390, amino acid 1 to at least about amino acid 400, amino acid 1 to at least about amino acid 410, amino acid 1 to at least about amino acid 420, amino acid 1 to at least about amino acid 430, amino acid 1 to at least about amino acid 440, amino acid 1 to at least about amino acid 450, amino acid 1 to at least about amino acid 460, amino acid 1 to at least about amino acid 470, amino acid 1 to at least about amino acid 480, amino acid 1 to at least about amino acid 490, amino acid 1 to at least about amino acid 500, amino acid 1 to at least about amino acid 510, amino acid 1 to at least about amino acid 520, amino acid 1 to at least about amino acid 530, amino acid 1 to at least about amino acid 540, amino acid 1 to at least about amino acid 550, amino acid 1 to at least about amino acid 560, amino acid 1 to at least about amino acid 570, amino acid 1 to at least about amino acid 580, amino acid 1 to at least about amino acid 590, amino acid 1 to at least about amino acid 600, amino acid 1 to at least about amino acid 610, amino acid 1 to at least about amino acid 620, amino acid 1 to at least about amino acid 630, amino acid 1 to at least about amino acid 640, amino acid 1 to at least about amino acid 650, amino acid 1 to at least about amino acid 660, amino acid 1 to at least about amino acid 670, amino acid 1 to at least about amino acid 680, amino acid 1 to at least about amino acid 690, amino acid 1 to at least about amino acid 700, amino acid 1 to at least about amino acid 710, amino acid 1 to at least about amino acid 720, amino acid 1 to at least about amino acid 730, amino acid 1 to at least about amino acid 740, amino acid 1 to at least about amino acid 750, amino acid 1 to at least about amino acid 760, amino acid 1 to at least about amino acid 770, amino acid 1 to at least about amino acid 780, amino acid 1 to at least about amino acid 790, amino acid 1 to at least about amino acid 800, amino acid 1 to at least about amino acid 810, or amino acid 1 to at least about amino acid 820) of an inner ear cell target protein (e.g., SEQ ID NO: 6 or 7). In some examples where the number of different constructs in the composition is two, an N-terminal portion of the precursor inner ear cell target protein can include a portion including at most amino acid position 1 to amino acid position 820 or any subrange of this range (e.g., amino acid 1 to at most about amino acid 10, amino acid 1 to at most about amino acid 20, amino acid 1 to at most about amino acid 30, amino acid 1 to at most about amino acid 60, amino acid 1 to at most about amino acid 70, amino acid 1 to at most about amino acid 80, amino acid 1 to at most about amino acid 90, amino acid 1 to at most about amino acid 100, amino acid 1 to at most about amino acid 110, amino acid 1 to at most about amino acid 120, amino acid 1 to at most about amino acid 130, amino acid 1 to at most about amino acid 140, amino acid 1 to at most about amino acid 150, amino acid 1 to at most about amino acid 160, amino acid 1 to at most about amino acid 170, amino acid 1 to at most about amino acid 180, amino acid 1 to at most about amino acid 190, amino acid 1 to at most about amino acid 200, amino acid 1 to at most about amino acid 210, amino acid 1 to at most about amino acid 220, amino acid 1 to at most about amino acid 230, amino acid 1 to at most about amino acid 240, amino acid 1 to at most about amino acid 250, amino acid 1 to at most about amino acid 260, amino acid 1 to at most about amino acid 270, amino acid 1 to at most about amino acid 280, amino acid 1 to at most about amino acid 290, amino acid 1 to at most about amino acid 300, amino acid 1 to at most about amino acid 310, amino acid 1 to at most about amino acid 320, amino acid 1 to at most about amino acid 330, amino acid 1 to at most about amino acid 340, amino acid 1 to at most about amino acid 350, amino acid 1 to at most about amino acid 360, amino acid 1 to at most about amino acid 370, amino acid 1 to at most about amino acid 380, amino acid 1 to at most about amino acid 390, amino acid 1 to at most about amino acid 400, amino acid 1 to at most about amino acid 410, amino acid 1 to at most about amino acid 420, amino acid 1 to at most about amino acid 430, amino acid 1 to at most about amino acid 440, amino acid 1 to at most about amino acid 450, amino acid 1 to at most about amino acid 460, amino acid 1 to at most about amino acid 470, amino acid 1 to at most about amino acid 480, amino acid 1 to at most about amino acid 490, amino acid 1 to at most about amino acid 500, amino acid 1 to at most about amino acid 510, amino acid 1 to at most about amino acid 520, amino acid 1 to at most about amino acid 530, amino acid 1 to at most about amino acid 540, amino acid 1 to at most about amino acid 550, amino acid 1 to at most about amino acid 560, amino acid 1 to at most about amino acid 570, amino acid 1 to at most about amino acid 580, amino acid 1 to at most about amino acid 590, amino acid 1 to at most about amino acid 600, amino acid 1 to at most about amino acid 610, amino acid 1 to at most about amino acid 620, amino acid 1 to at most about amino acid 630, amino acid 1 to at most about amino acid 640, amino acid 1 to at most about amino acid 650, amino acid 1 to at most about amino acid 660, amino acid 1 to at most about amino acid 670, amino acid 1 to at most about amino acid 680, amino acid 1 to at most about amino acid 690, amino acid 1 to at most about amino acid 700, amino acid 1 to at most about amino acid 710, amino acid 1 to at most about amino acid 720, amino acid 1 to at most about amino acid 730, amino acid 1 to at most about amino acid 740, amino acid 1 to at most about amino acid 750, amino acid 1 to at most about amino acid 760, amino acid 1 to at most about amino acid 770, amino acid 1 to at most about amino acid 780, amino acid 1 to at most about amino acid 790, amino acid 1 to at most about amino acid 800, amino acid 1 to at most about amino acid 810, or amino acid 1 to at most about amino acid 820) of an inner ear cell target protein (e.g., SEQ ID NO: 6 or 7)

In some examples where the number of different constructs in the composition is two, a C-terminal portion encoded by one of the two constructs can include a portion including the final amino acid (e.g., about amino acid position 820) to about amino acid position 1, or any subrange of this range (e.g., amino acid 820 to at least about amino acid 10, amino acid 820 to at least about amino acid 20, amino acid 820 to at least about amino acid 30, amino acid 820 to at least about amino acid 60, amino acid 820 to at least about amino acid 70, amino acid 820 to at least about amino acid 80, amino acid 820 to at least about amino acid 90, amino acid 820 to at least about amino acid 100, amino acid 820 to at least about amino acid 110, amino acid 820 to at least about amino acid 120, amino acid 820 to at least about amino acid 130, amino acid 820 to at least about amino acid 140, amino acid 820 to at least about amino acid 150, amino acid 820 to at least about amino acid 160, amino acid 820 to at least about amino acid 170, amino acid 820 to at least about amino acid 180, amino acid 820 to at least about amino acid 190, amino acid 820 to at least about amino acid 200, amino acid 820 to at least about amino acid 210, amino acid 820 to at least about amino acid 220, amino acid 820 to at least about amino acid 230, amino acid 820 to at least about amino acid 240, amino acid 820 to at least about amino acid 250, amino acid 820 to at least about amino acid 260, amino acid 820 to at least about amino acid 270, amino acid 820 to at least about amino acid 280, amino acid 820 to at least about amino acid 290, amino acid 820 to at least about amino acid 300, amino acid 820 to at least about amino acid 310, amino acid 820 to at least about amino acid 320, amino acid 820 to at least about amino acid 330, amino acid 820 to at least about amino acid 340, amino acid 820 to at least about amino acid 350, amino acid 820 to at least about amino acid 360, amino acid 820 to at least about amino acid 370, amino acid 820 to at least about amino acid 380, amino acid 820 to at least about amino acid 390, amino acid 820 to at least about amino acid 400, amino acid 820 to at least about amino acid 410, amino acid 820 to at least about amino acid 420, amino acid 820 to at least about amino acid 430, amino acid 820 to at least about amino acid 440, amino acid 820 to at least about amino acid 450, amino acid 820 to at least about amino acid 460, amino acid 820 to at least about amino acid 470, amino acid 820 to at least about amino acid 480, amino acid 820 to at least about amino acid 490, amino acid 820 to at least about amino acid 500, amino acid 820 to at least about amino acid 510, amino acid 820 to at least about amino acid 520, amino acid 820 to at least about amino acid 530, amino acid 820 to at least about amino acid 540, amino acid 820 to at least about amino acid 550, amino acid 820 to at least about amino acid 560, amino acid 820 to at least about amino acid 570, amino acid 820 to at least about amino acid 580, amino acid 820 to at least about amino acid 590, amino acid 820 to at least about amino acid 600, amino acid 820 to at least about amino acid 610, amino acid 820 to at least about amino acid 620, amino acid 820 to at least about amino acid 630, amino acid 820 to at least about amino acid 640, amino acid 820 to at least about amino acid 650, amino acid 820 to at least about amino acid 660, amino acid 820 to at least about amino acid 670, amino acid 820 to at least about amino acid 680, amino acid 820 to at least about amino acid 690, amino acid 820 to at least about amino acid 700, amino acid 820 to at least about amino acid 710, amino acid 820 to at least about amino acid 720, amino acid 820 to at least about amino acid 730, amino acid 820 to at least about amino acid 740, amino acid 820 to at least about amino acid 750, amino acid 820 to at least about amino acid 760, amino acid 820 to at least about amino acid 770, amino acid 820 to at least about amino acid 780, amino acid 820 to at least about amino acid 790, amino acid 820 to at least about amino acid 800, amino acid 820 to at least about amino acid 810, or amino acid 820 to at least about amino acid 820) of an inner ear cell target protein (e.g., SEQ ID NO: 6 or 7). In some examples where the number of different constructs in the composition is two, a C-terminal portion of the precursor inner ear cell target protein can include a portion including the final amino acid (e.g., about amino acid position 820) to at most about amino acid position 1, or any subrange of this range (e.g., amino acid 820 to at most about amino acid 10, amino acid 820 to at most about amino acid 20, amino acid 820 to at most about amino acid 30, amino acid 820 to at most about amino acid 60, amino acid 820 to at most about amino acid 70, amino acid 820 to at most about amino acid 80, amino acid 820 to at most about amino acid 90, amino acid 820 to at most about amino acid 100, amino acid 820 to at most about amino acid 110, amino acid 820 to at most about amino acid 120, amino acid 820 to at most about amino acid 130, amino acid 820 to at most about amino acid 140, amino acid 820 to at most about amino acid 150, amino acid 820 to at most about amino acid 160, amino acid 820 to at most about amino acid 170, amino acid 820 to at most about amino acid 180, amino acid 820 to at most about amino acid 190, amino acid 820 to at most about amino acid 200, amino acid 820 to at most about amino acid 210, amino acid 820 to at most about amino acid 220, amino acid 820 to at most about amino acid 230, amino acid 820 to at most about amino acid 240, amino acid 820 to at most about amino acid 250, amino acid 820 to at most about amino acid 260, amino acid 820 to at most about amino acid 270, amino acid 820 to at most about amino acid 280, amino acid 820 to at most about amino acid 290, amino acid 820 to at most about amino acid 300, amino acid 820 to at most about amino acid 310, amino acid 820 to at most about amino acid 320, amino acid 820 to at most about amino acid 330, amino acid 820 to at most about amino acid 340, amino acid 820 to at most about amino acid 350, amino acid 820 to at most about amino acid 360, amino acid 820 to at most about amino acid 370, amino acid 820 to at most about amino acid 380, amino acid 820 to at most about amino acid 390, amino acid 820 to at most about amino acid 400, amino acid 820 to at most about amino acid 410, amino acid 820 to at most about amino acid 420, amino acid 820 to at most about amino acid 430, amino acid 820 to at most about amino acid 440, amino acid 820 to at most about amino acid 450, amino acid 820 to at most about amino acid 460, amino acid 820 to at most about amino acid 470, amino acid 820 to at most about amino acid 480, amino acid 820 to at most about amino acid 490, amino acid 820 to at most about amino acid 500, amino acid 820 to at most about amino acid 510, amino acid 820 to at most about amino acid 520, amino acid 820 to at most about amino acid 530, amino acid 820 to at most about amino acid 540, amino acid 820 to at most about amino acid 550, amino acid 820 to at most about amino acid 560, amino acid 820 to at most about amino acid 570, amino acid 820 to at most about amino acid 580, amino acid 820 to at most about amino acid 590, amino acid 820 to at most about amino acid 600, amino acid 820 to at most about amino acid 610, amino acid 820 to at most about amino acid 620, amino acid 820 to at most about amino acid 630, amino acid 820 to at most about amino acid 640, amino acid 820 to at most about amino acid 650, amino acid 820 to at most about amino acid 660, amino acid 820 to at most about amino acid 670, amino acid 820 to at most about amino acid 680, amino acid 820 to at most about amino acid 690, amino acid 820 to at most about amino acid 700, amino acid 820 to at most about amino acid 710, amino acid 820 to at most about amino acid 720, amino acid 820 to at most about amino acid 730, amino acid 820 to at most about amino acid 740, amino acid 820 to at most about amino acid 750, amino acid 820 to at most about amino acid 760, amino acid 820 to at most about amino acid 770, amino acid 820 to at most about amino acid 780, amino acid 820 to at most about amino acid 790, amino acid 820 to at most about amino acid 800, amino acid 820 to at most about amino acid 810, or amino acid 820 to at most about amino acid 820, or any length sequence there between of an inner ear cell target protein (e.g., SEQ ID NO: 6 or 7).

In some embodiments, splice sites are involved in trans-splicing. In some embodiments, a splice donor site (Trapani et al. EMBO Mol. Med. 6(2):194-211, 2014, which is incorporated in its entirety herein by reference) follows the coding sequence in the N-terminal construct. In the C-terminal construct, a splice acceptor site may be subcloned just before the coding sequence for SLC26A4. In some embodiments, within the coding sequence, a silent mutation can be introduced, generating an additional site for restriction digestion.

In some embodiments, any of the constructs provided herein can be included in a composition suitable for administration to an animal for the amelioration of symptoms associated with syndromic and/or nonsyndromic hearing loss.

Pharmaceutical Compositions

Among other things, the present disclosure provides pharmaceutical compositions. In some embodiments compositions provided herein are suitable for administration to an animal for the amelioration of symptoms associated with syndromic and/or nonsyndromic hearing loss.

In some embodiments, pharmaceutical compositions of the present disclosure may comprise, e.g., a polynucleotide, e.g., one or more constructs, as described herein. In some embodiments, a pharmaceutical composition may comprise one or more AAV particles, e.g., one or more rAAV construct encapsidated by one or more AAV serotype capsids, as described herein.

In some embodiments, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some embodiments, formulations are in a dosage forms, such as injectable solutions, injectable gels, drug-release capsules, and the like.

In some embodiments, compositions of the present disclosure are formulated for intravenous administration. In some embodiments compositions of the present disclosure are formulated for intra-cochlear administration. In some embodiments, a therapeutic composition is formulated to comprise a lipid nanoparticle, a polymeric nanoparticle, a mini-circle DNA and/or a CELiD DNA.

In some embodiments, a therapeutic composition is formulated to comprise a synthetic perilymph solution. For example, in some embodiments, a synthetic perilymph solution includes 20-200 mM NaCl; 1-5 mM KCl; 0.1-10 mM CaCl₂); 1-10 mM glucose; and 2-50 mM HEPES, with a pH between about 6 and about 9. In some embodiments, a therapeutic composition is formulated to comprise a physiologically suitable solution. For example, in some embodiments, a physiologically suitable solution comprises commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some embodiments, alternative pluronic acids are utilized. In some embodiments, alternative ion concentrations are utilized.

In some embodiments, any of the pharmaceutical compositions described herein may further comprise one or more agents that promote the entry of a nucleic acid or any of the constructs described herein into a mammalian cell (e.g., a liposome or cationic lipid). In some embodiments, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some embodiments, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some embodiments, a composition provided herein can be, e.g., formulated to be compatible with their intended route of administration. A non-limiting example of an intended route of administration is local administration (e.g., intra-cochlear administration).

In some embodiments, a provided composition comprises one nucleic acid construct. In some embodiments, a provided composition comprises two or more different constructs. In some embodiments, a composition that include a single nucleic acid construct comprising a coding sequence that encodes a pendrin protein and/or a functional characteristic portion thereof. In some embodiments, compositions comprise a single nucleic acid construct comprising a coding sequence that encodes a pendrin protein and/or a functional characteristic portion thereof, which, when introduced into a mammalian cell, that coding sequence is integrated into the genome of the mammalian cell. In some embodiments, a composition comprising at least two different constructs, e.g., constructs comprise coding sequences that encode a different portion of a pendrin protein, the constructs can be combined to generate a sequence encoding an active pendrin protein (e.g., a full-length pendrin protein) in a mammalian cell, and thereby treat associated syndromic or nonsyndromic sensorineural hearing loss in a subject in need thereof.

Also provided are kits including any of the compositions described herein. In some embodiments, a kit can include a solid composition (e.g., a lyophilized composition including the at least two different constructs described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

Dosing and Volume of Administration

In some embodiments, a composition disclosed herein, e.g., one or a plurality of AAV vectors disclosed herein, is administered as a single dose or as a plurality of doses.

In some embodiments, a composition disclosed herein is administered as a single dose. In some embodiments, a composition disclosed herein is administered as a plurality of doses, e.g., 2, 3, 4, 5, 6, 7, 8, 9 or 10 doses.

In some embodiments, a composition disclosed herein (e.g., a composition comprising one or a plurality of rAAV constructs disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some embodiments, a composition disclosed herein is administered at a volume of about 0.01 mL. In some embodiments, a composition disclosed herein is administered at a volume of about 0.02 mL. In some embodiments, a composition disclosed herein is administered at a volume of about 0.03 mL. In some embodiments, a composition disclosed herein is administered at a volume of about 0.04 mL. In some embodiments, a composition disclosed herein is administered at a volume of about 0.05 mL. In some embodiments, a composition disclosed herein is administered at a volume of about 0.06 mL. In some embodiments, a composition disclosed herein is administered at a volume of about 0.07 mL. In some embodiments, a composition disclosed herein is administered at a volume of about 0.08 mL. In some embodiments, a composition disclosed herein is administered at a volume of about 0.09 mL. In some embodiments, a composition disclosed herein is administered at a volume of about 1.00 mL. In some embodiments, a composition disclosed herein is administered at a volume of about 1.10 mL. In some embodiments, a composition disclosed herein is administered at a volume of about 1.20 mL. In some embodiments, a composition disclosed herein is administered at a volume of about 1.30 mL. In some embodiments, a composition disclosed herein is administered at a volume of about 1.40 mL. In some embodiments, a composition disclosed herein is administered at a volume of about 1.50 mL. In some embodiments, a composition disclosed herein is administered at a volume of about 1.60 mL. In some embodiments, a composition disclosed herein is administered at a volume of about 1.70 mL. In some embodiments, a composition disclosed herein is administered at a volume of about 1.80 mL. In some embodiments, a composition disclosed herein is administered at a volume of about 1.90 mL. In some embodiments, a composition disclosed herein is administered at a volume of about 2.00 mL.

In some embodiments, a composition disclosed herein (e.g., a composition comprising one or a plurality of rAAV constructs disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some embodiments a composition disclosed herein (e.g., a composition comprising one or a plurality of rAAV constructs disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some embodiments, an animal cell is a human cell (e.g., a human supporting cell or a human hair cell). In other embodiments, an animal cell is a non-human mammal (e.g., Simian cell, Felidae cell, Canidae cell etc.). A person skilled in the art will appreciate that the nucleic acids and constructs described herein can be introduced into any animal cell (e.g., the supporting or hair cells of any animal suitable for veterinary intervention). Non-limiting examples of constructs and methods for introducing constructs into animal cells are described herein.

In some embodiments, an animal cell can be any cell of the inner ear, including hair and/or supporting cells. Non-limiting examples such cells include: Hensen's cells, Deiters' cells, cells of the endolymphatic sac and duct, transitional cells in the saccule, utricle, and ampulla, inner and outer hair cells, spiral ligament cells, spiral ganglion cells, spiral prominence cells, external saccule cells, marginal cells, intermediate cells, basal cells, inner pillar cells, outer pillar cells, Claudius cells, inner border cells, inner phalangeal cells, or cells of the stria vascularis.

In some embodiments, an animal cell is a specialized cell of the cochlea. In some embodiments, an animal cell is a hair cell. In some embodiments, an animal cell is a cochlear inner hair cell or a cochlear outer hair cell. In some embodiments, an animal cell is a cochlear inner hair cell. In some embodiments, an animal cell is a cochlear outer hair cell.

In some embodiments, an animal cell is in vitro. In some embodiments, an animal cell is of a cell type which is endogenously present in an animal, e.g., in a primate and/or human. In some embodiments, an animal cell is an autologous cell obtained from an animal and cultured ex vivo.

### Genetically Modified Model Animals

The present disclosure also provides an animal (e.g., a mammal, e.g., a rodent, e.g., a mouse or rat) that is suitable for testing any of the nucleic acids, constructs, or compositions described herein. In some embodiments, a nucleic acid and/or construct described herein can be introduced into any animal cell (e.g., the supporting or hair cells of any animal suitable for veterinary intervention). However, some animals will be better suited for controlled audiological analysis experiments. Non-limiting examples of animals suitable for introduction of constructs and methods for analyzing said constructs are described herein.

In some embodiments, the present disclosure provides a method of making an animal comprising genetically modifying the animal so that it comprises a mutant Slc26a4 gene. In some embodiments, an endogenous Slc26a4 gene is modified to so that a polypeptide encoded by the Slc26a4 gene comprises an L236P mutation when compared to SEQ ID NO: 56. In some embodiments, an endogenous Slc26a4 gene is modified to so that a polypeptide encoded by the Slc26a4 gene comprises or consists of a sequence according to SEQ ID NO: 57.

In some embodiments, an endogenous Slc26a4 gene is knocked-out or inhibited in a genetically modified animal. In some embodiments, a mutant Slc26a4 gene is knocked-in or added back to a genetically modified animal. In some embodiments, a mutant Slc26a4 gene encodes a polypeptide that comprises an L236P mutation when compared to SEQ ID NO: 56. In some embodiments, a mutant Slc26a4 gene encodes a polypeptide comprising or consisting of a sequence according to SEQ ID NO: 57.

In some embodiments, a mutant Slc26a4 gene is present at an endogenous loci of an Slc26a4 gene. In some embodiments, a genetically modified animal is homozygous for a mutant Slc26a4 gene. In some embodiments, a genetically modified animal is heterozygous for a mutant Slc26a4 gene.

Among other things, in some embodiments, the present disclosure provides a genetically modified mouse whose genome comprises a modified Slc26a4 gene that recapitulates one or more known human disease genotypes. In some embodiments, a mutant Slc26a4 gene encodes a polypeptide with an L236P mutation when compared to SEQ ID NO: 56. In some embodiments, a mutant Slc26a4 gene mutation encodes a polypeptide according to SEQ ID NO: 57.

In some embodiments, the present disclosure provides a genetically modified animal suitable for use in audiological analysis experiments. In some embodiments, a genetically modified animal is a genetically modified mouse. In some embodiments, a genetically modified mouse suitable for use in audiological analysis experiments is of FVB strain. In some embodiments, the genetically modified mouse suitable for use in audiological analysis experiments is of FVB, 129/Sv-+p+Tyr-c+Mgf-SIJ/J, A/HeJ, AKR/J, BALB/cByJ, BALB/cJ, BDP/J, BXSB/MpJ, C3H/HeJ, C3H/HeOuJ, C3HeB/FeJ, C57BL/10J, C57BL/10SnJ, C57BL/6ByJ, CASA/RK, CAST/Ei, CBA/J, CZECH II/Ei, DBA/2HaSmn, FVB/NJ, HRS/J hrl+, MOLD/Rk, MOLF/Ei, MOLG/Dn, NON/LtJ, NZB/B1NJ, NZO/NIJ, NZW/LacJ, PERA/camEi, PERC/Ei, PL/J, RBA/Dn, RBF/DnJ, RF/J, RHJ/Le hrrh-J/+, RIIIS/J, SEC/1ReJ, SENCARC/PtJ, SF/CamEi, SHR/GnEi, SJL/J, SM/J, SPRET/Ei, ST/bJ, or SWR/J strain (e.g., as described in Zheng et al., Assessment of hearing in 80 inbred strains of mice by ABR threshold analysis. Hear Res. 1999; which is incorporated in its entirety herein by reference). In some embodiments, a mutation comprised in a genetically modified mouse is created in a background suitable for audiological analysis experiments. In some embodiments, the genetically modified mouse is of a mouse strain suitable for use in coordination analysis experiments. In some embodiments, the genetically modified mouse is not of CBA/CaJ or CBA/J strain.

In some embodiments, a genetically modified animal is treated with AAV particles, constructs, or compositions described herein. In some embodiments, a genetically modified animal is injected with an AAV particle as described herein. In some embodiments, a genetically modified animal is injected with a composition as described herein. In some embodiments, an injection is performed through a perforation in a round window membrane in an email.

The present disclosure provides uses of a genetically modified animal as described herein for assessing and/or characterizing an AAV particle as described herein.

The present disclosure provides uses of a genetically modified animal as described herein for assessing and/or characterizing a composition as described herein.

In some embodiments, a use provided herein can be part of a release test.

### Methods

Among other things, the present disclosure provides methods. In some embodiments, a method comprises introducing a composition as described herein into the inner ear (e.g., a cochlea) of a subject. For example, provided herein are methods that in some embodiments include administering to an inner ear (e.g., cochlea) of a subject (e.g., an animal, e.g., a mammal, e.g., a primate, e.g., a human) a therapeutically effective amount of any composition described herein. In some embodiments of any of these methods, the subject has been previously identified as having a defective inner ear cell target gene (e.g., a supporting and/or hearing cell target gene having a mutation that results in a decrease in the expression and/or activity of a supporting and/or hearing cell target protein encoded by the gene). Some embodiments of any of these methods further include, prior to the introducing or administering step, determining that the subject has a defective inner ear cell target gene. Some embodiments of any of these methods can further include detecting a mutation in an inner ear cell target gene in a subject. Some embodiments of any of the methods can further include identifying or diagnosing a subject as having nonsyndromic or syndromic sensorineural hearing loss.

In some embodiments, provided herein are methods of correcting an inner ear cell target gene defect (e.g., a defect in SLC26A4 gene) in an inner ear of a subject, e.g., an animal, e.g., a mammal, e.g., a primate, e.g., a human. In some embodiments, methods include administering to the inner ear of a subject a therapeutically effective amount of any of the compositions described herein, where the administering repairs and or ameliorates the inner ear cell target gene defect in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of increasing the expression level of an inner ear cell target protein in any subset of inner ear cells of a subject (e.g., an animal, e.g., a mammal, e.g., a primate, e.g., a human,) that include: administering to the inner ear of the subject a therapeutically effective amount of any of the compositions described herein, where the administering results in an increase in the expression level of the inner ear cell target protein (e.g., pendrin protein) in any cell subset of the inner ear of a subject. In some embodiments, the inner ear target cell may be a sensory cell, e.g., a hair cell, and/or a non-sensory cell, e.g., a supporting cell, and/or all or any subset of inner ear cells.

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the compositions described herein.

Also provided herein are methods of restoring synapses and/or preserving spiral ganglion nerves in a subject identified or diagnosed as having an inner ear disorder that include: administering to the inner ear of the subject a therapeutically effective amount of any of the compositions described herein.

Also provided herein are methods of reducing the size of, and/or restoring the vestibular aqueduct to an appropriate size. Also provided herein are methods of restoring endolymphatic pH to an appropriate and/or acceptable level in a subject identified or diagnosed as having an inner ear disorder that include: administering to the inner ear of the subject a therapeutically effective amount of any of the compositions described herein.

Also provided herein are methods that include administering to an inner ear of a subject a therapeutically effective amount of any of the compositions described herein.

Also provided herein are surgical methods for treatment of hearing loss (e.g., nonsyndromic sensorineural hearing loss or syndromic sensorineural hearing loss). In some embodiments, the methods include the steps of: introducing into a cochlea of a subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any composition described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

In some embodiments, technologies of the present disclosure are used to treat subjects with or at risk of hearing loss. For example, in some embodiments, a subject has an autosomal recessive hearing loss attributed to at least one pathogenic variant of SLC26A4. It will be understood by those in the art that many different mutations in SLC26A4 can result in a pathogenic variant. In some such embodiments, a pathogenic variant causes or is at risk of causing hearing loss.

In some embodiments, a subject experiencing hearing loss will be evaluated to determine if and where one or more mutations may exist that may cause hearing loss. In some such embodiments, the status of SLC26A4 gene products or function (e.g., via protein or sequencing analyses) will be evaluated. In some embodiments of any of the methods described herein, the subject or animal is a mammal, in some embodiments the mammal is a domestic animal, a farm animal, a zoo animal, a non-human primate, or a human. In some embodiments of any of the methods described herein, the animal, subject, or mammal is an adult, a teenager, a juvenile, a child, a toddler, an infant, or a newborn. In some embodiments of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some embodiments a subject (e.g., an animal, e.g., a mammal, e.g., a human) has or is at risk of developing syndromic or nonsyndromic sensorineural hearing loss. In some embodiments a subject (e.g., an animal, e.g., a mammal, e.g., a human) has been previously identified as having a mutation in an SLC26A4 gene. In some embodiments a subject (e.g., an animal, e.g., a mammal, e.g., a human) has any of the mutations in an SLC26A4 gene that are described herein or are known in the art to be associated with syndromic or nonsyndromic sensorineural hearing loss.

In some embodiments, a subject (e.g., an animal, e.g., a mammal, e.g., a human) has been identified as being a carrier of a mutation in an SLC26A4 gene (e.g., via genetic testing). In some embodiments, a subject (e.g., an animal, e.g., a mammal, e.g., a human) has been identified as having a mutation in an SLC26A4 gene and has been diagnosed with syndromic or nonsyndromic sensorineural hearing loss. In some embodiments, a subject (e.g., an animal, e.g., a mammal, e.g., a human) has been identified as having syndromic or nonsyndromic sensorineural hearing loss.

In some embodiments, a subject (e.g., an animal, e.g., a mammal, e.g., a human) has been identified as being at risk of hearing loss (e.g., at risk of being a carrier of a gene mutation, e.g., an SLC26A4 mutation). In some such embodiments, a subject (e.g., an animal, e.g., a mammal, e.g., a human) may have certain risk factors of hearing loss or risk of hearing loss (e.g., known parental carrier, afflicted sibling, or symptoms of hearing loss). In some such embodiments, a subject (e.g., an animal, e.g., a mammal, e.g., a human) has been identified as being a carrier of a mutation in an SLC26A4 gene (e.g., via genetic testing) that has not previously been identified (i.e., is not a published or otherwise known variant of SLC26A4). In some such embodiments, identified mutations may be novel (i.e., not previously described in the literature), and methods of treatment for a subject suffering from or susceptible to hearing loss will be personalized to the mutation(s) of the particular patient.

In some embodiments, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments of any method provided herein, two or more doses of any composition described herein are introduced or administered into a cochlea of a subject. Some embodiments of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some embodiments of any method provided herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, in some embodiments, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any method provided herein, a subject has or is at risk of developing syndromic or nonsyndromic sensorineural hearing loss. In some embodiments of any method provided herein, a subject has been previously identified as having a mutation in an inner ear cell target gene, a gene which may be expressed in supporting cells and/or hair cells.

In some embodiments of any method provided herein, a subject has been identified as being a carrier of a mutation in an inner ear cell target gene (e.g., via genetic testing). In some embodiments of any method provided herein, a subject has been identified as having a mutation in an inner ear cell target gene and has been diagnosed with hearing loss (e.g., nonsyndromic sensorineural hearing loss or syndromic sensorineural hearing loss, e.g., Pendred syndrome or DFNB4). In some embodiments of any of the methods described herein, the subject has been identified as having hearing loss (e.g., nonsyndromic sensorineural hearing loss or syndromic sensorineural hearing loss). In some embodiments, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some embodiments, a subject cell is in vitro. In some embodiments, a subject cell is originally obtained from a subject and is cultured ex vivo. In some embodiments, a subject cell has previously been determined to have a defective inner ear cell target gene. In some embodiments, a subject cell has previously been determined to have a defective hair cell target gene. In some embodiments, a subject cell has previously been determined to have a defective supporting cell target gene.

In some embodiments of these methods, following treatment e.g., one or two or more administrations of compositions described herein, there is an increase in expression of an active inner ear cell target protein (e.g., pendrin protein). In some embodiments, an increase in expression of an active inner ear target protein as described herein (e.g., pendrin protein) is relative to a control level, e.g., as compared to the level of expression of an inner ear cell target protein prior to introduction of the compositions comprising any construct(s) as described herein.

Methods of detecting expression and/or activity of a target protein (e.g., pendrin protein) are known in the art. In some embodiments, a level of expression of an inner ear cell target protein can be detected directly (e.g., detecting inner ear cell target protein or target mRNA. Non-limiting examples of techniques that can be used to detect expression and/or activity of a target RNA or protein (e.g., an SLC26A4 gene product and/or pendrin protein or functional characteristic portion thereof) directly include: real-time PCR, Western blotting, immunoprecipitation, immunohistochemistry, mass spectrometry, or immunofluorescence. In some embodiments, expression of an inner ear cell target protein can be detected indirectly (e.g., through functional hearing tests).

Devices, Administration, and Surgical Methods

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some embodiments, a medical device includes a plurality of micro-needles.

Also provided herein are surgical methods for treatment of hearing loss (e.g., nonsyndromic sensorineural hearing loss or syndromic sensorineural hearing loss). In some embodiments, a method the steps of: introducing into a cochlea of a subject a first incision at a first incision point; and administering intra-cochlearly a therapeutically effective amount of any of the compositions provided herein. In some embodiments, a composition is administered to a subject at the first incision point. In some embodiments, a composition is administered to a subject into or through the first incision.

In some embodiments of any method provided herein, any of the compositions described herein is administered to the subject into or through the cochlea oval window membrane. In some embodiments of any method provided herein, any of the compositions described herein is administered to the subject into or through the cochlea round window membrane. In some embodiments of any method provided herein, the composition is administered using a medical device capable of creating a plurality of incisions in the round window membrane. In some embodiments, a medical device includes a plurality of micro-needles. In some embodiments, a medical device includes a plurality of micro-needles including a generally circular first aspect, where each micro-needle has a diameter of at least about 10 microns. In some embodiments, a medical device includes a base and/or a reservoir capable of holding a composition. In some embodiments, a medical device includes a plurality of hollow micro-needles individually including a lumen capable of transferring a composition. In some embodiments, a medical device includes a means for generating at least a partial vacuum.

In some embodiments, a composition disclosed herein is formulated as a sterile suspension for intracochlear administration. In some embodiments, a composition comprises constructs in an amount of at least 1E11, at least 5E11, at least 1E12, at least 5E12, at least 1E13, at least 2E13, at least 3E13, at least 4E13, at least 5E13, at least 6E13, at least 7E13, at least 8E13, at least 9E13, or at least 1E14 vector genomes (vg) per milliliter (mL). In some embodiments, a composition comprises constructs in an amount of at most 1E15, at most 5E14, at most 1E14, at most 5E13, at most 1E13, at most 9E12, at most 8E12, at most 7E12, at most 6E12, at most 5E12, at most 4E12, at most 3E12, at most 2E12, or at most 1E12 vector genomes (vg) per milliliter (mL). In some embodiments, a composition comprises constructs in an amount of 1E12 to 1E13, 5E12 to 5E13, or 1E13 to 2E13 vector genomes (vg) per milliliter (mL).

In some embodiments, a composition disclosed herein is administered in the surgical suite under controlled aseptic conditions by a board-certified surgeon experienced in performing otologic surgery. In some embodiments, an administration procedure is a microscope- or endoscope-assisted transcanal exploratory tympanotomy and laser-assisted microstapedotomy, followed by a round window injection to deliver 0.09 mL of solution containing a composition disclosed herein. Transcanal exploratory tympanotomy is a common procedure used to expose the structures of the middle ear; transcanal exploratory tympanotomy is often accompanied by laser-assisted stapedectomy (removal of the stapes footplate) or stapedotomy (creating a hole in the stapes footplate), e.g., for patients with otosclerosis. In some embodiment, the approximately 0.25 mm vent hole in the stapes footplate (made using an otologic laser) serves to prevent a potential deleterious rise in intralabyrinthine pressure.

In some embodiments, disclosed herein is a sterile, one-time use delivery device to administer a composition disclosed herein to the perilymph fluid of the inner ear through the round window membrane with a vent located in the stapes footplate. In some embodiments, this custom device affords advantages over available materials, both with respect to potential for safety and efficacy of a therapeutic agent, as it was specifically designed for the intracochlear route of administration. In some embodiments, design elements of the delivery device include: maintenance of sterility of injected fluid; minimization of air bubbles introduced to the inner ear; ability to precisely deliver small volumes at a controlled flow rate (coupled with the use of a standard pump); allowance for visualization of round window membrane and delivery through the external auditory canal by the surgeon; minimization of damage to the round window membrane, or to cochlear structures beyond the round window membrane; and/or minimization of efflux back out through round window membrane.

In some embodiments, any of the methods disclosed herein comprise a dose-escalation study to assess safety and tolerability in subjects, e.g., mammals, e.g., humans, e.g., patients, e.g., patients with DFNB4 or Pendred syndrome symptoms. In some embodiments, a composition disclosed herein is administered at a dosing regimen disclosed herein. In some embodiments, the dosing regimen comprises either unilateral or bilateral intracochlear administrations of a dose, e.g., as described herein, of a composition disclosed herein. In some embodiments, the dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some embodiments, the dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some embodiments, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

In some embodiments of any method provided herein, two or more doses of any composition described herein are introduced or administered into a cochlea of a subject. Some embodiments of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some embodiments of any method provided herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, a subject cell is in-vitro. In some embodiments, a subject cell is originally obtained from a subject and is cultured ex-vivo. In some embodiments, a subject cell is considered otherwise healthy, and is cultured and expanded ex-vivo. In some embodiments, a subject cell has previously been determined to have a defective inner ear cell target gene. In some embodiments, a subject cell has previously been determined to have a defective hair cell target gene. In some embodiments, a subject cell has previously been determined to have a defective supporting cell target gene.

In some embodiments, when the subject is a rodent, e.g., a mouse, a surgical approach described in Yoshimura et al., 2018, which comprises delivery through the round window membrane with fenestration of the posterior semicircular canal, which has demonstrated robust and reliable transduction, independent of the age of the animal at the time of injection is used (Yoshimura 2018, incorporated herein in its entirety by reference). In brief, a postauricular incision is made to access the temporal bone. A portion of the sternocleidomastoid muscle is divided to expose the otic bulla. A 0.5 to 0.6 mm diameter otologic drill is used to make a small hole in the bulla; the hole is then widened to visualize the stapedial artery and the round window membrane. Fenestration of the posterior semicircular canal is performed with the otologic drill (0.5 to 0.6 mm diameter) to serve as a vent the inner ear during cochlear administration. The round window membrane is penetrated with the mouse delivery device, which consists of a borosilicate capillary pipette and a 10 μL Hamilton syringe, and 1 μL of solution comprising viral particles (approximately 40 to 50% of the total inner ear volume) is delivered through the round window membrane, into the scala tympani, at rate of 300 nL/min.

In some embodiments, when the subject is a NHP, a postauricular incision is made and dissection of the soft tissue is performed down to the level of the periosteum. In some embodiments, the periosteum is incised and elevated to expose the mastoid bone. A cortical mastoidectomy is performed with a combination of high-speed cutting and diamond drill burs. The facial recess is then opened, allowing for adequate round window and oval window (OW) visualization. Fenestration of the stapes footplate in the OW is performed using a Rosen needle. As with other models, the fenestration allows for injection of a larger volume without damage to the inner ear; additionally, venting allows solution comprising rAAV particles to flow toward the apex of the cochlea. Thirty L of solution comprising rAAV particles (approximately 40 to 50% of the total inner ear volume) is delivered through the round window membrane at rate 30 μL/min.

In some embodiments, when the subject is a mammal, e.g., a human, a less invasive approach through the external auditory canal is used, e.g., since the relevant structures are relatively large, even at birth. In some embodiments, the clinical administration procedure is a transcanal exploratory tympanotomy and laser-assisted microstapedotomy (using a potassium titanyl phosphate [KTP] or C02 otologic laser to place a small vent hole [approximately 0.25 mm] in the stapes footplate), followed by a round window injection to deliver about 0.09 mL (or 90 μL, approximately 40 to 50% of the total inner ear volume) of solution comprising a composition disclosed herein, e.g., rAAV-SLC26A4 particles, through the round window membrane within a three-minute period. In some embodiments, venting serves to prevent a potential deleterious rise in intralabyrinthine pressure. Transcanal exploratory tympanotomy is a common procedure used to expose the structures of the middle ear; transcanal exploratory tympanotomy is often accompanied by laser-assisted stapedectomy (removal of the stapes footplate) or stapedotomy (creating a hole in the stapes footplate), e.g., for patients with otosclerosis.

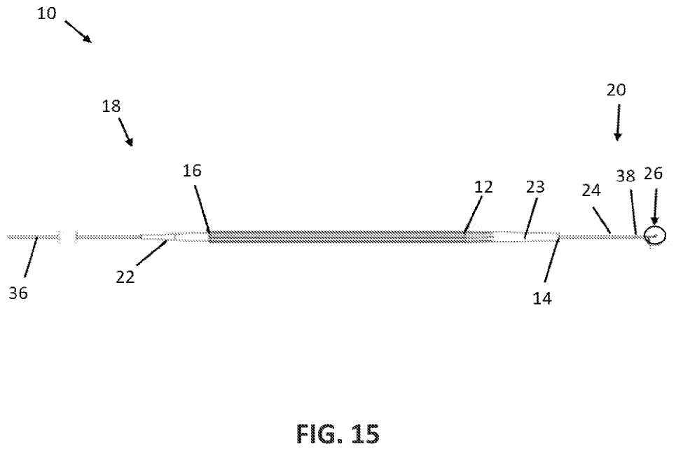

In some embodiments, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 15-18 (or microcatheter) to deliver a composition disclosed herein at a controlled flow rate and in a fixed volume, via the round window membrane.

In some embodiments, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some embodiments, absence of venting during surgery may result in lower AAV vector cochlear cell transduction rates when compared to AAV vector cochlear cell transduction rates following surgeries performed with venting. In some embodiments, venting facilitates transduction rates of about 75-100% of IHCs throughout the cochlea. In some embodiments, venting permits IHC transduction rates of about 50-70%, about 60-80%, about 70-90%, or about 80-100% at the base of the cochlea. In some embodiments, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some embodiments, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some embodiments, the flow rate is about 20 L/min, about 30 μL/min, about 40 L/min, about 50 μL/min, about 60 μL/min, about 70 μL/min, about 80 μL/min, about 90 μL/min or about 100 μL/min. In some embodiments, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some embodiments, the total volume of a composition disclosed herein that flows to the inner ear may be about 0.09 mL, or from about 0.08 mL to about 0.10 mL, or from about 0.07 mL to about 0.11 mL. In some embodiments, the total volume of a composition disclosed herein equates to from about 40% to about 50% of the volume of the inner ear.

Once the delivery has been completed, the device may be removed. In some embodiments, a device described herein, may be configured as a single-use disposable product. In other embodiments, a device described herein may be configured as a multi-use, sterilizable product, for example, with a replaceable and/or sterilizable needle sub-assembly. Single use devices may be appropriately discarded (for example, in a biohazard sharps container) after administration is complete.

In some embodiments, a composition disclosed herein can be administered to a subject with a surgical procedure. In some embodiments, administration, e.g., via a surgical procedure, comprises injecting a composition disclosed herein via a delivery device as described herein into the inner ear. In some embodiments, a surgical procedure disclosed herein comprises performing a transcanal tympanotomy; performing a laser-assisted micro-stapedotomy; and injecting a composition disclosed herein via a delivery device as described herein into the inner ear.

In some embodiments, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some embodiments, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some embodiments, performing a laser-assisted micro-stapedotomy includes using a KTP otologic laser and/or a CO2 otologic laser.

As another example, a composition disclosed herein is administered using a device and/or system specifically designed for intracochlear route of administration. In some embodiments, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

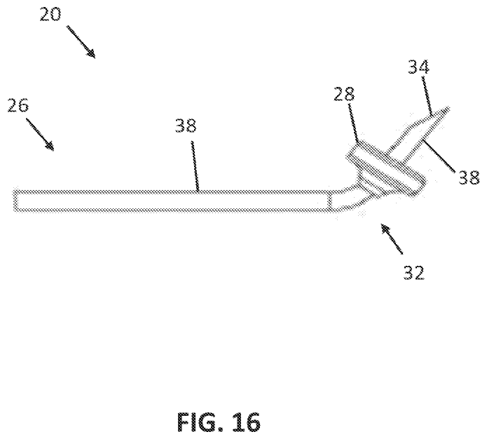

An exemplary device for use in any of the methods disclosed herein is described in FIGS. 15-18. FIG. 15 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 16) with stopper 28 (shown in FIG. 16) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 16) disposed there within.

Referring still to FIG. 15, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some embodiments, may be coupled to a control system and/or power supply (not shown)). In some embodiments, the bent needle 38 (shown in FIG. 16) extends from the distal end 20, through the telescoping hypotube needle support 24, through the tapered portion 23, through the knurled handle 12, and through the strain relief feature 22 and fluidly connects directly to the tubing 36. In other embodiments, the bent needle 38 fluidly connects with the hollow interior of the knurled handle (for example, via the telescoping hypotube needle support 24) which in turn fluidly connects at a proximal end 16 with tubing 36. In embodiments where the bent needle 38 does not extend all the way through the interior of the device 10, the contact area (for example, between overlapping nested hypotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

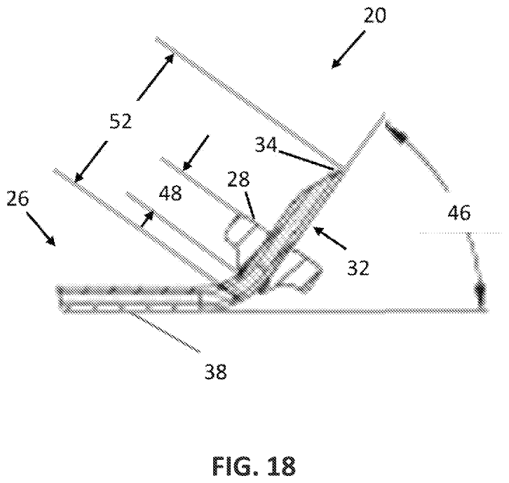

FIG. 16 illustrates a sideview of the bent needle sub-assembly 26, according to aspects of the present disclosed embodiments. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow there through. The angle 46 (as shown in FIG. 18) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

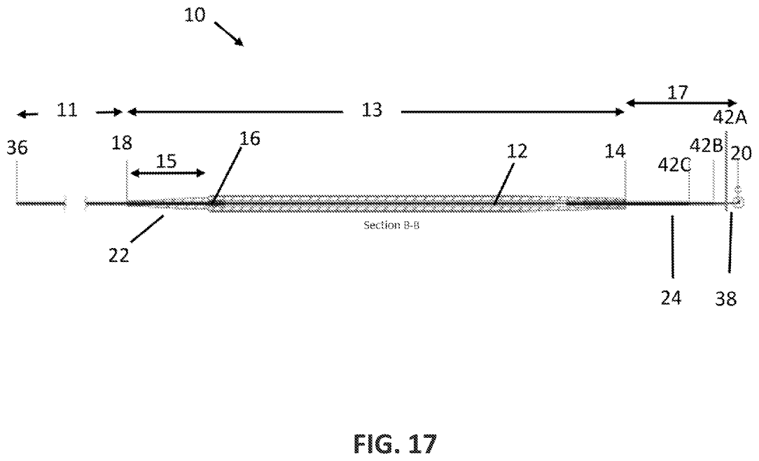

FIG. 17 illustrates a perspective view of exemplary device 10 for delivering fluid to an inner ear. Tubing 36 may be from about 1300 mm in length (dimension 11 in FIG. 17) to about 1600 mm, or from about 1400 mm to about 1500 mm, or from about 1430 mm to about 1450 mm. Strain release feature 22 may be from about 25 mm to about 30 mm in length (dimension 15 in FIG. 17), or from about 20 mm to about 35 mm in length. Handle 12 may be about 155.4 mm in length (dimension 13 in FIG. 17), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 31) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 18 illustrates a perspective view of bent needle sub-assembly 26 coupled to the distal end 20 of device 10, according to aspects of the present disclosed embodiments. As shown in FIG. 18, bent needle sub-assembly 26 may include a needle 38 coupled to a bent portion 32. In other embodiments, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 18, bent needle sub-assembly 26 may also include a stopper 28 disposed around bent portion 32. FIG. 18 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other embodiments, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 18 shows that stopper 28 may have a geometry that is cylindrical, disk-shaped, and/or dome-shaped. A person of ordinary skill will appreciate that other geometries could be used.

Evaluating Hearing Loss and Recovery

In some embodiments, hearing function is determined using auditory brainstem response measurements (ABR). In some embodiments, hearing is tested by measuring distortion product optoacoustic emissions (DPOAEs). In some such embodiments, measurements are taken from one or both ears of a subject. In some such embodiments, recordings are compared to prior recordings for the same subject and/or known thresholds on such response measurements used to define, e.g., hearing loss versus acceptable hearing ranges to be defined as normal hearing. In some embodiments, a subject has ABR and/or DPOAE measurements recorded prior to receiving any treatment. In some embodiments, a subject treated with one or more technologies described herein will have improvements on ABR and/or DPOAE measurements after treatment as compared to before treatment. In some embodiments, ABR and/or DPOAE measurements are taken after treatment is administered and at regular follow-up intervals post-treatment.

In some embodiments, hearing function is determined using speech pattern recognition or is determined by a speech therapist. In some embodiments, hearing function is determined by pure tone testing. In some embodiments, hearing function is determined by bone conduction testing. In some embodiments, hearing function is determined by acoustic reflex testing. In some embodiments hearing function is determined by tympanometry. In some embodiments, hearing function is determined by any combination of hearing analysis known in the art. In some such embodiments, measurements are taken holistically, and/or from one or both ears of a subject. In some such embodiments, recordings and/or professional analysis are compared to prior recordings and/or analysis for the same subject and/or known thresholds on such response measurements used to define, e.g., hearing loss versus acceptable hearing ranges to be defined as normal hearing. In some embodiments, a subject has speech pattern recognition, pure tone testing, bone conduction testing, acoustic reflex testing and/or tympanometry measurements and/or analysis conducted prior to receiving any treatment. In some embodiments a subject treated with one or more technologies described herein will have improvements on speech pattern recognition, pure tone testing, bone conduction testing, acoustic reflex testing and/or tympanometry measurements after treatment as compared to before treatment. In some embodiments, speech pattern recognition, pure tone testing, bone conduction testing, acoustic reflex testing and/or tympanometry measurements are taken after treatment is administered and at regular follow-up intervals post-treatment.

In some embodiments, hearing function in both a treated and a contralateral control ear may be significantly altered as a function of transduction with rAAV gene therapy products as described herein. In some embodiments, rAAV particles may crossover between the treated ear and the contralateral ear in neonatal and/or adult animals. In some embodiments, such a crossover may be due to the patent nature of the rodent cochlea (e.g., liquid communication between the perilymph, the CSF, and the contralateral ear's perilymph).

Methods of Characterizing

The term “mutation in an SLC26A4 gene” refers to a modification in a known consensus functional SLC26A4 gene that results in the production of a pendrin protein having one or more of: a deletion in one or more amino acids, one or more amino acid substitutions, and one or more amino acid insertions as compared to the consensus functional pendrin protein, and/or results in a decrease in the expressed level of the encoded pendrin protein in a mammalian cell as compared to the expressed level of the encoded pendrin protein in a mammalian cell not having a mutation. In some embodiments, a mutation can result in the production of a pendrin protein having a deletion in one or more amino acids (e.g., 2, 3, 4, 5, 6, 7, 8, 9, 10, 11, 12, 13, 14, 15 16, 17, 18, 19, 20, or more amino acids). In some embodiments, the mutation can result in a frameshift in the SLC26A4 gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding consensus functional protein) that may or may not be functional. In some embodiments, the mutation can result in the loss (or a decrease in the level) of expression of SLC26A4 mRNA or pendrin protein or both the mRNA and protein. In some embodiments, the mutation can result in the production of an altered pendrin protein having a loss or decrease in one or more biological activities (functions) as compared to a consensus functional pendrin protein.

In some embodiments, the mutation is an insertion of one or more nucleotides into an SLC26A4 gene. In some embodiments, the mutation is in a regulatory and/or control sequence of the pendrin gene, i.e., a portion of the gene that is not coding sequence. In some embodiments, a mutation in a regulatory and/or control sequence may be in a promoter or enhancer region and prevent or reduce the proper transcription of the SLC26A4 gene. In some embodiments, a mutation is in a known heterologous gene known to interact with a pendrin protein, or the SLC26A4 gene (e.g., FOXI1, or KCNJ10).

Methods of genotyping and/or detecting expression or activity of SLC26A4 mRNA and/or pendrin protein are known in the art (see e.g., Ito et al., World J Otorhinolaryngol. 2013 May 28; 3(2): 26-34, and Roesch et al., Int J Mol Sci. 2018 January; 19(1): 209, each of which is incorporated in its entirety herein by reference). In some embodiments, level of expression of SLC26A4 mRNA or pendrin protein may be detected directly (e.g., detecting pendrin protein, detecting SLC26A4 mRNA etc.). Non-limiting examples of techniques that can be used to detect expression and/or activity of SLC26A4 directly include, e.g., real-time PCR, quantitative real-time PCR, Western blotting, immunoprecipitation, immunohistochemistry, mass spectrometry, or immunofluorescence. In some embodiments, expression of SLC26A4 and/or pendrin protein can be detected indirectly (e.g., through functional hearing tests, ABRs, DPOAEs, etc.).

In some embodiments, tissue samples (e.g., comprising one or more inner ear cells, e.g., comprising one or more hair cells and/or one or more supporting cells) may be evaluated via morphological analysis to determine morphology of hair cells and/or support cells before and after administration of any agents (e.g., compositions, e.g., compositions comprising constructs, and/or particles, etc.) as described herein. In some such embodiments, standard immunohistochemical or histological analyses may be performed. In some embodiments, if cells are used in vitro or ex vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in vitro cell populations.

Methods of Treating a Subject

The present disclosure provides, among other things, that technologies described herein may be used to treat an underlying disease and/or symptoms in a subject suffering from or at risk of an otological disease characterized by mutation of SLC26A4 gene (e.g., DFNB4 and/or Pendred Syndrome).

In some embodiments, a method comprises administering a construct (e.g., an rAAV construct) described herein, a particle (e.g., an rAAV particle), or a composition described herein to a subject. In some embodiments, a method is a method of treatment. In some embodiments, a subject is a subject suffering from or at risk of an otological disease characterized by mutation of SLC26A4 gene (e.g., DFNB4 and/or Pendred Syndrome).

In some embodiments, administering a construct (e.g., an rAAV construct) described herein, a particle (e.g., an rAAV particle), or a composition described herein to a subject may alleviate and/or ameliorate one or more symptoms associated with an otological disease characterized by mutation of SLC26A4 gene (e.g., DFNB4 and/or Pendred Syndrome). Symptoms can include, for example, sensorineural hearing impairment, enlarged vestibular aqueduct, hypoplasia of the cochlea (e.g., too few turns of the cochlea), ataxia/loss of coordination, neurological speech impairment, and/or vertigo.

In some embodiments, a subject is genetically and/or symptomatically characterized as described herein, prior to, during, and/or after treatment with technologies described herein (e.g. via real-time PCR, quantitative real-time PCR, Western blotting, immunoprecipitation, immunohistochemistry, mass spectrometry, or immunofluorescence, indirect phenotypic determination of expression of a gene and/or protein (e.g., through functional hearing tests, ABRs, DPOAEs, etc.), etc.). In some embodiments, a subject suffering from or at risk of an otological disease characterized by mutation of SLC26A4 gene (e.g., DFNB4 and/or Pendred Syndrome) may have their associated disease state characterized through tissue sampling (e.g., comprising one or more inner ear cells, e.g., comprising one or more hair cells and/or one or more supporting cells). In some embodiments, tissues are evaluated via morphological analysis to determine morphology of hair cells and/or support cells before, during, and/or after administration of any technologies (e.g., methodologies, e.g., compositions, e.g., compositions comprising constructs, and/or particles, etc.) as described herein. In some such embodiments, standard immunohistochemical or histological analyses may be performed. In some embodiments, if cells are used in-vitro or ex-vivo, additional immunocytochemical or immunohistochemical analyses may be performed. In some embodiments, one or more assays of one or more proteins or transcripts (e.g., western blot, ELISA, polymerase chain reactions) may be performed on one or more samples from a subject or in-vitro cell populations.

In some embodiments, administering a construct (e.g., an rAAV construct) described herein, a particle (e.g., an rAAV particle), or a composition described herein to a subject improves a patients immunohistochemical evaluation (e.g., tests as described above) when compared to immunohistochemical tests performed prior to treatment with technologies described herein or when compared to a control population.

Production Methods

AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some embodiments, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under the control of an inducible promoter. In some embodiments, such component(s) may be under the control of a constitutive promoter. In some embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al, J. Virol., 70:520-532 (1993) and U.S. Pat. No. 5,478,745, which are incorporated in their entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, the AAV helper function construct supports efficient AAV construct production without generating any detectable wild type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some embodiments, viral construct titers post-purification are determined. In some embodiments, titers are determined using quantitative PCR. In certain embodiments, a TaqMan probe specific to a construct is utilized to determine construct levels. In certain embodiments, the TaqMan probe is represented by SEQ ID NO: 49, while forward and reverse amplifying primers are exemplified by SEQ ID NO: 54 and 55 respectively.

Exemplary TaqMan probe for quantification of
constructs
(SEQ ID NO: 49)
/56-FAM/TAATTCCAA/ZEN/CCAGCAGAGTCAGGGC/3IABkFQ/
Exemplary forward qPCR primer for quantification
of constructs
(SEQ ID NO: 54)
GATACAGCTAGAGTCCTGATTGC
Exemplary reverse qPCR primer for quantification
of constructs
(SEQ ID NO: 55)
GATCTGCCAAGTACCTCACTATG

As described herein, in some embodiments, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV11, and AAV Anc80, as well as variants thereof. In some embodiments, an AAV particle is an AAV2/6, AAV2/8, AAV2/9, or AAV2/Anc80 particle (e.g., with AAV6, AAV8, AAV9 or Anc80 capsid and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV particle may be used to deliver a coding sequence described herein. However, the serotypes have different tropisms, e.g., they preferentially infect different tissues. In some embodiments, an AAV construct is a self-complementary AAV construct.

The present disclosure provides, among other things, methods of making AAV-based constructs. In some embodiments, such methods include use of host cells. In some embodiments, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, to make and utilize technologies of the present disclosure.

### Example 1: Construction of Viral Constructs

This example provides a description of generating a viral construct as described herein. A recombinant AAV (rAAV) particle was generated by transfection with an adenovirus-free method as used by Xiao et al. J Virol. 73(5):3994-4003, 1999, which is incorporated in its entirety herein by reference. The cis plasmids with AAV ITRs, the trans plasmid with AAV Rep and Cap genes, and a helper plasmid with an essential region from an adenovirus genome were co-transfected in HEK293 cells. The rAAV construct expressed human pendrin under a single construct strategy using the constructs described. AAVAnc80 capsid was prepared to encapsulate a unique rAAV pendrin protein encoding construct.

Those of ordinary skill in the art will readily understand that similar constructs can be made in accordance with this example. For instance, rAAV constructs that express mammalian, primate, or human pendrin under single, dual, or multi construct strategies can be generated. AAV serotypes 1, 2, 3, 4, 5, 6, 7, 8, 9, rh8, rh10, rh39, rh43, and Anc80 can each be prepared to encapsulate four sets of pendrin constructs to test (i) a concatemerization-transplicing strategy, (ii) a hybrid intronic-homologous recombination-transplicing strategy, (iii) an exonic homologous recombination strategy, as summarized by Pryadkina et al., Meth. Clin. Devel. 2:15009, 2015, which is incorporated in its entirety herein by reference, and (iv) a single construct strategy.

### Example 2: Generating and Purifying Viral Particles

This example provides a description of purification of a viral particle (e.g., a particle created as described in Example 1). A recombinant AAV (rAAV) was produced using a standard triple transfection protocol and purified (e.g., by two sequential cesium chloride (CsCl) density gradients, as described by Pryadkina et al., Mol. Ther. 2:15009, 2015, which is incorporated in its entirety herein by reference). At the end of second centrifugation, 11 fractions of 500 μL were recovered from the CsCl density gradient tube and purified through dialysis in 1×PBS. The fractions were analyzed by dot blot to determine those containing rAAV genomes. The viral genome number (vg) of each preparation was determined by a quantitative real-time PCR-based titration method using primers and probe corresponding to the ITR region of the AAV construct genome (Bartoli et al. Gene. Ther. 13:20-28, 2006, which is incorporated in its entirety herein by reference). Those of ordinary skill in the art will readily understand that alternative production and/or purifying processes can be conducted in accordance with this example. For instance, rAAV particles may be purified using various column chromatography methods known in the art, and/or viral genomes may be quantified using alternative primer sets.

### Example 3: Formulation of Viral Particles

This example relates to the preparation of compositions comprising rAAV particles, and a physiologically acceptable solution. An rAAV was produced as described in Example 2 and purified to a titer of 4.45¹² vg/mL and was then prepared at dilutions of 6×10⁴, 1.3×10⁵, 1.8×10⁵, 4.5×10⁹, and 1.3×10¹⁰ vg/mL in physiologically acceptable solution (e.g., commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% pluronic Acid F68).

Those of ordinary skill in the art will readily understand that alternative formulations can be prepared in accordance with this example. For instance, rAAV particles may be purified to an alternative titer, prepared at alternative dilutions, and suspended in alternative suitable solutions. For example, an rAAV can be produced and purified to a quantified titer and prepared at appropriate dilutions in a physiologically acceptable solution (e.g., artificial perilymph comprising NaCl, 120 mM; KCl, 3.5 mM; CaCl₂), 1.5 mM; glucose, 5.5 mM; HEPES, 20 mM which is titrated with NaOH to adjust its pH to 7.5 (total Na⁺ concentration of 130 mM) as described in Chen et al., J Controlled Rel. 110:1-19, 2005, which is incorporated in its entirety herein by reference).

### Example 4: Device Description

This example relates to a device suitable for the delivery of rAAV particles (e.g., formulated as described in Example 3) to the inner ear. A composition comprising rAAV particles is delivered to the cochlea of a subject using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter may include at least one microneedle with a diameter from about 10 microns to about 1,000 microns, which produces perforations in the RWM that are sufficient to allow rAAV particles construct as described (e.g., comprising an rAAV construct of the present disclosure) to enter the cochlear perilymph of the scala tympani at a rate which does not damage the inner ear (e.g., a physiologically acceptable rate, e.g., a rate of approximately 30 μL/min to approximately 90 μL/min), but small enough to heal without surgical repair. The remaining portion of the microcatheter, proximal to the microneedle(s), is loaded with the rAAV/artificial perilymph formulation at a defined titer (e.g., approximately 1×10²¹ to 5×10¹³ vg/mL). The proximal end of the microcatheter is connected to a micromanipulator that allows for precise, low volume infusions of approximately 30 μL to approximately 100 μL.

### Example 5: In-Vitro Demonstration of SLC26A4 mRNA and Pendrin Protein Production (Anti-FLAG Antibody)

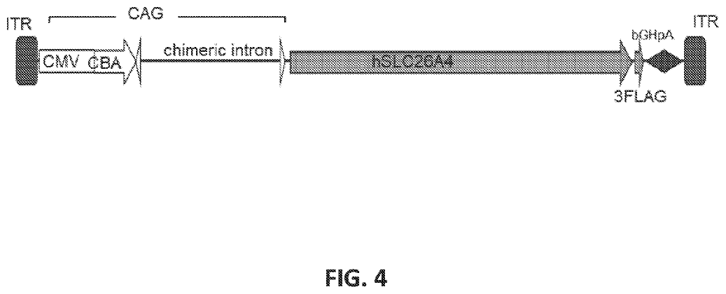

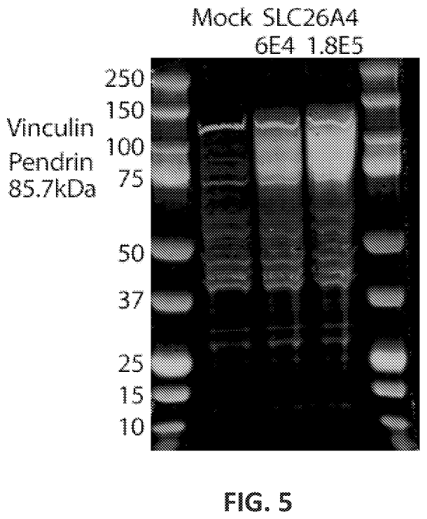

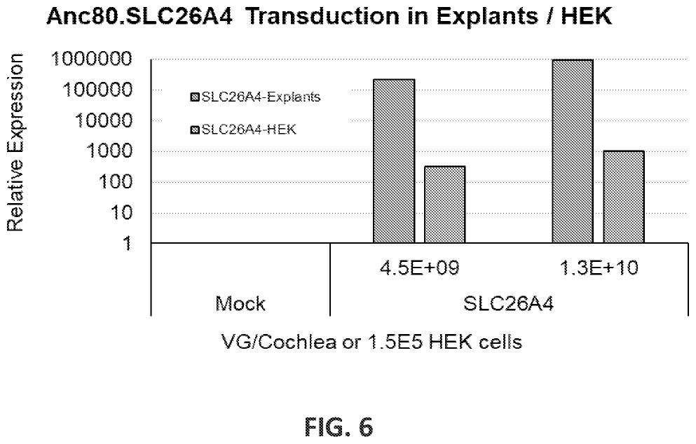

This example relates to the introduction, and expression analysis of rAAV particles (e.g., formulated as described in Example 3) expressing a construct comprising an SLC26A4 gene in mammalian cells grown in vitro or ex vivo. Mock rAAV particles or rAAV particles comprising rAAV constructs (as represented by FIG. 4) encapsidated by Anc80 capsids were prepared as described in Examples 1-3 above and transduced into HEK293FT cells seeded at a density of 1.5×10⁵ cells per well at a multiplicity of infection (MOI) of 6×10⁴ or 1.8×10⁵ vg/cell per well in a 24 well format. Cells were harvested 72 hours post transduction using 100 μL RIPA buffer (Thermo Scientific) per well or 350 μL RLT Plus RNA lysis buffer (Qiagen). For protein expression analysis, thirty microliters of samples were loaded into individual wells in a 4-12% Bis-Tris protein gel and standard western blotting procedures as known in the art were conducted. Banding patterns were determined using a fluorescent reader, with test anti-FLAG antibody and Vinculin as a control. The banding pattern of transgenic pendrin protein was determined (FIG. 5). For RNA expression analysis. RNA was extracted using RNeasy Mini Kit (Qiagen). Relative mRNA expression levels were determined using quantitative real-time PCR with hSLC26A4 specific amplifying primers and a TaqMan probe (SEQ ID NOs: 49, 54, and 55) relative to a human GAPDH TaqMan probe as control (Life Technologies). Robust and dose dependent SLC26A4 mRNA production was observed (FIG. 6).

Additionally, experiments were conducted to determine the mRNA expression level from rAAV constructs transduced into wild type explants (ex vivo). Mock rAAV particles or rAAV particles comprising rAAV constructs (as represented by FIG. 4) encapsidated by Anc80 capsids were prepared and transduced into explants at a MOI of 4.5×10⁹ vg/cochlea or 1.5×10¹⁰ vg/cochlea. Cells were harvested 72 hours post transduction using 350 μL RLT Plus RNA lysis buffer (Qiagen), and RNA samples were prepared using the RNeasy Micro Kit (Qiagen). Relative mRNA expression levels were determined using quantitative real-time PCR with human SLC26A4 specific amplifying primers and a TaqMan probe (SEQ ID NOs: 49, 54, and 55) relative to a mouse GAPDH probe as control (Life Technologies). Robust and dose dependent SLC26A4 mRNA production was observed (FIG. 6).

Those of ordinary skill in the art will readily understand that there are alternative methods of conducting the experiments associated with the current example, for instance, alternative viral titers, MOIs, cell concentrations, time to cellular harvest, reagents utilized for cellular harvesting or mRNA or protein analysis, AAV serotypes, and/or standard modifications to a construct comprising an SLC26A4 gene are practical and expected alterations of the current example.

### Example 6: Preliminary Hair Cell Tolerability Assessment of SLC26A4 Overexpression in Wild Type Neonate Cochlear Explants

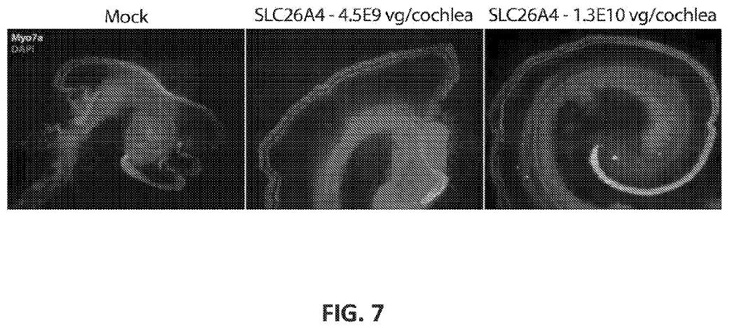

This example relates to the introduction, and expression analysis of rAAV particles comprising constructs for overexpressing an SLC26A4 gene (e.g., formulated as described in Example 3) in neonatal cochlear explants from wild type C1 mice at day P2-P3. Mock rAAV particles or rAAV particles comprising rAAV constructs (as represented by FIG. 4) encapsidated by Anc80 capsids were prepared as described in Examples 1-3 above and transduced into neonate cochlear explants at 4.5×10⁹ or 1.3×10¹⁰ vg/per cochlea. Explants were grown for 72 hours post transduction, and were then fixed using 4% PFA and prepared for immunofluorescence staining/imaging or RNA extraction. Overexpression was confirmed using quantitative PCR. Tolerability and lack of hair cell toxicity was determined using immunofluorescence staining/imaging, antibodies targeting Myo7a (Proteus Biosciences) were utilized to depict inner ear hair cells, while DAPI staining was used to define nuclear positioning. No hair cell (Myo7) toxicity was observed after SLC26A4 overexpression (FIG. 7).

### Example 7: Surgical Method in Aged Mice

### Example 7.1—Methods for rAAV Particle Introduction to Aged Mice

The current example relates to the introduction of constructs described herein to the inner ear of aged mice. rAAV particles comprising an AAV capsid and a construct encoding a pendrin protein or characteristic functional portion thereof are prepared in formulation buffer (e.g., artificial perilymph, or 1×PBS with pluronic acid F68) and then administered to the scala tympani in mice as described by Shu et al. (Human Gene Therapy, doi 10 1089/hum.2016 053, June 2016, which is incorporated in its entirety herein by reference). Male and female mice older than P15 are anesthetized using an intraperitoneal injection of xylazine (approximately 5-10 mg/kg) and ketamine (approximately 90-120 mg/kg). Body temperature is maintained at 37° C. using an electric heating pad. An incision is made from the right post-auricular region and the tympanic bulla and posterior semicircular canal are exposed. The bulla is perforated with a surgical needle and the small hole is expanded to provide access to the cochlea. The bone of the cochlear lateral wall of the scala tympani is thinned with a dental drill so that the membranous lateral wall is left intact. A small hole is then drilled in the posterior semicircular canal (PSCC). Patency of the canalostomy is confirmed by visualization of a slow leak of perilymph. A Nanoliter Microinjection System in conjunction with glass micropipette is used to deliver a total of approximately 1 μL of construct containing buffer (e.g., rAAV constructs described herein at approximately 4.5×10⁹ to 5×10¹⁰ vg/per cochlea in artificial perilymph or 1×PBS with pluronic acid F68) to the scala tympani at a rate of 2 nL/second. The glass micropipette is left in place for 5 minutes post-injection. Following cochleostomy and injection, the opening in the tympanic bulla and the PSCC are sealed with small pieces of fat, and the muscle and skin are sutured. The mice are allowed to awaken from anesthesia and their pain is controlled with 0.15 mg/kg buprenorphine hydrochloride for 3 days.

### Example 7.2—rAAV Particle Introduction to Aged Mice

Formulations comprising particles comprising constructs described herein (e.g., as described in Example 3) were administered to the inner ear of aged mice (e.g., aged Slc26a4^{L236P/L236P} mutant mice). rAAV particles comprising an AAV capsid and a construct encoding a pendrin protein or characteristic functional portion thereof were prepared in formulation buffer (e.g., artificial perilymph, or 1×PBS with pluronic acid F68) and then administered to the scala tympani in mice as described by Shu et al. (Human Gene Therapy, doi 10 1089/hum.2016 053, June 2016, which is incorporated in its entirety herein by reference). Male and female mice older than P15 (e.g., P23) were anesthetized using an intraperitoneal injection of xylazine (approximately 5-10 mg/kg) and ketamine (approximately 90-120 mg/kg). Body temperature was maintained at 37° C. using an electric heating pad. An incision was made from the right post-auricular region and the tympanic bulla and posterior semicircular canal were exposed. The bulla was perforated with a surgical needle and the small hole was expanded to provide access to the cochlea. The bone of the cochlear lateral wall of the scala tympani was thinned with a dental drill so that the membranous lateral wall was left intact. A small hole was then drilled in the posterior semicircular canal (PSCC). Patency of the canalostomy was confirmed by visualization of a slow leak of perilymph. A Nanoliter Microinjection System in conjunction with glass micropipette was used to deliver a total of approximately 1 μL of construct containing buffer (e.g., rAAV constructs described herein at approximately 4.5×10⁹ to 5×10¹⁰ vg/per cochlea in artificial perilymph or 1×PBS with pluronic acid F68) to the scala tympani at a rate of 2 nL/second. The glass micropipette was left in place for 5 minutes post-injection. Following cochleostomy and injection, the opening in the tympanic bulla and the PSCC were sealed with small pieces of fat, and the muscle and skin were sutured. The mice were allowed to awaken from anesthesia and their pain was controlled with 0.15 mg/kg buprenorphine hydrochloride for 3 days.

### Example 8: Transgenic Expression and Imaging of Pendrin Protein in SLC26A4^{tm1Dontuh/tm1Dontuh} Mice

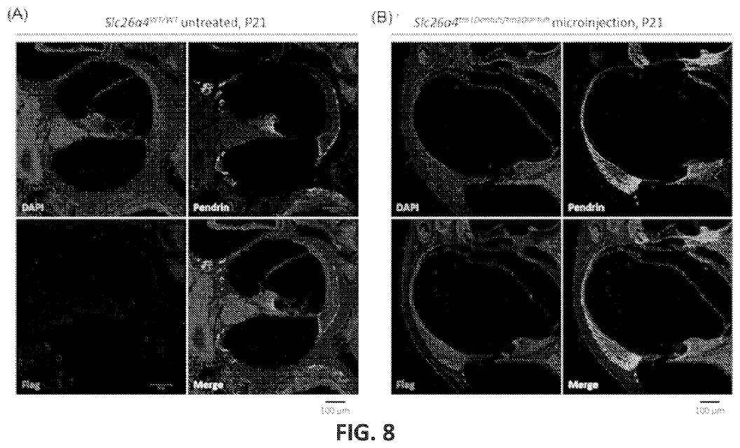

This example relates to the transgenic expression and analysis of transgenic pendrin protein in mice. Neonatal C57BL/6J wild type or Slc26a4^{tm1Dontuh/tm1Dontuh} mutant mice aged P3 were anesthetized by hyperthermia on ice to prepare for introduction of compositions described herein (see e.g., Example 3). Vehicle controls, mock rAAV particles, or rAAV constructs (as represented by FIG. 4) encapsidated by Anc80 capsids were prepared and introduced to the mouse inner ear through the round window membrane (RWM). Introduction of rAAV particles was performed through the following steps: A) preauricular incision to expose the cochlear bulla, B) glass micropipettes (cat #4878—WPI) pulled with a micropipette puller (cat #P87—Sutter instruments) to a final OD of about 10 m were used to manually deliver (micropipettes held by a Nanoliter 2000 micromanipulator—WPI) compositions containing rAAV particles into the scala tympanic, which allows access to inner ear cells, C) 1 μL of a composition described herein (e.g., rAAV constructs described herein at approximately 4.5×10⁹ to 5×10¹⁰ vg/per cochlea in 1×PBS with pluronic acid F68) was injected into each tested cochlea at a release rate of 0.3 μl/min (controlled by MICRO4 microinjection controller—WPI). Sham surgeries were performed as above with vehicle as a negative control. Mice were allowed to recover from surgery without additional intervention. At day P21 mice were harvested for immunofluorescence staining/imaging. Control or Slc26a4^{tm1Dontuh/tm1Dontuh} mutant mice cochlear sections were imaged using DAPI for nuclear expression, anti-Pendrin antibody (Santa Cruz Biotechnology), and anti-FLAG antibody (FIG. 8).

### Example 9: Phenotypic Analysis of Transgenic Expression of SLC26A4 mRNA and Pendrin Protein in SLC26A4^{tm1Dontuh/tm1Dontuh} Mice

The present example pertains to a phenotypic analysis of hearing in mice which were transgenically expressing SLC26A4 mRNA and pendrin protein in the inner ear. Neonatal C57BL/6J wild type or Slc26a4^{tm1Dontuh/tm1Dontuh} mutant mice (see e.g., as described in Example 8) aged P0 or P3 were anesthetized by hyperthermia on ice to prepare for introduction of compositions described herein. Mock rAAV particles or rAAV constructs (as represented by FIG. 4) encapsidated by Anc80 capsids were prepared and introduced to the mouse inner ear through the round window membrane (RWM). Introduction of rAAV particles was performed through the following steps: A) preauricular incision to expose the cochlear bulla, B) glass micropipettes (cat #4878—WPI) pulled with a micropipette puller (cat #P87—Sutter instruments) to a final OD of ˜10 m were used to manually deliver (micropipettes held by a Nanoliter 2000 micromanipulator—WPI) compositions containing rAAV particles into the scala tympanic, which allows access to inner ear cells, C) 1 μL of a composition described herein (e.g., rAAV constructs described herein at approximately 4.5×10⁹ to 5×10¹⁰ vg/per cochlea in 1×PBS with pluronic acid F68) was injected into each tested cochlea at a release rate of 0.3 l/min (controlled by MICRO4 microinjection controller—WPI). Sham surgeries were performed as above with vehicle as a negative control. Mice were allowed to recover from surgery without additional intervention

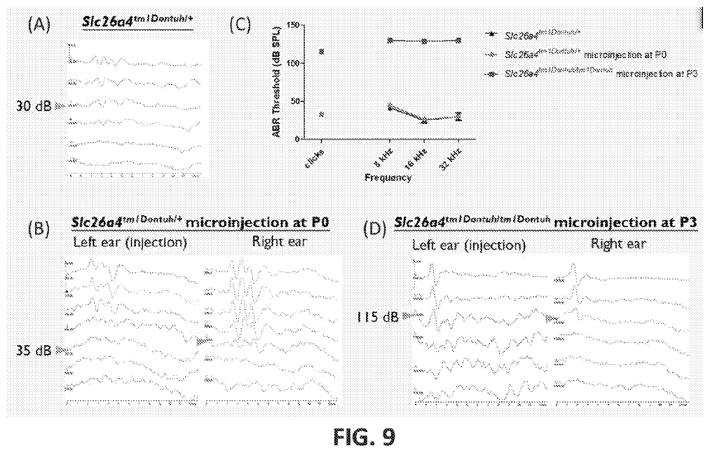

At day P21, mutant Slc26a4^{tm1Dontuh/tm1Dontuh} mice which had undergone unilateral composition injection were anesthetized with sodium pentobarbital (35 mg/kg) delivered intraperitoneally. Mice were then placed and maintained in a head-holder within a grounded and acoustically and electrically insulated test room. An evoked potential detection system (Smart EP 3.90, Intelligent Hearing Systems, Miami, Fla., USA) was used to measure the thresholds of the auditory brainstem response (ABR) in mice. Click sounds as well as 8, 16, and 32 kHz tone bursts at varying intensity (from 10 to 130 dB SPL) were used to evoke ABRs in test mice. The response signals were recorded with a subcutaneous needle electrode inserted ventrolaterally into the ears of the mice. This example confirms that the introduction of exemplary constructs as described herein (e.g., as depicted in FIG. 4) can rescue hearing loss, further analysis may assist in determining the exact administration timing window and how SLC26A4 rescue functions molecularly. Results are depicted throughout FIG. 9. FIG. 9 panel (A) depicts ABRs from control heterozygous Slc26a4^{tm1Dontuh/+} mice which retained the ability to respond to stimulation. FIG. 9 panel (B) depicts an exemplary recording result from day P21, C57BL/6J Slc26a4^{tm1Dontuh/tm1Dontuh} mice which were unilaterally injected with compositions as described herein at day P0, improvements in ABR performance were observed in test ears when compared to control ears. Improvements in response to stimuli were also observed in non-injected ears due to crossover from the injected ear to the un-injected ear in neonatal mice. FIG. 9 panel (C) depicts a graphical representation of the ABR threshold (at specific frequencies measured in dB SPL) required to generate a response in Slc26a4^{tm1Dontuh/+} mice compared to Slc26a4^{tm1Dontuh/tm1Dontuh} mice unilaterally injected on day P0 or day P3 respectively. FIG. 9 panel (D) depicts an exemplary recording result form day P21, C57BL/6J Slc26a4^{tm1Dontuh/tm1Dontuh} mice which were unilaterally injected with compositions as described herein at day P3, improvements in ABR performance were not observed and further analysis may be of relevance for determining administration timing windows.

### Example 10: Phenotypic Analysis of Transgenic Expression of SLC26A4 mRNA and Pendrin Protein in SLC26A4 Mutant Mice

### Example 10.1—Methods for Phenotypic Analysis of Transgenic Mice

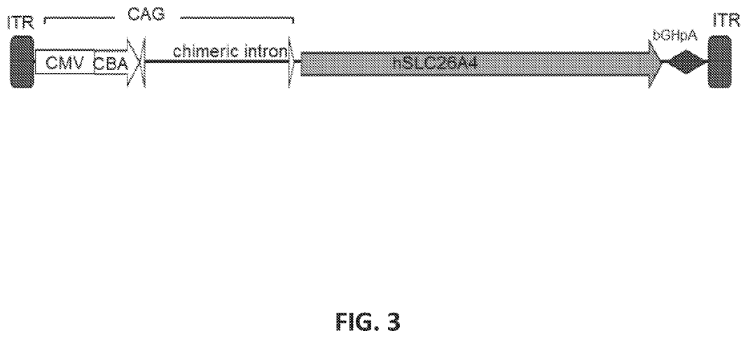

The present example pertains to a phenotypic analysis of hearing in mice which are transgenically expressing SLC26A4 mRNA and pendrin protein in the inner ear (see e.g., as described in Example 3). Neonatal FVB wild type or SLC26A4 mutant mice (e.g., mice mimicking the human L236P mutation, e.g., Slc26a4^{L236P/L236P+} mutant mice in CBA/CaJ strain as described in, e.g., Wen et al., Biochem and Biophys Research Communications, Vol 515, pg. 359-365, 2019, which is incorporated in its entirety herein by reference) aged P0, P1, P2, or P3 are anesthetized (e.g., by hyperthermia on ice) to prepare for introduction of compositions described herein. Vehicle control, mock rAAV particles, or rAAV constructs (e.g., as represented by FIG. 3 or FIG. 4) encapsidated by Anc80 capsids are prepared and introduced to the mouse inner ear through the round window membrane (RWM) (see e.g., Example 7). Introduction of rAAV particles is performed through the following steps: A) preauricular incision to expose the cochlear bulla, B) glass micropipettes (cat #4878—WPI) pulled with a micropipette puller (cat #P87—Sutter instruments) to a final OD of ˜10 m are used to manually deliver (micropipettes held by a Nanoliter 2000 micromanipulator—WPI) compositions containing rAAV particles into the scala tympanic, which allows access to inner ear cells, C) approximately 1 μL of a composition described herein (e.g., rAAV constructs described herein at approximately 4.5×10⁹ to 5×10¹⁰ vg/per cochlea in 1×PBS with pluronic acid F68) is injected into each tested cochlea at a release rate of approximately 0.3 μl/min (controlled by MICRO4 microinjection controller—WPI). Sham surgeries are performed as above with vehicle as a negative control. Mice are allowed recover from surgery without additional intervention

At defined dates post-surgery (e.g., day P21, P28, P30, P60, 2 months, P90, P120, P150, P180, 6 months, and/or 12 months), Slc26a4 mutant mice which have undergone unilateral composition injection are anesthetized with sodium pentobarbital (e.g., approximately 35 mg/kg) delivered intraperitoneally. Mice are then placed and maintained in a head-holder within a grounded and acoustically and electrically insulated test room. An evoked potential detection system (Smart EP 3.90, Intelligent Hearing Systems, Miami, Fla., USA) is used to measure the thresholds of the auditory brainstem response (ABR) in mice. Click sounds as well as 8, 16, and 32 kHz tone bursts at varying intensity (from 10 to 130 dB SPL) are used to evoke ABRs in test mice. The response signals are recorded with a subcutaneous needle electrode inserted ventrolaterally into the ears of the mice. Sham injected mice act as a negative control while the mock-injected ear may act as an internal control for ABR tests, improvements in ABR performance is observed in test ears when compared to control ears and/or animals.

Neonatal and/or Adult S1c26a4^{L236P/L236P} mutant mice in the FVB background undergo unilateral or bilateral surgery at day P1, day P2, day P23, or day P28 comprising delivery of rAAV particles as represented in FIG. 3 or FIG. 4 through the round window membrane injection with or without fenestration of the posterior semicircular canal (as described above). Prior to surgery, mice optionally undergo initial ABR readings as described above. At defined dates post-surgery (e.g., day P21, P28, P30, P60, 2 months, P90, P120, P150, P180, 6 months, and/or 12 months) mice are tested for phenotypic presentation, e.g., ABR readings are measured as described above and/or phenotypes acting as a proxy for coordination (e.g., gait, circling behavior, swimming, etc.) are measured. Results show that neonatal and/or adult gene therapy rescues: hearing loss phenotypes in treated and/or contralateral ears, and/or loss of coordination phenotypes.

### Example 10.2—Phenotypic Analysis of Transgenic Mice

Mutant Slc26a4 mice comprising the L236P mutation at the endogenous locus were generated using CRISPR/Cas integration in the FVB genetic background. Neonatal FVB S1c26a4^{L236P/L236P} mutant mice aged P2 were anesthetized by hyperthermia on ice to prepare for introduction of compositions described herein. Mutant mice underwent unilateral rAAV particles (as represented by FIG. 4) injection through the round window membrane (RWM) of a test ear (see e.g., Example 7). Introduction of rAAV particles was performed through the following steps: A) preauricular incision was created to expose the cochlear bulla, B) glass micropipettes (cat #4878—WPI) were pulled with a micropipette puller (cat #P87—Sutter instruments) to a final OD of ˜10 m and were used to manually deliver (micropipettes held by a Nanoliter 2000 micromanipulator—WPI) compositions containing rAAV particles into the scala tympanic, which allowed access to inner ear cells, C) approximately 1 μL of a composition described herein (e.g., rAAV particles described herein at approximately 8.2E12 vg/ml in 1×PBS with pluronic acid F68) was injected into each tested cochlea at a release rate of approximately 0.3 l/min (controlled by MICRO4 microinjection controller—WPI). Mice were allowed recover from surgery without additional intervention.

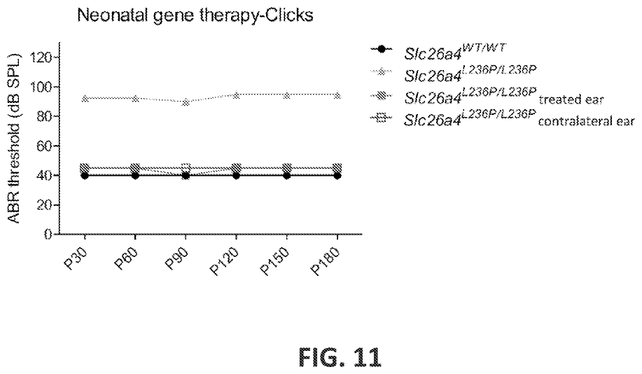

At defined dates following surgery at day P2 (e.g., at day P30, P60, P90, P120, P150, and P180), a control S1c26a4^{L236P/L236P} mutant mouse, a control Slc26a4^{WT/WT} mouse, and a S1c26a4^{L236P/L236P} mutant mouse which had undergone unilateral composition injection were anesthetized with sodium pentobarbital (e.g., approximately 35 mg/kg) delivered intraperitoneally. Mice were then placed and maintained in a head-holder within a grounded and acoustically and electrically insulated test room. An evoked potential detection system (Smart EP 3.90, Intelligent Hearing Systems, Miami, Fla., USA) was used to measure the thresholds of the auditory brainstem response (ABR) in said mice. Click sounds were used to evoke ABRs in test mice. The response signals were recorded with a subcutaneous needle electrode inserted ventrolaterally into the ears of the mice. Sham injected mutant mice and WT mice acted as controls for ABR tests, improvements in ABR performance was observed in test animals in both the treated and contralateral ears when compared to control animals (see FIG. 11). These results show that neonatal gene therapy rescued hearing loss phenotypes in both treated and contralateral ears.

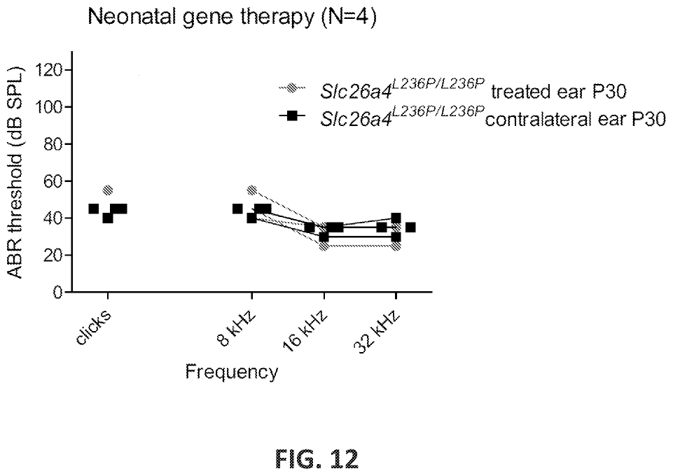

Another cohort of S1c26a4^{L236P/L236P} mutant mice (N=4) underwent unilateral surgery at day P2 as described above (e.g., RWM injection of 1 μL injectate comprising rAAV particles described herein at approximately 8.2E12 vg/ml in 1×PBS with pluronic acid F68). At day P30, mice were anesthetized with sodium pentobarbital (e.g., approximately 35 mg/kg) delivered intraperitoneally. Mice were then placed and maintained in a head-holder within a grounded and acoustically and electrically insulated test room. An evoked potential detection system (Smart EP 3.90, Intelligent Hearing Systems, Miami, Fla., USA) was used to measure the thresholds of the auditory brainstem response (ABR) in said mice. Click sounds as well as 8, 16, and 32 kHz tone bursts at varying intensity (from 10 to 130 dB SPL) are used to evoke ABRs in test mice. The response signals were recorded with a subcutaneous needle electrode inserted ventrolaterally into the ears of the mice. ABR performance indicating effective hearing was observed in test animals in both the treated and contralateral ears (see FIG. 12).

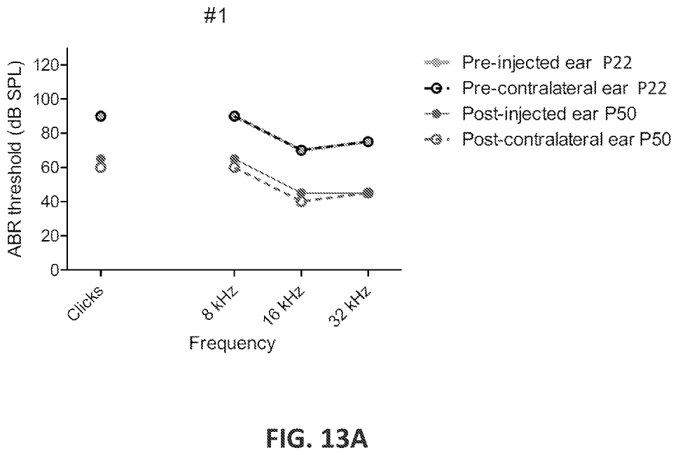

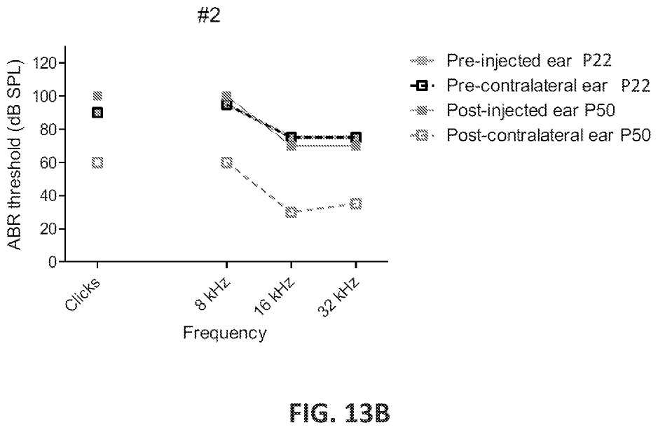

Two adult Slc26a4^{L236P/L236P} mutant mice underwent unilateral surgery at day P23 comprising delivery of rAAV particles as represented in FIG. 4 through the round window membrane with fenestration of the posterior semicircular canal (as described above; e.g., RWM injection of 1 μL injectate comprising rAAV particles described herein at approximately 8.2E12 vg/ml in 1×PBS with pluronic acid F68). Prior to surgery, at day P22, mice were anesthetized and ABR readings were measured as described above. Following surgery, at around day P49 or P50, mice were anesthetized and ABR readings were measured as described above. As shown in FIG. 13A and FIG. 13B, prior to rAAV particle injection, both mice displayed poor hearing when exposed to clicks or pure tones at noted frequencies. However, three out of four ears displayed improved ABR performance at post-injection test dates, indicating effective hearing and transgenic rescue of the S1c26a4^{L236P/L236P} hearing loss phenotype (observations of the mice indicated that post-injected ear P50 presented in FIG. 13B may have sustained damage as part of the injection process). In addition, the same mice were tested at day P50 using a swimming assay as a proxy for coordination. The swim test was conducted in accordance with the modified SHIRPA behavioral protocol for evaluation of vestibular abnormalities. Both WT and treated mice clearly presented swimming ability including directional movement of all 4 limbs, while mutant mice could stay above water (data not shown). Treated mice exhibited improved swimming capacity when compared to controls. These results showed that adult gene therapy rescued hearing loss phenotypes in both treated and contralateral ears, and restored coordination.

The S1c26a4^{L236P/L236P} mutant mice generated in-house displayed a range of phenotypic presentation (FIG. 14A), ranging from: severe congenital hearing loss (FIG. 14B), congenital hearing loss (FIG. 14C), degenerative hearing loss (FIG. 14D), and normal hearing levels (FIG. 14E) when measured using ABRs with click sounds as well as 8, 16, and 32 kHz tone bursts at varying intensity (from 10 to 130 dB SPL). All mutant animals had some level of phenotypic penetrance, but experienced phenotypic variability. This phenomena is not uncommon in mouse models for hearing loss and vestibular abnormalities and was previously observed in a similar mouse model described in Wen et al., Biochem and Biophys Research Communications, Vol 515, pg. 359-365, 2019. To control for this phenotypic variability mice were evaluated prior to surgery using ABR, and only mice with apparent hearing loss (>20 dB threshold increase compared to WT) were administered with the vector. The range of hearing phenotypes correlated with additional behavioral phenotypes that can act as a proxy for other DFNB4 and/or Pendred syndrome symptoms such as loss of coordination (e.g., represented by circling behaviors and/or inability to swim).

### Example 11: Human Clinical Example

This example relates to the chain of events leading to and resulting in treatment of SLC26A4 related syndromic or nonsyndromic hearing loss. A patient is diagnosed as having a pathogenic SLC26A4 gene. The patient is put under general anesthesia. The surgeon approaches the tympanic membrane from external auditory canal, makes a small incision at the inferior edge of the external auditory canal where it meets the tympani membrane, and lifts the tympanic membrane as a flap to expose the middle ear space. A surgical laser is used to make a small opening (approximately 2 mm) in the stapes footplate. The surgeon then penetrates the round window membrane with a microcatheter loaded with a solution of a mixture of at least one AAV-based constructs comprising SLC26A4 gene sequence, prepared in a physiologically suitable buffer (e.g., artificial perilymph) at an appropriate titer (e.g., approximately 1×10¹² to 5×10¹³ vg/mL). The microcatheter is connected to a micromanipulator that infuses a physiologically acceptable volume of mixture (e.g., approximately 50 μL, to approximately 100 L) at a steady but appreciable rate (e.g., approximately 30 μL/min to 90 μL/min). At the conclusion of the infusion, the surgeon withdraws the microcatheter and patches the holes in the stapes foot plate and RWM with a gel foam patch. The procedure concludes with replacement of the tympanic membrane flap before the patient is allowed to withdraw and recover from the anesthesia.

### Example 12: Non-Invasive Prenatal Testing of Maternal Blood to Detect SLC26A4 Mutation

This example relates to the testing of maternal blood to determine an offspring's SLC26A4 genotype prior to birth to facilitate swift and efficacious therapeutic intervention. Maternal blood samples (20-40 mL) are collected into Cell-free DNA (cfDNA) tubes. At least 7 mL of plasma is isolated from each sample via a double centrifugation protocol of 2,000 g for 20 minutes, followed by 3,220 g for 30 minutes, with supernatant transfer following the first spin. cfDNA is isolated from 7-20 mL plasma using a QIAGEN QIAmp Circulating Nuclei Acid kit and eluted in 45 μL TE buffer. Pure maternal genomic DNA is isolated from the buffy coat obtained following the first centrifugation.

By combining thermodynamic modeling of the assays to select probes with minimized likelihood of probe-probe interaction with amplification approaches described previously (Stiller et al. 2009 Genome Res 19(10):1843-1848, which is incorporated in its entirety herein by reference), multiplexing of 11,000 assays can be achieved. Maternal cfDNA and maternal genomic DNA samples are pre-amplified for 15 cycles using 11,000 target-specific assays and an aliquot is transferred to a second PCR reaction of 15 cycles using nested primers. Samples are prepared for sequencing by adding barcoded tags in a third 12-cycle round of PCR. The targets include SNPs corresponding to the greater than 200 mutations in SLC26A4 known to lead to Pendred syndrome or DFNB4, and/or sequences that cover all exons of SLC26A4, in order to detect any presently unknown but potentially pathogenic variant. Optionally, sequences corresponding to FOXI1 and/or KCNJ10 are amplified to identify possible heterologous digenic cases of DFNB4 or Pendred syndrome. The amplicons are then sequenced using an Illumina HiSeq sequencer. Genome sequence alignment is performed using commercially available software.

### EXEMPLARY EMBODIMENTS

Embodiment 1. A construct comprising a coding sequence operably linked to a promoter, wherein the coding sequence encodes a pendrin protein.

Embodiment 2. The construct of embodiment 1, wherein the coding sequence is an SLC26A4 gene.

Embodiment 3. The construct of embodiment 2, wherein the SLC26A4 gene is a primate SLC26A4 gene.

Embodiment 4. The construct of embodiment 2 or 3, wherein the SLC26A4 gene is a human SLC26A4 gene.

Embodiment 5. The construct of embodiment 4, wherein the human SLC26A4 gene comprises a nucleic acid sequence according to SEQ ID NO: 1, SEQ ID NO: 2, or SEQ ID NO: 3.

Embodiment 6. The construct of embodiment 4 or 5, wherein the human SLC26A4 gene comprises a nucleic acid sequence according to SEQ ID NO: 1.

Embodiment 7. The construct of embodiment 1, wherein the pendrin protein is a primate pendrin protein.

Embodiment 8. The construct of embodiment 1 or 7, wherein the pendrin protein is a human pendrin protein.

Embodiment 9. The construct of embodiment 8, wherein the pendrin protein comprises an amino acid sequence according to SEQ ID NO: 6.

Embodiment 10. The construct of any one of embodiments 1-9, wherein the promoter is an inducible promoter, a constitutive promoter, or a tissue-specific promoter.

Embodiment 11. The construct of any one of embodiments 1-10, wherein the promotor is an inner ear cell-specific promoter.

Embodiment 12. The construct of embodiment 11, wherein the inner ear cell-specific promoter is a GJB2 promoter, a GJB6 promoter, a SLC26A4 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, or a S100b promoter.

Embodiment 13. The construct of any one of embodiments 1-10, wherein the promoter is a CAG promoter, a CBA promoter, a CMV promoter, or a CB7 promoter.

Embodiment 14. The construct of embodiment 13, wherein the promoter comprises a nucleic acid sequence according to SEQ ID NO: 43.

Embodiment 15. The construct of any one of embodiments 1-14, further comprising two AAV inverted terminal repeats (ITRs), wherein the two AAV ITRs flank the coding sequence and promoter.

Embodiment 16. The construct of embodiment 15, wherein the two AAV ITRs are or are derived from AAV2 ITRs.

Embodiment 17. The construct of embodiment 15, wherein the two AAV ITRs comprise: (i) a 5′ ITR comprising a nucleic acid sequence according to SEQ ID NO: 10 and a 3′ ITR comprising a nucleic acid sequence according to SEQ ID NO: 11; or (ii) a 5′ ITR comprising a nucleic acid sequence according to SEQ ID NO: 12 and a 3′ ITR comprising a nucleic acid sequence according to SEQ ID NO: 13.

Embodiment 18. The construct of embodiment 1, wherein the construct comprises a nucleic acid sequence according to SEQ ID NO: 39.

Embodiment 19. The construct of embodiment 1, wherein the construct comprises a nucleic acid sequence according to SEQ ID NO: 40.

Embodiment 20. An AAV particle comprising the construct of any one of embodiments 1-19.

Embodiment 21. The AAV particle of embodiment 20, further comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

Embodiment 22. The AAV particle of embodiment 21, wherein the AAV capsid is an AAV Anc80 capsid.

Embodiment 23. A composition comprising the construct of any one of embodiments 1-19.

Embodiment 24. A composition comprising the AAV particle of any one of embodiments 20-22.

Embodiment 25. The composition of embodiment 23 or 24, wherein the composition is a pharmaceutical composition.

Embodiment 26. The composition of embodiment 25, further comprising a pharmaceutically acceptable carrier.

Embodiment 27. An ex vivo cell comprising a composition of any one of embodiments 23-26.

Embodiment 28. A method comprising, transfecting an ex vivo cell with: (i) a construct of any one of embodiments 15-19; and (ii) one or more helper plasmids collectively comprising an AAV Rep gene, AAV Cap gene, AAV VA gene, AAV E2a gene, and AAV E4 gene.

Embodiment 29. A method comprising introducing a composition of embodiment 25 or 26 into the inner ear of a subject.

Embodiment 30. A method of treatment comprising introducing a composition of embodiment 25 or 26 into the inner ear of a subject.

Embodiment 31. The method of embodiment 29 or 30, wherein the composition of embodiment 25 or 26 is introduced into the cochlea of the subject.

Embodiment 32. The method of any one of embodiments 29-31, wherein the composition of embodiment 25 or 26 is introduced via a round window membrane injection.

Embodiment 33. The method of any one of embodiments 29-32, further comprising measuring a hearing level of the subject.

Embodiment 34. The method of embodiment 33, wherein a hearing level is measured by performing an auditory brainstem response (ABR) test.

Embodiment 35. The method of embodiment 33 or 34, further comprising comparing the hearing level of the subject to a reference hearing level.

Embodiment 36. The method of embodiment 35, wherein the reference hearing level is a published or historical reference hearing level.

Embodiment 37. The method of embodiment 35, wherein the hearing level of the subject is measured after the composition of embodiment 25 or 26 is introduced, and the reference hearing level is a hearing level of the subject that was measured before the composition of embodiment 25 or 26 was introduced.

Embodiment 38. The method of any one of embodiments 29-37, further comprising measuring a level of pendrin protein in the subject.

Embodiment 39. The method of embodiment 38, wherein the level of pendrin protein is measured in the inner ear of the subject.

Embodiment 40. The method of embodiment 38 or 39, wherein the level of pendrin protein is measured in the cochlea of the subject.

Embodiment 41. The method of any one of embodiments 38-40, further comprising comparing the level of pendrin protein in the subject to a reference pendrin protein level.

Embodiment 42. The method of embodiment 41, wherein the reference pendrin protein level is a published or historical reference pendrin protein level.

Embodiment 43. The method of embodiment 41, wherein the level of pendrin protein in the subject is measured after the composition of embodiment 25 or 26 is introduced, and the reference pendrin protein level is a pendrin protein level of the subject that was measured before the composition of embodiment 25 or 26 was introduced.

Embodiment 44. Use of a construct of any one of embodiments 1-19, an AAV particle of any one of embodiments 20-22, or a composition of any one of embodiments 23-27 for the treatment of hearing loss in a subject suffering from or at risk of hearing loss.

Embodiment 45. Use of a construct of any one of embodiments 1-19, an AAV particle of any one of embodiments 20-22, or a composition of any one of embodiments 23-27 in the manufacture of a medicament for the treatment of hearing loss.

Embodiment 46. A construct of any one of embodiments 1-19, an AAV particle of any one of embodiments 20-22, or a composition of any one of embodiments 23-27 for use as a medicament.

Embodiment 47. A construct of any one of embodiments 1-19, an AAV particle of any one of embodiments 20-22, or a composition of any one of embodiments 23-27 for use in the treatment of hearing loss.

Embodiment 48. A genetically modified mouse whose genome comprises a modified Slc26a4 gene encoding polypeptide according to SEQ ID NO: 57, and wherein the genetically modified mouse is a genetically modified version of a mouse strain suitable for use in audiological analysis experiments.

Embodiment 49. The genetically modified mouse of embodiment 48, wherein the mouse strain suitable for use in audiological analysis experiments is not CBA/CaJ or CBA/J.

Embodiment 50. The genetically modified mouse of embodiment 48, wherein the mouse strain suitable for use in audiological analysis experiments is FVB, 129/Sv-+p+Tyr-c+Mgf-SIJ/J, A/HeJ, AKR/J, BALB/cByJ, BALB/cJ, BDP/J, BXSB/MpJ, C3H/HeJ, C3H/HeOuJ, C3HeB/FeJ, C57BL/10J, C57BL/10SnJ, C57BL/6ByJ, CASA/RK, CAST/Ei, CBA/J, CZECH II/Ei, DBA/2HaSmn, FVB/NJ, HRS/J hrl+, MOLD/Rk, MOLF/Ei, MOLG/Dn, NON/LtJ, NZB/B1NJ, NZO/NIJ, NZW/LacJ, PERA/camEi, PERC/Ei, PL/J, RBA/Dn, RBF/DnJ, RF/J, RHJ/Le hrrh-J/+, RIIIS/J, SEC/1ReJ, SENCARC/PtJ, SF/CamEi, SHR/GnEi, SJL/J, SM/J, SPRET/Ei, ST/bJ, or SWR/J strain.

Embodiment 51. A method comprising injection of a composition according to any one of embodiments 1-19, an AAV particle according to any one of embodiments 20-22, or a composition according to any one of embodiments 23-26 through a perforation in a round window membrane in a mouse according to any one of embodiments 48-50.

Embodiment 52. A method of treating hearing loss comprising introducing a composition according to any one of embodiments 1-19, an AAV particle according to any one of embodiments 20-22, or a composition according to any one of embodiments 23-26 into the inner ear of a subject.

Embodiment 53. The method of embodiment 52, wherein the composition is introduced via a round window membrane injection.

Embodiment 54. The method of embodiment 52 or 53, wherein the hearing loss is associated with a mutation in a SLC26A4 gene.

Embodiment 55. The method of any one of embodiments 52-54, wherein the hearing loss and treatment of hearing loss are characterized as a function of ABR and/or Distortion Product Otoacoustic Emissions (DPOAE) measurements recorded prior to receiving any treatment and compared to ABR and/or DPOAE measurements after treatment.

Embodiment 56. A kit comprising a composition that comprises a construct of any one of embodiments 1-19, a composition that comprises an AAV particle of any one of embodiments 20-22, or a composition of any one of embodiments 23-27.

Embodiment 57. The kit of embodiment 56, wherein the composition is pre-loaded in a device.

Embodiment 58. The kit of embodiment 57, wherein the device is a microcatheter.

Embodiment 59. The kit of embodiment 58, wherein the microcatheter is shaped such that it can enter the middle ear cavity via the external auditory canal and contact the end of the microcatheter with the RWM.

Embodiment 60. The kit of embodiments 57 or 58, wherein a distal end of the microcatheter is comprised of at least one microneedle with diameter of between 10 and 1,000 microns.

Embodiment 61. The kit of embodiment 56, further comprising a device.

Embodiment 62. The kit of embodiment 61, wherein the device is a device described in FIGS. 15-18 or a device as described herein.

Embodiment 63. The kit of embodiment 62, wherein the device comprises a needle comprising a bent portion and an angled tip.

### EQUIVALENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims. All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A construct comprising a coding sequence operably linked to a promoter, wherein the coding sequence encodes a pendrin protein.

2. The construct of claim 1, wherein the coding sequence is an SLC26A4 gene.

3. The construct of claim 2, wherein the SLC26A4 gene is a primate SLC26A4 gene.

4. The construct of claim 2 or 3, wherein the SLC26A4 gene is a human SLC26A4 gene.

5. The construct of claim 4, wherein the human SLC26A4 gene comprises a nucleic acid sequence according to SEQ ID NO: 1, SEQ ID NO: 2, or SEQ ID NO: 3.

6. The construct of claim 4 or 5, wherein the human SLC26A4 gene comprises a nucleic acid sequence according to SEQ ID NO: 1.

7. The construct of claim 1, wherein the pendrin protein is a primate pendrin protein.

8. The construct of claim 1 or 7, wherein the pendrin protein is a human pendrin protein.

9. The construct of claim 8, wherein the pendrin protein comprises an amino acid sequence according to SEQ ID NO: 6.

10. The construct of any one of claims 1-9, wherein the promoter is an inducible promoter, a constitutive promoter, or a tissue-specific promoter.

11. The construct of any one of claims 1-10, wherein the promotor is an inner ear cell-specific promoter.

12. The construct of claim 11, wherein the inner ear cell-specific promoter is a GJB2 promoter, a GJB6 promoter, a SLC26A4 promoter, a TECTA promoter, a DFNA5 promoter, a COCH promoter, a NDP promoter, a SYN1 promoter, a GFAP promoter, a PLP promoter, a TAK1 promoter, a SOX21 promoter, a SOX2 promoter, a FGFR3 promoter, a PROX1 promoter, a GLAST1 promoter, a LGR5 promoter, a HES1 promoter, a HES5 promoter, a NOTCH1 promoter, a JAG1 promoter, a CDKN1A promoter, a CDKN1B promoter, a SOX10 promoter, a P75 promoter, a CD44 promoter, a HEY2 promoter, a LFNG promoter, or a S100b promoter.

13. The construct of any one of claims 1-10, wherein the promoter is a CAG promoter, a CBA promoter, a CMV promoter, or a CB7 promoter.

14. The construct of claim 13, wherein the promoter comprises a nucleic acid sequence according to SEQ ID NO: 43.

15. The construct of any one of claims 1-14, further comprising two AAV inverted terminal repeats (ITRs), wherein the two AAV ITRs flank the coding sequence and promoter.

16. The construct of claim 15, wherein the two AAV ITRs are or are derived from AAV2 ITRs.

17. The construct of claim 15, wherein the two AAV ITRs comprise:
(i) a 5′ ITR comprising a nucleic acid sequence according to SEQ ID NO: 10 and a 3′ ITR comprising a nucleic acid sequence according to SEQ ID NO: 11; or
(ii) a 5′ ITR comprising a nucleic acid sequence according to SEQ ID NO: 12 and a 3′ ITR comprising a nucleic acid sequence according to SEQ ID NO: 13.

18. The construct of claim 1, wherein the construct comprises a nucleic acid sequence according to SEQ ID NO: 39.

19. The construct of claim 1, wherein the construct comprises a nucleic acid sequence according to SEQ ID NO: 40.

20. An AAV particle comprising the construct of any one of claims 1-19.

21. The AAV particle of claim 20, further comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

22. The AAV particle of claim 21, wherein the AAV capsid is an AAV Anc80 capsid.

23. A composition comprising the construct of any one of claims 1-19.

24. A composition comprising the AAV particle of any one of claims 20-22.

25. The composition of claim 23 or 24, wherein the composition is a pharmaceutical composition.

26. The composition of claim 25, further comprising a pharmaceutically acceptable carrier.

27. An ex vivo cell comprising a composition of any one of claims 23-26.

28. A method comprising, transfecting an ex vivo cell with:
(i) a construct of any one of claims 15-19; and
(ii) one or more helper plasmids collectively comprising an AAV Rep gene, AAV Cap gene, AAV VA gene, AAV E2a gene, and AAV E4 gene.

29. A method comprising:
introducing a composition of claim 25 or 26 into the inner ear of a subject.

30. A method of treatment comprising:
introducing a composition of claim 25 or 26 into the inner ear of a subject.

31. The method of claim 29 or 30, wherein the composition of claim 25 or 26 is introduced into the cochlea of the subject.

32. The method of claim 29-31, wherein the composition of claim 25 or 26 is introduced via a round window membrane injection.

33. The method of any one of claims 29-32, further comprising measuring a hearing level of the subject.

34. The method of claim 33, wherein a hearing level is measured by performing an auditory brainstem response (ABR) test.

35. The method of claim 33 or 34, further comprising comparing the hearing level of the subject to a reference hearing level.

36. The method of claim 35, wherein the reference hearing level is a published or historical reference hearing level.

37. The method of claim 35, wherein the hearing level of the subject is measured after the composition of claim 25 or 26 is introduced, and the reference hearing level is a hearing level of the subject that was measured before the composition of claim 25 or 26 was introduced.

38. The method of any one of claims 29-37, further comprising measuring a level of pendrin protein in the subject.

39. The method of claim 38, wherein the level of pendrin protein is measured in the inner ear of the subject.

40. The method of claim 38 or 39, wherein the level of pendrin protein is measured in the cochlea of the subject.

41. The method of any one of claims 38-40, further comprising comparing the level of pendrin protein in the subject to a reference pendrin protein level.

42. The method of claim 41, wherein the reference pendrin protein level is a published or historical pendrin protein level.

43. The method of claim 41, wherein the level of pendrin protein in the subject is measured after the composition of claim 25 or 26 is introduced, and the reference pendrin protein level is a pendrin protein level of the subject that was measured before the composition of claim 25 or 26 was introduced.

44. Use of a construct of any one of claims 1-19, an AAV particle of any one of claims 20-22, or a composition of any one of claims 23-27 for the treatment of hearing loss in a subject suffering from or at risk of hearing loss.

45. Use of a construct of any one of claims 1-19, an AAV particle of any one of claims 20-22, or a composition of any one of claims 23-27 in the manufacture of a medicament for the treatment of hearing loss.

46. A construct of any one of claims 1-19, an AAV particle of any one of claims 20-22, or a composition of any one of claims 23-27 for use as a medicament.

47. A construct of any one of claims 1-19, an AAV particle of any one of claims 20-22, or a composition of any one of claims 23-27 for use in the treatment of hearing loss.

48. A genetically modified mouse whose genome comprises a modified Slc26a4 gene encoding polypeptide according to SEQ ID NO: 57, and wherein the genetically modified mouse is a genetically modified version of a mouse strain suitable for use in audiological analysis experiments.

49. The genetically modified mouse of claim 48, wherein the mouse strain suitable for use in audiological analysis experiments is not CBA/CaJ or CBA/J.

50. The genetically modified mouse of claim 48, wherein the mouse strain suitable for use in audiological analysis experiments is FVB, 129/Sv-+p+Tyr-c+Mgf-SIJ/J, A/HeJ, AKR/J, BALB/cByJ, BALB/cJ, BDP/J, BXSB/MpJ, C3H/HeJ, C3H/HeOuJ, C3HeB/FeJ, C57BL/10J, C57BL/10SnJ, C57BL/6ByJ, CASA/RK, CAST/Ei, CBA/J, CZECH II/Ei, DBA/2HaSmn, FVB/NJ, HRS/J hrl+, MOLD/Rk, MOLF/Ei, MOLG/Dn, NON/LtJ, NZB/B1NJ, NZO/NIJ, NZW/LacJ, PERA/camEi, PERC/Ei, PL/J, RBA/Dn, RBF/DnJ, RF/J, RHJ/Le hrrh-J/+, RIIIS/J, SEC/1ReJ, SENCARC/PtJ, SF/CamEi, SHR/GnEi, SJL/J, SM/J, SPRET/Ei, ST/bJ, or SWR/J strain.

51. A method comprising,
injection of a composition according to any one of claims 1-19, an AAV particle according to any one of claims 20-22, or a composition according to any one of claims 23-26 through a perforation in a round window membrane in a mouse according to any one of claims 48-50.

52. A method of treating hearing loss comprising,
introducing a composition according to any one of claims 1-19, an AAV particle according to any one of claims 20-22, or a composition according to any one of claims 23-26 into the inner ear of a subject.

53. The method of claim 52, wherein the composition is introduced via a round window membrane injection.

54. The method of claim 52 or 53, wherein the hearing loss is associated with a mutation in a SLC26A4 gene.

55. The method of any one of claims 52-54, wherein the hearing loss and treatment of hearing loss are characterized as a function of ABR and/or Distortion Product Otoacoustic Emissions (DPOAE) measurements recorded prior to receiving any treatment and compared to ABR and/or DPOAE measurements after treatment.

56. A kit comprising a composition that comprises a construct of any one of claims 1-19, a composition that comprises an AAV particle of any one of claims 20-22, or a composition of any one of claims 23-27.

57. The kit of claim 56, wherein the composition is pre-loaded in a device.

58. The kit of claim 57, wherein the device is a microcatheter.

59. The kit of claim 58, wherein the microcatheter is shaped such that it can enter the middle ear cavity via the external auditory canal and contact the end of the microcatheter with the RWM.

60. The kit of claim 57 or 58, wherein a distal end of the microcatheter is comprised of at least one microneedle with diameter of between 10 and 1,000 microns.

61. The kit of claim 56, further comprising a device.

62. The kit of claim 61, wherein the device is a device described in FIGS. 15-18 or a device as described herein.

63. The kit of claim 62, wherein the device comprises a needle comprising a bent portion and an angled tip.
